(19) United States
(12) Patent Application Publication
(10) Pub. No.: US 2023/0225192 A1
(43) Pub. Date: Jul. 13, 2023

(54) LIGHT EMITTING ELEMENT AND POLYCYCLIC COMPOUND FOR THE SAME

(71) Applicant: Samsung Display Co., Ltd., Yongin-si (KR)

(72) Inventors: YOUNGJIN PARK, Suwon-si (KR); HYEONGMIN KIM, Suwon-si (KR); HEECHOON AHN, Seoul (KR); HYOYOUNG LEE, Suwon-si (KR); JIYONG CHOI, Cheonan-si (KR)

(21) Appl. No.: 18/050,000

(22) Filed: Oct. 26, 2022

(30) Foreign Application Priority Data
Jan. 7, 2022 (KR) 10-2022-0003015

(51) Int. Cl. H01L 51/00; C09K 11/06; C07F 7/08; C07D 487/04

(52) U.S. Cl. CPC H01L 51/0094; C09K 11/06; C07F 7/0812; C07D 487/04; H01L 51/0067; H01L 51/0072; H01L 51/5012

(57) ABSTRACT

A light emitting element that includes a first electrode, a second electrode on the first electrode, and at least one functional layer between the first electrode and the second electrode is provided. The at least one functional layer includes a polycyclic compound represented by a specific chemical structure, and has a high efficiency and low driving voltage properties.

### CROSS-REFERENCE TO RELATED APPLICATIONS

This application claims priority to and the benefit of Korean Patent Application No. 10-2022-0003015, filed on Jan. 7, 2022, in the Korean Intellectual Property Office, the entire content of which is hereby incorporated by reference.

### BACKGROUND

### 1. Field

Aspects of one or more embodiments of the present disclosure herein relate to a light emitting element and a polycyclic compound used therein, and for example, to a light emitting element including a polycyclic compound used as a light emitting material.

### 2. Description of Related Art

Recently, the development of an organic electroluminescence display device as an image display device is being actively conducted. The organic electroluminescence display device is a display device of a self-luminescent type in which holes and electrons injected from a first electrode and a second electrode recombine in an emission layer so that a light emitting material in the emission layer emits light to achieve display.

In the application of a light emitting element device to a display device, the decrease of a driving voltage and the increase of emission efficiency and lifetime of the light emitting element are required, and development on materials for a light emitting element, stably achieving the requirements is being consistently required (sought).

### SUMMARY

An aspect of one or more embodiments of the present disclosure is directed toward a light emitting element having improved driving voltage properties and emission efficiency.

An aspect of one or more embodiments of the present disclosure also is directed toward a polycyclic compound which may improve the driving voltage properties and emission efficiency of a light emitting element.

Additional aspects will be set forth in part in the description which follows and, in part, will be apparent from the description, or may be learned by practice of the presented embodiments of the disclosure.

An embodiment of the present disclosure provides a light emitting element including: a first electrode; a second electrode on the first electrode; and at least one functional layer between the first electrode and the second electrode, and including a polycyclic compound represented by Formula 1.

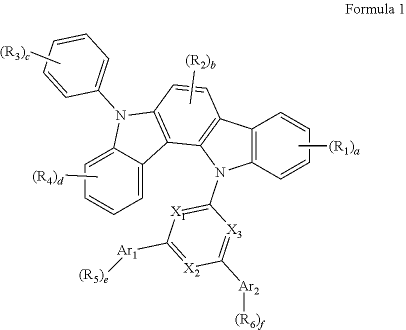

In Formula 1, X₁ to X₃ may each independently be C or N, where at least two or more selected from among X₁ to X₃ are N, Ar₁ and Ar₂ may each independently be a substituted or unsubstituted arylene group of 6 to 60 ring-forming carbon atoms, or a substituted or unsubstituted heteroarylene group of 2 to 60 ring-forming carbon atoms, R₁ to R₆ may each independently be a hydrogen atom, a deuterium atom, or a cyano group, or represented by Formula 2, in which at least one selected from among R₁ to R₆ is represented by Formula 2, “a” and “d” may each independently be an integer from 0 to 4, “b” is an integer from 0 to 2, and “c” to “f” are integers from 0 to 5.

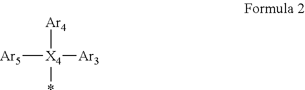

In Formula 2, X₄ is C, Si or Ge, Ar₃ to Ar₅ may each independently be a substituted or unsubstituted aryl group of 6 to 60 ring-forming carbon atoms, a substituted or unsubstituted cycloalkyl group of 3 to 60 ring-forming carbon atoms, or a substituted or unsubstituted heterocyclic group of 2 to 60 ring-forming carbon atoms, or combined with an adjacent group to form a ring, and * represents a connection position.

In the light emitting element of an embodiment, the at least one functional layer may include an emission layer, a hole transport region between the first electrode and the emission layer, and an electron transport region between the emission layer and the second electrode, and at least one selected from among the emission layer and the electron transport region may include the polycyclic compound.

In an embodiment, the emission layer may emit delayed fluorescence or phosphorescence.

In an embodiment, the emission layer may include a host and a dopant, and the host may include the polycyclic compound.

In an embodiment, the electron transport region may include an electron transport layer on the emission layer, and an electron injection layer on the electron transport layer, and the electron transport layer may include the polycyclic compound.

In an embodiment, the emission layer may emit light having a central wavelength of about 430 nm to about 480 nm.

In the light emitting element of an embodiment, the polycyclic compound represented by Formula 1 may be represented by any one selected from among Formula 3-1 to Formula 3-4.

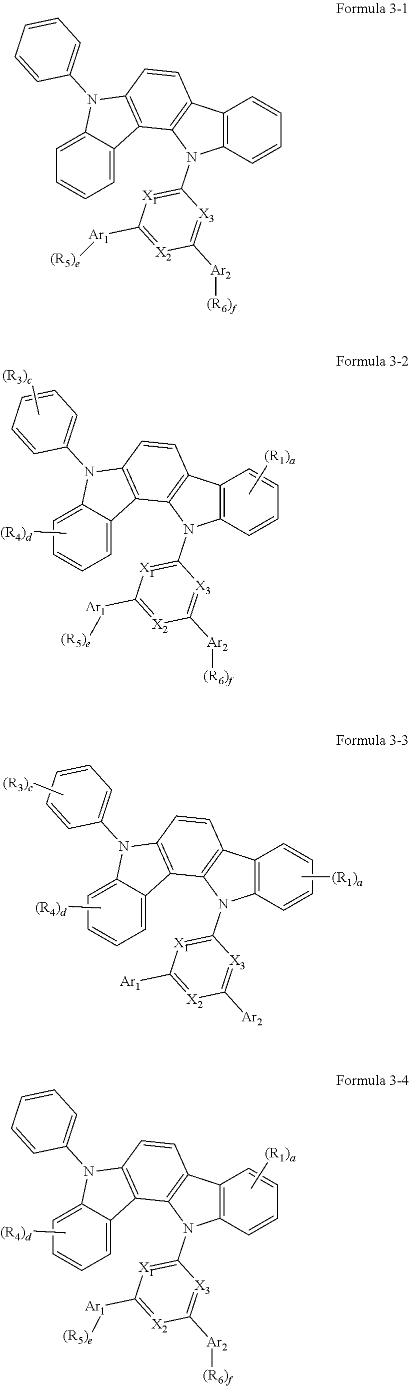

In Formula 3-1 to Formula 3-4, R₁ to R₆, Ar₁, Ar₂, and X₁ to X₃ are the same as defined in Formula 1. In Formula 3-1, at least one selected from among R₅ and R₆ is represented by Formula 2, in Formula 3-2, at least one selected from among R₁ and R₃, and at least one selected from among R₅ and R₆ are represented by Formula 2, in Formula 3-3, at least one selected from among R₁, R₃ and R₄ is represented by Formula 2, and in Formula 3-4, at least one selected from among R₁, R₄, R₅ and R₆ is represented by Formula 2.

In an embodiment, Formula 2 may be represented by any one selected from among 2-1 to 2-6.

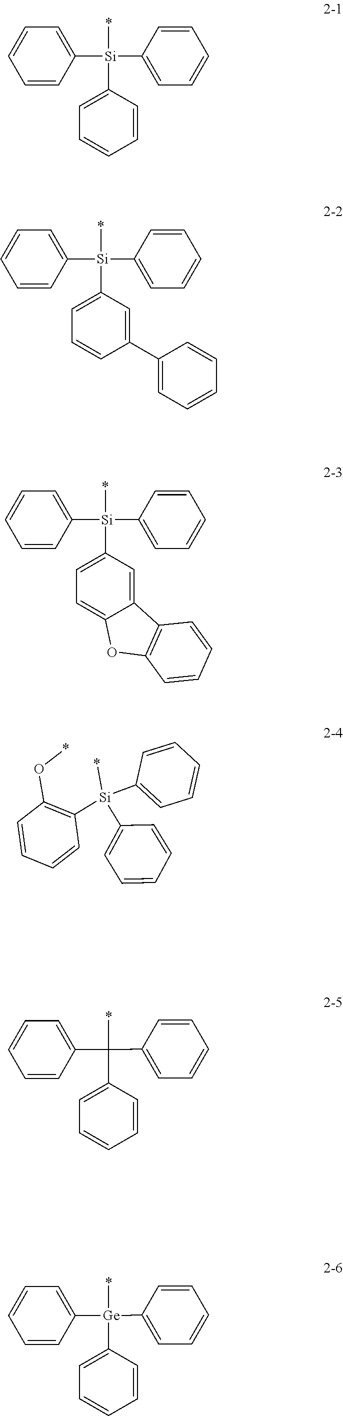

In an embodiment, the polycyclic compound represented by Formula 1 may be represented by any one selected from among 4-1 to 4-19.

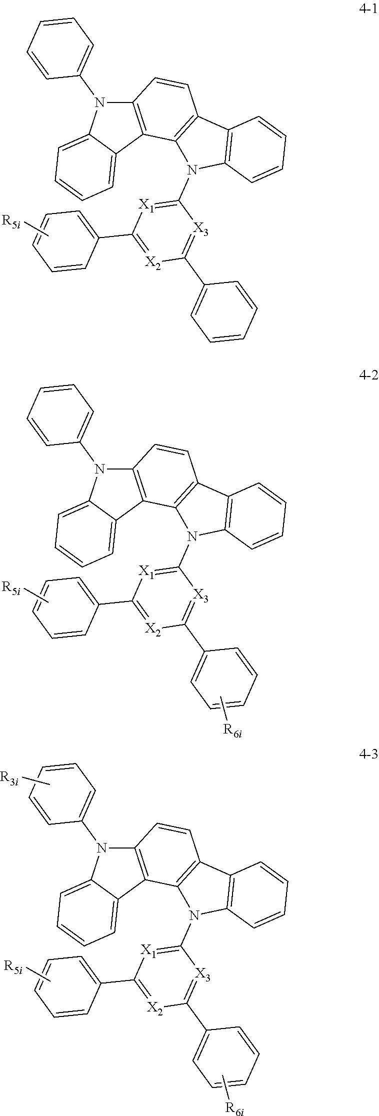

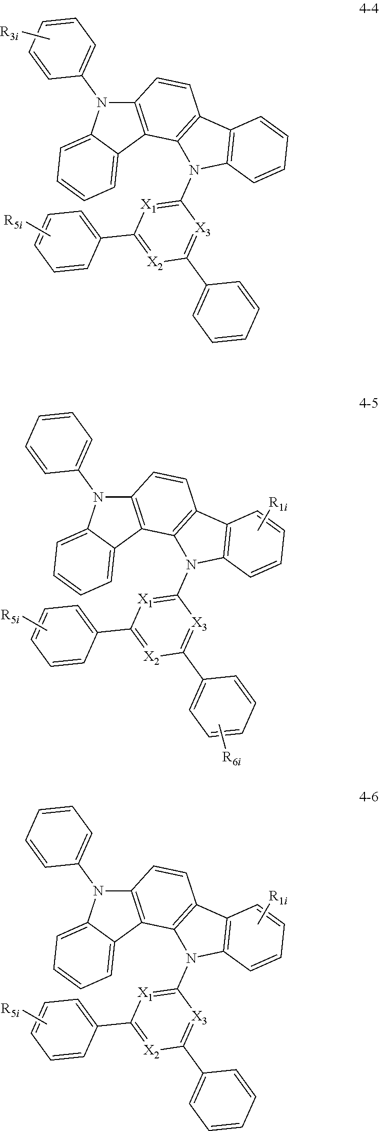

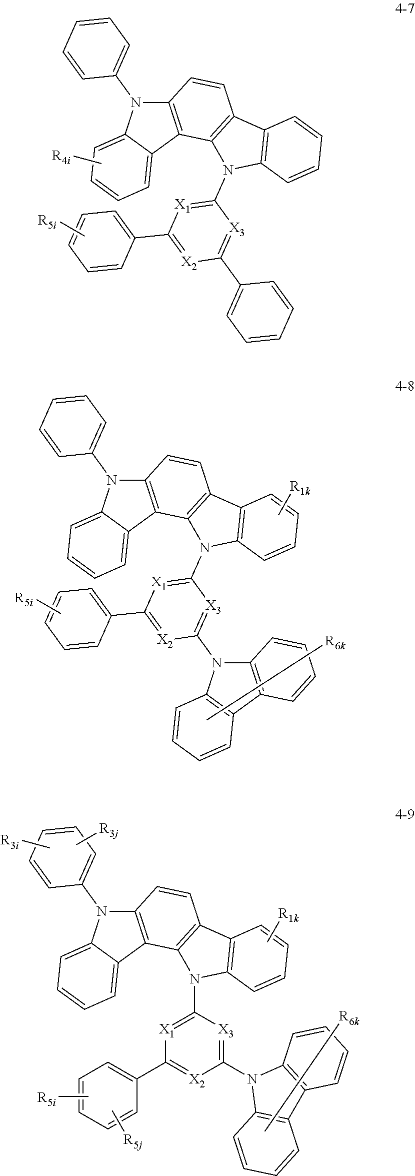

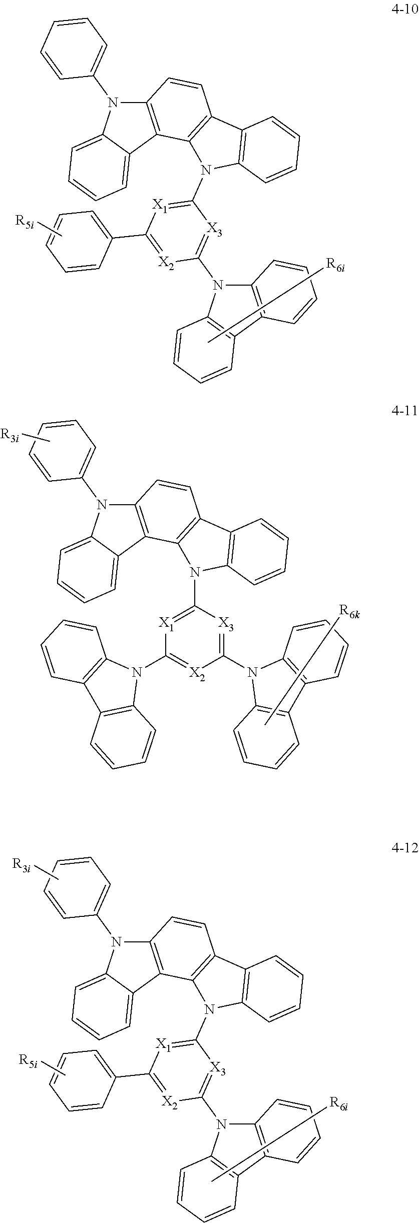

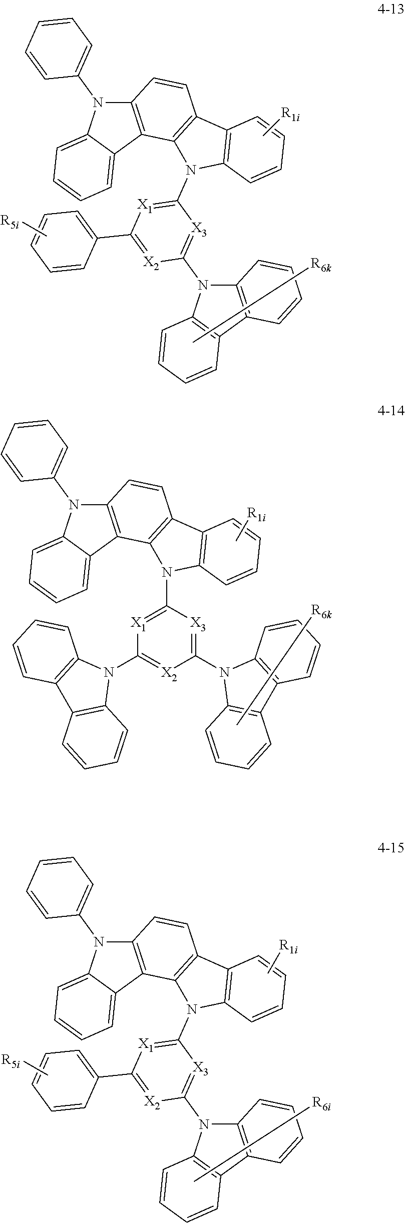

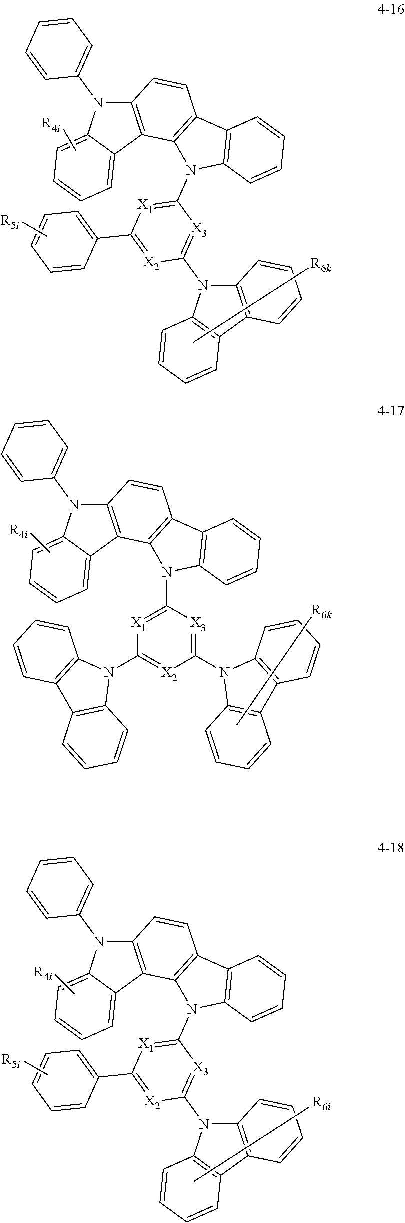

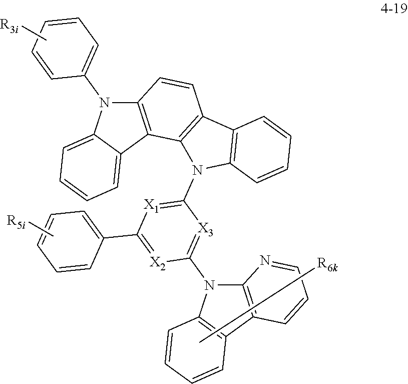

In the polycyclic compounds represented by 4-1 to 4-19, R₁ᵢ, R₃ᵢ, R₄ᵢ, R₅ᵢ and R₆ᵢ may each independently be represented by Formula 2, R₁ⱼ and R₅ⱼ may each independently be a hydrogen atom or represented by Formula 2, R₁ₖ and R₆ₖ may each independently be a hydrogen atom or a cyano group, and X₁ to X₃ are the same as defined in Formula 1.

### BRIEF DESCRIPTION OF THE DRAWINGS

The accompanying drawings are included to provide a further understanding of the present disclosure and are incorporated in and constitute a part of this specification. The drawings illustrate embodiments of the present disclosure and, together with the description, serve to explain principles of the present disclosure. In the drawings:

FIG. 1 is a plan view showing a display apparatus according to an embodiment;

FIG. 2 is a cross-sectional view showing a part corresponding to line I-I′ in FIG. 1;

FIG. 3 is a cross-sectional view schematically showing a light emitting element of an embodiment;

FIG. 4 is a cross-sectional view schematically showing a light emitting element of an embodiment;

FIG. 5 is a cross-sectional view schematically showing a light emitting element of an embodiment;

FIG. 6 is a cross-sectional view schematically showing a light emitting element of an embodiment;

FIG. 7 is a cross-sectional view showing a display apparatus of to an embodiment;

FIG. 8 is a cross-sectional view showing a display apparatus of an embodiment;

FIG. 9 is a cross-sectional view showing a display apparatus of an embodiment; and

FIG. 10 is a cross-sectional view showing a display apparatus of an embodiment.

### DETAILED DESCRIPTION

The present disclosure may have one or more suitable modifications and may be embodied in different forms, and example embodiments will be explained in more detail with reference to the accompanying drawings. The present disclosure may, however, be embodied in different forms and should not be construed as limited to the embodiments set forth herein. Rather, all modifications, equivalents, and substituents which are included in the spirit and technical scope of the present disclosure should be included in the present disclosure.

In the disclosure, it will be understood that when an element (or region, layer, part, etc.) is referred to as being “on”, “connected to” or “coupled to” another element, it can be directly on, connected or coupled to the other element or a third intervening elements may be present.

Like reference numerals refer to like elements throughout. In addition, in the drawings, the thickness, the ratio, and the dimensions of constituent elements may be exaggerated for effective explanation of technical contents. The term “and/or” includes one or more combinations which may be defined by relevant elements.

It will be understood that, although the terms first, second, etc. may be used herein to describe various elements, these elements should not be limited by these terms. These terms are only used to distinguish one element from another element. Thus, a first element could be termed a second element without departing from the teachings of the present invention. Similarly, a second element could be termed a first element. As used herein, the singular forms are intended to include the plural forms as well, unless the context clearly indicates otherwise.

In addition, the terms “below”, “beneath”, “on” and “above” are used for explaining the relation of elements shown in the drawings. The terms are relative concept and are explained based on the direction shown in the drawing.

It will be further understood that the terms “comprises” and/or “comprising,” when used in this specification, specify the presence of stated features, numerals, steps, operations, elements, parts, or the combination thereof, but do not preclude the presence or addition of one or more other features, numerals, steps, operations, elements, parts, or the combination thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

In the disclosure, when a layer, a film, a region, a plate, etc. is referred to as being “on” or “above” another part, it can be “directly on” the other part, or intervening layers may also be present. In contrast, when a layer, a film, a region, a plate, etc. is referred to as being “under” or “below” another part, it can be “directly under” the other part, or intervening layers may also be present. Also, when an element is referred to as being disposed “on” another element, it can be disposed under the other element.

In the disclosure, the term “substituted or unsubstituted” corresponds to substituted or unsubstituted with at least one substituent selected from the group including (e.g., consisting of) a deuterium atom, a halogen atom, a cyano group, a nitro group, an amine group, a silyl group, an oxy group, a thio group, a sulfinyl group, a sulfonyl group, a carbonyl group, a boron group, a phosphine oxide group, a phosphine sulfide group, an alkyl group, an alkenyl group, an alkynyl group, an alkoxy group, a hydrocarbon ring group, an aryl group, and a heterocyclic group. In addition, each of the exemplified substituents may be substituted or unsubstituted. For example, a biphenyl group may be interpreted as an aryl group or a phenyl group substituted with a phenyl group.

In the disclosure, the term “forming a ring via the combination with an adjacent group” may refer to forming a substituted or unsubstituted hydrocarbon ring, or a substituted or unsubstituted heterocycle via the combination with an adjacent group. The hydrocarbon ring includes an aliphatic hydrocarbon ring and an aromatic hydrocarbon ring. The heterocycle includes an aliphatic heterocycle and an aromatic heterocycle. The hydrocarbon ring and the heterocycle may be monocycles or polycycles. In addition, the ring formed via the combination with an adjacent group may be combined with another ring to form a spiro structure.

In the disclosure, the term “adjacent group” may refer to a substituent substituted for an atom which is directly combined with an atom substituted with a corresponding substituent, another substituent substituted for an atom which is substituted with a corresponding substituent, or a substituent sterically positioned at the nearest position to a corresponding substituent. For example, in 1,2-dimethylbenzene, two methyl groups may be interpreted as “adjacent groups” to each other, and in 1,1-diethylcyclopentene, two ethyl groups may be interpreted as “adjacent groups” to each other. In addition, in 4,5-dimethylphenanthrene, two methyl groups may be interpreted as “adjacent groups” to each other.

In the disclosure, a halogen atom may be a fluorine atom, a chlorine atom, a bromine atom or an iodine atom.

In the disclosure, an alkyl group may be a linear, branched, or cyclic type. The carbon number of the alkyl group may be 1 to 60, 1 to 50, 1 to 30, 1 to 20, 1 to 10, or 1 to 6. Examples of the alkyl group may include methyl, ethyl, n-propyl, isopropyl, n-butyl, s-butyl, t-butyl, i-butyl, 2-ethylbutyl, 3,3-dimethylbutyl, n-pentyl, i-pentyl, neopentyl, t-pentyl, cyclopentyl, 1-methylpentyl, 3-methylpentyl, 2-ethylpentyl, 4-methyl-2-pentyl, n-hexyl, 1-methylhexyl, 2-ethylhexyl, 2-butylhexyl, cyclohexyl, 4-methylcyclohexyl, 4-t-butylcyclohexyl, n-heptyl, 1-methylheptyl, 2,2-dimethylheptyl, 2-ethylheptyl, 2-butylheptyl, n-octyl, t-octyl, 2-ethyloctyl, 2-butyloctyl, 2-hexyloctyl, 3,7-dimethyloctyl, cyclooctyl, n-nonyl, n-decyl, adamantyl, 2-ethyldecyl, 2-butyldecyl, 2-hexyldecyl, 2-octyldecyl, n-undecyl, n-dodecyl, 2-ethyldodecyl, 2-butyldodecyl, 2-hexyldocecyl, 2-octyldodecyl, n-tridecyl, n-tetradecyl, n-pentadecyl, n-hexadecyl, 2-ethylhexadecyl, 2-butylhexadecyl, 2-hexylhexadecyl, 2-octylhexadecyl, n-heptadecyl, n-octadecyl, n-nonadecyl, n-eicosyl, 2-ethyleicosyl, 2-butyleicosyl, 2-hexyleicosyl, 2-octyleicosyl, n-heneicosyl, n-docosyl, n-tricosyl, n-tetracosyl, n-pentacosyl, n-hexacosyl, n-heptacosyl, n-octacosyl, n-nonacosyl, n-triacontyl, etc., without limitation.

In the disclosure, a hydrocarbon ring group refers to an optional functional group or substituent derived from an aliphatic hydrocarbon ring or a fused ring of an aliphatic hydrocarbon ring group and an aromatic hydrocarbon ring group. The number of the ring-forming carbon of the hydrocarbon ring group may be 5 to 60, 5 to 30, or 6 to 30.

In the disclosure, an aryl group refers to an optional functional group or substituent derived from an aromatic hydrocarbon ring. The aryl group may be a monocyclic aryl group or a polycyclic aryl group. The carbon number for forming rings in the aryl group may be 6 to 60, 6 to 30, 6 to 20, or 6 to 15. Examples of the aryl group may include phenyl, naphthyl, fluorenyl, anthracenyl, phenanthryl, biphenyl, terphenyl, quaterphenyl, quinquephenyl, sexiphenyl, triphenylenyl, pyrenyl, benzofluoranthenyl, chrysenyl, etc., without limitation.

In the disclosure, a heterocyclic group refers to an optional functional group or substituent derived from a ring including one or more selected from among B, O, N, P, Se, Si, and S as heteroatoms. The heterocyclic group may include an aliphatic heterocyclic group and/or an aromatic heterocyclic group. The aromatic heterocyclic group may be a heteroaryl group. The aliphatic heterocyclic group and the aromatic heterocyclic group may be a monocycle or a polycycle.

When the heterocyclic group includes two or more heteroatoms, the two or more heteroatoms may be the same or different. The heterocyclic group may be a monocyclic heterocyclic group or a polycyclic heterocyclic group, and has the concept including a heteroaryl group. The carbon number for forming rings of the heterocyclic group may be 2 to 60, 2 to 30, 2 to 20, or 2 to 10.

In the disclosure, an aliphatic heterocyclic group may include one or more selected from among B, O, N, P, Se, Si, and S as heteroatoms. The number of ring-forming carbon atoms of the aliphatic heterocyclic group may be 2 to 30, 2 to 20, or 2 to 10. Examples of the aliphatic heterocyclic group may include an oxirane group, a thiirane group, a pyrrolidine group, a piperidine group, a tetrahydrofuran group, a tetrahydrothiophene group, a thiane group, a tetrahydropyran group, a 1,4-dioxane group, etc., without limitation.

In the disclosure, a heteroaryl group may include one or more selected from among B, O, N, P, Se, Si, and S as heteroatoms. When the heteroaryl group includes two or more heteroatoms, the two or more heteroatoms may be the same or different. The heteroaryl group may be a monocyclic heterocyclic group or polycyclic heterocyclic group. The carbon number for forming rings of the heteroaryl group may be 2 to 30, 2 to 20, or 2 to 10. Examples of the heteroaryl group may include thiophene, furan, pyrrole, imidazole, pyridine, bipyridine, pyrimidine, triazine, triazole, acridyl, pyridazine, pyrazinyl, quinoline, quinazoline, quinoxaline, phenoxazine, phthalazine, pyrido pyrimidine, pyrido pyrazine, pyrazino pyrazine, isoquinoline, indole, carbazole, N-arylcarbazole, N-heteroarylcarbazole, N-alkylcarbazole, benzoxazole, benzoimidazole, benzothiazole, benzocarbazole, benzothiophene, dibenzothiophene, thienothiophene, benzofuran, phenanthroline, thiazole, isoxazole, oxazole, oxadiazole, thiadiazole, phenothiazine, dibenzosilole, dibenzofuran, etc., without limitation.

In the disclosure, the same explanation on the above-described aryl group may be applied to an arylene group except that the arylene group is a divalent group. The same explanation on the above-described heteroaryl group may be applied to a heteroarylene group except that the heteroarylene group is a divalent group.

In the disclosure, a fluorenyl group may be substituted, and two substituents may be combined with each other to form a spiro structure. Examples of a substituted fluorenyl group are as follows, but an embodiment of the present disclosure is not limited thereto.

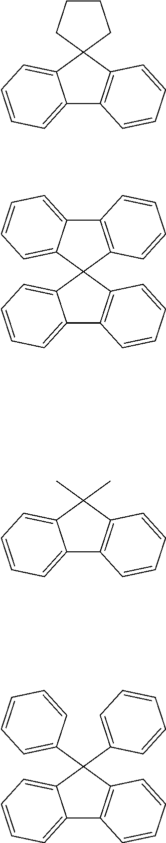

In the disclosure, a silyl group includes an alkyl silyl group and/or an aryl silyl group. Examples of the silyl group include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group, etc., without limitation.

In the disclosure, a thio group may include an alkyl thio group and/or an aryl thio group. The thio group may refer to the above-defined alkyl group or aryl group combined with a sulfur atom. Examples of the thio group include a methylthio group, an ethylthio group, a propylthio group, a pentylthio group, a hexylthio group, an octylthio group, a dodecylthio group, a cyclopentylthio group, a cyclohexylthio group, a phenylthio group, a naphthylthio group, etc., without limitation.

In the disclosure, an oxy group may refer to the above-defined alkyl group or aryl group which is combined with an oxygen atom. The oxy group may include an alkoxy group and/or an aryl oxy group. The alkoxy group may be a linear, branched or cyclic chain. The carbon number of the alkoxy group is not limited but may be, for example, 1 to 60, 1 to 20 or 1 to 10. The number of ring-forming carbon atoms of the aryl oxy group is not limited, but may be, for example, 6 to 60, 6 to 30, or 6 to 20. Examples of the oxy group may include methoxy, ethoxy, n-propoxy, isopropoxy, butoxy, pentyloxy, hexyloxy, octyloxy, nonyloxy, decyloxy, benzyloxy, etc. However, an embodiment of the present disclosure is not limited thereto.

In the disclosure, a boron group may refer to the above-defined alkyl group or aryl group, combined with a boron atom. The boron group includes an alkyl boron group and/or an aryl boron group. Examples of the boron group include a dimethylboron group, a diethylboron group, a t-butylmethylboron group, a diphenylboron group, a phenylboron group, etc., without limitation.

In the disclosure, the carbon number of an amine group is not limited, and may be 1 to 30. The amine group may include an alkyl amine group and/or an aryl amine group. Examples of the amine group include a methylamine group, a dimethylamine group, a phenylamine group, a diphenylamine group, a naphthylamine group, a 9-methyl-anthracenylamine group, a triphenylamine group, etc., without limitation.

In the disclosure, alkyl groups in an alkylthio group, alkylsulfoxy group, alkylaryl group, alkylamino group, alkylboron group, alkyl silyl group, and alkyl amine group may be the same as the examples of the above-described alkyl group.

In the disclosure, aryl groups in an aryloxy group, arylthio group, arylsulfoxy group, aryl amino group, arylboron group, and aryl silyl group may be the same as the examples of the above-described aryl group.

In the disclosure, a direct linkage may refer to a single bond. In some embodiments, in the disclosure,

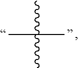

and “-*” refer to positions to be connected.

Hereinafter, the light emitting element of an embodiment will be explained referring to the drawings.

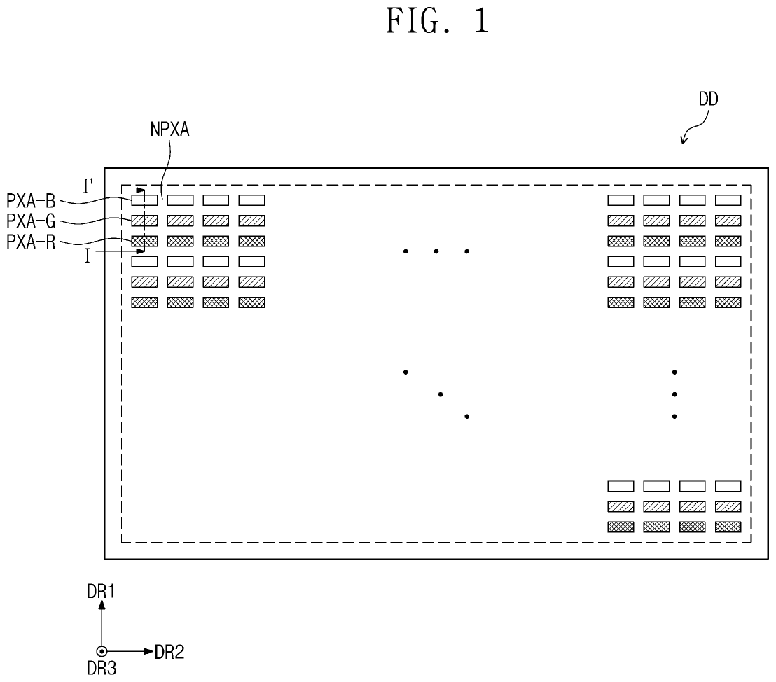

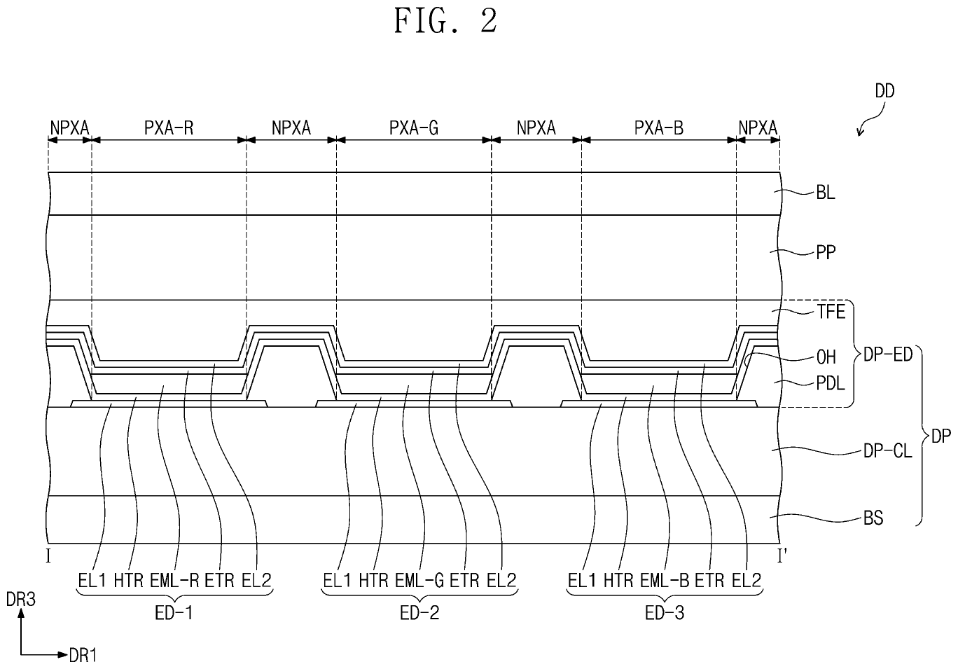

FIG. 1 is a plan view showing an embodiment of a display apparatus DD. FIG. 2 is a cross-sectional view of a display apparatus DD of an embodiment. FIG. 2 is a cross-sectional view showing a part corresponding to line I-I′ of FIG. 1.

The display apparatus DD may include a display panel DP and an optical layer PP on the display panel DP. The display panel DP may include light emitting elements ED-1, ED-2 and ED-3. The display apparatus DD may include multiple light emitting elements ED-1, ED-2 and ED-3. The optical layer PP may be on the display panel DP and control reflected light by external light at the display panel DP. The optical layer PP may include, for example, a polarization layer or a color filter layer. In some embodiments, the optical layer PP may be omitted in the display apparatus DD of an embodiment.

On the optical layer PP, a base substrate BL may be disposed. The base substrate BL may be a member providing a base surface where the optical layer PP is disposed. The base substrate BL may be a glass substrate, a metal substrate, a plastic substrate, etc. However, an embodiment of the present disclosure is not limited thereto, and the base substrate BL may be an inorganic layer, an organic layer or a composite material layer. In some embodiments, the base substrate BL may not be provided.

The display apparatus DD according to an embodiment may further include a plugging layer. The plugging layer may be between a display element layer DP-ED and a base substrate BL. The plugging layer may be an organic layer. The plugging layer may include at least one selected from among an acrylic resin, a silicon-based resin and an epoxy-based resin.

The display panel DP may include a base layer BS, a circuit layer DP-CL provided on the base layer BS and a display element layer DP-ED. The display element layer DP-ED may include a pixel definition layer PDL, light emitting elements ED-1, ED-2 and ED-3 in the pixel definition layer PDL, and an encapsulating layer TFE on the light emitting elements ED-1, ED-2 and ED-3.

The base layer BS may be a member providing a base surface in which the display element layer DP-ED is disposed. The base layer BS may be a glass substrate, a metal substrate, a plastic substrate, etc. However, an embodiment of the present disclosure is not limited thereto, and the base layer BS may be an inorganic layer, an organic layer or a composite material layer.

In an embodiment, the circuit layer DP-CL is on the base layer BS, and the circuit layer DP-CL may include multiple transistors. Each of the transistors may include a control electrode, an input electrode, and an output electrode. For example, the circuit layer DP-CL may include switching transistors and driving transistors for driving the light emitting elements ED-1, ED-2 and ED-3 of the display element layer DP-ED.

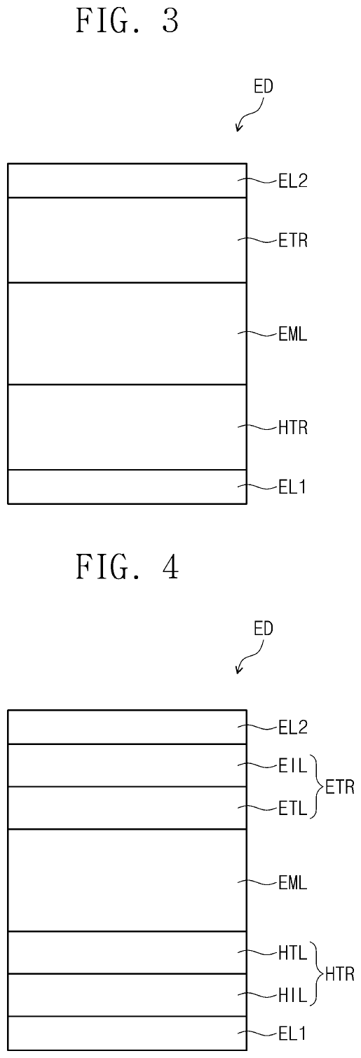

Each of the light emitting elements ED-1, ED-2 and ED-3 may have the structures of light emitting elements ED of embodiments according to FIG. 3 to FIG. 6, which will be explained in more detail. Each of the light emitting elements ED-1, ED-2 and ED-3 may include a first electrode EL1, a hole transport region HTR, emission layers EML-R, EML-G and EML-B, an electron transport region ETR, and a second electrode EL2.

In FIG. 2, shown is an embodiment in which the emission layers EML-R, EML-G and EML-B of light emitting elements ED-1, ED-2 and ED-3 are in opening portions OH defined in a pixel definition layer PDL, and a hole transport region HTR, an electron transport region ETR and a second electrode EL2 are provided as common layers in all light emitting elements ED-1, ED-2 and ED-3. However, an embodiment of the present disclosure is not limited thereto. In an embodiment, the hole transport region HTR and the electron transport region ETR may be patterned and provided in the opening portions OH defined in the pixel definition layer PDL. For example, in an embodiment, the hole transport region HTR, the emission layers EML-R, EML-G and EML-B, and the electron transport region ETR of the light emitting elements ED-1, ED-2 and ED-3 may be patterned by an ink jet printing method and provided.

An encapsulating layer TFE may cover the light emitting elements ED-1, ED-2 and ED-3. The encapsulating layer TFE may encapsulate the display element layer DP-ED. The encapsulating layer TFE may be a thin film encapsulating layer. The encapsulating layer TFE may be one layer or a stacked layer of multiple layers. The encapsulating layer TFE includes at least one insulating layer. The encapsulating layer TFE according to an embodiment may include at least one inorganic layer (hereinafter, encapsulating inorganic layer). In some embodiments, the encapsulating layer TFE may include at least one organic layer (hereinafter, encapsulating organic layer) and at least one encapsulating inorganic layer.

The encapsulating inorganic layer protects (reduces the amount of moisture/oxygen) the display element layer DP-ED from moisture/oxygen, and the encapsulating organic layer protects (reduces the amount of foreign materials) the display element layer DP-ED from foreign materials such as dust particles. The encapsulating inorganic layer may include silicon nitride, silicon oxy nitride, silicon oxide, titanium oxide, or aluminum oxide, without specific limitation. The encapsulating organic layer may include an acrylic compound, an epoxy-based compound, etc. The encapsulating organic layer may include a photopolymerizable organic material, without limitation.

The encapsulating layer TFE may be on the second electrode EL2 and may be disposed while filling the opening portion OH.

Referring to FIG. 1 and FIG. 2, the display apparatus DD may include a non-luminous area NPXA and luminous areas (light emitting regions) PXA-R, PXA-G and PXA-B. The luminous areas PXA-R, PXA-G and PXA-B may be areas emitting light produced from the light emitting elements ED-1, ED-2 and ED-3, respectively. The luminous areas PXA-R, PXA-G and PXA-B may be separated from each other on a plane.

The luminous areas PXA-R, PXA-G and PXA-B may be areas separated by the pixel definition layer PDL. The non-luminous areas NPXA may be areas between neighboring luminous areas PXA-R, PXA-G and PXA-B and may be areas corresponding to the pixel definition layer PDL. In some embodiments, in the disclosure, each of the luminous areas PXA-R, PXA-G and PXA-B may correspond to each pixel. The pixel definition layer PDL may divide the light emitting elements ED-1, ED-2 and ED-3. The emission layers EML-R, EML-G and EML-B of the light emitting elements ED-1, ED-2 and ED-3 may be disposed and divided in the opening portions OH defined in the pixel definition layer PDL.

The luminous areas PXA-R, PXA-G and PXA-B may be divided into multiple groups according to the color of light produced from the light emitting elements ED-1, ED-2 and ED-3. In the display apparatus DD of an embodiment, shown in FIG. 1 and FIG. 2, three luminous areas PXA-R, PXA-G and PXA-B emitting red light, green light and blue light are illustrated as an embodiment. For example, the display apparatus DD of an embodiment may include a red luminous area PXA-R, a green luminous area PXA-G and a blue luminous area PXA-B, which are separated from each other.

In the display apparatus DD according to an embodiment, multiple light emitting elements ED-1, ED-2 and ED-3 may emit light having different wavelength regions. For example, in an embodiment, the display apparatus DD may include a first light emitting element ED-1 emitting red light, a second light emitting element ED-2 emitting green light, and a third light emitting element ED-3 emitting blue light. For example, each of the red luminous area PXA-R, the green luminous area PXA-G, and the blue luminous area PXA-B of the display apparatus DD may correspond to the first light emitting element ED-1, the second light emitting element ED-2, and the third light emitting element ED-3.

However, an embodiment of the present disclosure is not limited thereto, and the first to third light emitting elements ED-1, ED-2 and ED-3 may emit light in the same wavelength region, or at least one thereof may emit light in a different wavelength region. For example, all the first to third light emitting elements ED-1, ED-2 and ED-3 may emit blue light.

The luminous areas PXA-R, PXA-G and PXA-B in the display apparatus DD according to an embodiment may be arranged in a stripe shape. Referring to FIG. 1, multiple red luminous areas PXA-R may be arranged with each other along a second direction axis DR2, multiple green luminous areas PXA-G may be arranged with each other along a second direction axis DR2, and multiple blue luminous areas PXA-B may be arranged with each other along a second direction axis DR2. In some embodiments, the red luminous area PXA-R, the green luminous area PXA-G and the blue luminous area PXA-B may be arranged by turns (alternately arranged) along a first direction axis DR1. (DR3 is a third direction which is normal or perpendicular to the plane defined by the first direction DR1 and the second direction DR2).

In FIG. 1 and FIG. 2, the areas of the luminous areas PXA-R, PXA-G and PXA-B are shown similar, but an embodiment of the present disclosure is not limited thereto. The areas of the luminous areas PXA-R, PXA-G and PXA-B may be different from each other according to the wavelength region of light emitted. In some embodiments, the areas of the luminous areas PXA-R, PXA-G and PXA-B may refer to areas on a plane defined by the first direction axis DR1 and the second direction axis DR2.

In some embodiments, the arrangement type of the luminous areas PXA-R, PXA-G and PXA-B is not limited to the configuration shown in FIG. 1, and the arrangement order of the red luminous areas PXA-R, the green luminous areas PXA-G and the blue luminous areas PXA-B may be provided in one or more suitable combinations according to the properties of display quality required for the display apparatus DD. For example, the arrangement type or kind of the luminous areas PXA-R, PXA-G and PXA-B may be a PENTILE® arrangement type (PENTILE® arrangement form, for example, an RGBG matrix, an RGBG structure, or RGBG matrix structure), or a Diamond Pixel™ arrangement type (Diamond Pixel™ arrangement form). PENTILE® is a duly registered trademark of Samsung Display Co., Ltd. Diamond Pixel™ is a trademark of Samsung Display Co., Ltd.

In some embodiments, the areas of the luminous areas PXA-R, PXA-G and PXA-B may be different from each other. For example, in an embodiment, the area of the green luminous area PXA-G may be smaller than the area of the blue luminous area PXA-B, but an embodiment of the present disclosure is not limited thereto.

Hereinafter, FIG. 3 to FIG. 6 are cross-sectional views schematically showing light emitting elements according to embodiments. The light emitting element ED according to an embodiment may include a first electrode EL1, a second electrode EL2 oppositely disposed to the first electrode EL1, and at least one functional layer between the first electrode EL1 and the second electrode EL2. The light emitting element ED of an embodiment may include the polycyclic compound of an embodiment, which will be explained in more detail, in the at least one functional layer.

The light emitting element ED may include a hole transport region HTR, an emission layer EML, an electron transport region ETR, or the like, stacked in order as the at least one functional layer. For example, the light emitting element ED of an embodiment may include a first electrode EL1, a hole transport region HTR, an emission layer EML, an electron transport region ETR, and a second electrode EL2, stacked in order.

The light emitting element ED of an embodiment may include the polycyclic compound of an embodiment, which will be explained later, in an emission layer EML or an electron transport region ETR. However, an embodiment of the present disclosure is not limited thereto, and the light emitting element ED of an embodiment may include the polycyclic compound according to an embodiment, which will be explained in more detail, in a hole transport region HTR among multiple functional layers between the first electrode EL1 and the second electrode EL2, in addition to the emission layer EML and the electron transport region ETR, or may include the polycyclic compound according to an embodiment, which will be explained in more detail, in a capping layer CPL on the second electrode EL2.

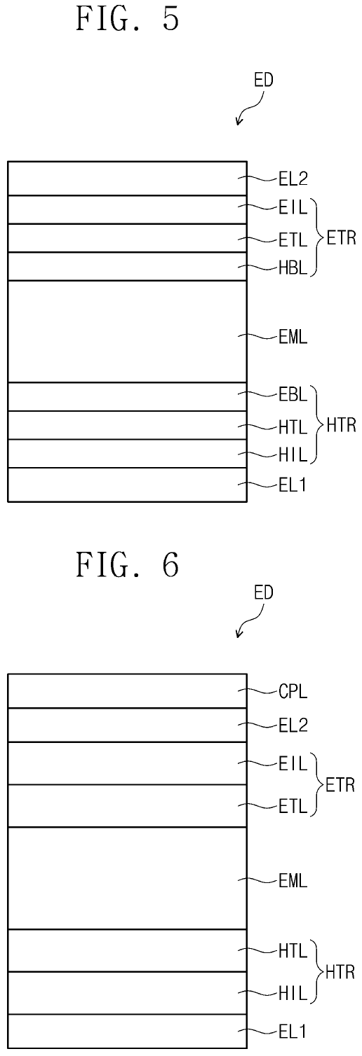

When compared with FIG. 3, FIG. 4 shows the cross-sectional view of a light emitting element ED of an embodiment, wherein a hole transport region HTR includes a hole injection layer HIL and a hole transport layer HTL, and an electron transport region ETR includes an electron injection layer EIL and an electron transport layer ETL. In some embodiments, when compared with FIG. 3, FIG. 5 shows the cross-sectional view of a light emitting element ED of an embodiment, wherein a hole transport region HTR includes a hole injection layer HIL, a hole transport layer HTL, and an electron blocking layer EBL, and an electron transport region ETR includes an electron injection layer EIL, an electron transport layer ETL, and a hole blocking layer HBL. When compared with FIG. 4, FIG. 6 shows the cross-sectional view of a light emitting element ED of an embodiment, including a capping layer CPL on the second electrode EL2.

In the light emitting element ED according to an embodiment, the first electrode EL1 has conductivity (e.g., is a conductor). The first electrode EL1 may be formed using a metal material, a metal alloy or a conductive compound. The first electrode EL1 may be an anode or a cathode. However, an embodiment of the present disclosure is not limited thereto. In some embodiments, the first electrode EL1 may be a pixel electrode. The first electrode EL1 may be a transmissive electrode, a transflective electrode, or a reflective electrode. The first electrode EL1 may include at least one selected from among Ag, Mg, Cu, Al, Pt, Pd, Au, Ni, Nd, Ir, Cr, Li, Ca, LiF, Mo, Ti, W, In, Sn, Zn, compounds comprising one or more of the foregoing elements, combinations of two or more of the foregoing elements or compounds, mixtures of two or more of the foregoing elements or compounds, and/or oxides thereof.

When the first electrode EL1 is the transmissive electrode, the first electrode EL1 may include a transparent metal oxide such as indium tin oxide (ITO), indium zinc oxide (IZO), zinc oxide (ZnO), and/or indium tin zinc oxide (ITZO). When the first electrode EL1 is the transflective electrode or the reflective electrode, the first electrode EL1 may include Ag, Mg, Cu, Al, Pt, Pd, Au, Ni, Nd, Ir, Cr, Li, Ca, LiF/Ca (a stacked structure of LiF and Ca), LiF/Al (a stacked structure of LiF and Al), Mo, Ti, W, one or more compounds thereof, or one or more mixtures thereof (for example, a mixture of Ag and Mg). In some embodiments, the first electrode EL1 may have a structure including multiple layers including a reflective layer or a transflective layer formed using the above materials, and a transmissive conductive layer formed using ITO, IZO, ZnO, or ITZO. For example, the first electrode EL1 may have a three-layer structure of ITO/Ag/ITO. However, an embodiment of the present disclosure is not limited thereto. The first electrode EL1 may include the above-described metal materials, combinations of two or more metal materials selected from the above-described metal materials, or oxides of the above-described metal materials. The thickness of the first electrode EL1 may be from about 700 Å to about 10,000 Å. For example, the thickness of the first electrode EL1 may be from about 1,000 Å to about 3,000 Å.

The hole transport region HTR is provided on the first electrode EL1. The hole transport region HTR may include at least one of a hole injection layer HIL, a hole transport layer HTL, a buffer layer or an emission auxiliary layer, or an emission blocking layer EBL. The thickness of the hole transport region HTR may be, for example, about 50 Å to about 15,000 Å.

The hole transport region HTR may have a single layer formed using a single material, a single layer formed using multiple different materials, or a multilayer structure including multiple layers formed using multiple different materials.

For example, the hole transport region HTR may have the structure of a single layer of a hole injection layer HIL or a hole transport layer HTL, and may have a structure of a single layer formed using a hole injection material and a hole transport material. In some embodiments, the hole transport region HTR may have a structure of a single layer formed using multiple different materials, or a structure stacked from the first electrode EL1 of hole injection layer HIL/hole transport layer HTL, hole injection layer HIL/hole transport layer HTL/buffer layer, hole injection layer HIL/buffer layer, hole transport layer HTL/buffer layer, or hole injection layer HIL/hole transport layer HTL/electron blocking layer EBL, without limitation.

The hole transport region HTR may be formed using one or more suitable methods such as a vacuum deposition method, a spin coating method, a cast method, a Langmuir-Blodgett (LB) method, an inkjet printing method, a laser printing method, and/or a laser induced thermal imaging (LITI) method.

The hole transport region HTR may include a compound represented by Formula H-1.Formula H-1

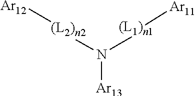

In Formula H-1, L₁ and L₂ may each independently be a direct linkage, a substituted or unsubstituted arylene group of 6 to 30 ring-forming carbon atoms, or a substituted or unsubstituted heteroarylene group of 2 to 30 ring-forming carbon atoms. n1 and n2 may each independently be an integer from 0 to 10. In some embodiments, when n1 or n2 is an integer of 2 or more, multiple L₁ and L₂ may each independently be a substituted or unsubstituted arylene group of 6 to 30 ring-forming carbon atoms, or a substituted or unsubstituted heteroarylene group of 2 to 30 ring-forming carbon atoms.

In Formula H-1, Ar₁₁ and Ar₁₂ may each independently be a substituted or unsubstituted aryl group of 6 to 30 ring-forming carbon atoms, or a substituted or unsubstituted heteroaryl group of 2 to 30 ring-forming carbon atoms. In some embodiments, in Formula H-1, Ar₁₃ may be a substituted or unsubstituted aryl group of 6 to 30 ring-forming carbon atoms.

The compound represented by Formula H-1 may be a monoamine compound. Or the compound represented by Formula H-1 may be a diamine compound in which at least one selected from among Ar₁₁ to Ar₁₃ includes an amine group as a substituent. In some embodiments, the compound represented by Formula H-1 may be a carbazole-based compound in which at least one selected from among Ar₁₁ and Ar₁₂ includes a substituted or unsubstituted carbazole group, or a fluorene-based compound in which at least one selected from among Ar₁₁ and Ar₁₂ includes a substituted or unsubstituted fluorene group.

The compound represented by Formula H-1 may be represented by any one selected from among the compounds in Compound Group H. However, the compounds shown in Compound Group H are merely examples, and the compound represented by Formula H-1 is not limited to the compounds represented in Compound Group H.

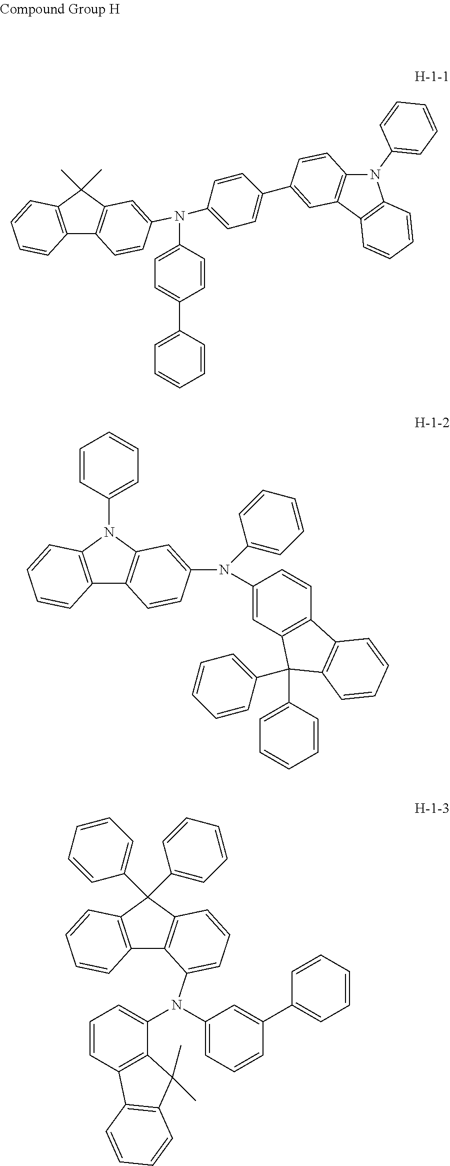 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(N(c2ccc3c(c2)C(c2ccccc2)(c2ccccc2)c2ccccc2-3)c2ccc3c4ccccc4n(-c4ccccc4)c3c2)cc1 ; CC1(C)c2ccccc2-c2cccc(N(c3cccc(-c4ccccc4)c3)c3cccc4c3-c3ccccc3C4(c3ccccc3)c3ccccc3)c21]

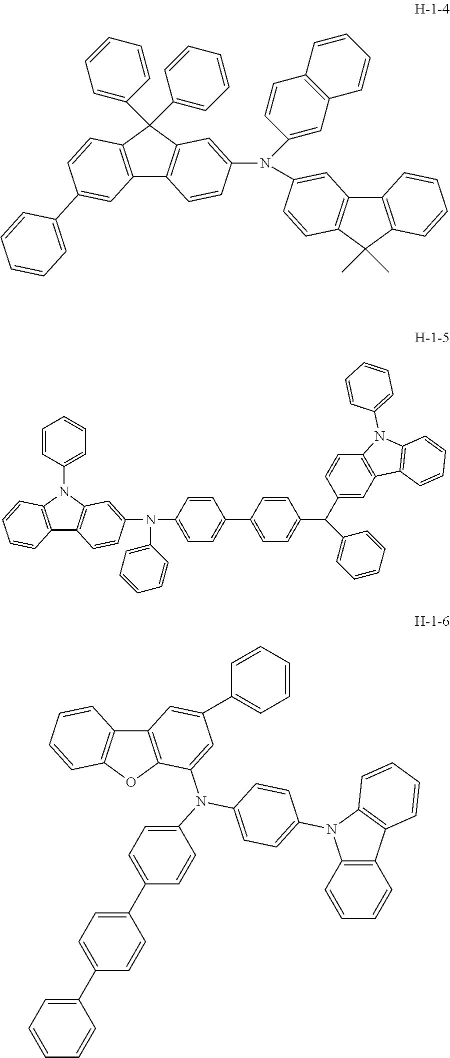

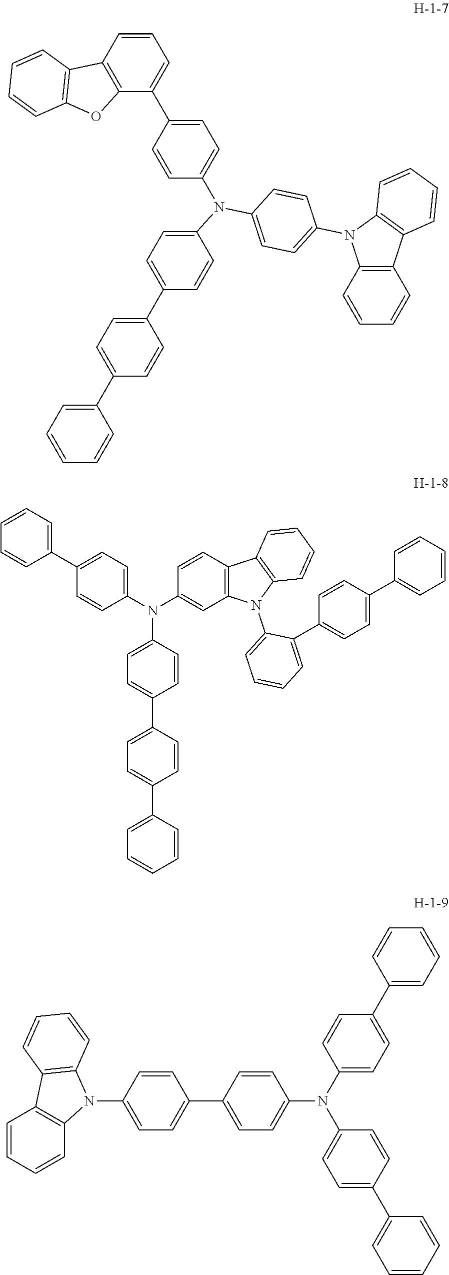 [SMILES: c1ccc(-c2ccc(-c3ccc(N(c4ccc(-c5cccc6c5oc5ccccc56)cc4)c4ccc(-n5c6ccccc6c6ccccc65)cc4)cc3)cc2)cc1 ; c1ccc(-c2ccc(-c3ccc(N(c4ccc(-c5ccccc5)cc4)c4ccc5c6ccccc6n(-c6ccccc6-c6ccc(-c7ccccc7)cc6)c5c4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1]

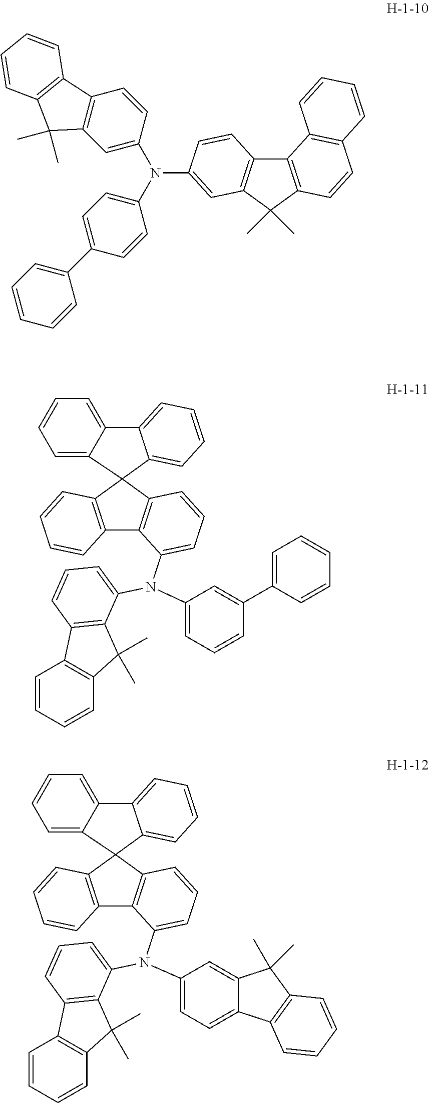 [SMILES: CC1(C)c2ccccc2-c2ccc(N(c3ccc(-c4ccccc4)cc3)c3ccc4c(c3)C(C)(C)c3ccc5ccccc5c3-4)cc21 ; CC1(C)c2ccccc2-c2cccc(N(c3cccc(-c4ccccc4)c3)c3cccc4c3-c3ccccc3C43c4ccccc4-c4ccccc43)c21 ; CC1(C)c2ccccc2-c2ccc(N(c3cccc4c3-c3ccccc3C43c4ccccc4-c4ccccc43)c3cccc4c3C(C)(C)c3ccccc3-4)cc21]

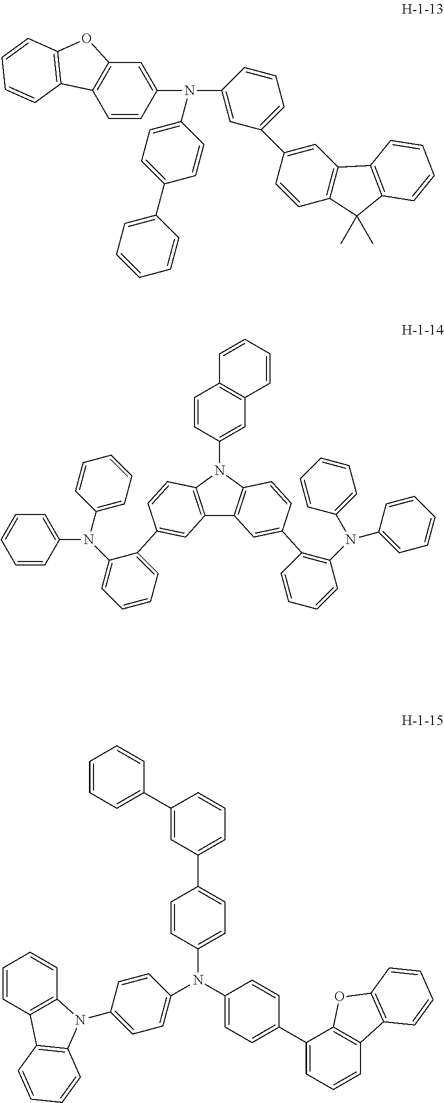 [SMILES: CC1(C)c2ccccc2-c2cc(-c3cccc(N(c4ccc(-c5ccccc5)cc4)c4ccc5c(c4)oc4ccccc45)c3)ccc21 ; c1ccc(N(c2ccccc2)c2ccccc2-c2ccc3c(c2)c2cc(-c4ccccc4N(c4ccccc4)c4ccccc4)ccc2n3-c2ccc3ccccc3c2)cc1 ; c1ccc(-c2cccc(-c3ccc(N(c4ccc(-c5cccc6c5oc5ccccc56)cc4)c4ccc(-n5c6ccccc6c6ccccc65)cc4)cc3)c2)cc1]

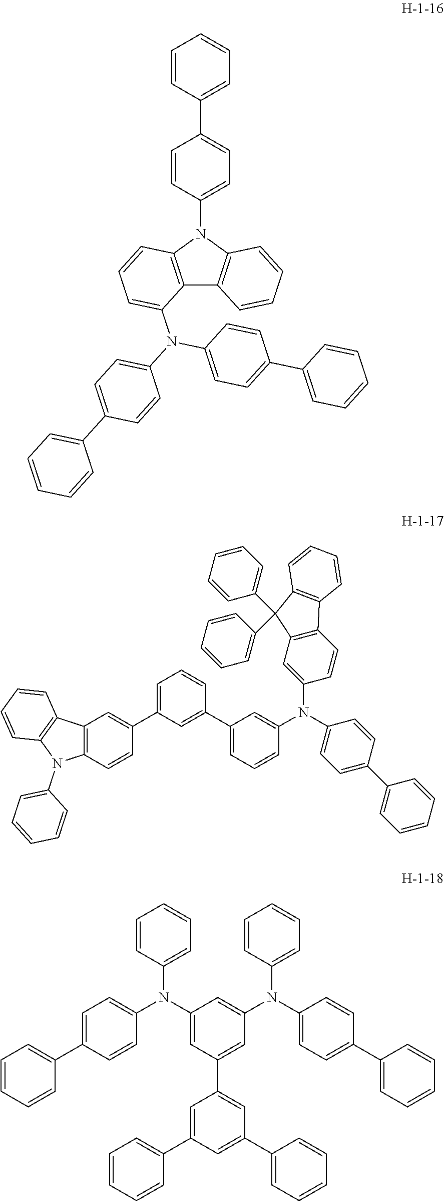 [SMILES: c1ccc(-c2ccc(N(c3ccc(-c4ccccc4)cc3)c3cccc4c3c3ccccc3n4-c3ccc(-c4ccccc4)cc3)cc2)cc1 ; c1ccc(-c2ccc(N(c3cccc(-c4cccc(-c5ccc6c(c5)c5ccccc5n6-c5ccccc5)c4)c3)c3ccc4c(c3)C(c3ccccc3)(c3ccccc3)c3ccccc3-4)cc2)cc1 ; c1ccc(-c2ccc(N(c3ccccc3)c3cc(-c4cc(-c5ccccc5)cc(-c5ccccc5)c4)cc(N(c4ccccc4)c4ccc(-c5ccccc5)cc4)c3)cc2)cc1]

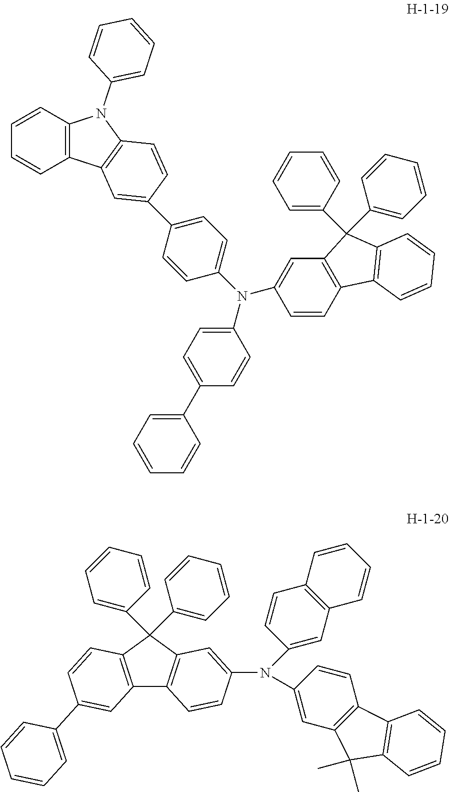

The hole transport region HTR may include a phthalocyanine compound such as copper phthalocyanine, N¹,N¹′-([1,1′-biphenyl]-4,4′-diyl)bis(N¹-phenyl-N⁴,N⁴-di-m-tolylbenzene-1,4-diamine) (DNTPD), 4,4′,4″-[tris(3-methylphenyl)phenylamino] triphenylamine (m-MTDATA), 4,4′,4″-tris(N,N-diphenylamino)triphenylamine (TDATA), 4,4′,4″-tris[N(2-naphthyl)-N-phenylamino]-triphenylamine (2-TNATA), poly(3,4-ethylenedioxythiophene)/poly(4-styrenesulfonate) (PEDOT/PSS), polyaniline/dodecylbenzenesulfonic acid (PANI/DBSA), polyaniline/camphor sulfonic acid (PANI/CSA), polyaniline/poly(4-styrenesulfonate) (PANI/PSS), N,N′-di(1-naphthalene-1-yl)-N,N′-diphenyl-benzidine (NPB or NPD), triphenylamine-containing polyetherketone (TPAPEK), 4-isopropyl-4′-methyldiphenyliodonium [tetrakis(pentafluorophenyl)borate], and/or dipyrazino[2,3-f:2′,3′-h] quinoxaline-2,3,6,7,10,11-hexacarbonitrile (HATCN).

In some embodiments, the hole transport region HTR may include 9-(4-tert-butylphenyl)-3,6-bis(triphenylsilyl)-9H-carbazole (CzSi), 9-phenyl-9H-3,9′-bicarbazole (CCP), 1,3-bis(1,8-dimethyl-9H-carbazol-9-yl)benzene (mDCP), etc.

The hole transport region HTR may include the compounds of the hole transport region in at least one selected from among the hole injection layer HIL, hole transport layer HTL, and electron blocking layer EBL.

The thickness of the hole transport region HTR may be from about 100 Å to about 10,000 Å, for example, from about 100 Å to about 5,000 Å. In an embodiment in which the hole transport region HTR includes a hole injection layer HIL, the thickness of the hole injection region HIL may be, for example, from about 30 Å to about 1,000 Å. In an embodiment in which the hole transport region HTR includes a hole transport layer HTL, the thickness of the hole transport layer HTL may be from about 30 Å to about 1,000 Å. For example, in an embodiment in which the hole transport region HTR includes an electron blocking layer, the thickness of the electron blocking layer EBL may be from about 10 Å to about 1,000 Å. When the thicknesses of the hole transport region HTR, the hole injection layer HIL, the hole transport layer HTL and the electron blocking layer EBL satisfy the above-described ranges, satisfactory (suitable) hole transport properties may be achieved without substantial increase of a driving voltage.

The hole transport region HTR may further include a charge generating material to increase conductivity in addition to the above-described materials. The charge generating material may be dispersed substantially uniformly or non-uniformly in the hole transport region HTR. The charge generating material may be, for example, a p-dopant. The p-dopant may include at least one of metal halide compounds, quinone derivatives, metal oxides, and cyano group-containing compounds, without limitation. For example, the p-dopant may include metal halide compounds such as CuI and RbI, quinone derivatives such as tetracyanoquinodimethane (TCNQ) and 2,3,5,6-tetrafluoro-7,7′,8,8-tetracyanoquinodimethane (F4-TCNQ), metal oxides such as tungsten oxide and molybdenum oxide, cyano group-containing compounds such as dipyrazino[2,3-f: 2′,3′-h] quinoxaline-2,3,6,7,10,11-hexacarbonitrile (HATCN) and 4-[[2,3-bis[cyano-(4-cyano-2,3,5,6-tetrafluorophenyl)methylidene]cyclopropylidene]-cyanomethyl]-2,3,5,6-tetrafluorobenzonitrile (NDP9), etc., without limitation.

As described above, the hole transport region HTR may further include at least one selected from among a buffer layer and an electron blocking layer EBL in addition to the hole injection layer HIL and the hole transport layer HTL. The buffer layer may compensate for a resonance distance according to the wavelength of light emitted from an emission layer EML and may increase emission efficiency. Materials which may be included in the hole transport region HTR may also be used in the buffer layer. The electron blocking layer EBL is a layer playing the role of (functioning as) blocking (reducing) the injection of electrons from the electron transport region ETR to the hole transport region HTR.

The emission layer EML is provided on the hole transport region HTR. The emission layer EML may have a thickness of, for example, about 100 Å to about 1,000 Å or about 100 Å to about 300 Å. The emission layer EML may have a single layer formed using a single material, a single layer formed using multiple different materials, or a multilayer structure having multiple layers formed using multiple different materials.

The emission layer EML may include the polycyclic compound of an embodiment. The polycyclic compound of an embodiment may include a structure in which a nitrogen-containing ring is bonded to an indolocarbazole skeleton. For example, the polycyclic compound of an embodiment may be a compound in which a nitrogen-containing ring is bonded, and one or more specific substituents containing a heteroatom such as Si, C and/or Ge, is bonded to the skeleton structure of 5-phenyl-5,12-dihydroindolo[3,2-a]carbazole.

For example, the polycyclic compound of an embodiment includes the indolocarbazole skeleton of 5,12-dihydroindolo[3,2-a]carbazole and may have a relatively high lowest excitation triplet energy level (T1 level) in contrast to a polycyclic compound including the indolocarbazole skeleton of 5,7-dihydroindolo[2,3-b]carbazole. Accordingly, the polycyclic compound according to an embodiment may have a lowest excitation triplet energy level suitable to apply as the host material of an emission layer EML included in a light emitting element emitting thermally activated delayed fluorescence or phosphorescence, and the reduction of a driving voltage of an element by reinforcing bipolar properties in a molecule in contrast to a compound including a carbazole skeleton. In some embodiments, the polycyclic compound of an embodiment includes at least one or more bulky specific substituents, for example, substituents represented by Formula 2, and high efficiency may be achieved through the reduction of the interaction with a dopant.

The polycyclic compound of an embodiment may be represented by Formula 1.

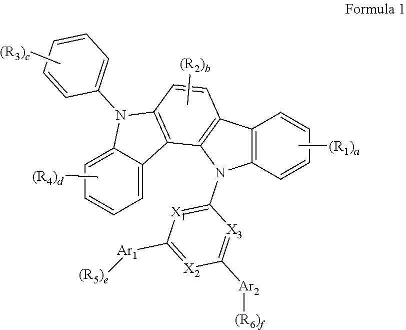

In Formula 1, X₁ to X₃ may each independently be C or N, in which at least two or more selected from among X₁ to X₃ may be N. For example, X₁ to X₃ may be all N, or X₁ and X₃ may be N, and X₂ may be C. For example, in Formula 1, the nitrogen-containing ring bonded to the indolocarbazole skeleton may be pyrimidine or 1,3,5-triazine.

In Formula 1, Ar₁ and Ar₂ may each independently be a substituted or unsubstituted arylene group of 6 to 60 ring-forming carbon atoms, or a substituted or unsubstituted heteroarylene group of 2 to 60 ring-forming carbon atoms. For example, Ar₁ and Ar₂ may each independently be a substituted or unsubstituted arylene group of 6 to 20 ring-forming carbon atoms, or a substituted or unsubstituted heteroarylene group of 2 to 20 ring-forming carbon atoms. In some embodiments, Ar₁ and Ar₂ may each independently be a substituted or unsubstituted phenylene group, a substituted or unsubstituted divalent pyridoindole group, or a substituted or unsubstituted divalent carbazole group. In an embodiment, when Ar₁ and Ar₂ may each independently be a substituted divalent carbazole group, a substituent may be a cyano group. However, an embodiment of the present disclosure is not limited thereto.

In Formula 1, R₁ to R₆ may each independently be a hydrogen atom, a deuterium atom, or a cyano group, or represented by Formula 2. At least one selected from among R₁ to R₆ is a substituent represented by Formula 2. In an embodiment, one to four, or one to two selected from among R₁ to R₆ may be represented by Formula 2. For example, any one selected from among R₁, R₃, R₄, R₅ and R₆ may be represented by Formula 2. Or two or more selected from among R₁, R₃, R₄, R₅ and R₆ may be represented by Formula 2. However, an embodiment of the present disclosure is not limited thereto.

In Formula 1, “a” and “d” may each independently be an integer from 0 to 4. “b” may be an integer from 0 to 2, and “c” to “f” may be integers from 0 to 5. For example, an embodiment in which “a” is 0, may be substantially the same as an embodiment in which “a” is 1, and R₁ is a hydrogen atom. An embodiment in which “b” is 0, may be substantially the same as an embodiment in which “b” is 1, and R₂ is a hydrogen atom. An embodiment in which “c” is 0, may be substantially the same as an embodiment in which “c” is 1, and R₃ is a hydrogen atom. An embodiment in which “d” is 0, may be substantially the same as an embodiment in which “d” is 1, and R₄ is a hydrogen atom. In addition, an embodiment in which “e” is 0, may be substantially the same as an embodiment in which “e” is 1, and R₅ is a hydrogen atom, and an embodiment in which “f” is 0, may be substantially the same as an embodiment in which “f” is 1, and R₆ is a hydrogen atom. In some embodiments, when R₅ and R₆ are hydrogen atoms, or “e” and “f” are 0, Ar₁ and Ar₂ may be monovalent substituents.

When “a” is an integer of 2 or more, multiple R₁ may be the same, or at least one may be different from the remainder. When “b” is an integer of 2, two R₂ may be the same, or different from each other. When “c” is an integer of 2 or more, multiple R₃ may be the same, or at least one may be different from the remainder. When “d” is an integer of 2 or more, multiple R₄ may be the same, or at least one may be different from the remainder. When “e” is an integer of 2 or more, multiple R₅ may be the same, or at least one may be different from the remainder. When “f” is an integer of 2 or more, multiple R₆ may be the same, or at least one may be different from the remainder.

In the present disclosure, the polycyclic compound of an embodiment, represented by Formula 1 may include one or more substituents represented by Formula 2.

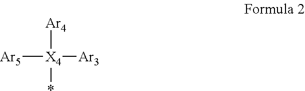

In Formula 2, X₄ may be C, Si or Ge. Ar₃ to Ar₅ may each independently be a substituted or unsubstituted aryl group of 6 to 60 ring-forming carbon atoms, a substituted or unsubstituted cycloalkyl group of 3 to 60 ring-forming carbon atoms, or a substituted or unsubstituted heterocyclic group of 2 to 60 ring-forming carbon atoms, or combined with an adjacent group to form a ring.

For example, Ar₃ to Ar₅ may each independently be a substituted or unsubstituted aryl group of 6 to 20 ring-forming carbon atoms, or a substituted or unsubstituted heteroaryl group of 2 to 20 ring-forming carbon atoms, or combined with an adjacent group to form a ring. For example, Ar₃ to Ar₅ may each independently be a substituted or unsubstituted phenyl group, or a substituted or unsubstituted dibenzofuran group, or combined with an adjacent group to form a heterocycle containing an oxygen atom (O) and a silicon atom (Si) as ring-forming atoms. However, an embodiment of the present disclosure is not limited thereto.

In an embodiment, the substituent represented by Formula 2 may be represented by any one selected from among 2-1 to 2-6. For example, the polycyclic compound of an embodiment, represented by Formula 1 may include one or more substituents represented by any one selected from among 2-1 to 2-6. However, an embodiment of the present disclosure is not limited thereto. In 2-1 to 2-6, “*-” is a position bonded to the benzene ring, the nitrogen-containing ring, Ar₁ or Ar₂ of Formula

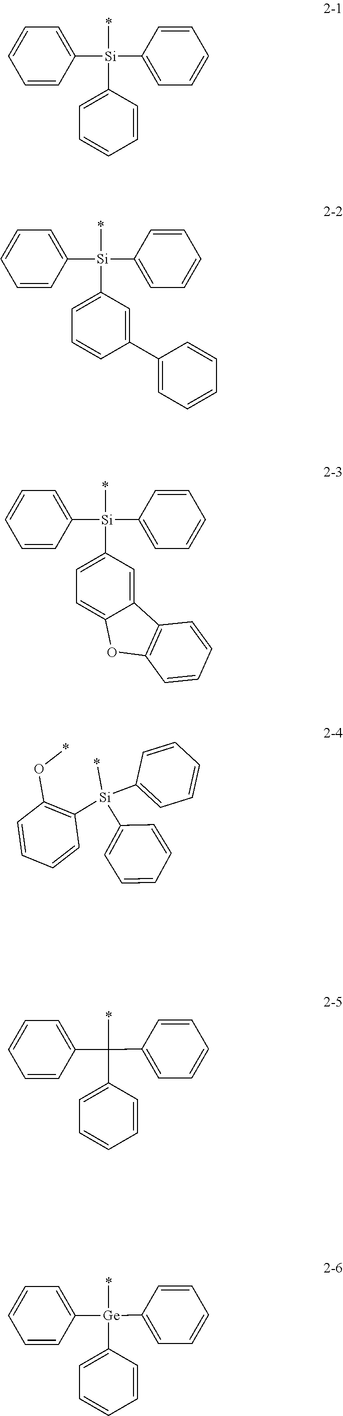

In an embodiment, the polycyclic compound represented by Formula 1 may be represented by any one selected from among Formula 3-1 to Formula 3-4. In Formula 3-1 to Formula 3-4, the same explanation for R₁ to R₆, Ar₁, Ar₂ and X₁ to X₃ referring to Formula 1 may be applied.

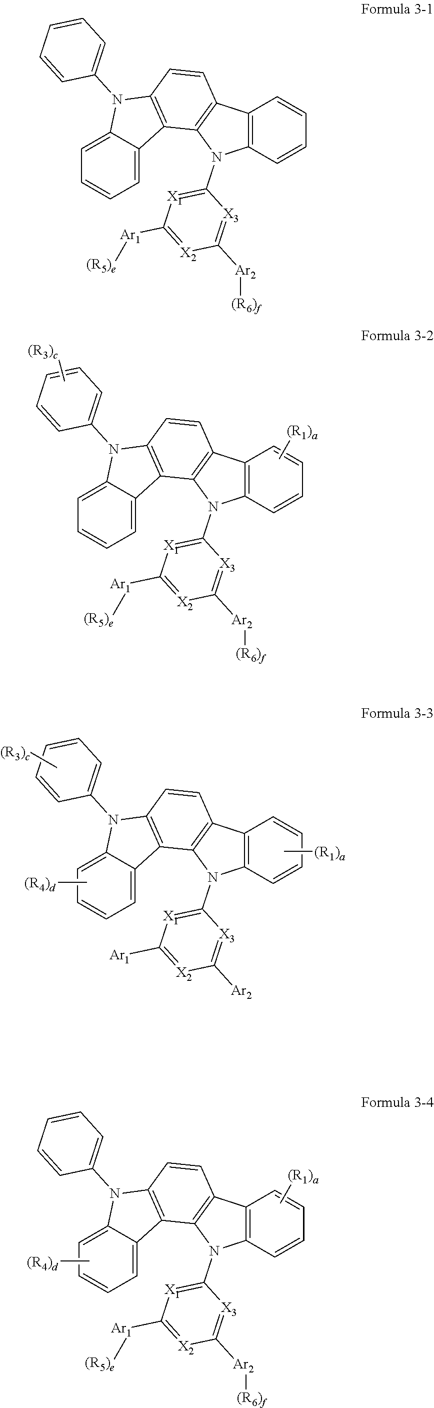

Formula 3-1 to Formula 3-4 correspond to Formula 1 in which a position at which the substituent represented by Formula 2 may be connected.

Formula 3-1 represents an embodiment of Formula 1 in which R₁ to R₄ are all hydrogen atoms (or “a” to “d” are 0), and at least one selected from among R₅ and R₆ is a substituent represented by Formula 2. Formula 3-2 represents an embodiment of Formula 1 in which R₂ and R₄ are all hydrogen atoms (or “b” and “d” are 0), and at least one selected from among R₁ and R₃, and at least one selected from among R₅ and R₆ are substituents represented by Formula 2. Formula 3-3 represents an embodiment of Formula 1 in which R₅ and R₆ are all hydrogen atoms (or “e” and “f” are 0), and at least one selected from among R₁, R₃ and R₄ of Formula 1 is represented by Formula 2. Formula 3-4 represents an embodiment of Formula 1 in which R₂ and R₃ are all hydrogen atoms (or “b” and “c” are 0), and at least one selected from among R₁, R₄, R₅ and R₆ of Formula 1 is represented by Formula 2. In some embodiments, when Ar₁ and Ar₂ are substituted in Formula 3-1 and Formula 3-4, a cyano group may be included as a substituent.

In an embodiment, the polycyclic compound represented by Formula 1 may be represented by any one selected from among 4-1 to 4-19.

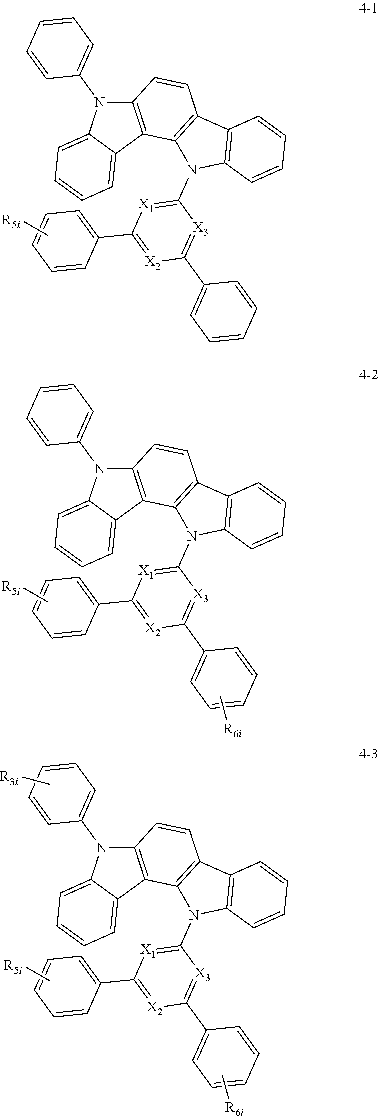

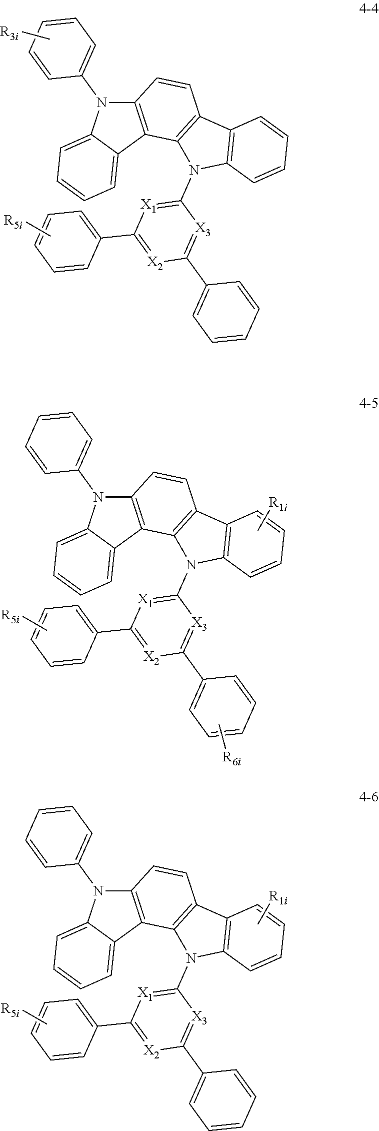

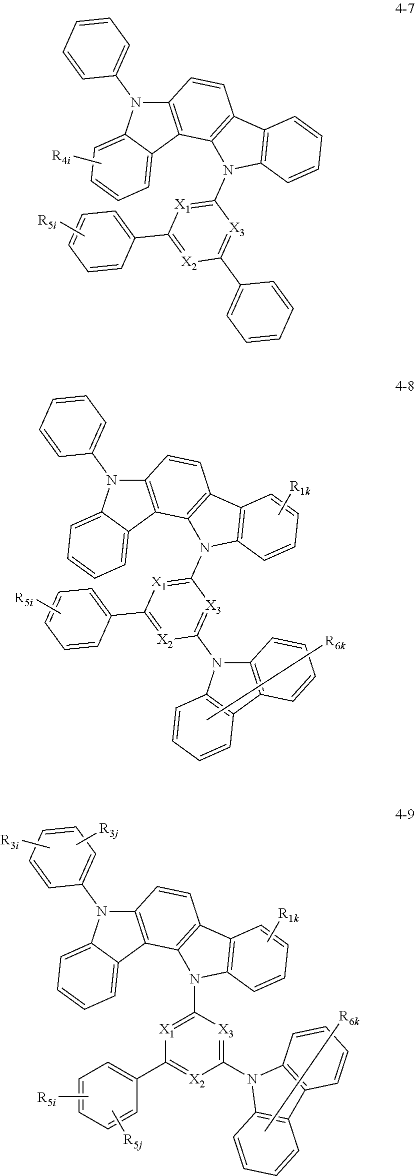

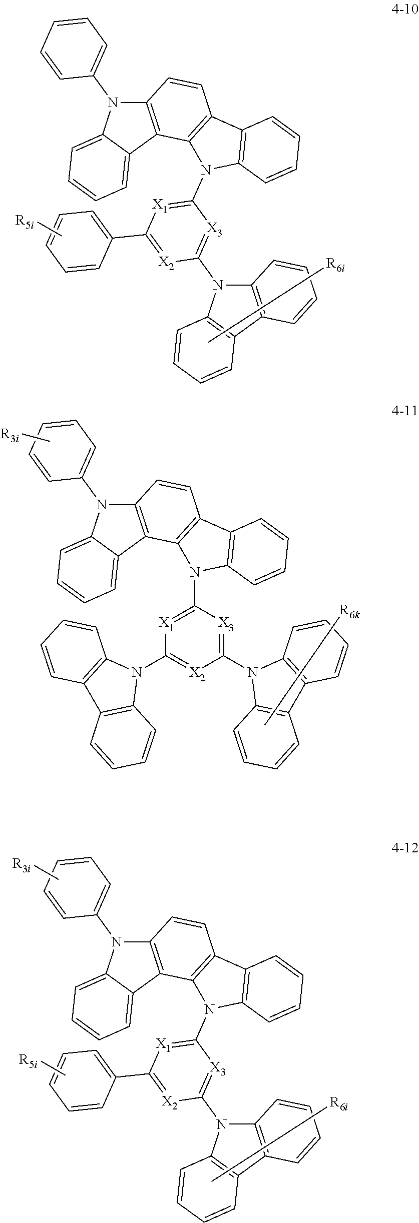

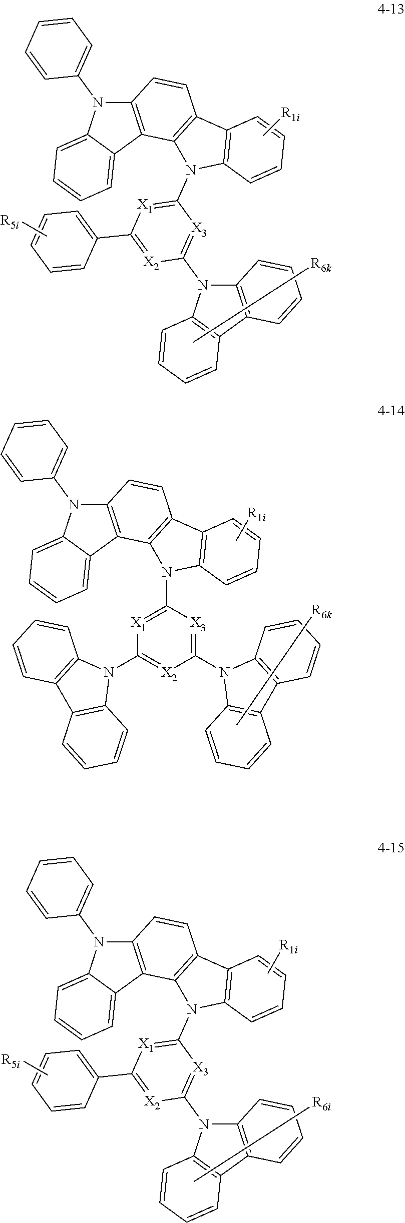

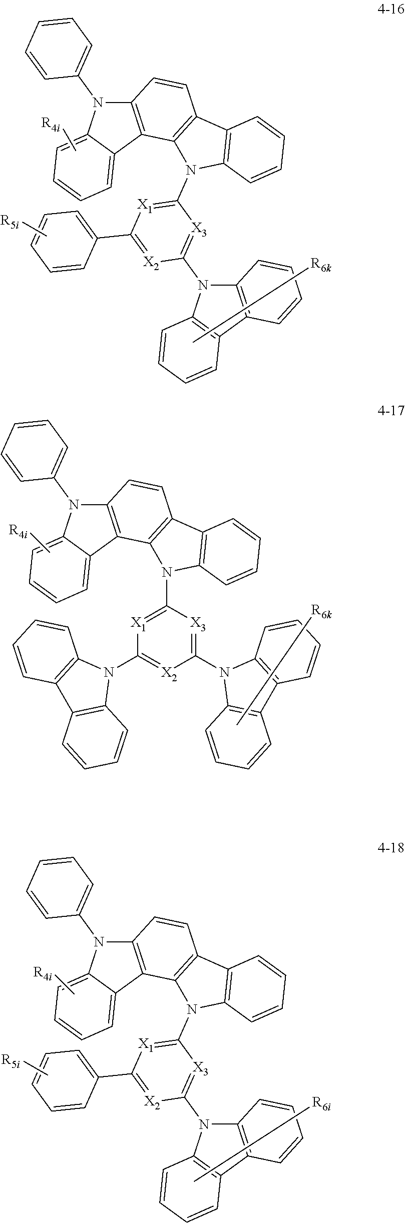

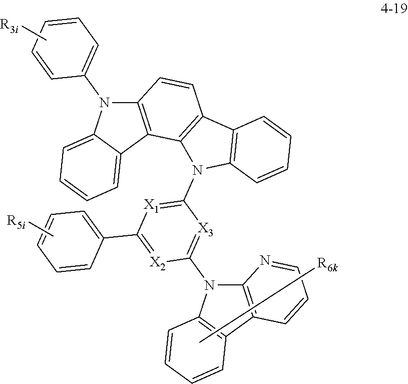

The polycyclic compounds 4-1 to 4-19 correspond to Formula 1 in which Ar₁ and Ar₂ are embodied, and the position connected of the substituent represented by Formula 2 is embodied. The polycyclic compounds represented by 4-1 to 4-6 represent Formula 1 in which Ar₁ and Ar₂ are phenylene groups, the polycyclic compounds represented by 4-7 to 4-10, 4-12 to 4-13, 4-15 to 4-16 and 4-18 represent Formula 1 in which Ar₁ of Formula 1 is a phenylene group, and Ar₂ of Formula 1 is a monovalent or divalent carbazole group. The polycyclic compounds represented by 4-11, 4-14 and 4-17 represent Formula 1 in which Ar₁ and Ar₂ are divalent carbazole groups. The polycyclic compound represented by 4-19 represents Formula 1 in which Ar₁ of Formula 1 is a phenylene group, and Ar₂ of Formula 1 is a pyridoindole group. In some embodiments, in 4-1 to 4-19, Ar₁ and Ar₂ may each independently be a substituted phenyl group, a monovalent pyridoindole group or a substituted monovalent carbazole group. In this embodiment, each of Ar₁ and Ar₂ may be substituted with Formula 2 or a cyano group.

In the polycyclic compound represented by 4-1 to 4-19, R₁ᵢ, R₃ᵢ, R₄ᵢ, R₅ᵢ and R₆ᵢ may each independently be represented by Formula 2, R₁ⱼ and R₅ⱼ may each independently be a hydrogen atom or represented by Formula 2, R₁ₖ and R₆ₖ may each independently be a hydrogen atom or a cyano group. X₁ to X₃ may be the same as defined in Formula 1.

The polycyclic compound of an embodiment, represented by Formula may be any one selected from among the compounds represented in Compound Group 1. The light emitting element ED of an embodiment may include at least one polycyclic compound selected from among the polycyclic compounds in Compound Group 1 in an emission layer EML or an electron transport region ETR.

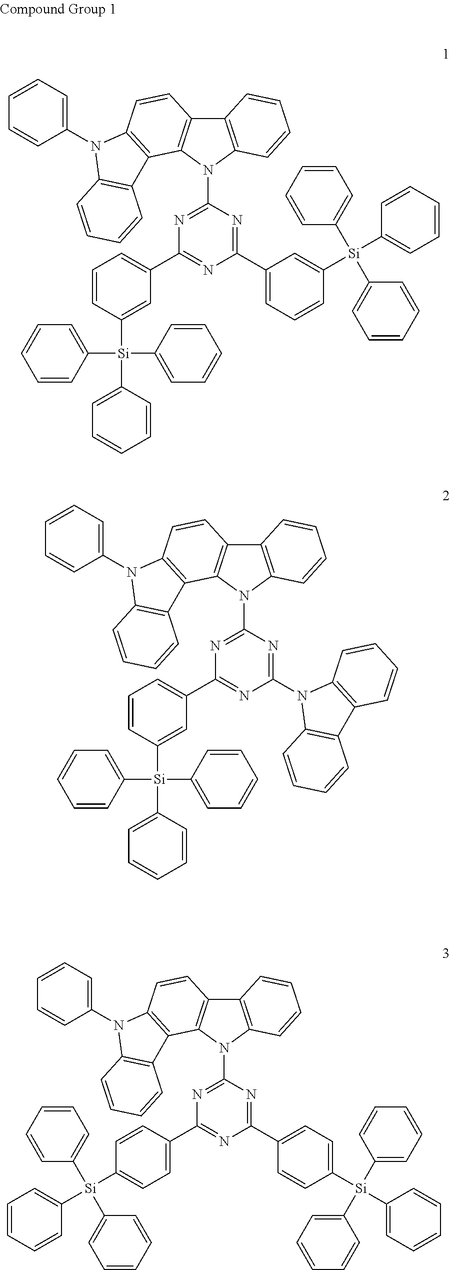 [SMILES: c1ccc(-n2c3ccccc3c3c2ccc2c4ccccc4n(-c4nc(-c5cccc([Si](c6ccccc6)(c6ccccc6)c6ccccc6)c5)nc(-c5cccc([Si](c6ccccc6)(c6ccccc6)c6ccccc6)c5)n4)c23)cc1 ; c1ccc(-n2c3ccccc3c3c2ccc2c4ccccc4n(-c4nc(-c5cccc([Si](c6ccccc6)(c6ccccc6)c6ccccc6)c5)nc(-n5c6ccccc6c6ccccc65)n4)c23)cc1 ; c1ccc(-n2c3ccccc3c3c2ccc2c4ccccc4n(-c4nc(-c5ccc([Si](c6ccccc6)(c6ccccc6)c6ccccc6)cc5)nc(-c5ccc([Si](c6ccccc6)(c6ccccc6)c6ccccc6)cc5)n4)c23)cc1]

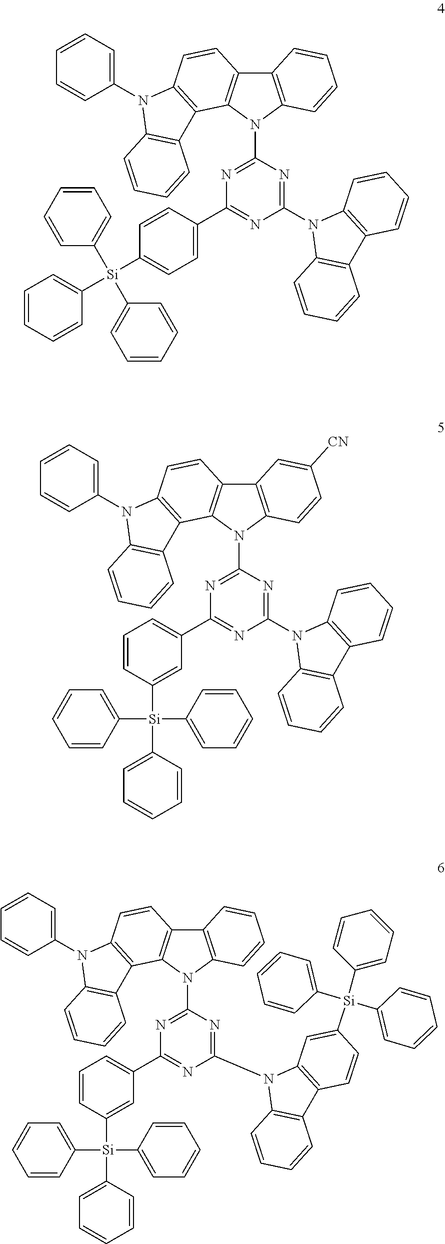 [SMILES: c1ccc(-n2c3ccccc3c3c2ccc2c4ccccc4n(-c4nc(-c5ccc([Si](c6ccccc6)(c6ccccc6)c6ccccc6)cc5)nc(-n5c6ccccc6c6ccccc65)n4)c23)cc1 ; N#Cc1ccc2c(c1)c1ccc3c(c4ccccc4n3-c3ccccc3)c1n2-c1nc(-c2cccc([Si](c3ccccc3)(c3ccccc3)c3ccccc3)c2)nc(-n2c3ccccc3c3ccccc32)n1 ; c1ccc(-n2c3ccccc3c3c2ccc2c4ccccc4n(-c4nc(-c5cccc([Si](c6ccccc6)(c6ccccc6)c6ccccc6)c5)nc(-n5c6ccccc6c6ccc([Si](c7ccccc7)(c7ccccc7)c7ccccc7)cc65)n4)c23)cc1]

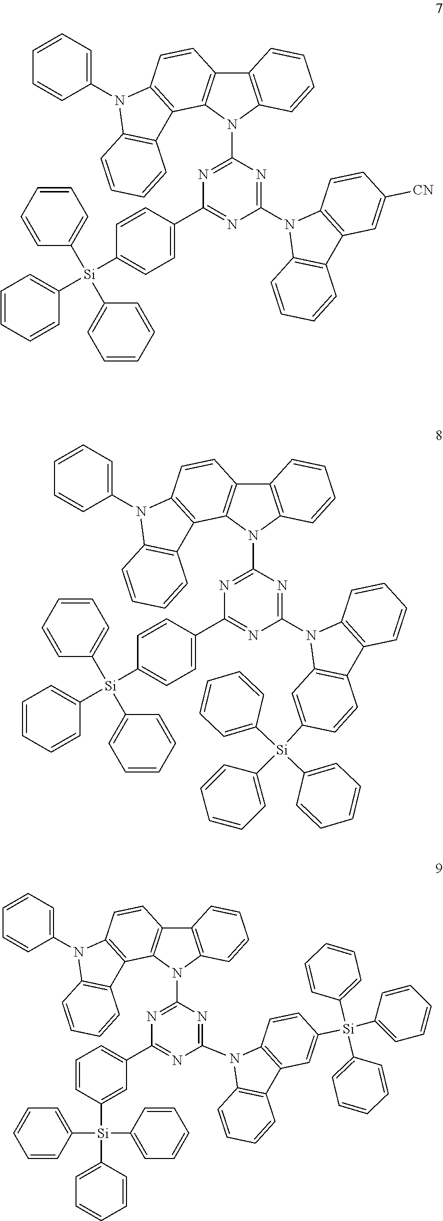 [SMILES: N#Cc1ccc2c(c1)c1ccccc1n2-c1nc(-c2ccc([Si](c3ccccc3)(c3ccccc3)c3ccccc3)cc2)nc(-n2c3ccccc3c3ccc4c(c5ccccc5n4-c4ccccc4)c32)n1 ; c1ccc(-n2c3ccccc3c3c2ccc2c4ccccc4n(-c4nc(-c5ccc([Si](c6ccccc6)(c6ccccc6)c6ccccc6)cc5)nc(-n5c6ccccc6c6ccc([Si](c7ccccc7)(c7ccccc7)c7ccccc7)cc65)n4)c23)cc1 ; c1ccc(-n2c3ccccc3c3c2ccc2c4ccccc4n(-c4nc(-c5cccc([Si](c6ccccc6)(c6ccccc6)c6ccccc6)c5)nc(-n5c6ccccc6c6cc([Si](c7ccccc7)(c7ccccc7)c7ccccc7)ccc65)n4)c23)cc1]

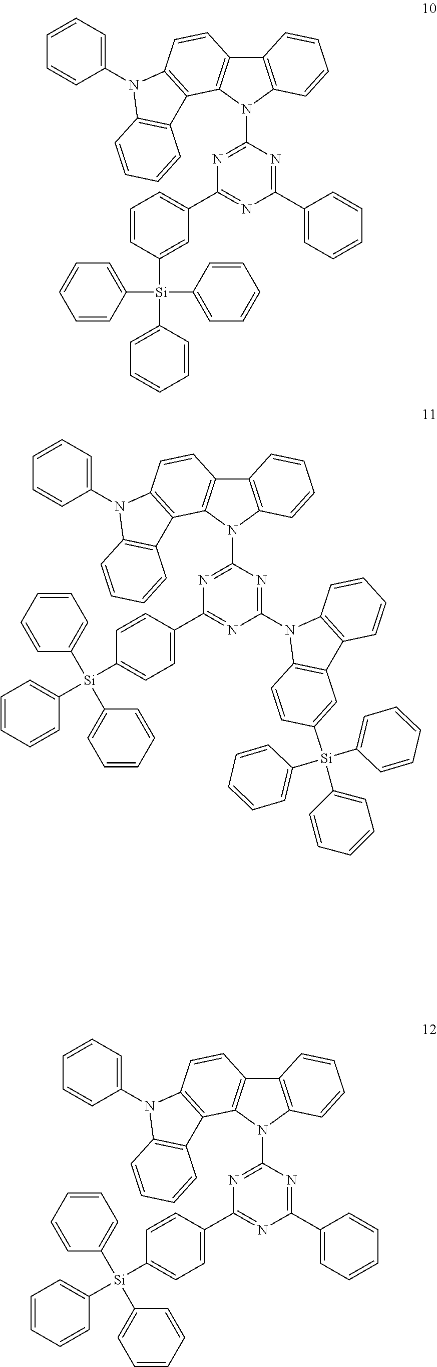 [SMILES: c1ccc(-c2nc(-c3cccc([Si](c4ccccc4)(c4ccccc4)c4ccccc4)c3)nc(-n3c4ccccc4c4ccc5c(c6ccccc6n5-c5ccccc5)c43)n2)cc1 ; c1ccc(-n2c3ccccc3c3c2ccc2c4ccccc4n(-c4nc(-c5ccc([Si](c6ccccc6)(c6ccccc6)c6ccccc6)cc5)nc(-n5c6ccccc6c6cc([Si](c7ccccc7)(c7ccccc7)c7ccccc7)ccc65)n4)c23)cc1 ; c1ccc(-c2nc(-c3ccc([Si](c4ccccc4)(c4ccccc4)c4ccccc4)cc3)nc(-n3c4ccccc4c4ccc5c(c6ccccc6n5-c5ccccc5)c43)n2)cc1]

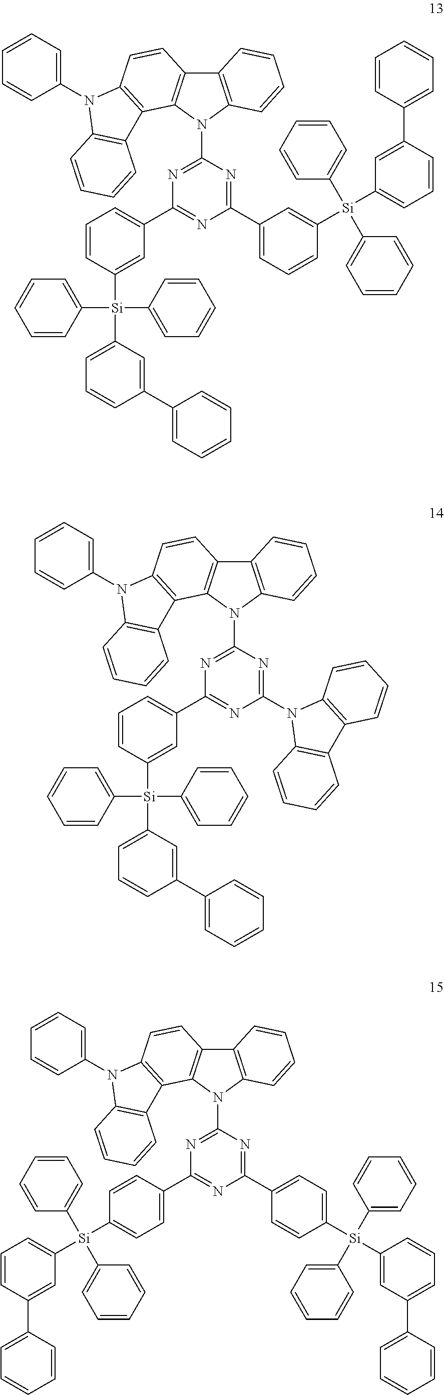 [SMILES: c1ccc(-c2cccc([Si](c3ccccc3)(c3ccccc3)c3cccc(-c4nc(-c5cccc([Si](c6ccccc6)(c6ccccc6)c6cccc(-c7ccccc7)c6)c5)nc(-n5c6ccccc6c6ccc7c(c8ccccc8n7-c7ccccc7)c65)n4)c3)c2)cc1 ; c1ccc(-c2cccc([Si](c3ccccc3)(c3ccccc3)c3cccc(-c4nc(-n5c6ccccc6c6ccccc65)nc(-n5c6ccccc6c6ccc7c(c8ccccc8n7-c7ccccc7)c65)n4)c3)c2)cc1 ; c1ccc(-c2cccc([Si](c3ccccc3)(c3ccccc3)c3ccc(-c4nc(-c5ccc([Si](c6ccccc6)(c6ccccc6)c6cccc(-c7ccccc7)c6)cc5)nc(-n5c6ccccc6c6ccc7c(c8ccccc8n7-c7ccccc7)c65)n4)cc3)c2)cc1]

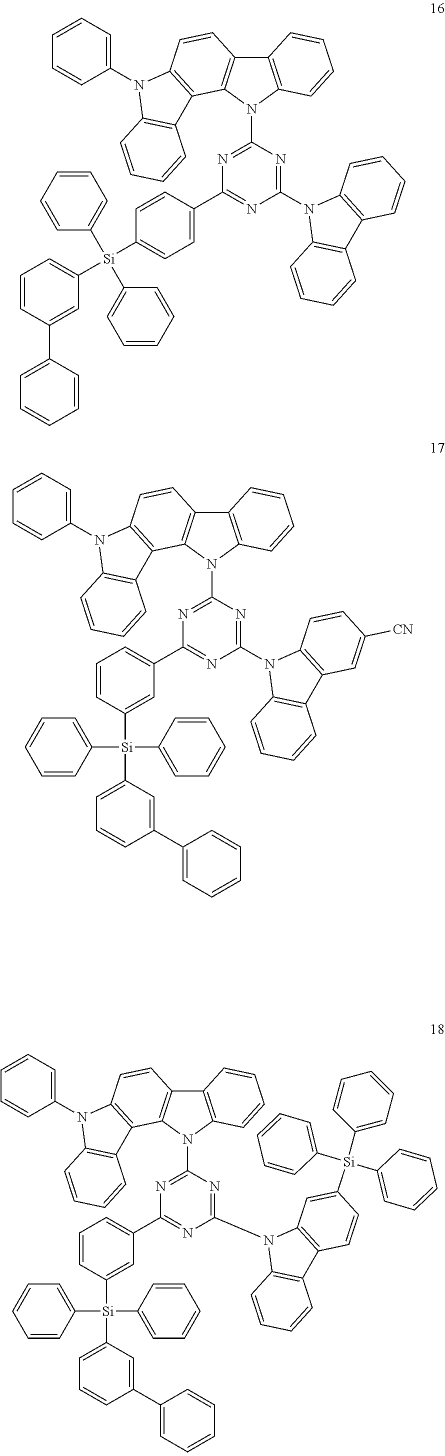

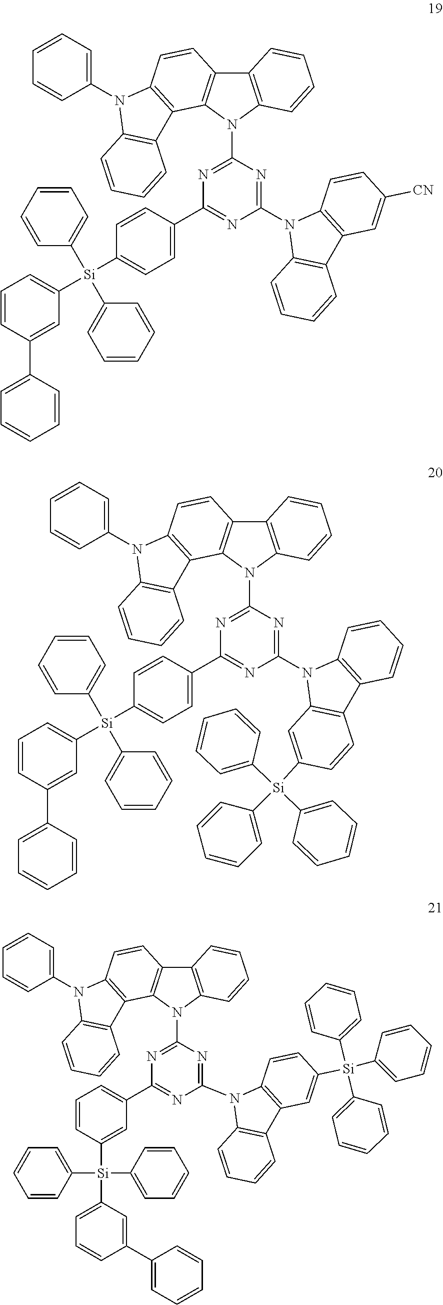 [SMILES: N#Cc1ccc2c(c1)c1ccccc1n2-c1nc(-c2ccc([Si](c3ccccc3)(c3ccccc3)c3cccc(-c4ccccc4)c3)cc2)nc(-n2c3ccccc3c3ccc4c(c5ccccc5n4-c4ccccc4)c32)n1 ; c1ccc(-c2cccc([Si](c3ccccc3)(c3ccccc3)c3ccc(-c4nc(-n5c6ccccc6c6ccc([Si](c7ccccc7)(c7ccccc7)c7ccccc7)cc65)nc(-n5c6ccccc6c6ccc7c(c8ccccc8n7-c7ccccc7)c65)n4)cc3)c2)cc1 ; c1ccc(-c2cccc([Si](c3ccccc3)(c3ccccc3)c3cccc(-c4nc(-n5c6ccccc6c6cc([Si](c7ccccc7)(c7ccccc7)c7ccccc7)ccc65)nc(-n5c6ccccc6c6ccc7c(c8ccccc8n7-c7ccccc7)c65)n4)c3)c2)cc1]

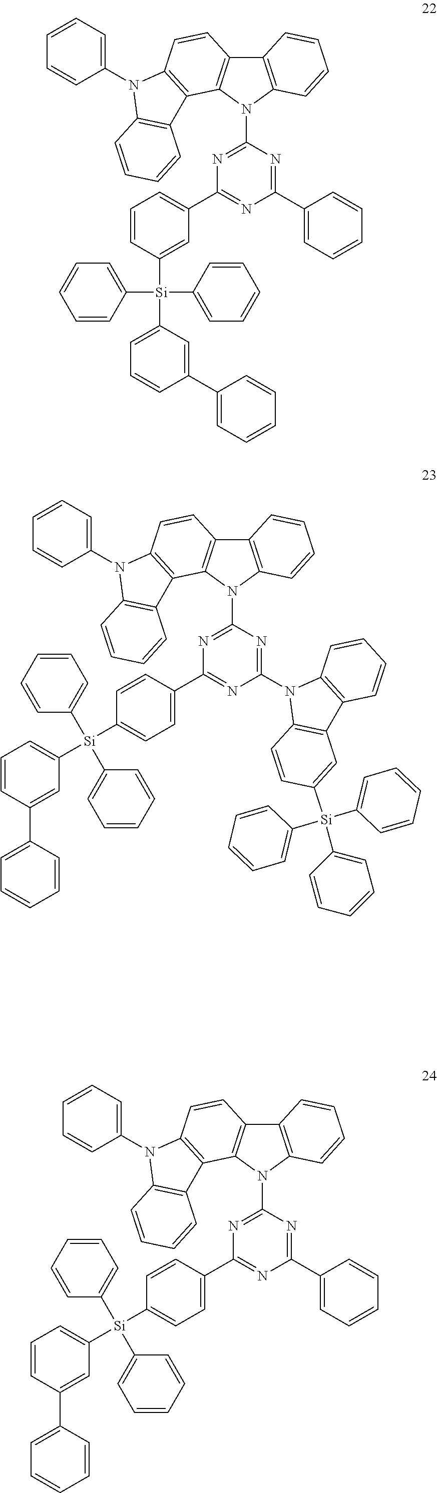 [SMILES: c1ccc(-c2cccc([Si](c3ccccc3)(c3ccccc3)c3cccc(-c4nc(-c5ccccc5)nc(-n5c6ccccc6c6ccc7c(c8ccccc8n7-c7ccccc7)c65)n4)c3)c2)cc1 ; c1ccc(-c2cccc([Si](c3ccccc3)(c3ccccc3)c3ccc(-c4nc(-n5c6ccccc6c6cc([Si](c7ccccc7)(c7ccccc7)c7ccccc7)ccc65)nc(-n5c6ccccc6c6ccc7c(c8ccccc8n7-c7ccccc7)c65)n4)cc3)c2)cc1 ; c1ccc(-c2cccc([Si](c3ccccc3)(c3ccccc3)c3ccc(-c4nc(-c5ccccc5)nc(-n5c6ccccc6c6ccc7c(c8ccccc8n7-c7ccccc7)c65)n4)cc3)c2)cc1]

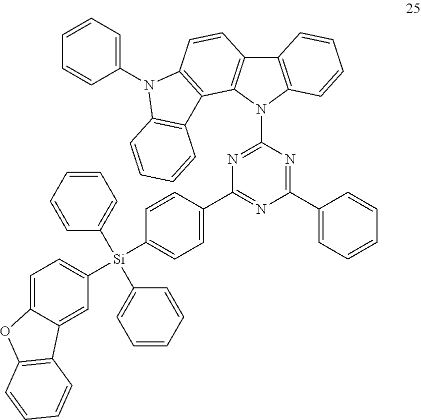 [SMILES: c1ccc(-c2nc(-c3ccc([Si](c4ccccc4)(c4ccccc4)c4ccc5oc6ccccc6c5c4)cc3)nc(-n3c4ccccc4c4ccc5c(c6ccccc6n5-c5ccccc5)c43)n2)cc1]

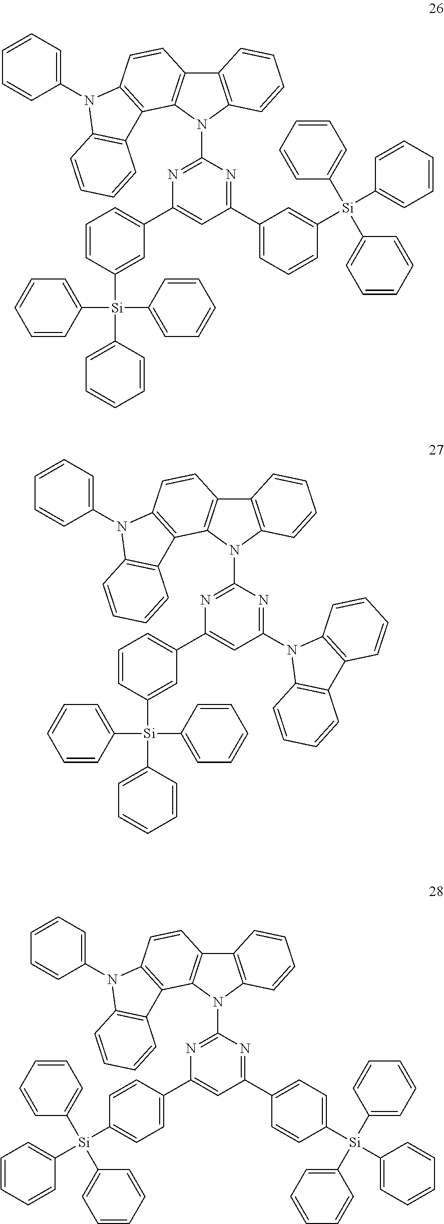 [SMILES: c1ccc(-n2c3ccccc3c3c2ccc2c4ccccc4n(-c4nc(-c5cccc([Si](c6ccccc6)(c6ccccc6)c6ccccc6)c5)cc(-c5cccc([Si](c6ccccc6)(c6ccccc6)c6ccccc6)c5)n4)c23)cc1 ; c1ccc(-n2c3ccccc3c3c2ccc2c4ccccc4n(-c4nc(-c5cccc([Si](c6ccccc6)(c6ccccc6)c6ccccc6)c5)cc(-n5c6ccccc6c6ccccc65)n4)c23)cc1 ; c1ccc(-n2c3ccccc3c3c2ccc2c4ccccc4n(-c4nc(-c5ccc([Si](c6ccccc6)(c6ccccc6)c6ccccc6)cc5)cc(-c5ccc([Si](c6ccccc6)(c6ccccc6)c6ccccc6)cc5)n4)c23)cc1]

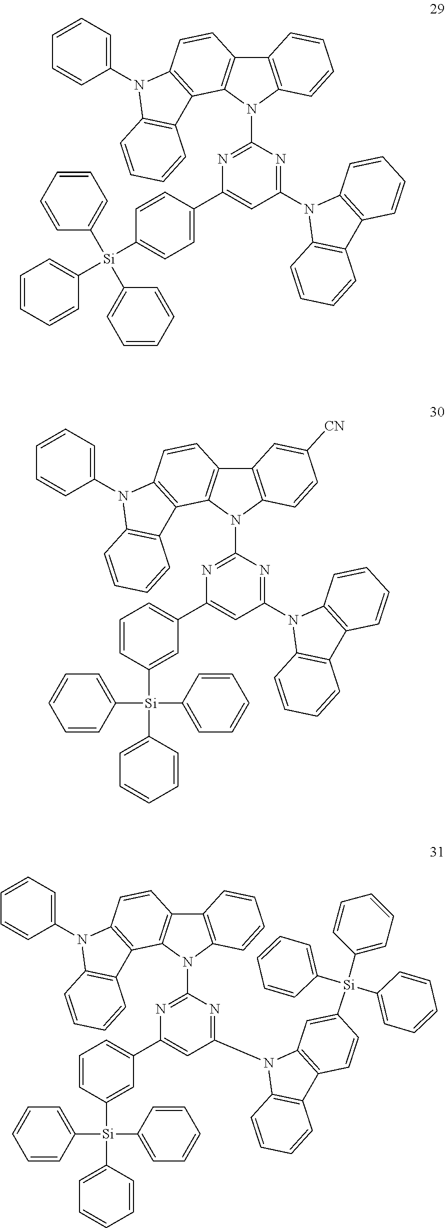 [SMILES: c1ccc(-n2c3ccccc3c3c2ccc2c4ccccc4n(-c4nc(-c5ccc([Si](c6ccccc6)(c6ccccc6)c6ccccc6)cc5)cc(-n5c6ccccc6c6ccccc65)n4)c23)cc1 ; N#Cc1ccc2c(c1)c1ccc3c(c4ccccc4n3-c3ccccc3)c1n2-c1nc(-c2cccc([Si](c3ccccc3)(c3ccccc3)c3ccccc3)c2)cc(-n2c3ccccc3c3ccccc32)n1 ; c1ccc(-n2c3ccccc3c3c2ccc2c4ccccc4n(-c4nc(-c5cccc([Si](c6ccccc6)(c6ccccc6)c6ccccc6)c5)cc(-n5c6ccccc6c6ccc([Si](c7ccccc7)(c7ccccc7)c7ccccc7)cc65)n4)c23)cc1]

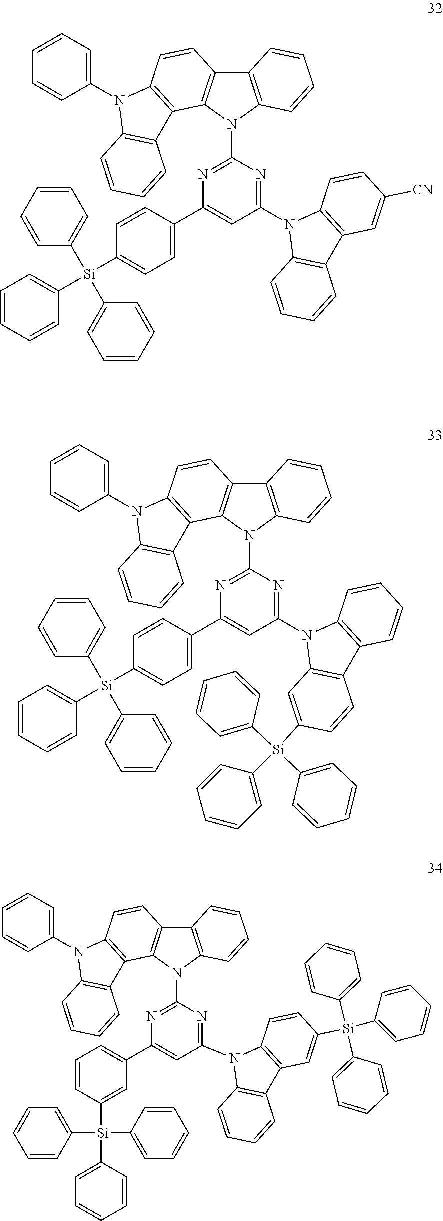 [SMILES: N#Cc1ccc2c(c1)c1ccccc1n2-c1cc(-c2ccc([Si](c3ccccc3)(c3ccccc3)c3ccccc3)cc2)nc(-n2c3ccccc3c3ccc4c(c5ccccc5n4-c4ccccc4)c32)n1 ; c1ccc(-n2c3ccccc3c3c2ccc2c4ccccc4n(-c4nc(-c5ccc([Si](c6ccccc6)(c6ccccc6)c6ccccc6)cc5)cc(-n5c6ccccc6c6ccc([Si](c7ccccc7)(c7ccccc7)c7ccccc7)cc65)n4)c23)cc1 ; c1ccc(-n2c3ccccc3c3c2ccc2c4ccccc4n(-c4nc(-c5cccc([Si](c6ccccc6)(c6ccccc6)c6ccccc6)c5)cc(-n5c6ccccc6c6cc([Si](c7ccccc7)(c7ccccc7)c7ccccc7)ccc65)n4)c23)cc1]

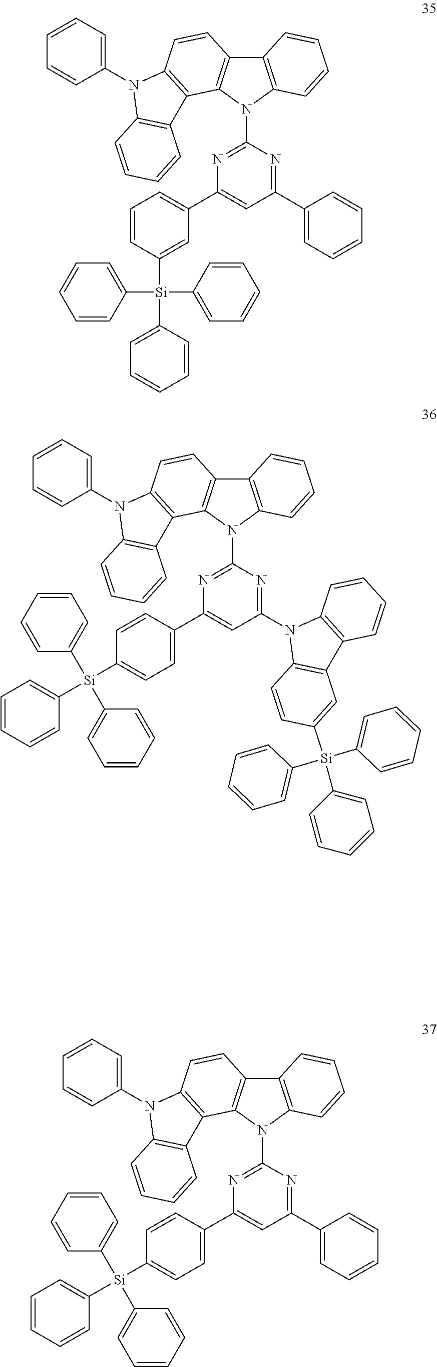 [SMILES: c1ccc(-c2cc(-c3cccc([Si](c4ccccc4)(c4ccccc4)c4ccccc4)c3)nc(-n3c4ccccc4c4ccc5c(c6ccccc6n5-c5ccccc5)c43)n2)cc1 ; c1ccc(-n2c3ccccc3c3c2ccc2c4ccccc4n(-c4nc(-c5ccc([Si](c6ccccc6)(c6ccccc6)c6ccccc6)cc5)cc(-n5c6ccccc6c6cc([Si](c7ccccc7)(c7ccccc7)c7ccccc7)ccc65)n4)c23)cc1 ; c1ccc(-c2cc(-c3ccc([Si](c4ccccc4)(c4ccccc4)c4ccccc4)cc3)nc(-n3c4ccccc4c4ccc5c(c6ccccc6n5-c5ccccc5)c43)n2)cc1]

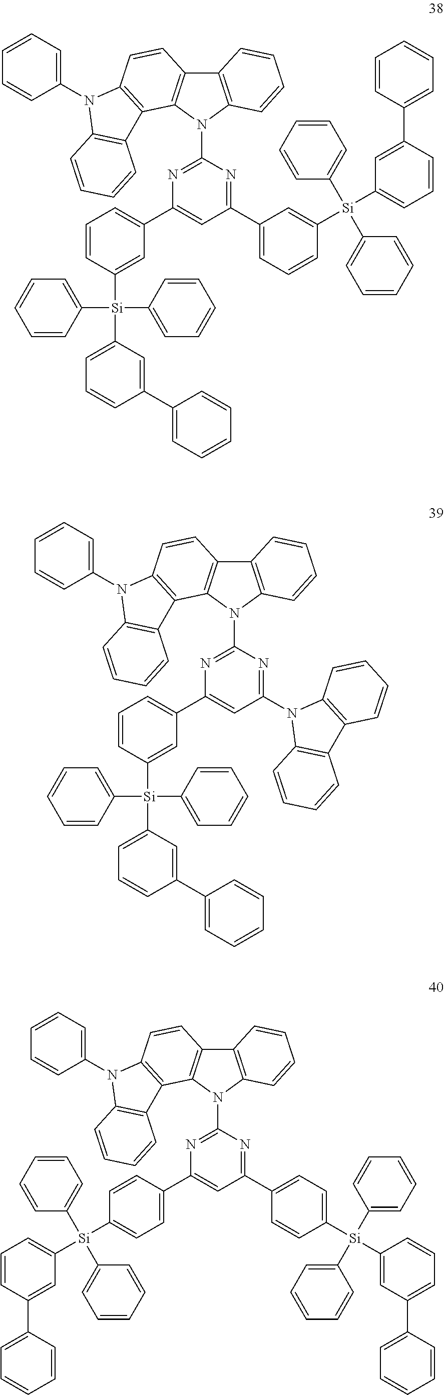 [SMILES: c1ccc(-c2cccc([Si](c3ccccc3)(c3ccccc3)c3cccc(-c4cc(-c5cccc([Si](c6ccccc6)(c6ccccc6)c6cccc(-c7ccccc7)c6)c5)nc(-n5c6ccccc6c6ccc7c(c8ccccc8n7-c7ccccc7)c65)n4)c3)c2)cc1 ; c1ccc(-c2cccc([Si](c3ccccc3)(c3ccccc3)c3cccc(-c4cc(-n5c6ccccc6c6ccccc65)nc(-n5c6ccccc6c6ccc7c(c8ccccc8n7-c7ccccc7)c65)n4)c3)c2)cc1 ; c1ccc(-c2cccc([Si](c3ccccc3)(c3ccccc3)c3ccc(-c4cc(-c5ccc([Si](c6ccccc6)(c6ccccc6)c6cccc(-c7ccccc7)c6)cc5)nc(-n5c6ccccc6c6ccc7c(c8ccccc8n7-c7ccccc7)c65)n4)cc3)c2)cc1]

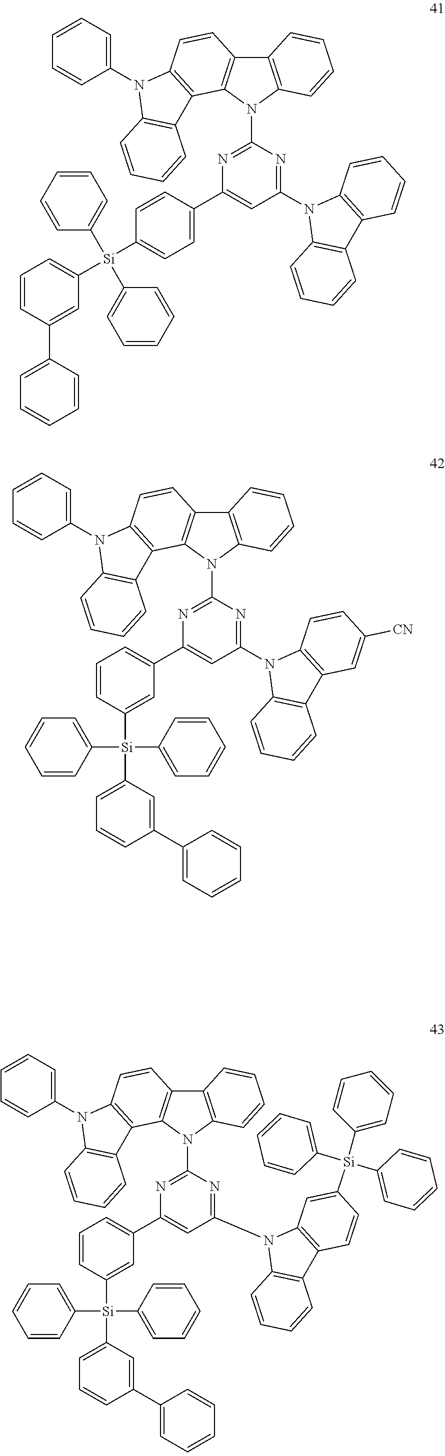

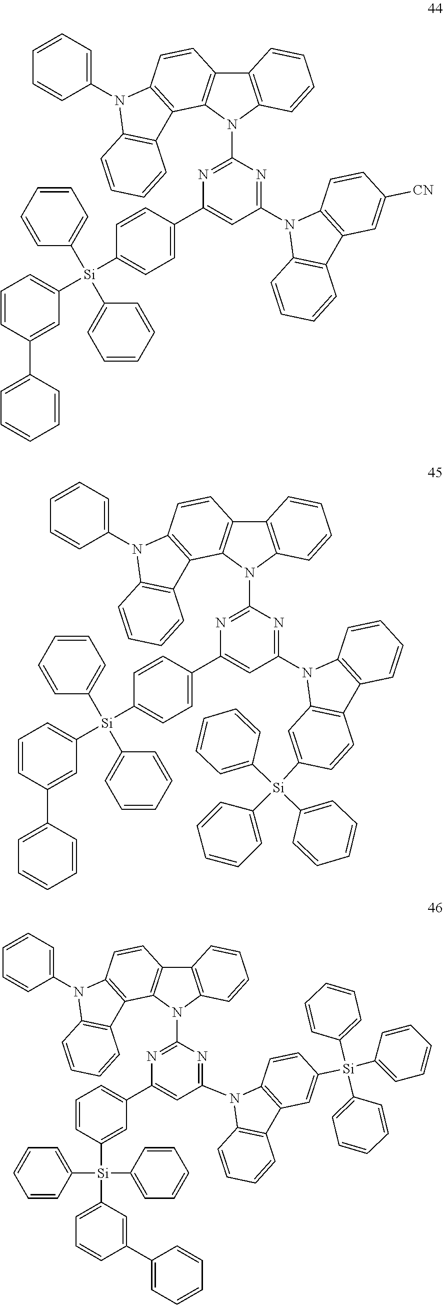 [SMILES: N#Cc1ccc2c(c1)c1ccccc1n2-c1cc(-c2ccc([Si](c3ccccc3)(c3ccccc3)c3cccc(-c4ccccc4)c3)cc2)nc(-n2c3ccccc3c3ccc4c(c5ccccc5n4-c4ccccc4)c32)n1 ; c1ccc(-c2cccc([Si](c3ccccc3)(c3ccccc3)c3ccc(-c4cc(-n5c6ccccc6c6ccc([Si](c7ccccc7)(c7ccccc7)c7ccccc7)cc65)nc(-n5c6ccccc6c6ccc7c(c8ccccc8n7-c7ccccc7)c65)n4)cc3)c2)cc1 ; c1ccc(-c2cccc([Si](c3ccccc3)(c3ccccc3)c3cccc(-c4cc(-n5c6ccccc6c6cc([Si](c7ccccc7)(c7ccccc7)c7ccccc7)ccc65)nc(-n5c6ccccc6c6ccc7c(c8ccccc8n7-c7ccccc7)c65)n4)c3)c2)cc1]

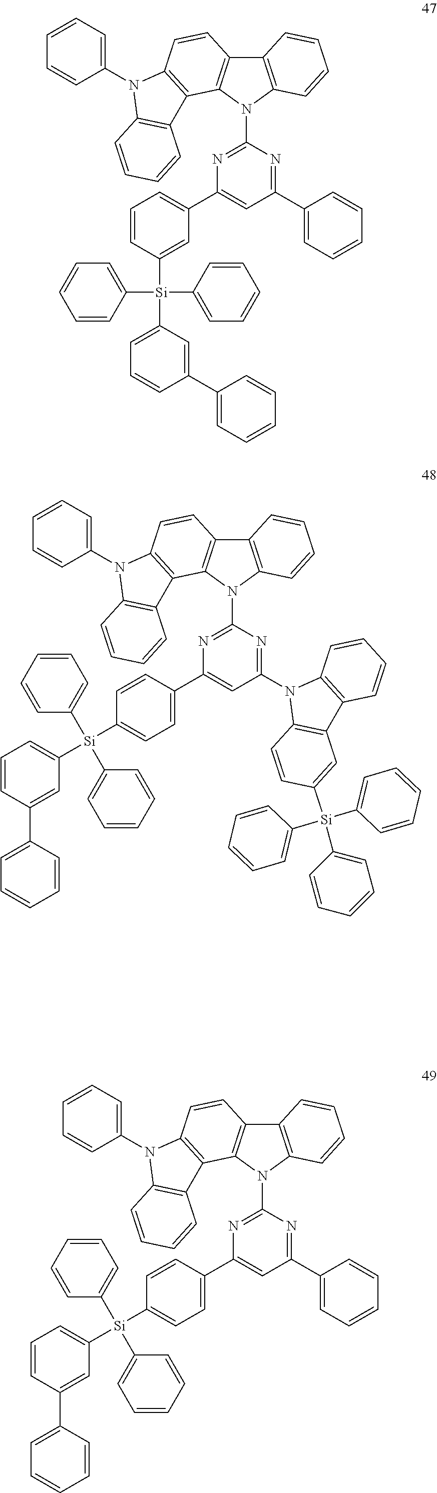 [SMILES: c1ccc(-c2cccc([Si](c3ccccc3)(c3ccccc3)c3cccc(-c4cc(-c5ccccc5)nc(-n5c6ccccc6c6ccc7c(c8ccccc8n7-c7ccccc7)c65)n4)c3)c2)cc1 ; c1ccc(-c2cccc([Si](c3ccccc3)(c3ccccc3)c3ccc(-c4cc(-n5c6ccccc6c6cc([Si](c7ccccc7)(c7ccccc7)c7ccccc7)ccc65)nc(-n5c6ccccc6c6ccc7c(c8ccccc8n7-c7ccccc7)c65)n4)cc3)c2)cc1 ; c1ccc(-c2cccc([Si](c3ccccc3)(c3ccccc3)c3ccc(-c4cc(-c5ccccc5)nc(-n5c6ccccc6c6ccc7c(c8ccccc8n7-c7ccccc7)c65)n4)cc3)c2)cc1]

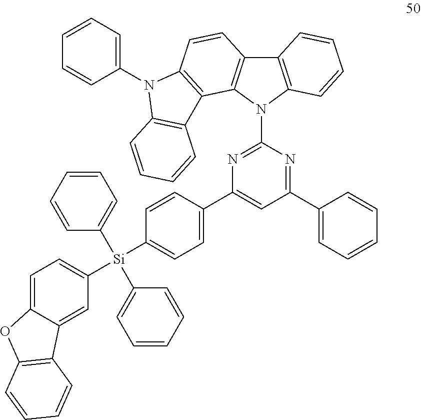 [SMILES: c1ccc(-c2cc(-c3ccc([Si](c4ccccc4)(c4ccccc4)c4ccc5oc6ccccc6c5c4)cc3)nc(-n3c4ccccc4c4ccc5c(c6ccccc6n5-c5ccccc5)c43)n2)cc1]

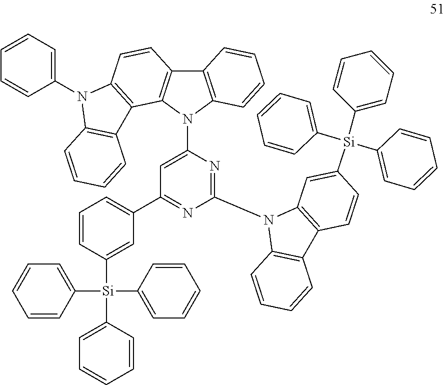 [SMILES: c1ccc(-n2c3ccccc3c3c2ccc2c4ccccc4n(-c4cc(-c5cccc([Si](c6ccccc6)(c6ccccc6)c6ccccc6)c5)nc(-n5c6ccccc6c6ccc([Si](c7ccccc7)(c7ccccc7)c7ccccc7)cc65)n4)c23)cc1]

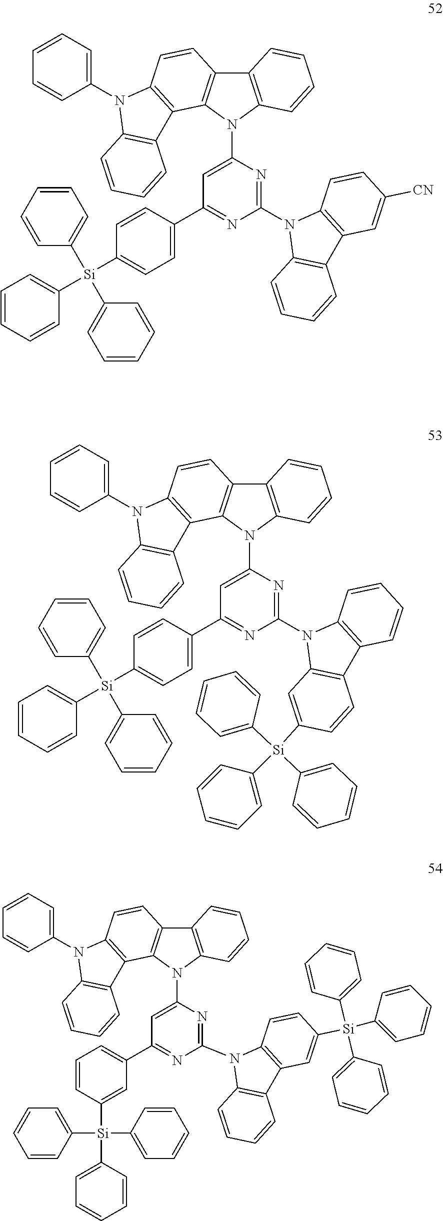 [SMILES: N#Cc1ccc2c(c1)c1ccccc1n2-c1nc(-c2ccc([Si](c3ccccc3)(c3ccccc3)c3ccccc3)cc2)cc(-n2c3ccccc3c3ccc4c(c5ccccc5n4-c4ccccc4)c32)n1 ; c1ccc(-n2c3ccccc3c3c2ccc2c4ccccc4n(-c4cc(-c5ccc([Si](c6ccccc6)(c6ccccc6)c6ccccc6)cc5)nc(-n5c6ccccc6c6ccc([Si](c7ccccc7)(c7ccccc7)c7ccccc7)cc65)n4)c23)cc1 ; c1ccc(-n2c3ccccc3c3c2ccc2c4ccccc4n(-c4cc(-c5cccc([Si](c6ccccc6)(c6ccccc6)c6ccccc6)c5)nc(-n5c6ccccc6c6cc([Si](c7ccccc7)(c7ccccc7)c7ccccc7)ccc65)n4)c23)cc1]

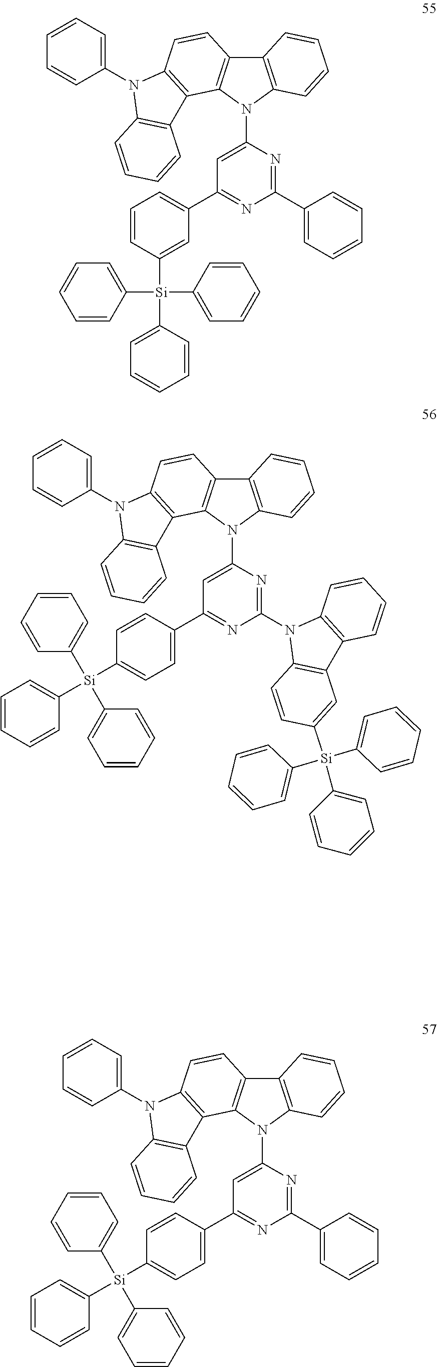 [SMILES: c1ccc(-c2nc(-c3cccc([Si](c4ccccc4)(c4ccccc4)c4ccccc4)c3)cc(-n3c4ccccc4c4ccc5c(c6ccccc6n5-c5ccccc5)c43)n2)cc1 ; c1ccc(-n2c3ccccc3c3c2ccc2c4ccccc4n(-c4cc(-c5ccc([Si](c6ccccc6)(c6ccccc6)c6ccccc6)cc5)nc(-n5c6ccccc6c6cc([Si](c7ccccc7)(c7ccccc7)c7ccccc7)ccc65)n4)c23)cc1 ; c1ccc(-c2nc(-c3ccc([Si](c4ccccc4)(c4ccccc4)c4ccccc4)cc3)cc(-n3c4ccccc4c4ccc5c(c6ccccc6n5-c5ccccc5)c43)n2)cc1]

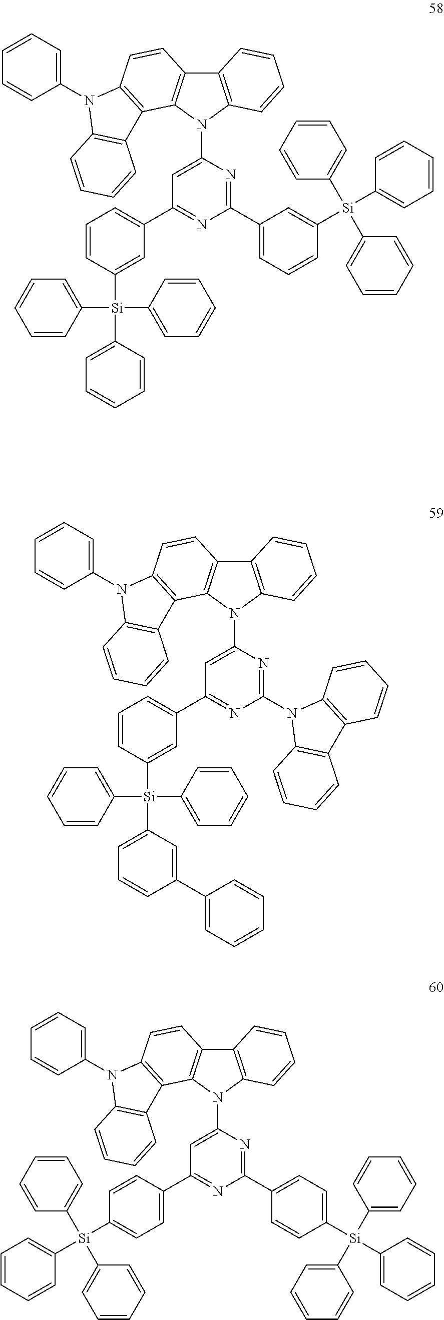 [SMILES: c1ccc(-n2c3ccccc3c3c2ccc2c4ccccc4n(-c4cc(-c5cccc([Si](c6ccccc6)(c6ccccc6)c6ccccc6)c5)nc(-c5cccc([Si](c6ccccc6)(c6ccccc6)c6ccccc6)c5)n4)c23)cc1 ; c1ccc(-c2cccc([Si](c3ccccc3)(c3ccccc3)c3cccc(-c4cc(-n5c6ccccc6c6ccc7c(c8ccccc8n7-c7ccccc7)c65)nc(-n5c6ccccc6c6ccccc65)n4)c3)c2)cc1 ; c1ccc(-n2c3ccccc3c3c2ccc2c4ccccc4n(-c4cc(-c5ccc([Si](c6ccccc6)(c6ccccc6)c6ccccc6)cc5)nc(-c5ccc([Si](c6ccccc6)(c6ccccc6)c6ccccc6)cc5)n4)c23)cc1]

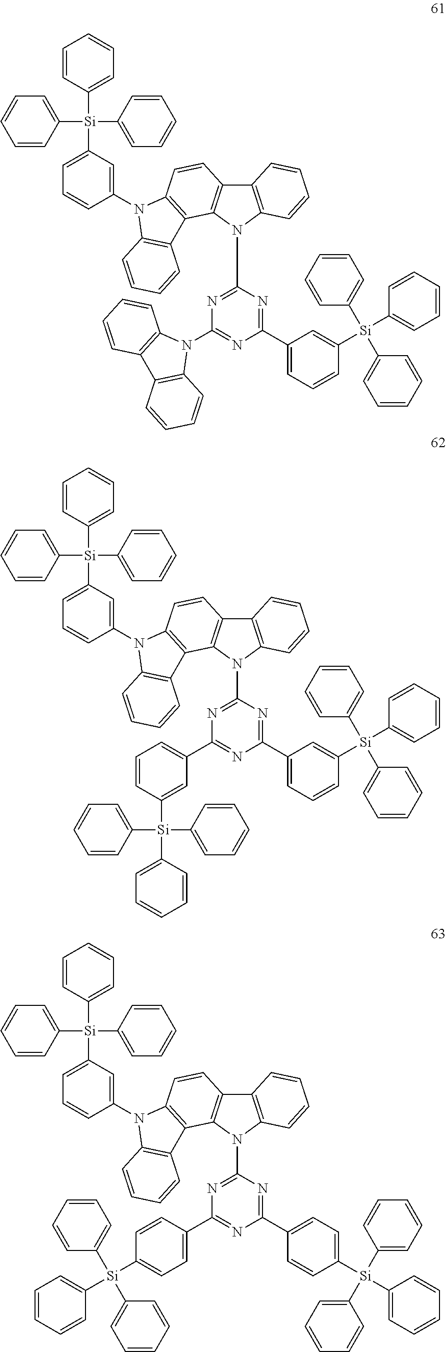

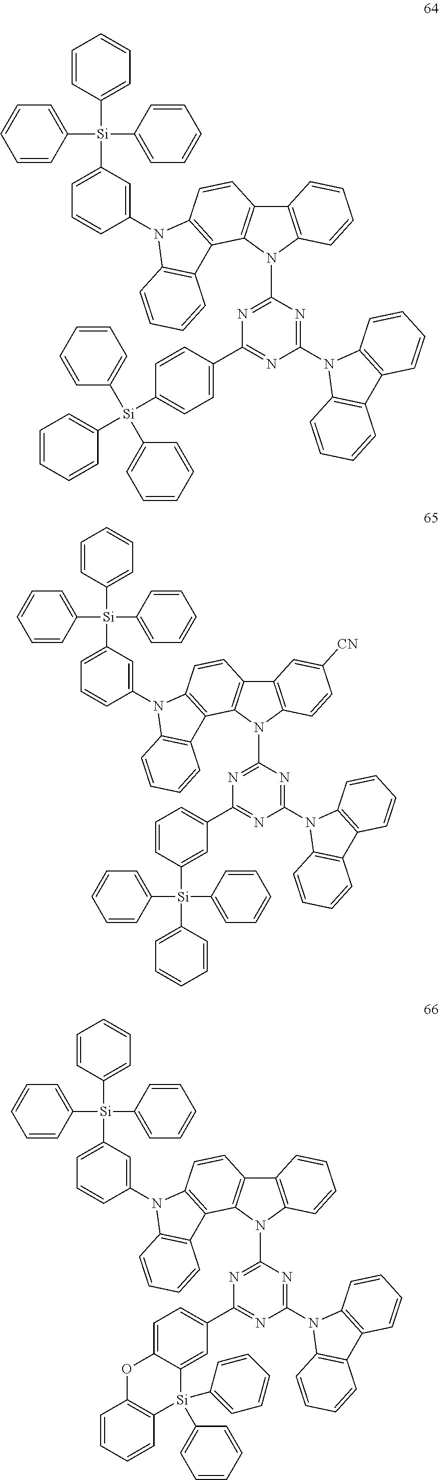 [SMILES: c1ccc([Si](c2ccccc2)(c2ccccc2)c2ccc(-c3nc(-n4c5ccccc5c5ccccc54)nc(-n4c5ccccc5c5ccc6c(c7ccccc7n6-c6cccc([Si](c7ccccc7)(c7ccccc7)c7ccccc7)c6)c54)n3)cc2)cc1 ; N#Cc1ccc2c(c1)c1ccc3c(c4ccccc4n3-c3cccc([Si](c4ccccc4)(c4ccccc4)c4ccccc4)c3)c1n2-c1nc(-c2cccc([Si](c3ccccc3)(c3ccccc3)c3ccccc3)c2)nc(-n2c3ccccc3c3ccccc32)n1 ; c1ccc([Si](c2ccccc2)(c2ccccc2)c2cccc(-n3c4ccccc4c4c3ccc3c5ccccc5n(-c5nc(-c6ccc7c(c6)[Si](c6ccccc6)(c6ccccc6)c6ccccc6O7)nc(-n6c7ccccc7c7ccccc76)n5)c34)c2)cc1]

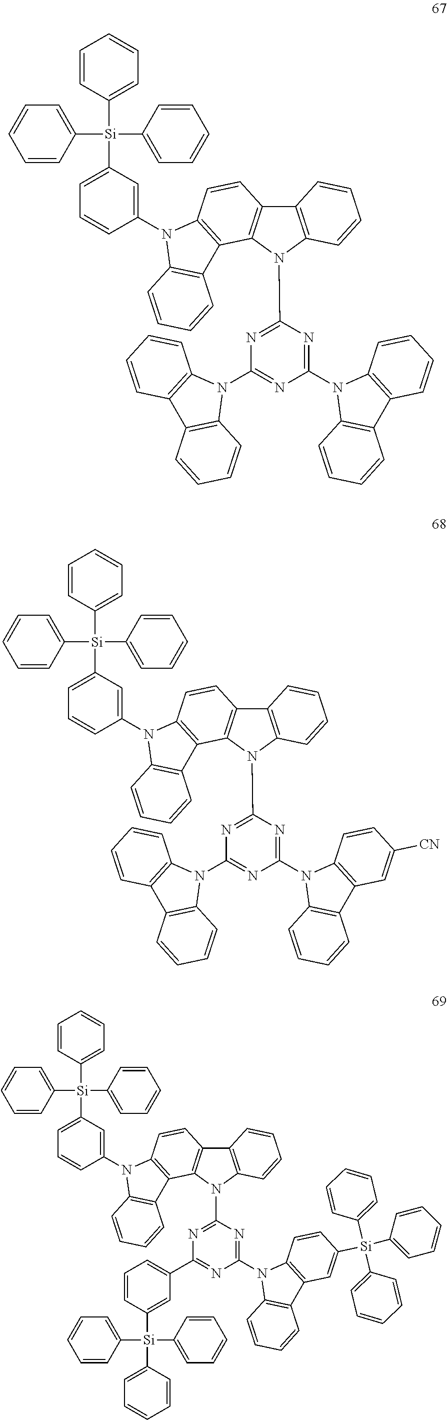 [SMILES: c1ccc([Si](c2ccccc2)(c2ccccc2)c2cccc(-n3c4ccccc4c4c3ccc3c5ccccc5n(-c5nc(-n6c7ccccc7c7ccccc76)nc(-n6c7ccccc7c7ccccc76)n5)c34)c2)cc1 ; N#Cc1ccc2c(c1)c1ccccc1n2-c1nc(-n2c3ccccc3c3ccccc32)nc(-n2c3ccccc3c3ccc4c(c5ccccc5n4-c4cccc([Si](c5ccccc5)(c5ccccc5)c5ccccc5)c4)c32)n1 ; c1ccc([Si](c2ccccc2)(c2ccccc2)c2cccc(-c3nc(-n4c5ccccc5c5cc([Si](c6ccccc6)(c6ccccc6)c6ccccc6)ccc54)nc(-n4c5ccccc5c5ccc6c(c7ccccc7n6-c6cccc([Si](c7ccccc7)(c7ccccc7)c7ccccc7)c6)c54)n3)c2)cc1]

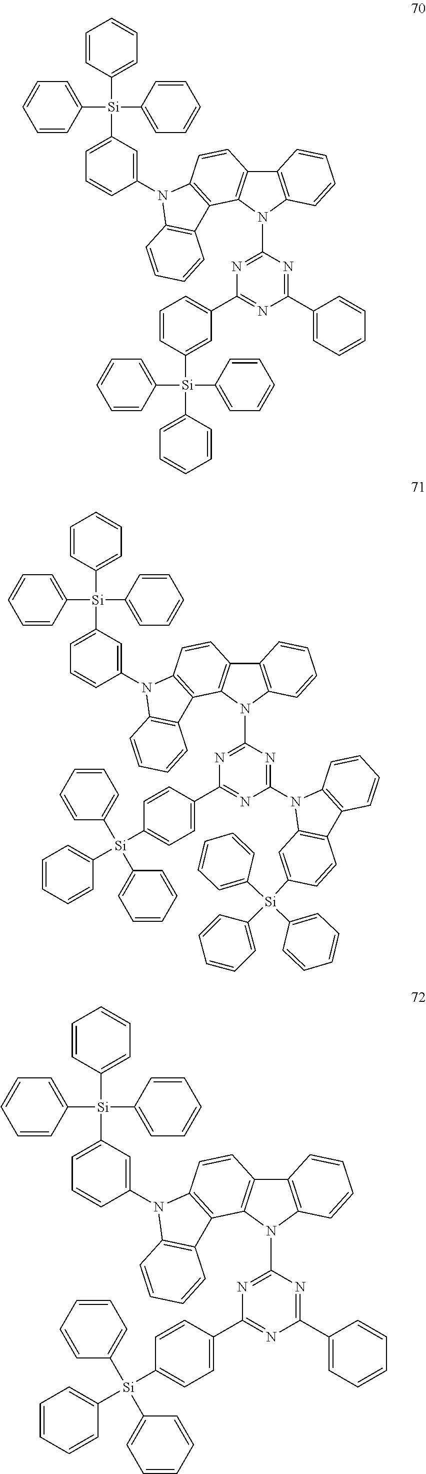 [SMILES: c1ccc(-c2nc(-c3cccc([Si](c4ccccc4)(c4ccccc4)c4ccccc4)c3)nc(-n3c4ccccc4c4ccc5c(c6ccccc6n5-c5cccc([Si](c6ccccc6)(c6ccccc6)c6ccccc6)c5)c43)n2)cc1 ; c1ccc([Si](c2ccccc2)(c2ccccc2)c2ccc(-c3nc(-n4c5ccccc5c5ccc([Si](c6ccccc6)(c6ccccc6)c6ccccc6)cc54)nc(-n4c5ccccc5c5ccc6c(c7ccccc7n6-c6cccc([Si](c7ccccc7)(c7ccccc7)c7ccccc7)c6)c54)n3)cc2)cc1 ; c1ccc(-c2nc(-c3ccc([Si](c4ccccc4)(c4ccccc4)c4ccccc4)cc3)nc(-n3c4ccccc4c4ccc5c(c6ccccc6n5-c5cccc([Si](c6ccccc6)(c6ccccc6)c6ccccc6)c5)c43)n2)cc1]

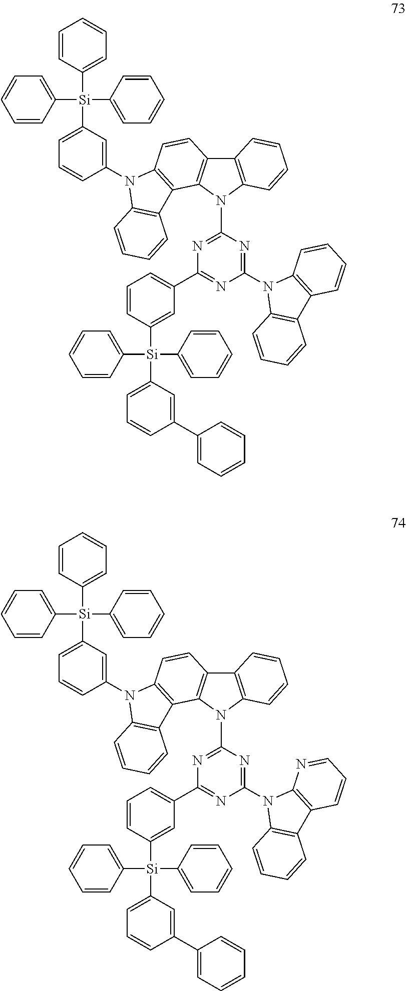 [SMILES: c1ccc(-c2cccc([Si](c3ccccc3)(c3ccccc3)c3cccc(-c4nc(-n5c6ccccc6c6ccccc65)nc(-n5c6ccccc6c6ccc7c(c8ccccc8n7-c7cccc([Si](c8ccccc8)(c8ccccc8)c8ccccc8)c7)c65)n4)c3)c2)cc1 ; c1ccc(-c2cccc([Si](c3ccccc3)(c3ccccc3)c3cccc(-c4nc(-n5c6ccccc6c6cccnc65)nc(-n5c6ccccc6c6ccc7c(c8ccccc8n7-c7cccc([Si](c8ccccc8)(c8ccccc8)c8ccccc8)c7)c65)n4)c3)c2)cc1]

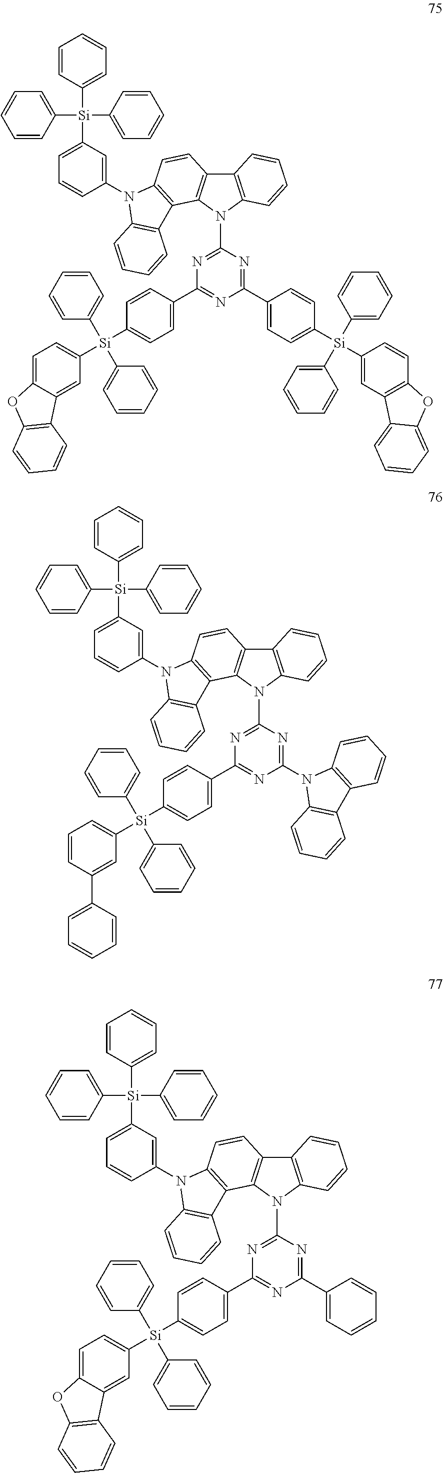

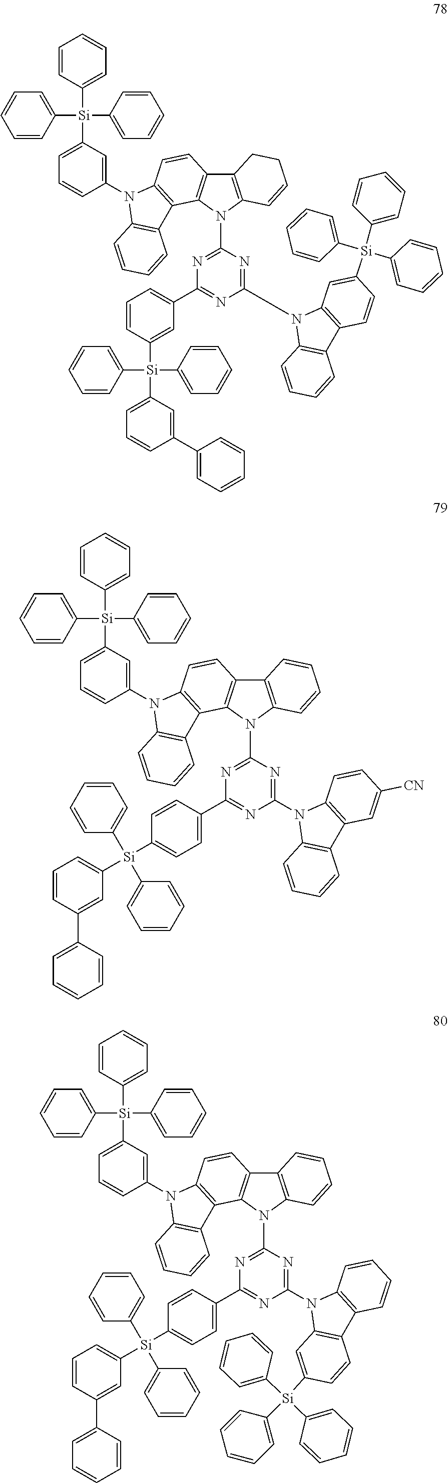

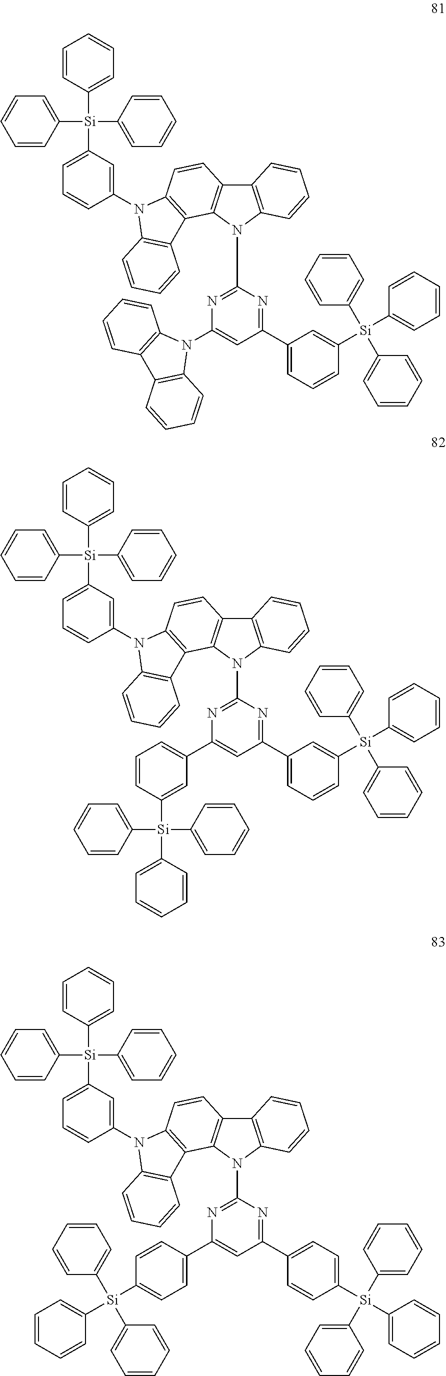

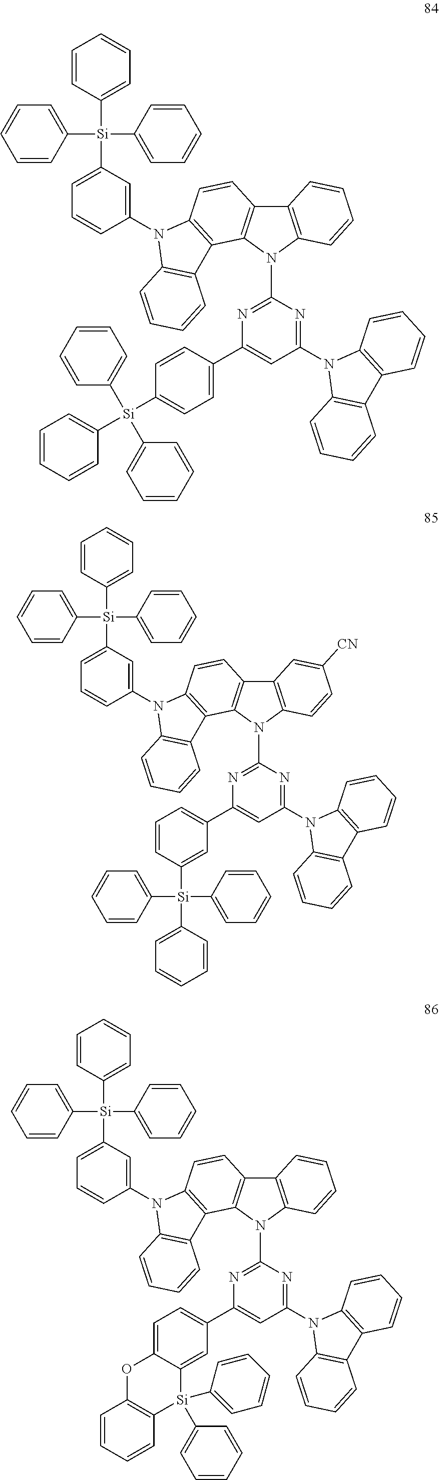 [SMILES: c1ccc([Si](c2ccccc2)(c2ccccc2)c2ccc(-c3cc(-n4c5ccccc5c5ccccc54)nc(-n4c5ccccc5c5ccc6c(c7ccccc7n6-c6cccc([Si](c7ccccc7)(c7ccccc7)c7ccccc7)c6)c54)n3)cc2)cc1 ; N#Cc1ccc2c(c1)c1ccc3c(c4ccccc4n3-c3cccc([Si](c4ccccc4)(c4ccccc4)c4ccccc4)c3)c1n2-c1nc(-c2cccc([Si](c3ccccc3)(c3ccccc3)c3ccccc3)c2)cc(-n2c3ccccc3c3ccccc32)n1 ; c1ccc([Si](c2ccccc2)(c2ccccc2)c2cccc(-n3c4ccccc4c4c3ccc3c5ccccc5n(-c5nc(-c6ccc7c(c6)[Si](c6ccccc6)(c6ccccc6)c6ccccc6O7)cc(-n6c7ccccc7c7ccccc76)n5)c34)c2)cc1]

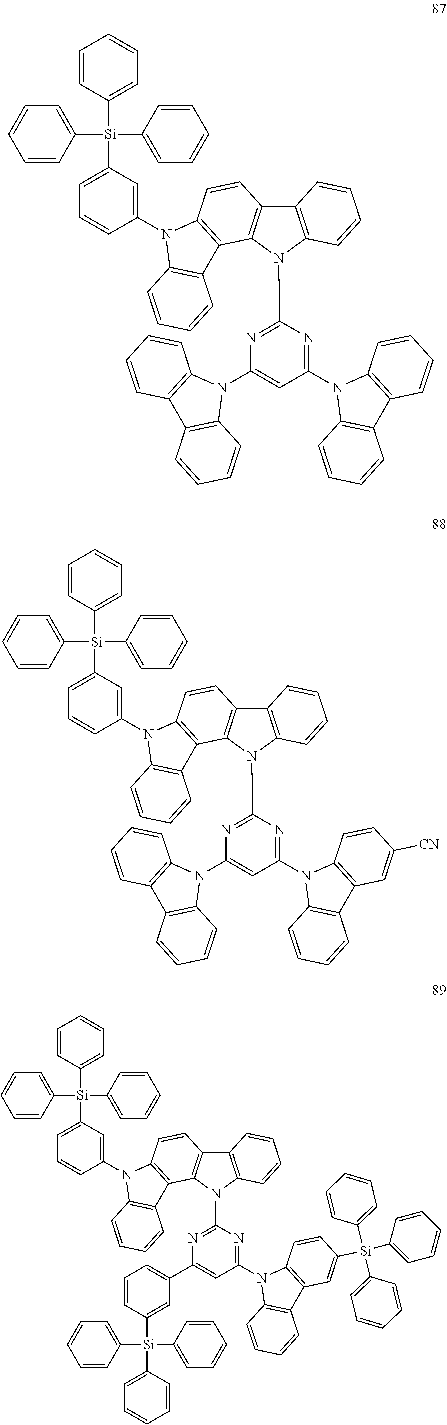 [SMILES: c1ccc([Si](c2ccccc2)(c2ccccc2)c2cccc(-n3c4ccccc4c4c3ccc3c5ccccc5n(-c5nc(-n6c7ccccc7c7ccccc76)cc(-n6c7ccccc7c7ccccc76)n5)c34)c2)cc1 ; N#Cc1ccc2c(c1)c1ccccc1n2-c1cc(-n2c3ccccc3c3ccccc32)nc(-n2c3ccccc3c3ccc4c(c5ccccc5n4-c4cccc([Si](c5ccccc5)(c5ccccc5)c5ccccc5)c4)c32)n1 ; c1ccc([Si](c2ccccc2)(c2ccccc2)c2cccc(-c3cc(-n4c5ccccc5c5cc([Si](c6ccccc6)(c6ccccc6)c6ccccc6)ccc54)nc(-n4c5ccccc5c5ccc6c(c7ccccc7n6-c6cccc([Si](c7ccccc7)(c7ccccc7)c7ccccc7)c6)c54)n3)c2)cc1]

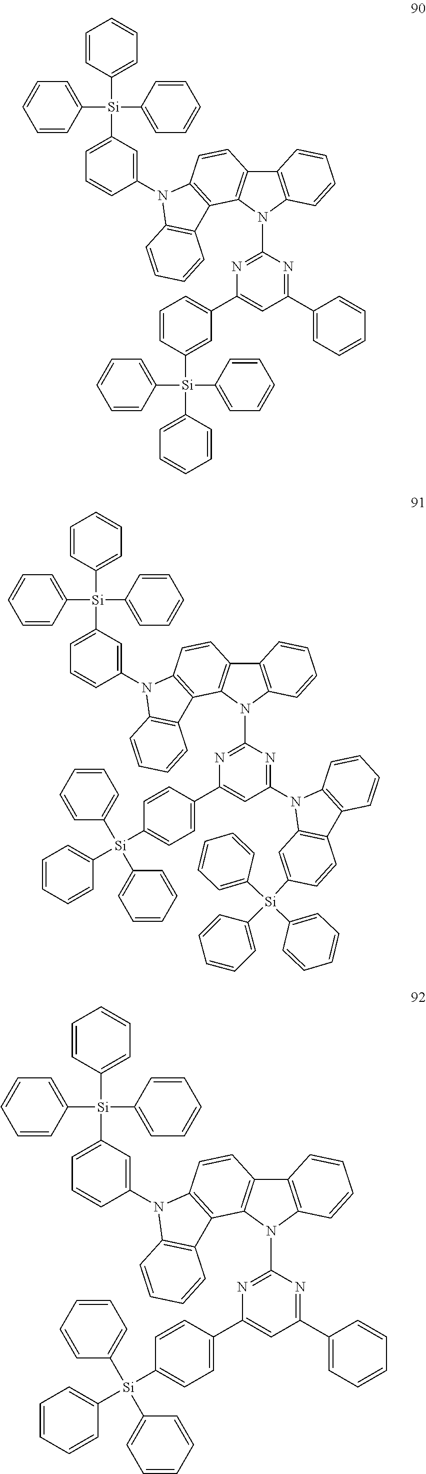 [SMILES: c1ccc(-c2cc(-c3cccc([Si](c4ccccc4)(c4ccccc4)c4ccccc4)c3)nc(-n3c4ccccc4c4ccc5c(c6ccccc6n5-c5cccc([Si](c6ccccc6)(c6ccccc6)c6ccccc6)c5)c43)n2)cc1 ; c1ccc([Si](c2ccccc2)(c2ccccc2)c2ccc(-c3cc(-n4c5ccccc5c5ccc([Si](c6ccccc6)(c6ccccc6)c6ccccc6)cc54)nc(-n4c5ccccc5c5ccc6c(c7ccccc7n6-c6cccc([Si](c7ccccc7)(c7ccccc7)c7ccccc7)c6)c54)n3)cc2)cc1 ; c1ccc(-c2cc(-c3ccc([Si](c4ccccc4)(c4ccccc4)c4ccccc4)cc3)nc(-n3c4ccccc4c4ccc5c(c6ccccc6n5-c5cccc([Si](c6ccccc6)(c6ccccc6)c6ccccc6)c5)c43)n2)cc1]

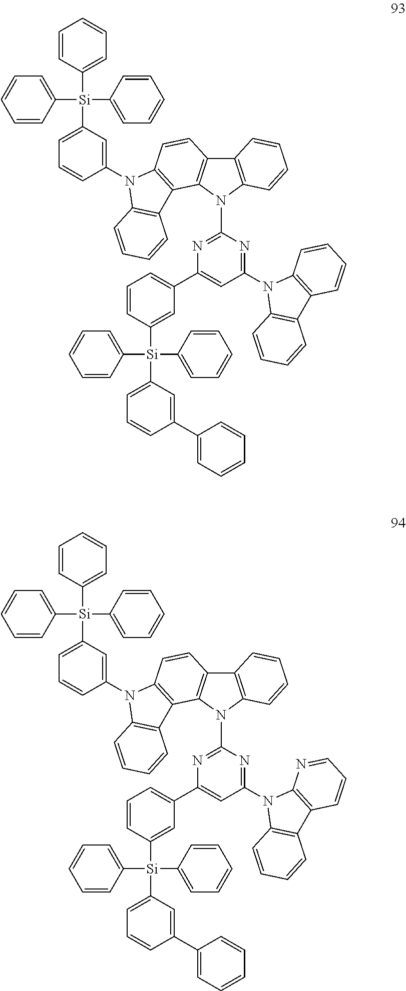 [SMILES: c1ccc(-c2cccc([Si](c3ccccc3)(c3ccccc3)c3cccc(-c4cc(-n5c6ccccc6c6ccccc65)nc(-n5c6ccccc6c6ccc7c(c8ccccc8n7-c7cccc([Si](c8ccccc8)(c8ccccc8)c8ccccc8)c7)c65)n4)c3)c2)cc1 ; c1ccc(-c2cccc([Si](c3ccccc3)(c3ccccc3)c3cccc(-c4cc(-n5c6ccccc6c6cccnc65)nc(-n5c6ccccc6c6ccc7c(c8ccccc8n7-c7cccc([Si](c8ccccc8)(c8ccccc8)c8ccccc8)c7)c65)n4)c3)c2)cc1]

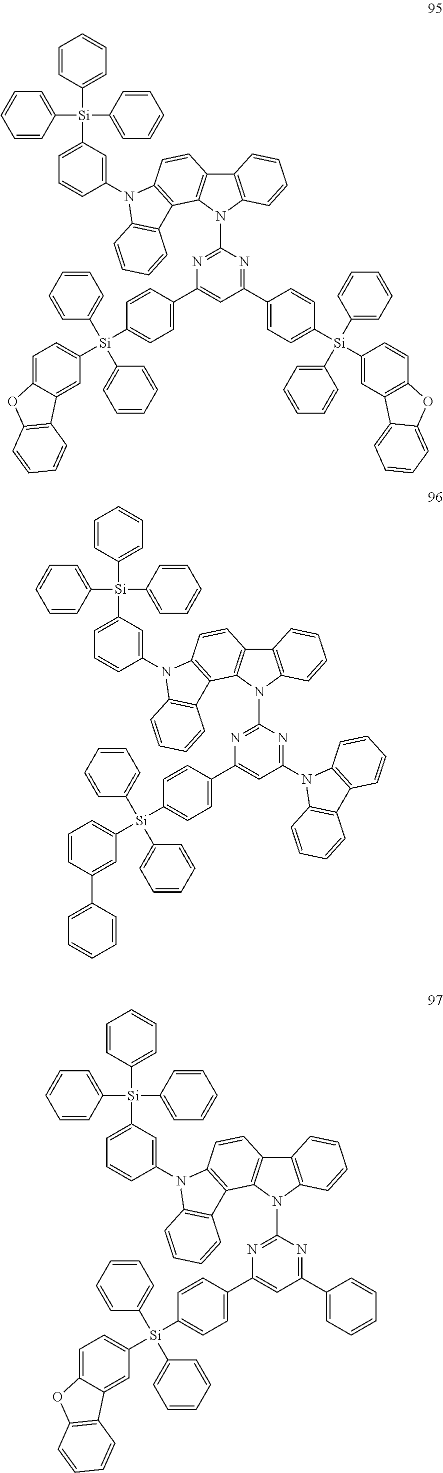

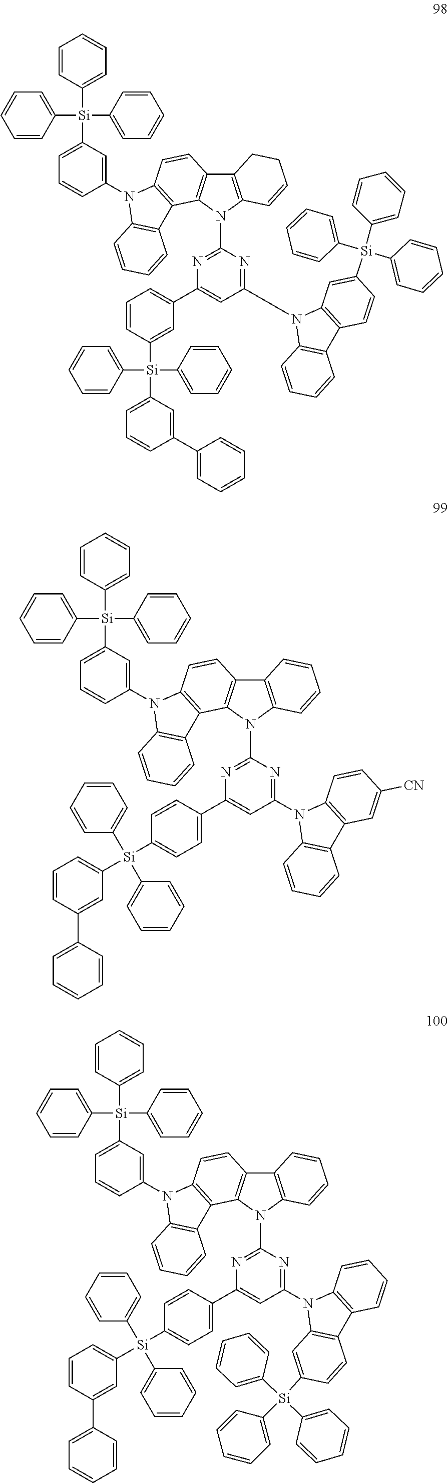

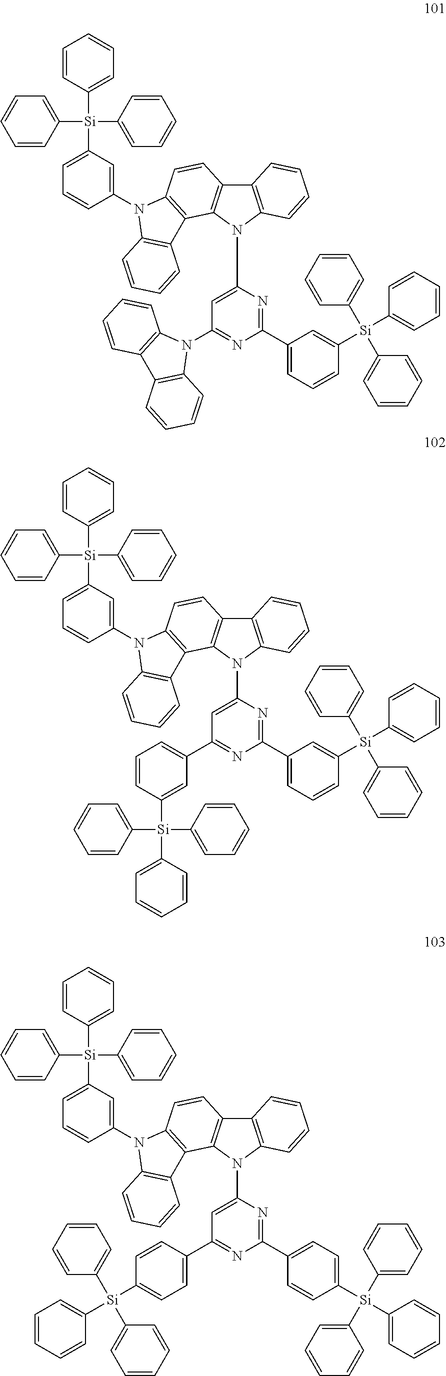

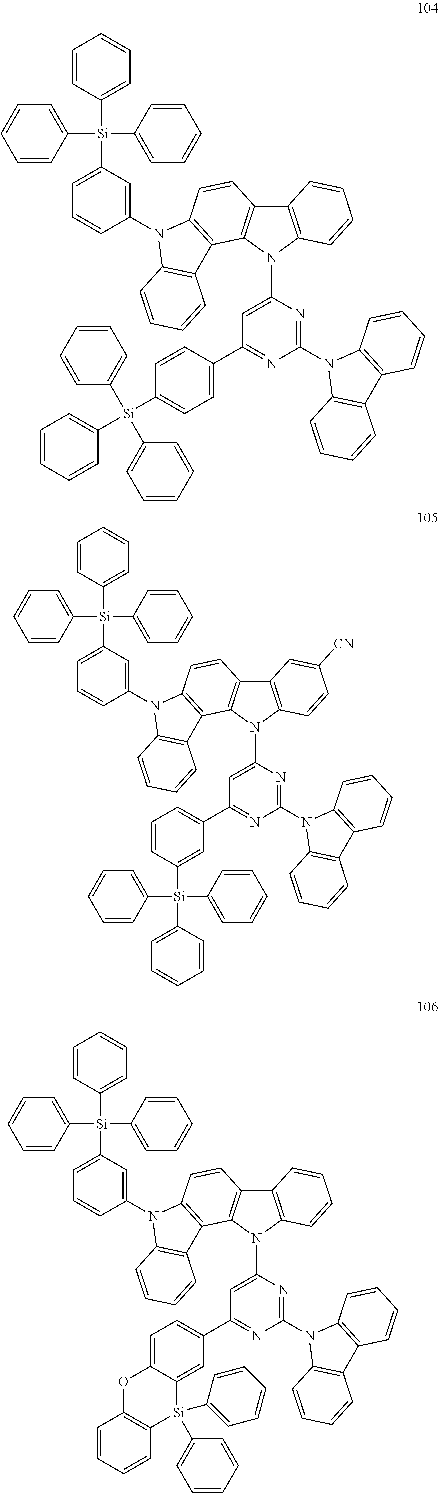 [SMILES: c1ccc([Si](c2ccccc2)(c2ccccc2)c2ccc(-c3cc(-n4c5ccccc5c5ccc6c(c7ccccc7n6-c6cccc([Si](c7ccccc7)(c7ccccc7)c7ccccc7)c6)c54)nc(-n4c5ccccc5c5ccccc54)n3)cc2)cc1 ; N#Cc1ccc2c(c1)c1ccc3c(c4ccccc4n3-c3cccc([Si](c4ccccc4)(c4ccccc4)c4ccccc4)c3)c1n2-c1cc(-c2cccc([Si](c3ccccc3)(c3ccccc3)c3ccccc3)c2)nc(-n2c3ccccc3c3ccccc32)n1 ; c1ccc([Si](c2ccccc2)(c2ccccc2)c2cccc(-n3c4ccccc4c4c3ccc3c5ccccc5n(-c5cc(-c6ccc7c(c6)[Si](c6ccccc6)(c6ccccc6)c6ccccc6O7)nc(-n6c7ccccc7c7ccccc76)n5)c34)c2)cc1]

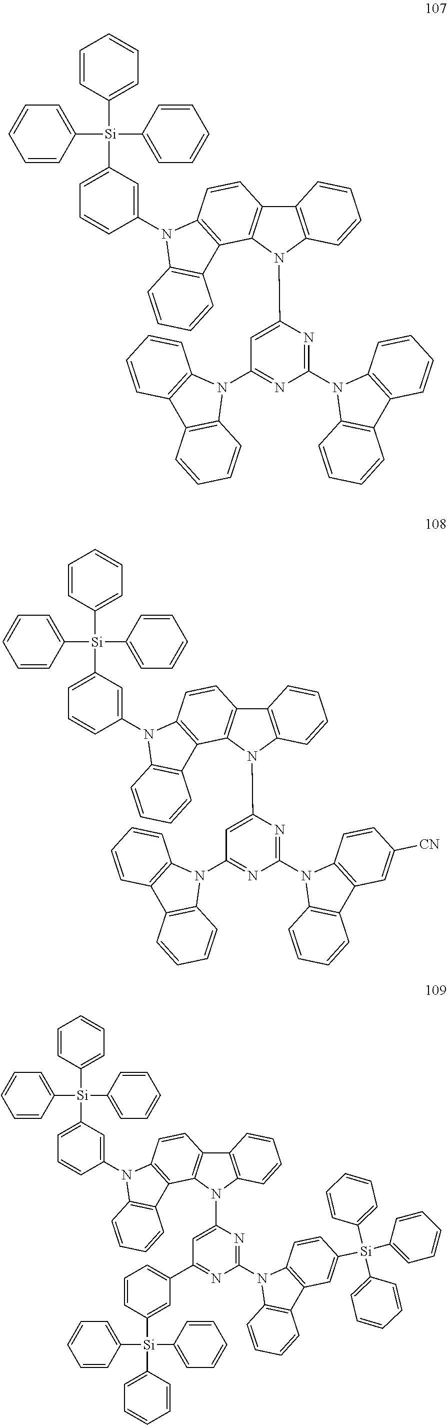 [SMILES: c1ccc([Si](c2ccccc2)(c2ccccc2)c2cccc(-n3c4ccccc4c4c3ccc3c5ccccc5n(-c5cc(-n6c7ccccc7c7ccccc76)nc(-n6c7ccccc7c7ccccc76)n5)c34)c2)cc1 ; N#Cc1ccc2c(c1)c1ccccc1n2-c1nc(-n2c3ccccc3c3ccccc32)cc(-n2c3ccccc3c3ccc4c(c5ccccc5n4-c4cccc([Si](c5ccccc5)(c5ccccc5)c5ccccc5)c4)c32)n1 ; c1ccc([Si](c2ccccc2)(c2ccccc2)c2cccc(-c3cc(-n4c5ccccc5c5ccc6c(c7ccccc7n6-c6cccc([Si](c7ccccc7)(c7ccccc7)c7ccccc7)c6)c54)nc(-n4c5ccccc5c5cc([Si](c6ccccc6)(c6ccccc6)c6ccccc6)ccc54)n3)c2)cc1]

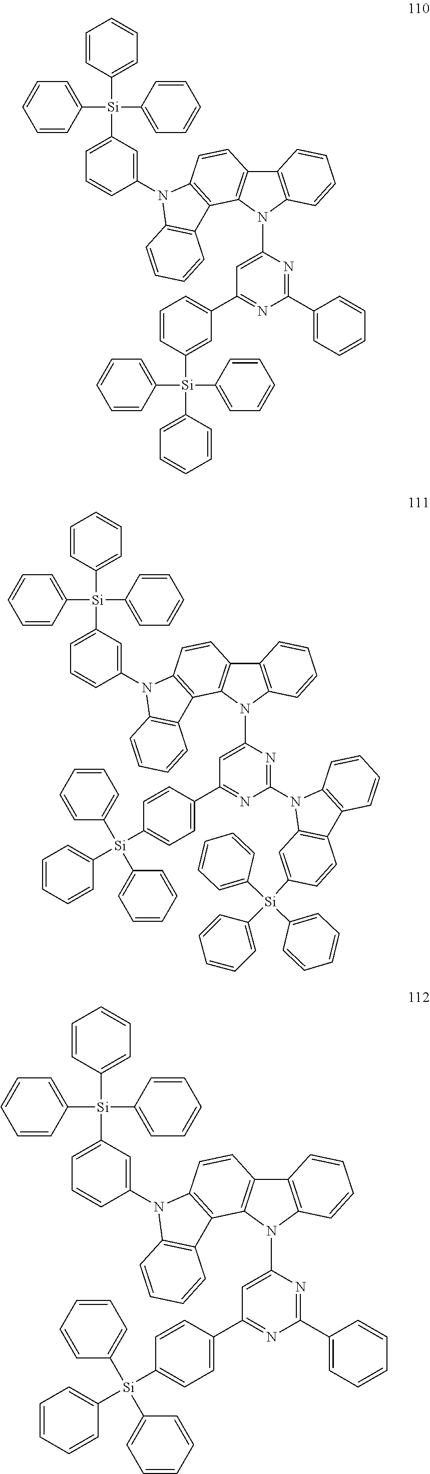 [SMILES: c1ccc(-c2nc(-c3cccc([Si](c4ccccc4)(c4ccccc4)c4ccccc4)c3)cc(-n3c4ccccc4c4ccc5c(c6ccccc6n5-c5cccc([Si](c6ccccc6)(c6ccccc6)c6ccccc6)c5)c43)n2)cc1 ; c1ccc([Si](c2ccccc2)(c2ccccc2)c2ccc(-c3cc(-n4c5ccccc5c5ccc6c(c7ccccc7n6-c6cccc([Si](c7ccccc7)(c7ccccc7)c7ccccc7)c6)c54)nc(-n4c5ccccc5c5ccc([Si](c6ccccc6)(c6ccccc6)c6ccccc6)cc54)n3)cc2)cc1 ; c1ccc(-c2nc(-c3ccc([Si](c4ccccc4)(c4ccccc4)c4ccccc4)cc3)cc(-n3c4ccccc4c4ccc5c(c6ccccc6n5-c5cccc([Si](c6ccccc6)(c6ccccc6)c6ccccc6)c5)c43)n2)cc1]

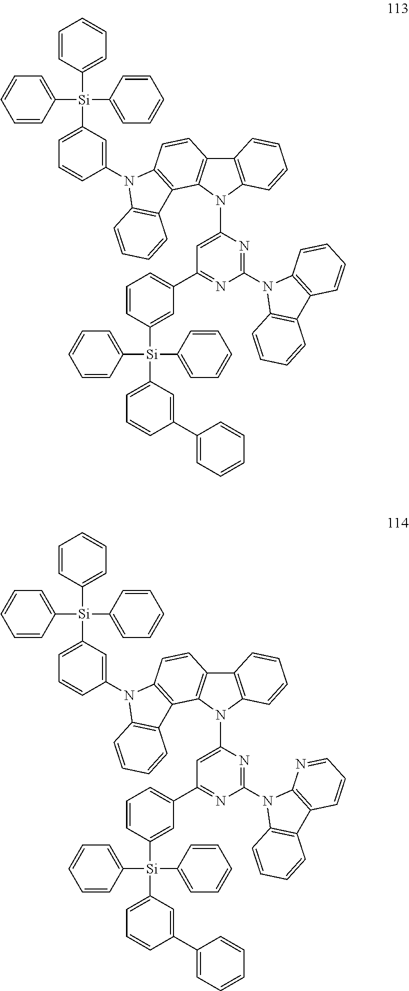 [SMILES: c1ccc(-c2cccc([Si](c3ccccc3)(c3ccccc3)c3cccc(-c4cc(-n5c6ccccc6c6ccc7c(c8ccccc8n7-c7cccc([Si](c8ccccc8)(c8ccccc8)c8ccccc8)c7)c65)nc(-n5c6ccccc6c6ccccc65)n4)c3)c2)cc1 ; c1ccc(-c2cccc([Si](c3ccccc3)(c3ccccc3)c3cccc(-c4cc(-n5c6ccccc6c6ccc7c(c8ccccc8n7-c7cccc([Si](c8ccccc8)(c8ccccc8)c8ccccc8)c7)c65)nc(-n5c6ccccc6c6cccnc65)n4)c3)c2)cc1]

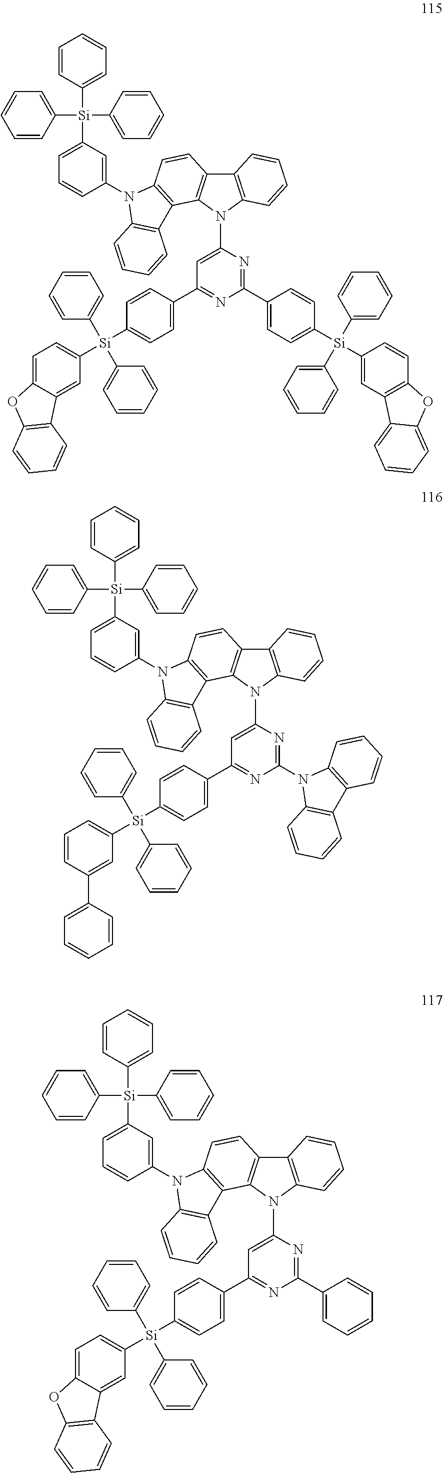

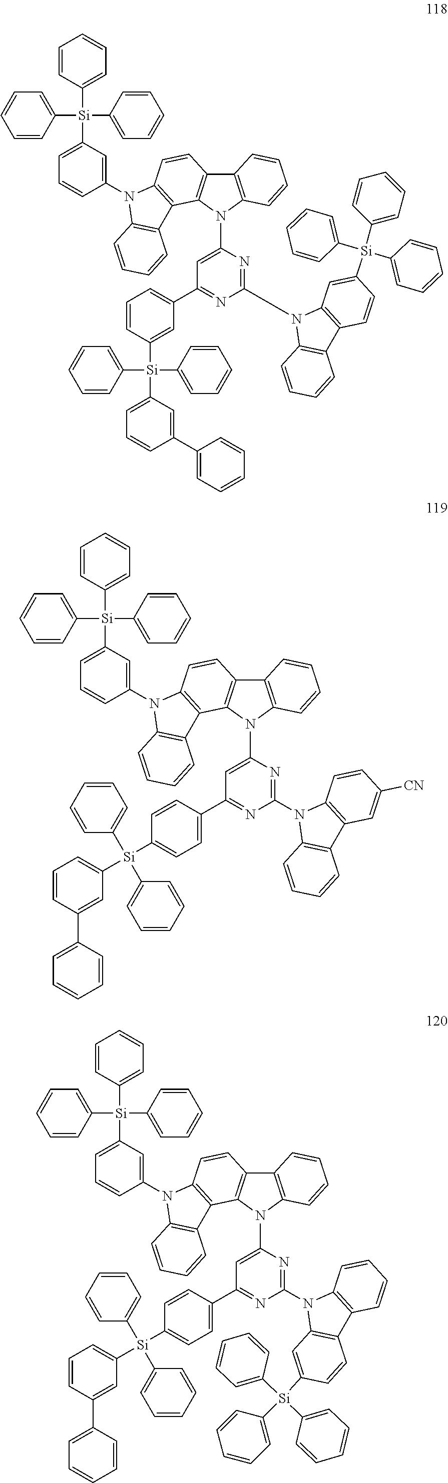

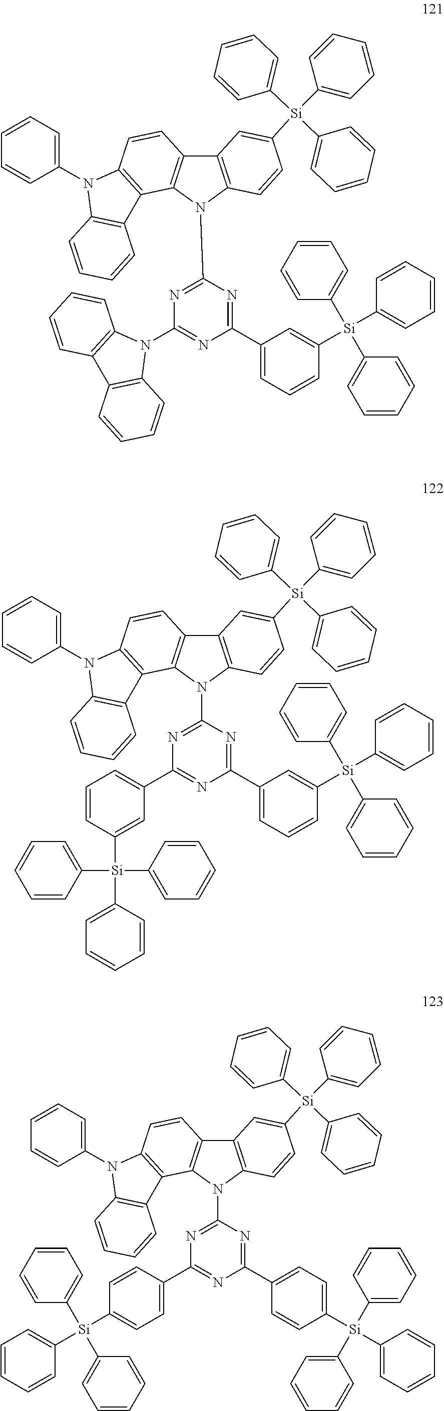

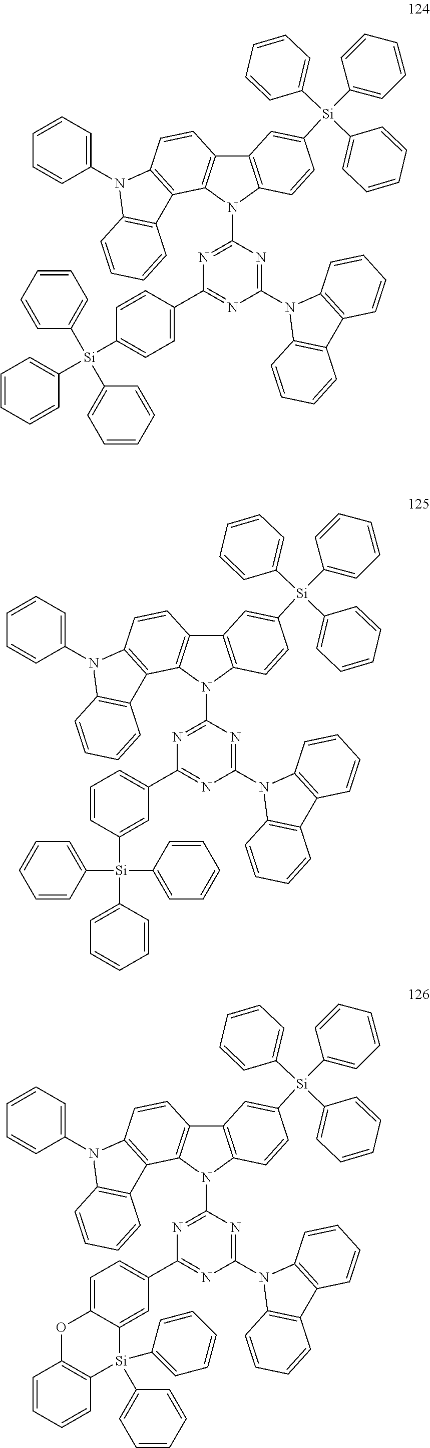 [SMILES: c1ccc(-n2c3ccccc3c3c2ccc2c4cc([Si](c5ccccc5)(c5ccccc5)c5ccccc5)ccc4n(-c4nc(-c5ccc([Si](c6ccccc6)(c6ccccc6)c6ccccc6)cc5)nc(-n5c6ccccc6c6ccccc65)n4)c23)cc1 ; c1ccc(-n2c3ccccc3c3c2ccc2c4cc([Si](c5ccccc5)(c5ccccc5)c5ccccc5)ccc4n(-c4nc(-c5cccc([Si](c6ccccc6)(c6ccccc6)c6ccccc6)c5)nc(-n5c6ccccc6c6ccccc65)n4)c23)cc1 ; c1ccc(-n2c3ccccc3c3c2ccc2c4cc([Si](c5ccccc5)(c5ccccc5)c5ccccc5)ccc4n(-c4nc(-c5ccc6c(c5)[Si](c5ccccc5)(c5ccccc5)c5ccccc5O6)nc(-n5c6ccccc6c6ccccc65)n4)c23)cc1]

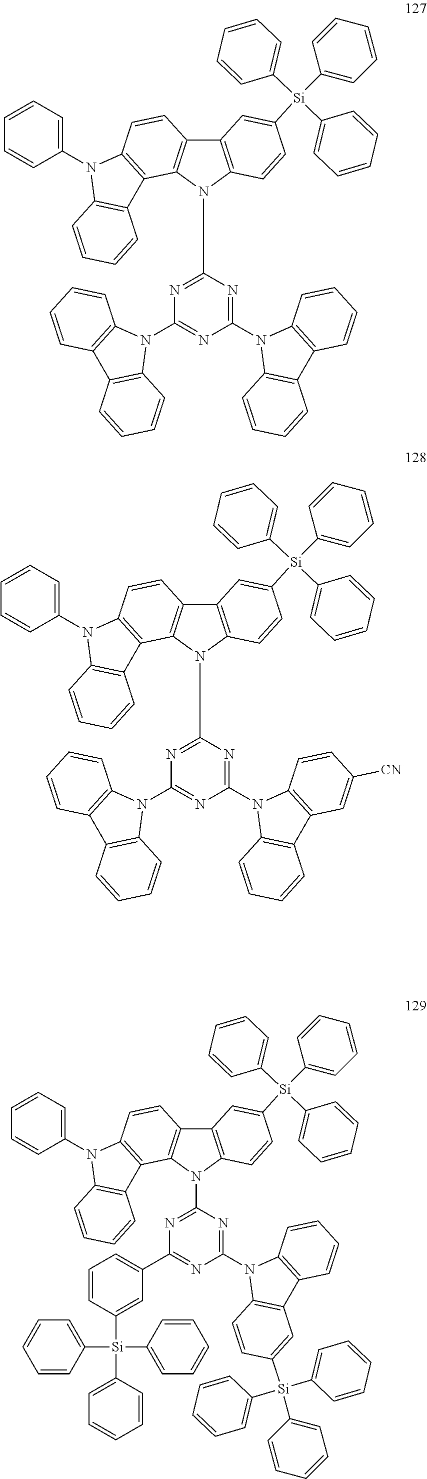 [SMILES: c1ccc(-n2c3ccccc3c3c2ccc2c4cc([Si](c5ccccc5)(c5ccccc5)c5ccccc5)ccc4n(-c4nc(-n5c6ccccc6c6ccccc65)nc(-n5c6ccccc6c6ccccc65)n4)c23)cc1 ; N#Cc1ccc2c(c1)c1ccccc1n2-c1nc(-n2c3ccccc3c3ccccc32)nc(-n2c3ccc([Si](c4ccccc4)(c4ccccc4)c4ccccc4)cc3c3ccc4c(c5ccccc5n4-c4ccccc4)c32)n1 ; c1ccc(-n2c3ccccc3c3c2ccc2c4cc([Si](c5ccccc5)(c5ccccc5)c5ccccc5)ccc4n(-c4nc(-c5cccc([Si](c6ccccc6)(c6ccccc6)c6ccccc6)c5)nc(-n5c6ccccc6c6cc([Si](c7ccccc7)(c7ccccc7)c7ccccc7)ccc65)n4)c23)cc1]

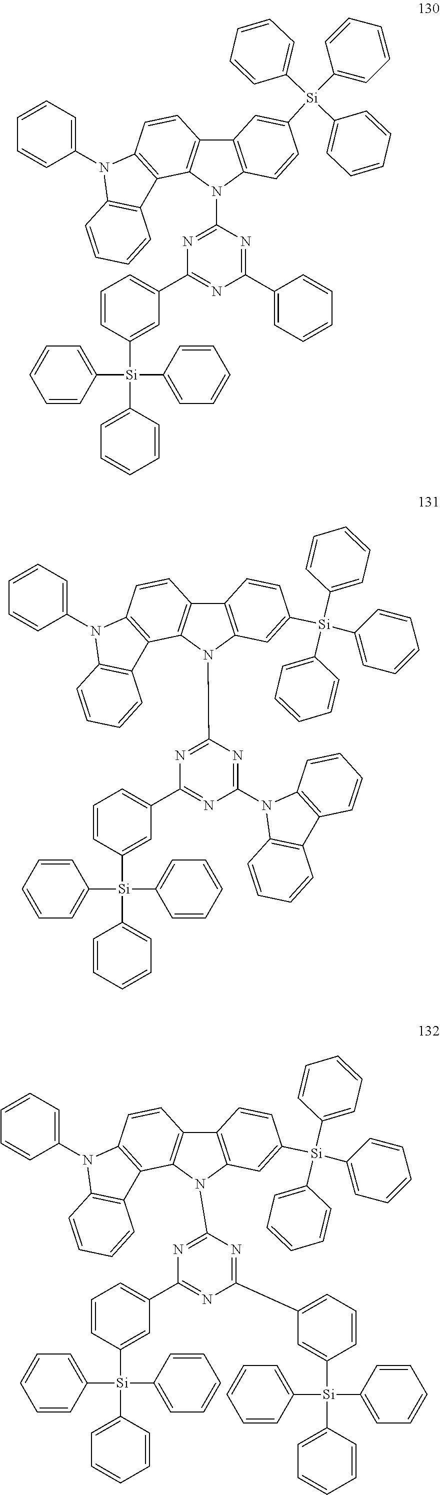 [SMILES: c1ccc(-c2nc(-c3cccc([Si](c4ccccc4)(c4ccccc4)c4ccccc4)c3)nc(-n3c4ccc([Si](c5ccccc5)(c5ccccc5)c5ccccc5)cc4c4ccc5c(c6ccccc6n5-c5ccccc5)c43)n2)cc1 ; c1ccc(-n2c3ccccc3c3c2ccc2c4ccc([Si](c5ccccc5)(c5ccccc5)c5ccccc5)cc4n(-c4nc(-c5cccc([Si](c6ccccc6)(c6ccccc6)c6ccccc6)c5)nc(-n5c6ccccc6c6ccccc65)n4)c23)cc1 ; c1ccc(-n2c3ccccc3c3c2ccc2c4ccc([Si](c5ccccc5)(c5ccccc5)c5ccccc5)cc4n(-c4nc(-c5cccc([Si](c6ccccc6)(c6ccccc6)c6ccccc6)c5)nc(-c5cccc([Si](c6ccccc6)(c6ccccc6)c6ccccc6)c5)n4)c23)cc1]

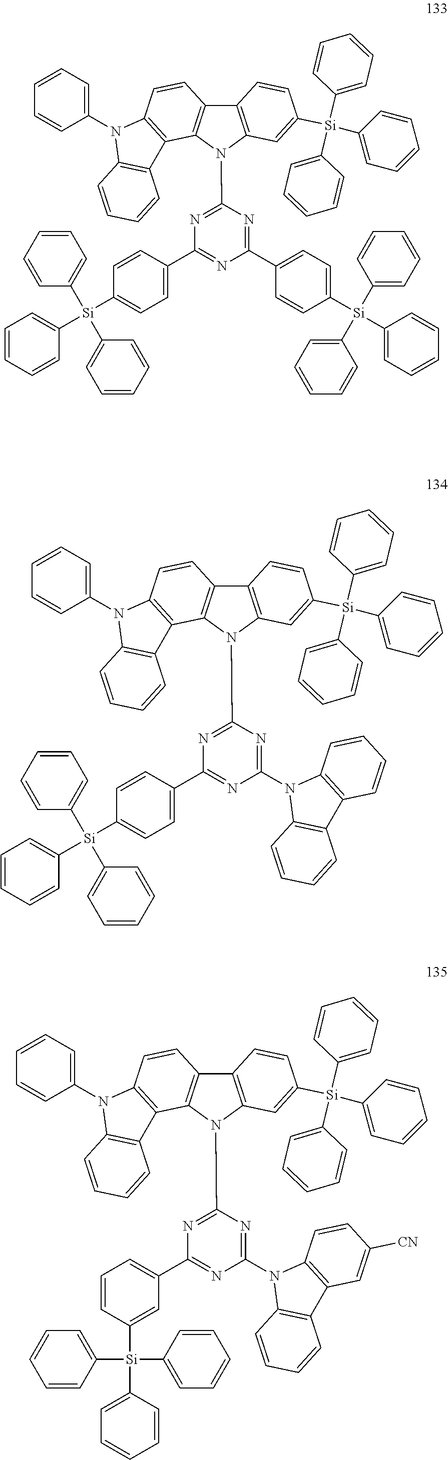 [SMILES: c1ccc(-n2c3ccccc3c3c2ccc2c4ccc([Si](c5ccccc5)(c5ccccc5)c5ccccc5)cc4n(-c4nc(-c5ccc([Si](c6ccccc6)(c6ccccc6)c6ccccc6)cc5)nc(-c5ccc([Si](c6ccccc6)(c6ccccc6)c6ccccc6)cc5)n4)c23)cc1 ; c1ccc(-n2c3ccccc3c3c2ccc2c4ccc([Si](c5ccccc5)(c5ccccc5)c5ccccc5)cc4n(-c4nc(-c5ccc([Si](c6ccccc6)(c6ccccc6)c6ccccc6)cc5)nc(-n5c6ccccc6c6ccccc65)n4)c23)cc1 ; N#Cc1ccc2c(c1)c1ccccc1n2-c1nc(-c2cccc([Si](c3ccccc3)(c3ccccc3)c3ccccc3)c2)nc(-n2c3cc([Si](c4ccccc4)(c4ccccc4)c4ccccc4)ccc3c3ccc4c(c5ccccc5n4-c4ccccc4)c32)n1]

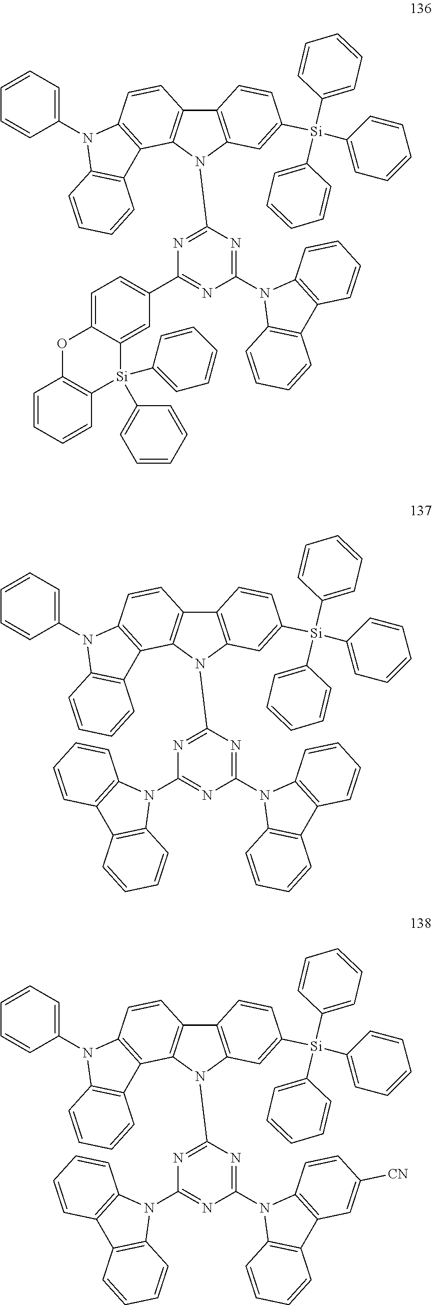 [SMILES: c1ccc(-n2c3ccccc3c3c2ccc2c4ccc([Si](c5ccccc5)(c5ccccc5)c5ccccc5)cc4n(-c4nc(-c5ccc6c(c5)[Si](c5ccccc5)(c5ccccc5)c5ccccc5O6)nc(-n5c6ccccc6c6ccccc65)n4)c23)cc1 ; c1ccc(-n2c3ccccc3c3c2ccc2c4ccc([Si](c5ccccc5)(c5ccccc5)c5ccccc5)cc4n(-c4nc(-n5c6ccccc6c6ccccc65)nc(-n5c6ccccc6c6ccccc65)n4)c23)cc1 ; N#Cc1ccc2c(c1)c1ccccc1n2-c1nc(-n2c3ccccc3c3ccccc32)nc(-n2c3cc([Si](c4ccccc4)(c4ccccc4)c4ccccc4)ccc3c3ccc4c(c5ccccc5n4-c4ccccc4)c32)n1]

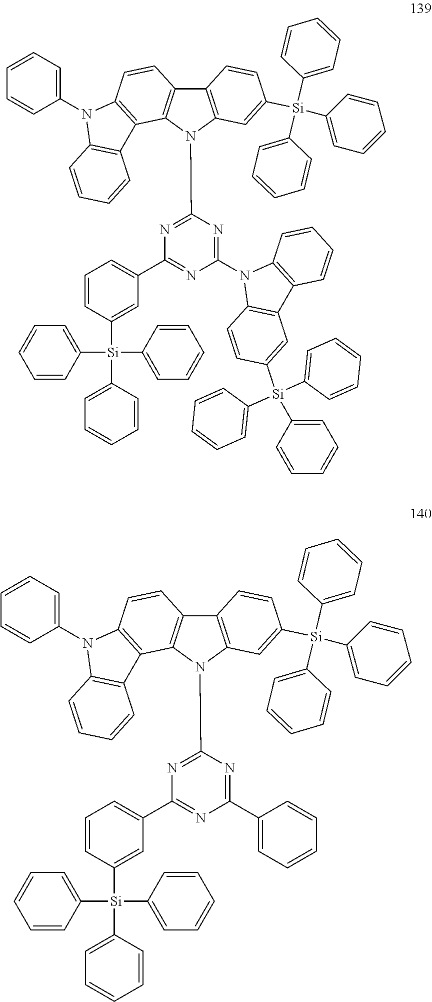 [SMILES: c1ccc(-n2c3ccccc3c3c2ccc2c4ccc([Si](c5ccccc5)(c5ccccc5)c5ccccc5)cc4n(-c4nc(-c5cccc([Si](c6ccccc6)(c6ccccc6)c6ccccc6)c5)nc(-n5c6ccccc6c6cc([Si](c7ccccc7)(c7ccccc7)c7ccccc7)ccc65)n4)c23)cc1 ; c1ccc(-c2nc(-c3cccc([Si](c4ccccc4)(c4ccccc4)c4ccccc4)c3)nc(-n3c4cc([Si](c5ccccc5)(c5ccccc5)c5ccccc5)ccc4c4ccc5c(c6ccccc6n5-c5ccccc5)c43)n2)cc1]

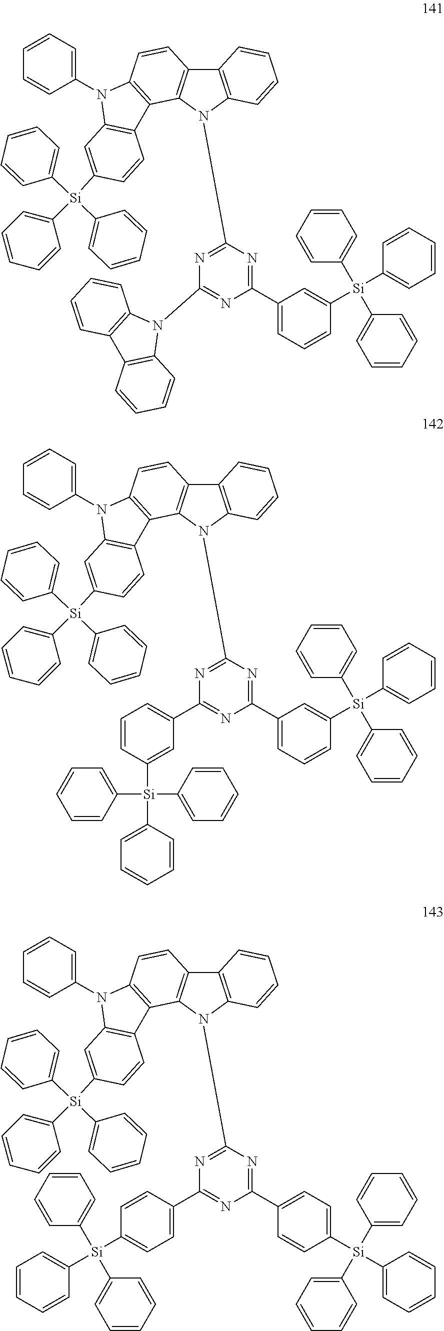

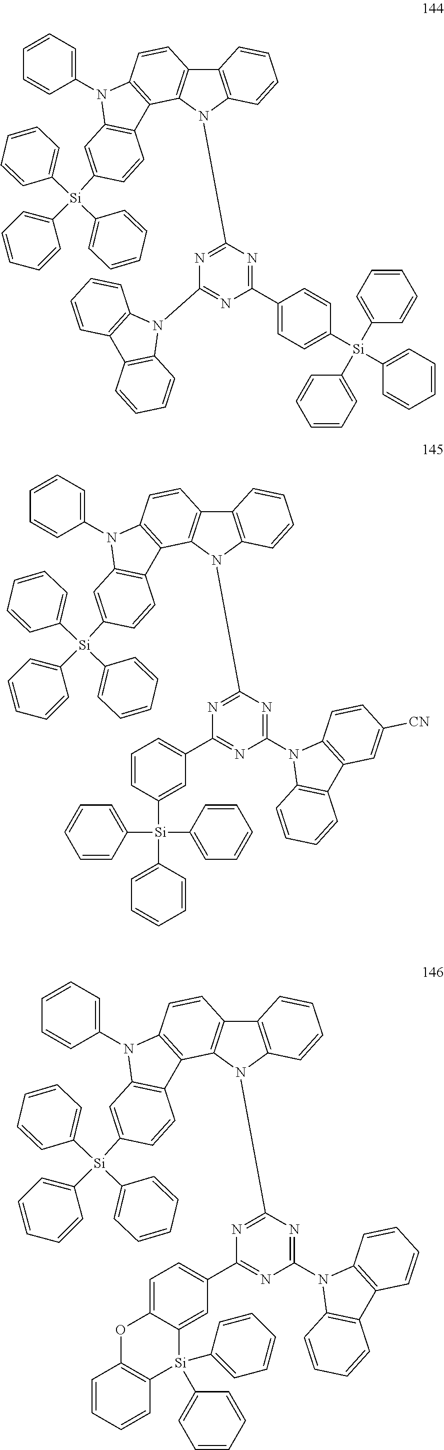 [SMILES: c1ccc(-n2c3cc([Si](c4ccccc4)(c4ccccc4)c4ccccc4)ccc3c3c2ccc2c4ccccc4n(-c4nc(-c5ccc([Si](c6ccccc6)(c6ccccc6)c6ccccc6)cc5)nc(-n5c6ccccc6c6ccccc65)n4)c23)cc1 ; N#Cc1ccc2c(c1)c1ccccc1n2-c1nc(-c2cccc([Si](c3ccccc3)(c3ccccc3)c3ccccc3)c2)nc(-n2c3ccccc3c3ccc4c(c5ccc([Si](c6ccccc6)(c6ccccc6)c6ccccc6)cc5n4-c4ccccc4)c32)n1 ; c1ccc(-n2c3cc([Si](c4ccccc4)(c4ccccc4)c4ccccc4)ccc3c3c2ccc2c4ccccc4n(-c4nc(-c5ccc6c(c5)[Si](c5ccccc5)(c5ccccc5)c5ccccc5O6)nc(-n5c6ccccc6c6ccccc65)n4)c23)cc1]

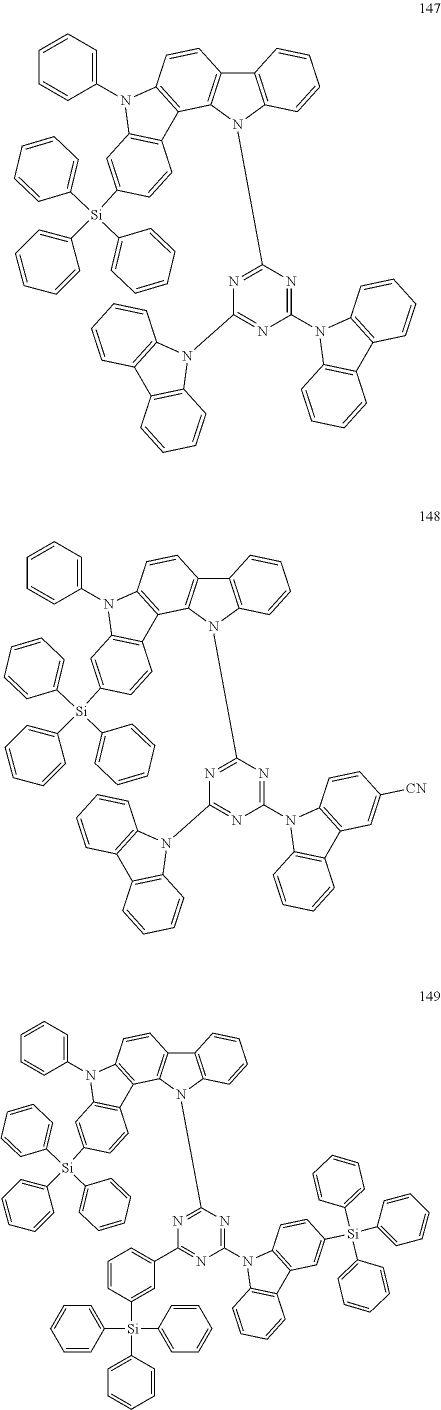 [SMILES: c1ccc(-n2c3cc([Si](c4ccccc4)(c4ccccc4)c4ccccc4)ccc3c3c2ccc2c4ccccc4n(-c4nc(-n5c6ccccc6c6ccccc65)nc(-n5c6ccccc6c6ccccc65)n4)c23)cc1 ; N#Cc1ccc2c(c1)c1ccccc1n2-c1nc(-n2c3ccccc3c3ccccc32)nc(-n2c3ccccc3c3ccc4c(c5ccc([Si](c6ccccc6)(c6ccccc6)c6ccccc6)cc5n4-c4ccccc4)c32)n1 ; c1ccc(-n2c3cc([Si](c4ccccc4)(c4ccccc4)c4ccccc4)ccc3c3c2ccc2c4ccccc4n(-c4nc(-c5cccc([Si](c6ccccc6)(c6ccccc6)c6ccccc6)c5)nc(-n5c6ccccc6c6cc([Si](c7ccccc7)(c7ccccc7)c7ccccc7)ccc65)n4)c23)cc1]

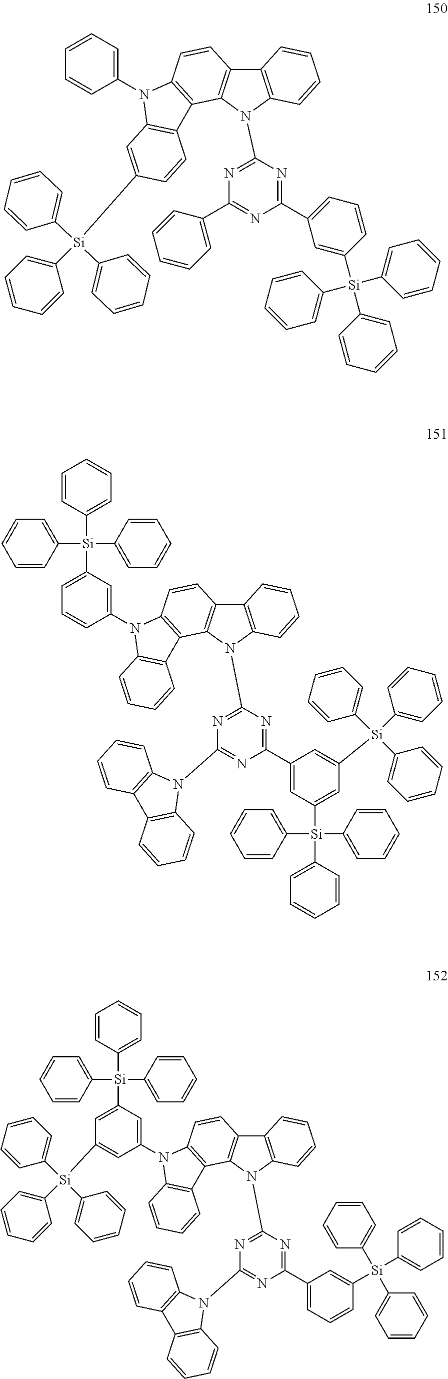

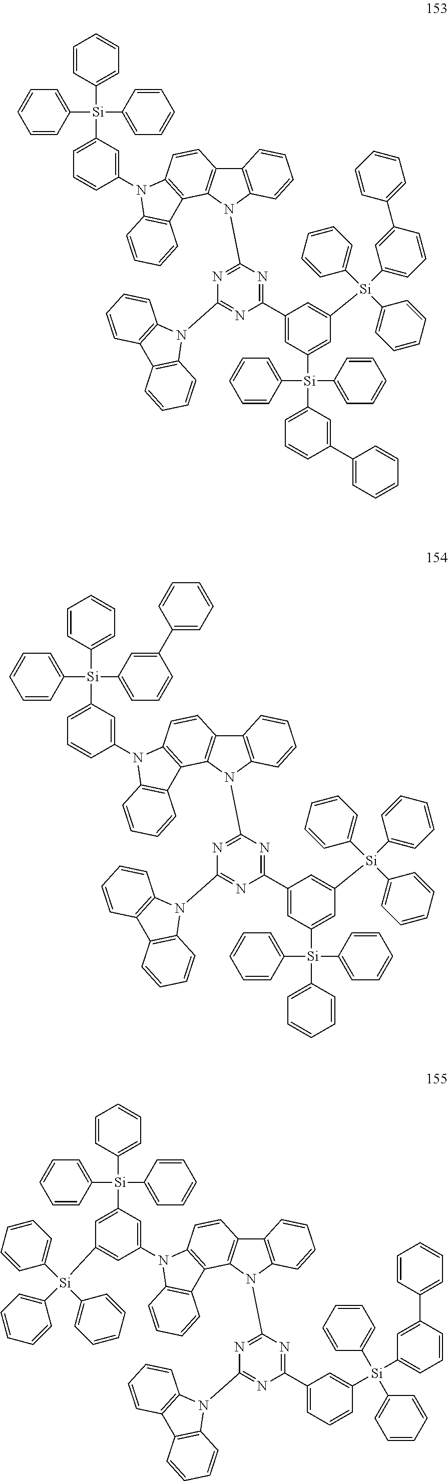

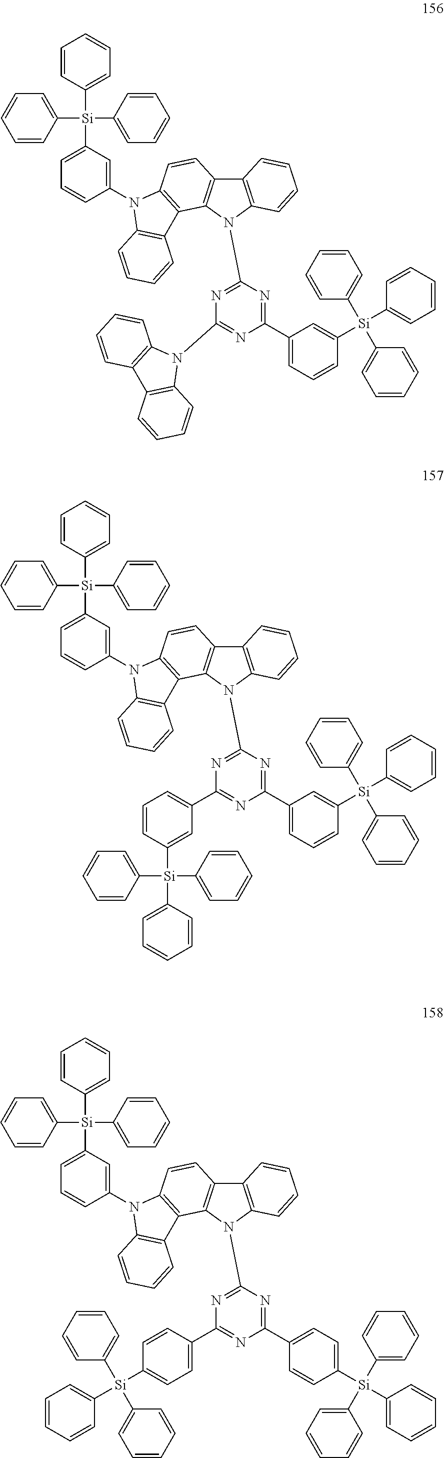

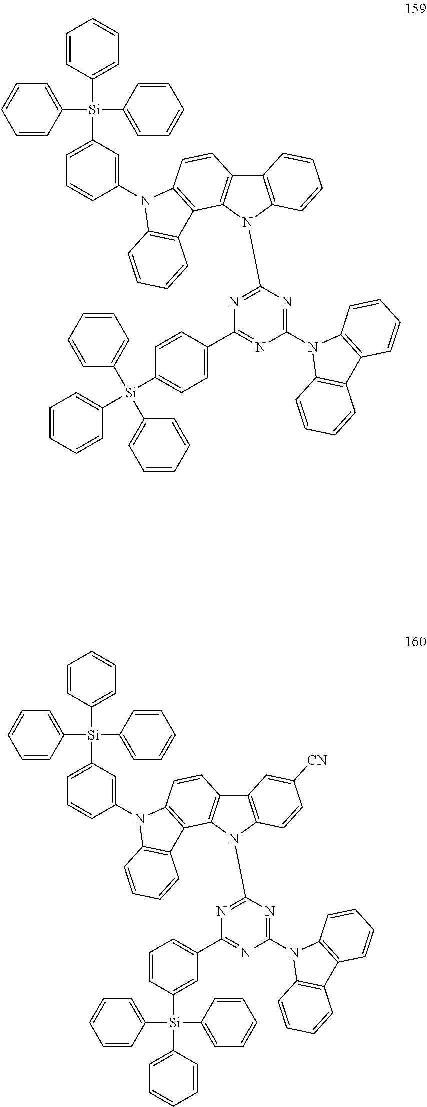 [SMILES: c1ccc([Si](c2ccccc2)(c2ccccc2)c2ccc(-c3nc(-n4c5ccccc5c5ccccc54)nc(-n4c5ccccc5c5ccc6c(c7ccccc7n6-c6cccc([Si](c7ccccc7)(c7ccccc7)c7ccccc7)c6)c54)n3)cc2)cc1 ; N#Cc1ccc2c(c1)c1ccc3c(c4ccccc4n3-c3cccc([Si](c4ccccc4)(c4ccccc4)c4ccccc4)c3)c1n2-c1nc(-c2cccc([Si](c3ccccc3)(c3ccccc3)c3ccccc3)c2)nc(-n2c3ccccc3c3ccccc32)n1]

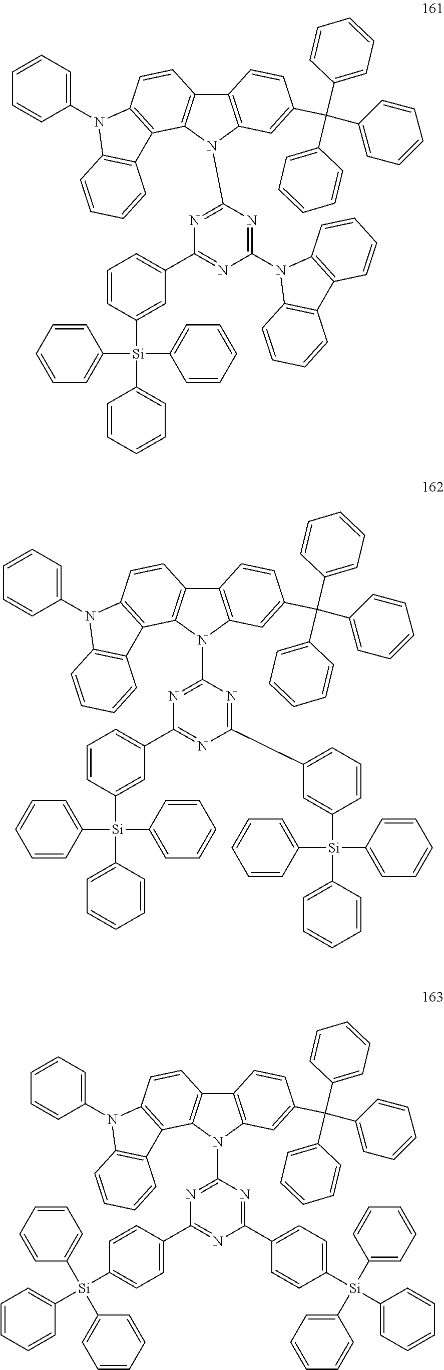

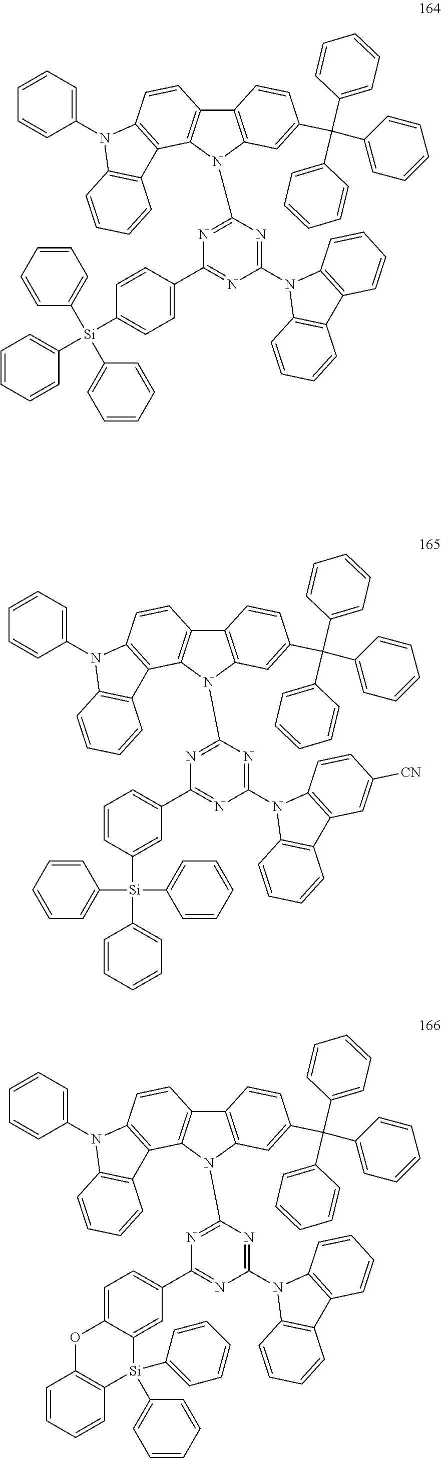 [SMILES: c1ccc(-n2c3ccccc3c3c2ccc2c4ccc(C(c5ccccc5)(c5ccccc5)c5ccccc5)cc4n(-c4nc(-c5ccc([Si](c6ccccc6)(c6ccccc6)c6ccccc6)cc5)nc(-n5c6ccccc6c6ccccc65)n4)c23)cc1 ; N#Cc1ccc2c(c1)c1ccccc1n2-c1nc(-c2cccc([Si](c3ccccc3)(c3ccccc3)c3ccccc3)c2)nc(-n2c3cc(C(c4ccccc4)(c4ccccc4)c4ccccc4)ccc3c3ccc4c(c5ccccc5n4-c4ccccc4)c32)n1 ; c1ccc(-n2c3ccccc3c3c2ccc2c4ccc(C(c5ccccc5)(c5ccccc5)c5ccccc5)cc4n(-c4nc(-c5ccc6c(c5)[Si](c5ccccc5)(c5ccccc5)c5ccccc5O6)nc(-n5c6ccccc6c6ccccc65)n4)c23)cc1]

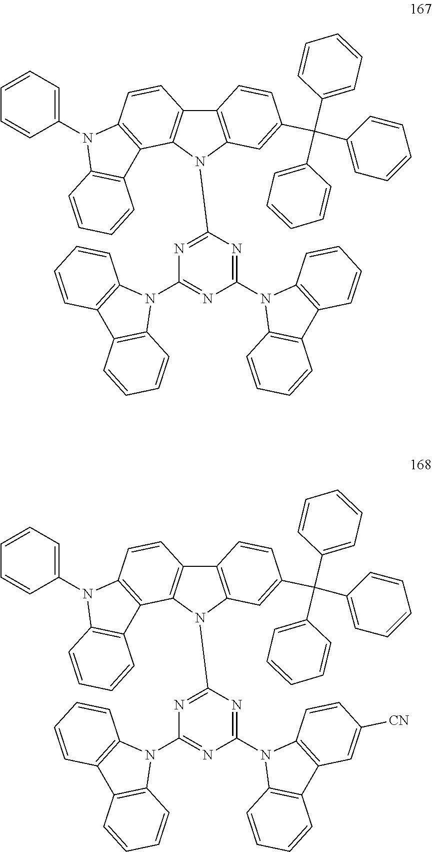 [SMILES: c1ccc(-n2c3ccccc3c3c2ccc2c4ccc(C(c5ccccc5)(c5ccccc5)c5ccccc5)cc4n(-c4nc(-n5c6ccccc6c6ccccc65)nc(-n5c6ccccc6c6ccccc65)n4)c23)cc1 ; N#Cc1ccc2c(c1)c1ccccc1n2-c1nc(-n2c3ccccc3c3ccccc32)nc(-n2c3cc(C(c4ccccc4)(c4ccccc4)c4ccccc4)ccc3c3ccc4c(c5ccccc5n4-c4ccccc4)c32)n1]

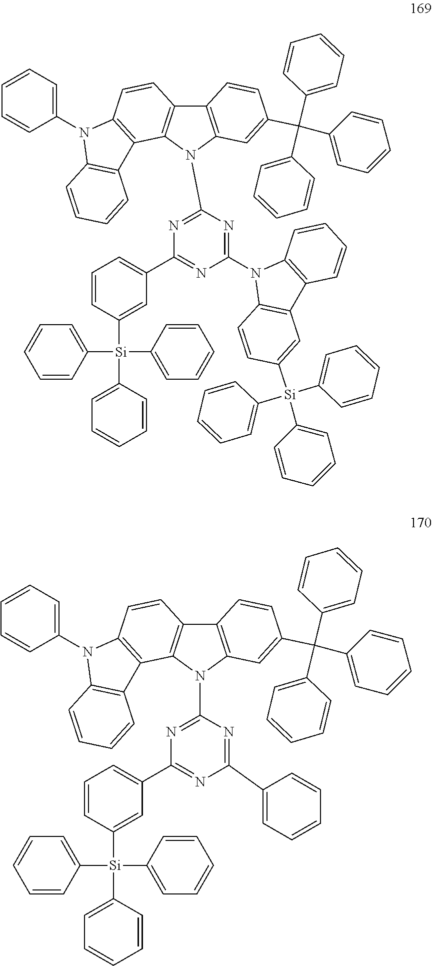 [SMILES: c1ccc(-n2c3ccccc3c3c2ccc2c4ccc(C(c5ccccc5)(c5ccccc5)c5ccccc5)cc4n(-c4nc(-c5cccc([Si](c6ccccc6)(c6ccccc6)c6ccccc6)c5)nc(-n5c6ccccc6c6cc([Si](c7ccccc7)(c7ccccc7)c7ccccc7)ccc65)n4)c23)cc1 ; c1ccc(-c2nc(-c3cccc([Si](c4ccccc4)(c4ccccc4)c4ccccc4)c3)nc(-n3c4cc(C(c5ccccc5)(c5ccccc5)c5ccccc5)ccc4c4ccc5c(c6ccccc6n5-c5ccccc5)c43)n2)cc1]

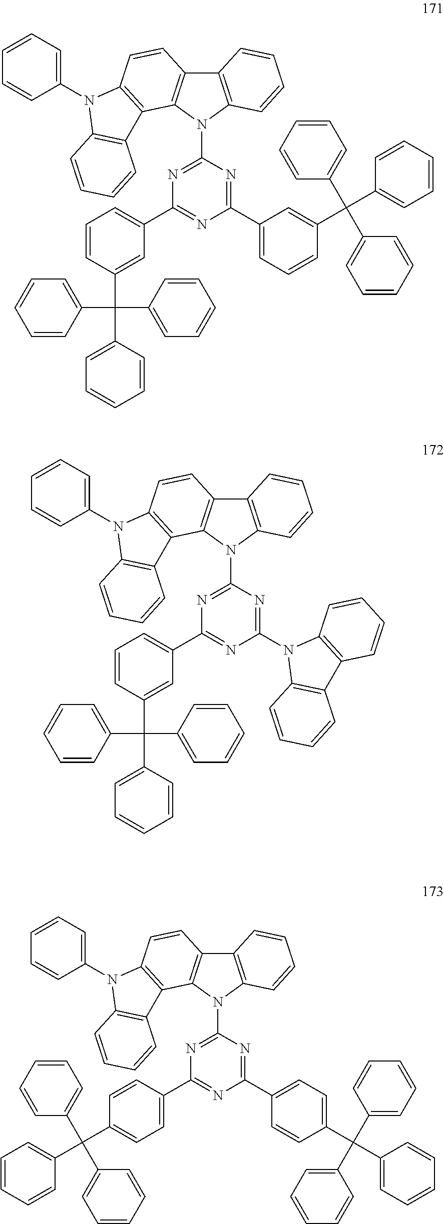 [SMILES: c1ccc(-n2c3ccccc3c3c2ccc2c4ccccc4n(-c4nc(-c5cccc(C(c6ccccc6)(c6ccccc6)c6ccccc6)c5)nc(-c5cccc(C(c6ccccc6)(c6ccccc6)c6ccccc6)c5)n4)c23)cc1 ; c1ccc(-n2c3ccccc3c3c2ccc2c4ccccc4n(-c4nc(-c5cccc(C(c6ccccc6)(c6ccccc6)c6ccccc6)c5)nc(-n5c6ccccc6c6ccccc65)n4)c23)cc1 ; c1ccc(-n2c3ccccc3c3c2ccc2c4ccccc4n(-c4nc(-c5ccc(C(c6ccccc6)(c6ccccc6)c6ccccc6)cc5)nc(-c5ccc(C(c6ccccc6)(c6ccccc6)c6ccccc6)cc5)n4)c23)cc1]

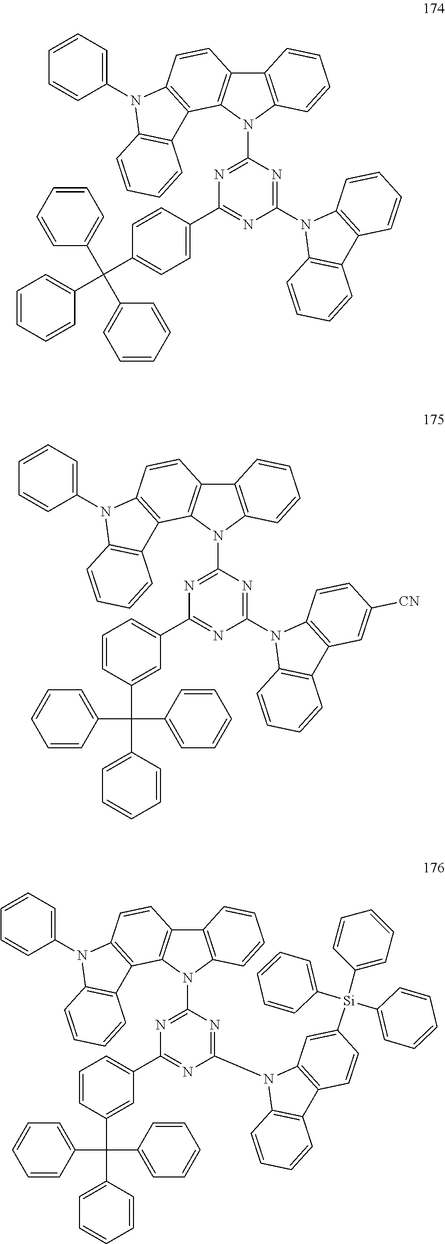 [SMILES: c1ccc(-n2c3ccccc3c3c2ccc2c4ccccc4n(-c4nc(-c5ccc(C(c6ccccc6)(c6ccccc6)c6ccccc6)cc5)nc(-n5c6ccccc6c6ccccc65)n4)c23)cc1 ; N#Cc1ccc2c(c1)c1ccccc1n2-c1nc(-c2cccc(C(c3ccccc3)(c3ccccc3)c3ccccc3)c2)nc(-n2c3ccccc3c3ccc4c(c5ccccc5n4-c4ccccc4)c32)n1 ; c1ccc(-n2c3ccccc3c3c2ccc2c4ccccc4n(-c4nc(-c5cccc(C(c6ccccc6)(c6ccccc6)c6ccccc6)c5)nc(-n5c6ccccc6c6ccc([Si](c7ccccc7)(c7ccccc7)c7ccccc7)cc65)n4)c23)cc1]

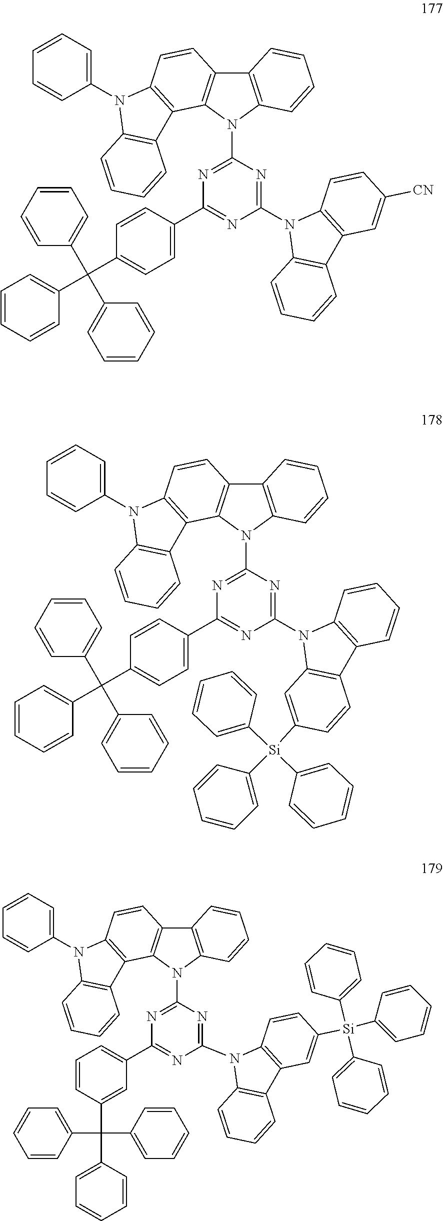 [SMILES: N#Cc1ccc2c(c1)c1ccccc1n2-c1nc(-c2ccc(C(c3ccccc3)(c3ccccc3)c3ccccc3)cc2)nc(-n2c3ccccc3c3ccc4c(c5ccccc5n4-c4ccccc4)c32)n1 ; c1ccc(-n2c3ccccc3c3c2ccc2c4ccccc4n(-c4nc(-c5ccc(C(c6ccccc6)(c6ccccc6)c6ccccc6)cc5)nc(-n5c6ccccc6c6ccc([Si](c7ccccc7)(c7ccccc7)c7ccccc7)cc65)n4)c23)cc1 ; c1ccc(-n2c3ccccc3c3c2ccc2c4ccccc4n(-c4nc(-c5cccc(C(c6ccccc6)(c6ccccc6)c6ccccc6)c5)nc(-n5c6ccccc6c6cc([Si](c7ccccc7)(c7ccccc7)c7ccccc7)ccc65)n4)c23)cc1]

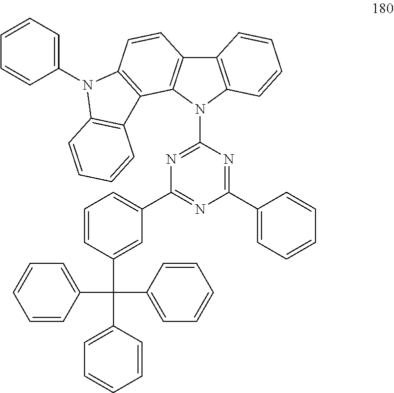 [SMILES: c1ccc(-c2nc(-c3cccc(C(c4ccccc4)(c4ccccc4)c4ccccc4)c3)nc(-n3c4ccccc4c4ccc5c(c6ccccc6n5-c5ccccc5)c43)n2)cc1]

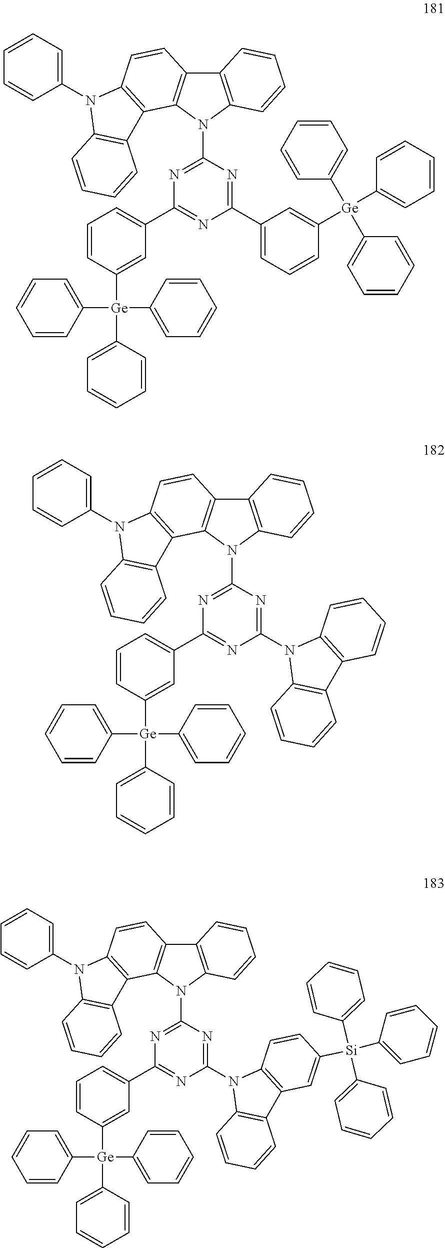

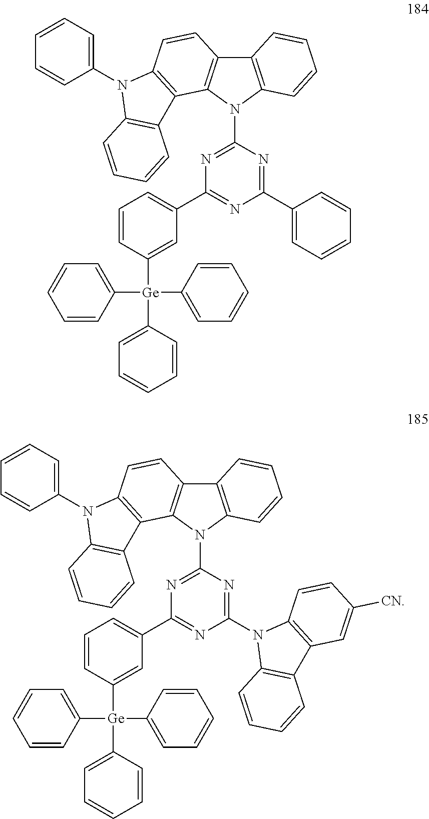

The polycyclic compound of an embodiment, represented by Formula 1 may be a light emitting material having a light emitting central wavelength in a wavelength region of about 430 nm to about 480 nm. The emission layer EML of the light emitting element ED includes the polycyclic compound of an embodiment, represented by Formula 1, and may emit blue light. For example, the emission layer EML of the light emitting element ED of an embodiment may emit blue light in a region of about 480 nm or less. However, an embodiment of the present disclosure is not limited thereto, and the emission layer EML may also emit green light or red light.

In addition, in an embodiment, the emission layer EML may include a host and a dopant, and may include the above-described polycyclic compound as the host. The polycyclic compound of an embodiment, represented by Formula 1 may be a host material of the emission layer.

For example, in the light emitting element ED of an embodiment, the emission layer EML may include a host for emitting phosphorescence and a dopant for emitting phosphorescence, and may include the polycyclic compound of an embodiment as the host for emitting phosphorescence. Or in the light emitting element ED of an embodiment, the emission layer EML may include a host for emitting fluorescence and a dopant for emitting fluorescence, and may include the polycyclic compound of an embodiment as the host for emitting fluorescence.

In the light emitting element ED of an embodiment, the emission layer EML may include a host for emitting delayed fluorescence and a dopant for emitting delayed fluorescence, and may include the polycyclic compound of an embodiment as the host for emitting delayed fluorescence. In the light emitting element ED of an embodiment, the emission layer EML may include a host for emitting blue thermally activated delayed fluorescence (TADF) and a dopant for emitting blue thermally activated delayed fluorescence, and may include the polycyclic compound of an embodiment as the host for emitting blue thermally activated delayed fluorescence. The emission layer EML may include at least one selected from among the polycyclic compounds represented in Compound Group 1 as the host material of the emission layer.

In some embodiments, in the light emitting element ED, the emission layer EML may further include a known material. The emission layer EML may include anthracene derivatives, pyrene derivatives, fluoranthene derivatives, chrysene derivatives, dihydrobenzanthracene derivatives, or triphenylene derivatives. The emission layer EML may include anthracene derivatives or pyrene derivatives.

In the light emitting elements ED of embodiments, shown in FIG. 3 to FIG. 6, the emission layer EML may include a host and a dopant, and the emission layer EML may include a compound represented by Formula E-1. The compound represented by Formula E-1 may be used as a fluorescence host material or a delayed fluorescence host material.

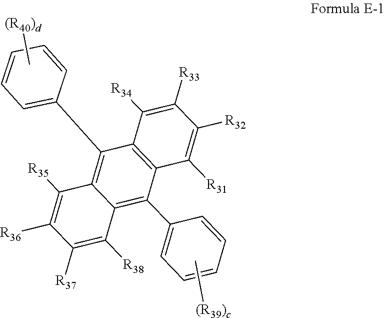

In Formula E-1, R₃₁ to R₄₀ may each independently be a hydrogen atom, a deuterium atom, a halogen atom, a substituted or unsubstituted silyl group, a substituted or unsubstituted thio group, a substituted or unsubstituted oxy group, a substituted or unsubstituted alkyl group of 1 to 10 carbon atoms, a substituted or unsubstituted alkenyl group of 1 to 10 carbon atoms, a substituted or unsubstituted aryl group of 6 to 30 ring-forming carbon atoms, or a substituted or unsubstituted heteroaryl group of 2 to 30 ring-forming carbon atoms, or may be combined with an adjacent group to form a ring. In some embodiments, R₃₁ to R₄₀ may be combined with an adjacent group to form a saturated hydrocarbon ring, an unsaturated hydrocarbon ring, a saturated heterocycle, or an unsaturated heterocycle.

In Formula E-1, “c” and “d” may each independently be an integer from 0 to 5.

Formula E-1 may be represented by any one selected from among the compounds represented in Compound Group E-1.

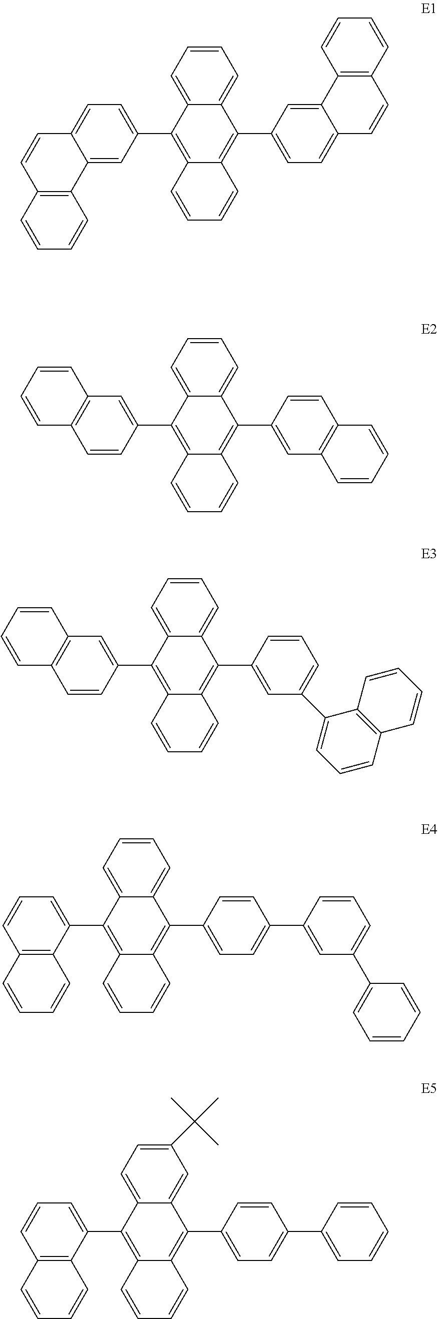

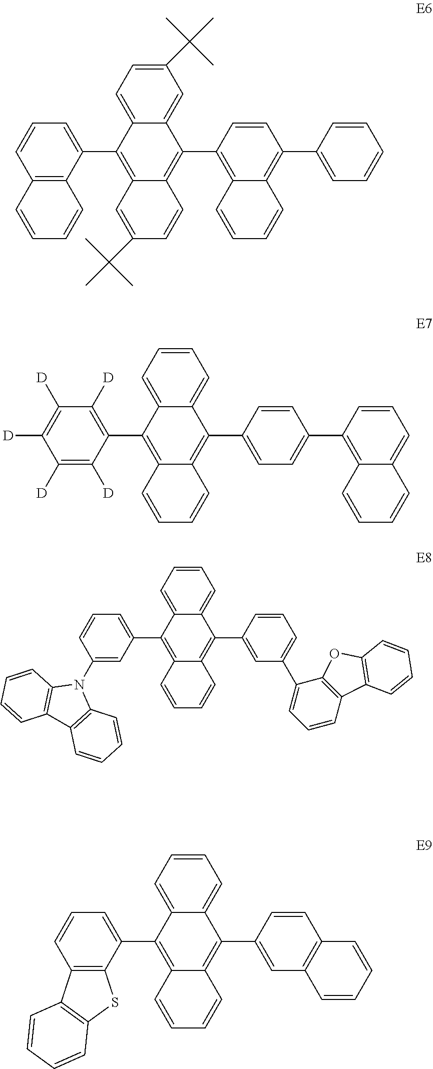

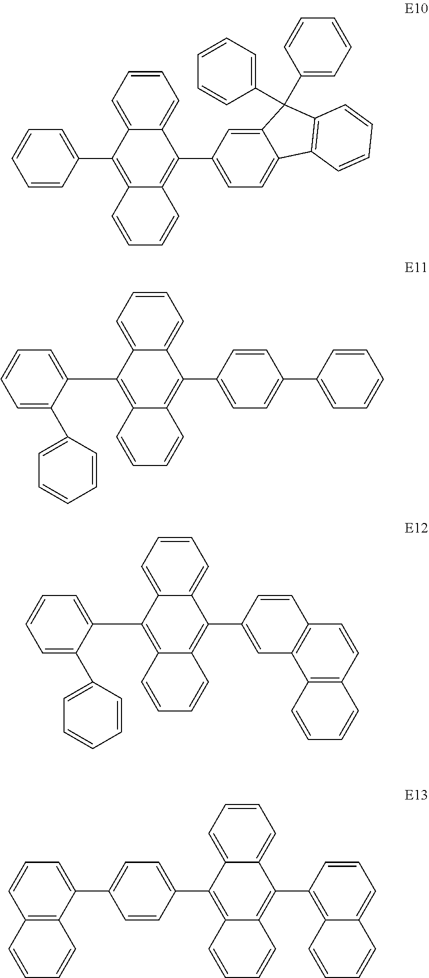

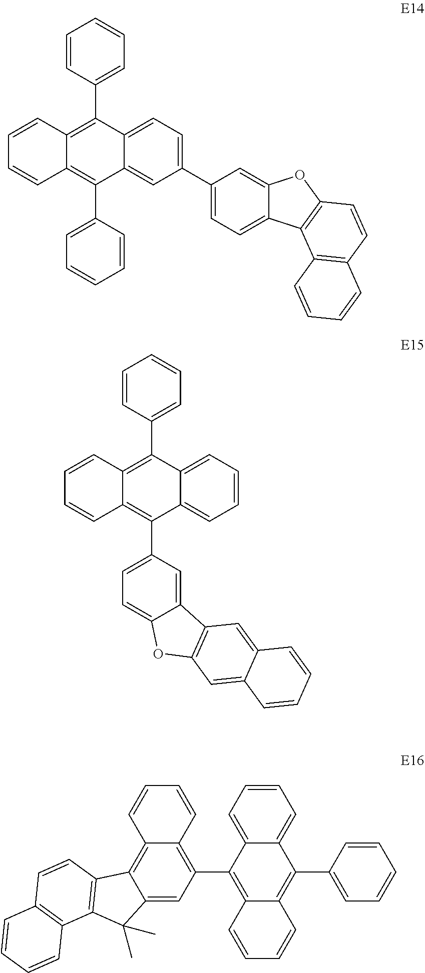

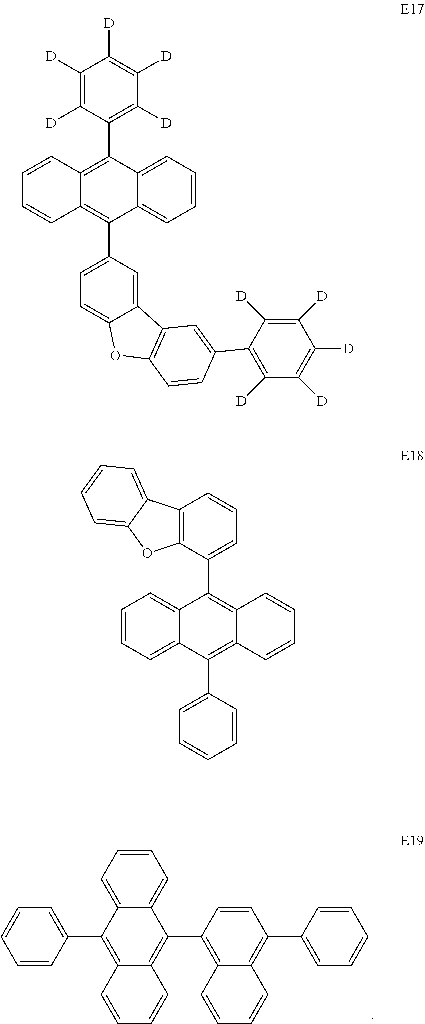

In an embodiment, the emission layer EML may include a compound represented by Formula E-2a or Formula E-2b. The compound represented by Formula E-2a or Formula E-2b may be used as a phosphorescence host material or a delayed fluorescence host material.

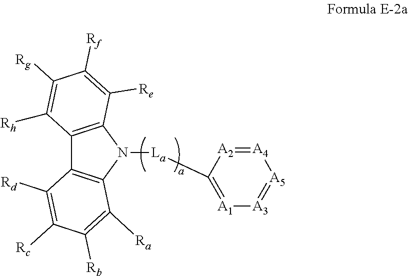

In Formula E-2a, “a” may be an integer from 0 to 10, La may be a direct linkage, a substituted or unsubstituted arylene group of 6 to 30 ring-forming carbon atoms, or a substituted or unsubstituted heteroarylene group of 2 to 30 ring-forming carbon atoms. In some embodiments, when “a” is an integer of 2 or more, multiple La may each independently be a substituted or unsubstituted arylene group of 6 to 30 ring-forming carbon atoms, or a substituted or unsubstituted heteroarylene group of 2 to 30 ring-forming carbon atoms.

In some embodiments, in Formula E-2a, A₁ to A₅ may each independently be N or CRᵢ. Rₐ to Rᵢ may each independently be a hydrogen atom, a deuterium atom, a substituted or unsubstituted amine group, a substituted or unsubstituted thio group, a substituted or unsubstituted oxy group, a substituted or unsubstituted phosphine oxide group, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, or may be combined with an adjacent group to form a ring. Rₐ to Rᵢ may be combined with an adjacent group to form a hydrocarbon ring or a heterocycle including N, O, S, etc. as a ring-forming atom.

In some embodiments, in Formula E-2a, two or three selected from A₁ to A₅ may be N, and the remainder may be CR.

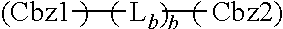

In Formula E-2b, Cbz1 and Cbz2 may each independently be an unsubstituted carbazole group, or a carbazole group substituted with an aryl group of 6 to 30 ring-forming carbon atoms. L_{b} may be a direct linkage, a substituted or unsubstituted arylene group of 6 to 30 ring-forming carbon atoms, or a substituted or unsubstituted heteroarylene group of 2 to 30 ring-forming carbon atoms. “b” is an integer from 0 to 10, and when “b” is an integer of 2 or more, multiple L_{b} may each independently be a substituted or unsubstituted arylene group of 6 to 30 ring-forming carbon atoms, or a substituted or unsubstituted heteroarylene group of 2 to 30 ring-forming carbon atoms.

The compound represented by Formula E-2a or Formula E-2b may be represented by any one selected from among the compounds in Compound Group E-2. However, the compounds shown in Compound Group E-2 are merely examples, and the compound represented by Formula E-2a or Formula E-2b is not limited to the compounds represented in Compound Group E-2.

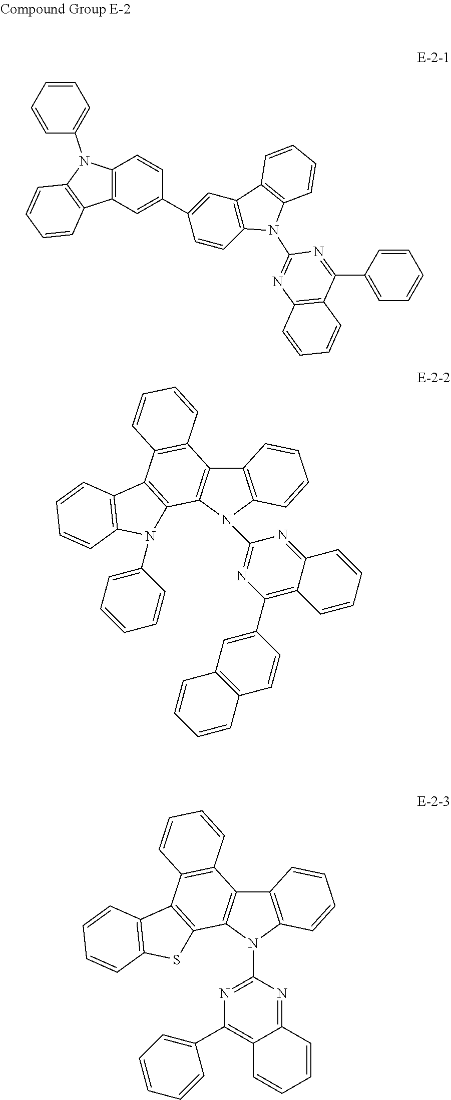 [SMILES: c1ccc(-c2nc(-n3c4ccccc4c4cc(-c5ccc6c(c5)c5ccccc5n6-c5ccccc5)ccc43)nc3ccccc23)cc1 ; c1ccc(-n2c3ccccc3c3c4ccccc4c4c5ccccc5n(-c5nc(-c6ccc7ccccc7c6)c6ccccc6n5)c4c32)cc1 ; c1ccc(-c2nc(-n3c4ccccc4c4c5ccccc5c5c6ccccc6sc5c43)nc3ccccc23)cc1]

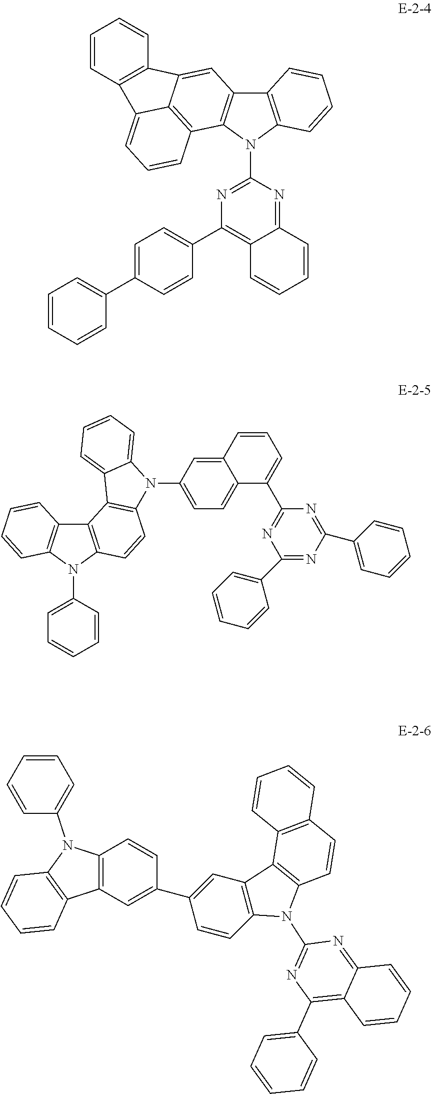 [SMILES: c1ccc(-c2ccc(-c3nc(-n4c5ccccc5c5cc6c7c(cccc7c54)-c4ccccc4-6)nc4ccccc34)cc2)cc1 ; c1ccc(-c2nc(-c3ccccc3)nc(-c3cccc4cc(-n5c6ccccc6c6c7c8ccccc8n(-c8ccccc8)c7ccc65)ccc34)n2)cc1 ; c1ccc(-c2nc(-n3c4ccc(-c5ccc6c(c5)c5ccccc5n6-c5ccccc5)cc4c4c5ccccc5ccc43)nc3ccccc23)cc1]

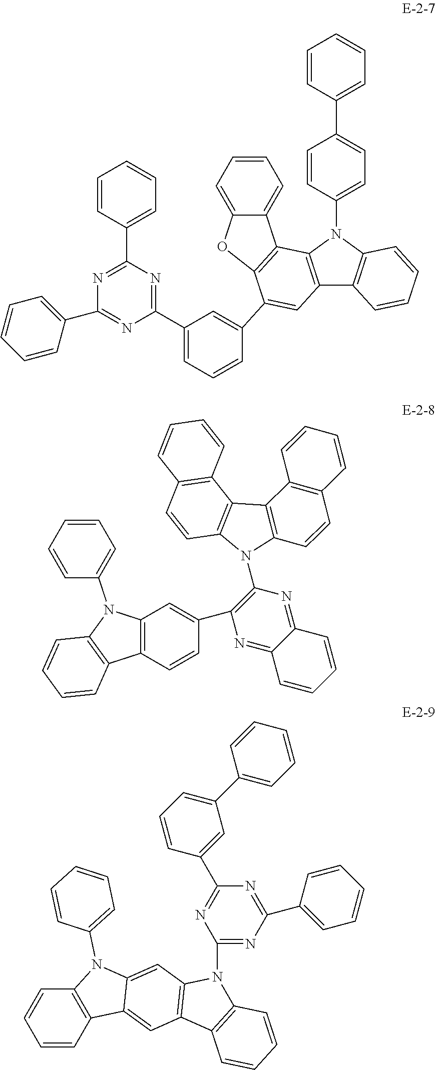

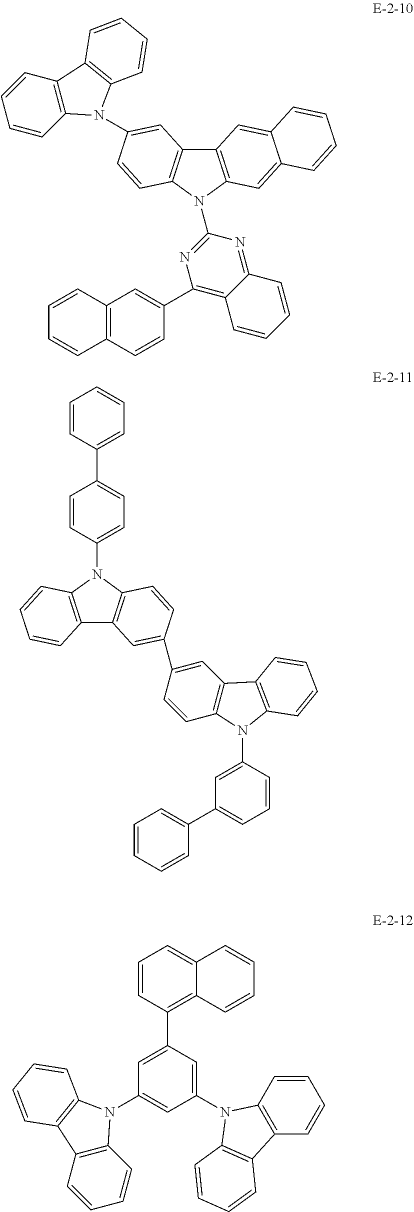

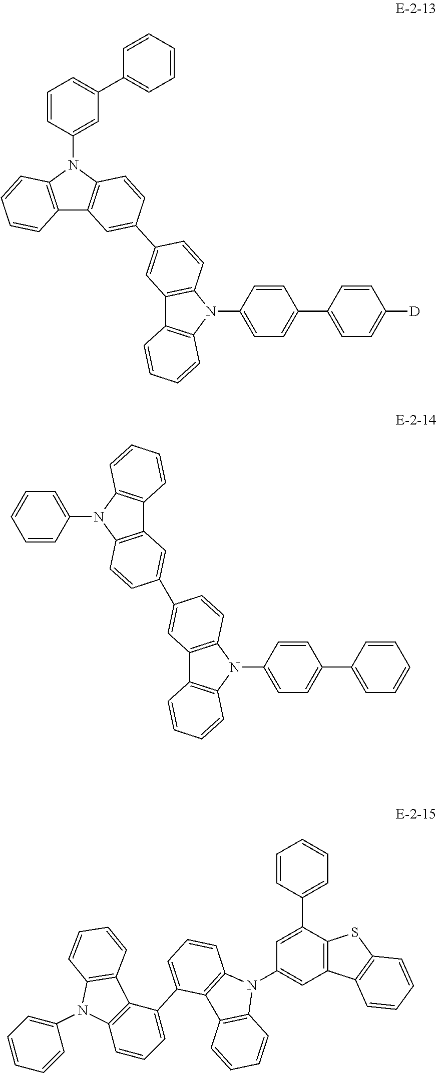 [SMILES: [2H]c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5ccccc5)ccc43)cc2)cc1 ; c1ccc(-c2cc(-n3c4ccccc4c4c(-c5cccc6c5c5ccccc5n6-c5ccccc5)cccc43)cc3c2sc2ccccc23)cc1]

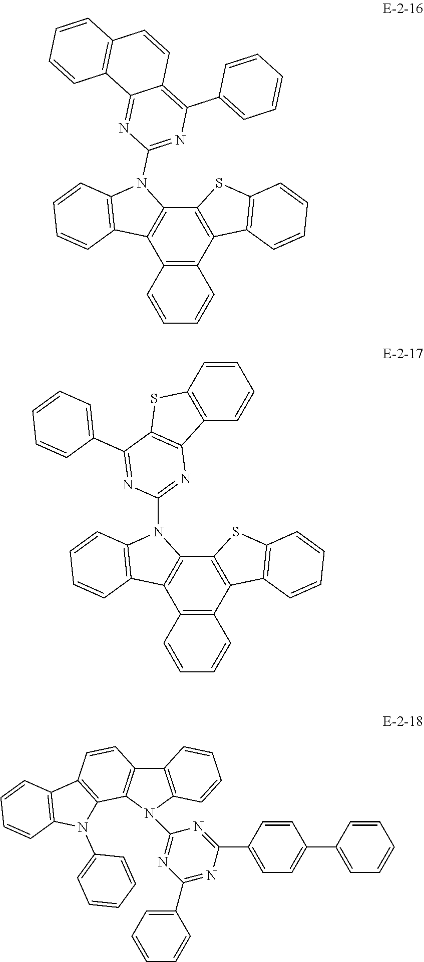

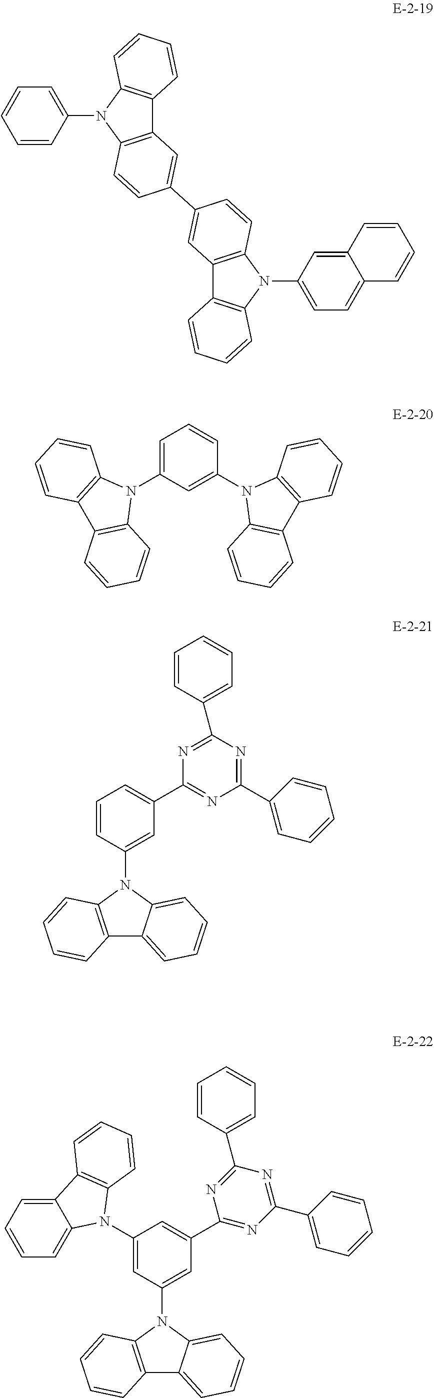 [SMILES: c1ccc(-n2c3ccccc3c3cc(-c4ccc5c(c4)c4ccccc4n5-c4ccc5ccccc5c4)ccc32)cc1 ; c1cc(-n2c3ccccc3c3ccccc32)cc(-n2c3ccccc3c3ccccc32)c1 ; c1ccc(-c2nc(-c3ccccc3)nc(-c3cccc(-n4c5ccccc5c5ccccc54)c3)n2)cc1 ; c1ccc(-c2nc(-c3ccccc3)nc(-c3cc(-n4c5ccccc5c5ccccc54)cc(-n4c5ccccc5c5ccccc54)c3)n2)cc1]

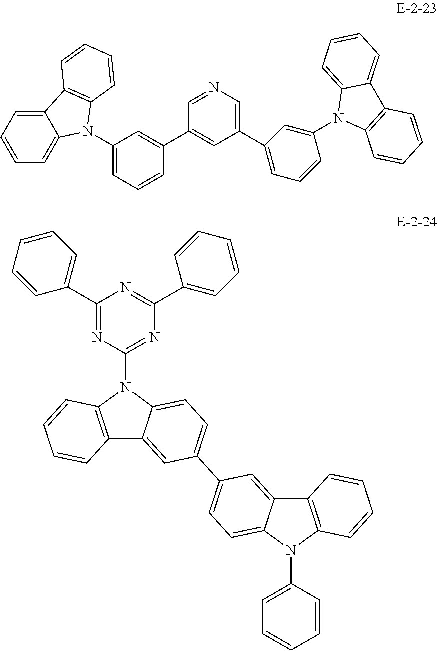 [SMILES: c1cc(-c2cncc(-c3cccc(-n4c5ccccc5c5ccccc54)c3)c2)cc(-n2c3ccccc3c3ccccc32)c1 ; c1ccc(-c2nc(-c3ccccc3)nc(-n3c4ccccc4c4cc(-c5ccc6c(c5)c5ccccc5n6-c5ccccc5)ccc43)n2)cc1]

The emission layer EML may further include a common material generally used/generally available in the art as a host material. For example, the emission layer EML may include as a host material, at least one of bis (4-(9H-carbazol-9-yl) phenyl) diphenylsilane (BCPDS), (4-(1-(4-(diphenylamino) phenyl) cyclohexyl) phenyl) diphenyl-phosphine oxide (POPCPA), bis[2-(diphenylphosphino)phenyl] ether oxide (DPEPO), 4,4′-bis(carbazol-9-yl)biphenyl (CBP), 1,3-bis(carbazol-9-yl)benzene (mCP), 2,8-bis(diphenylphosphoryl)dibenzo[b,d]furan (PPF), 4,4′,4″-tris(carbazol-9-yl)-triphenylamine (TCTA), or 1,3,5-tris(1-phenyl-1H-benzo[d]imidazole-2-yl)benzene (TPBi). However, an embodiment of the present disclosure is not limited thereto. For example, tris(8-hydroxyquinolino)aluminum (Alq₃), 9,10-di(naphthalene-2-yl)anthracene (ADN), 2-tert-butyl-9,10-di(naphth-2-yl)anthracene (TBADN), distyrylarylene (DSA), 4,4′-bis(9-carbazolyl)-2,2′-dimethyl-biphenyl (CDBP), 2-methyl-9,10-bis(naphthalen yl)anthracene (MADN), hexaphenyl cyclotriphosphazene (CP1), 1,4-bis(triphenylsilyl)benzene (UGH2), hexaphenylcyclotrisiloxane (DPSiO₃), octaphenylcyclotetra siloxane (DPSiO₄), etc. may also be used as the host material.

The emission layer EML may include a compound represented by Formula M-a or Formula M-b. The compound represented by Formula M-a or Formula M-b may be used as a phosphorescence dopant material. In some embodiments, the compound represented by Formula M-a or Formula M-b may be used as an auxiliary dopant material.

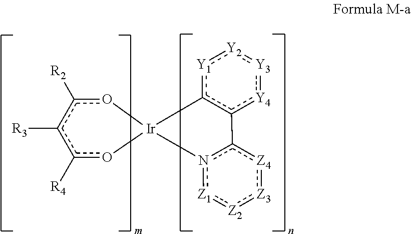

In Formula M-a, Y₁ to Y₄, and Z₁ to Z₄ may each independently be CR₁ or N, and R₁ to R₄ may each independently be a hydrogen atom, a deuterium atom, a substituted or unsubstituted amine group, a substituted or unsubstituted thio group, a substituted or unsubstituted oxy group, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, or may be combined with an adjacent group to form a ring. In Formula M-a, “m” is 0 or 1, and “n” is 2 or 3. In Formula M-a, when “m” is 0, “n” is 3, and when “m” is 1, “n” is 2.

The compound represented by Formula M-a may be represented by any one selected from among Compounds M-a1 to M-a25. However, Compounds M-a1 to M-a25 are merely examples, and the compound represented by Formula M-a is not limited to the compounds represented by Compounds M-a1 to M-a25.

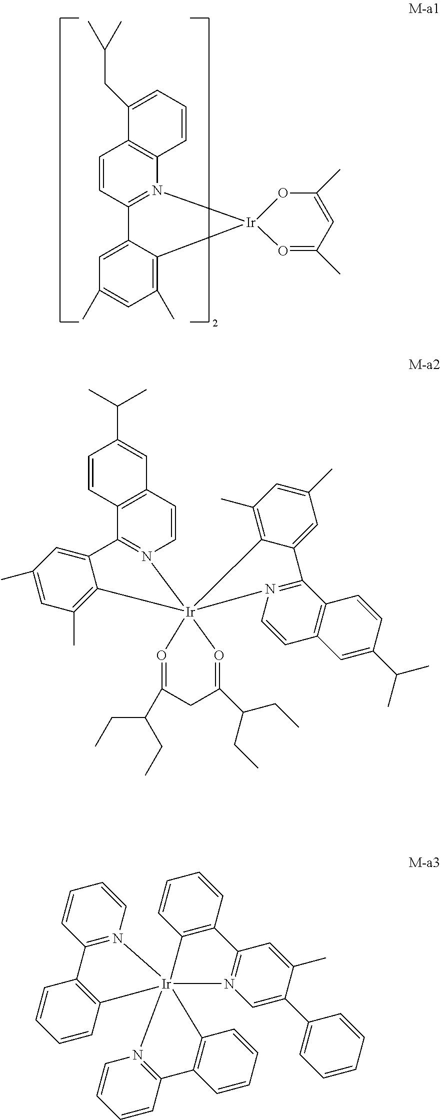

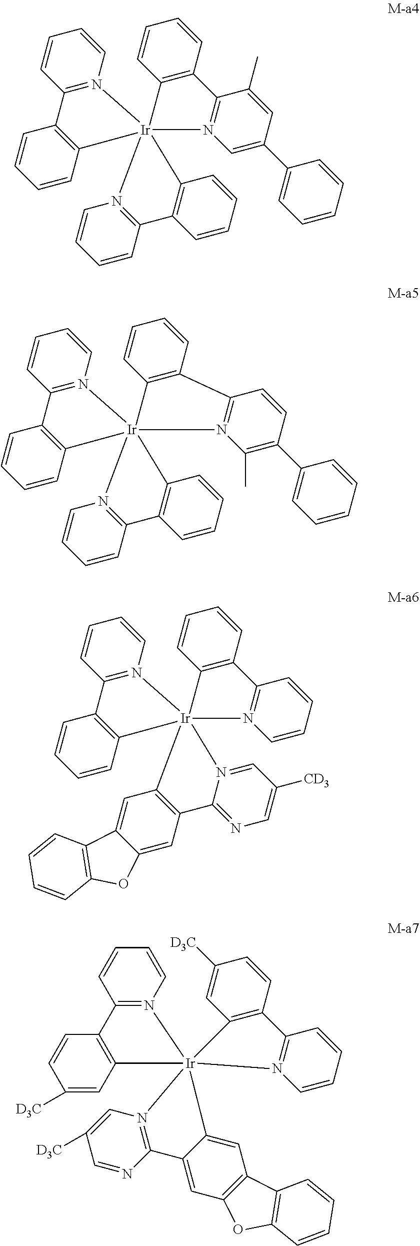

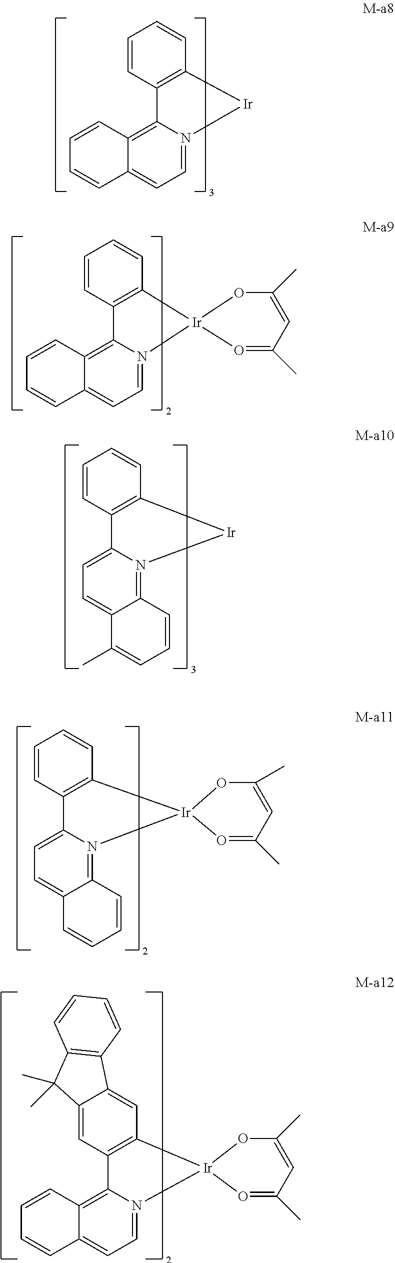

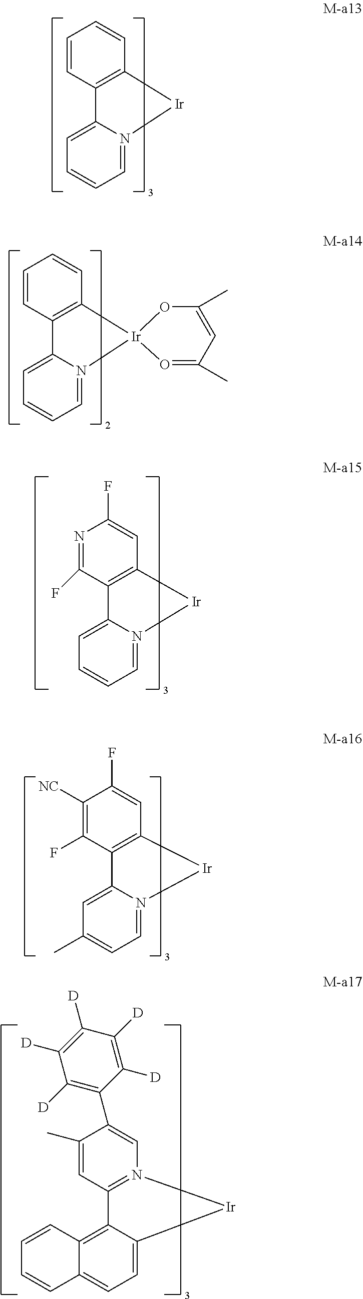

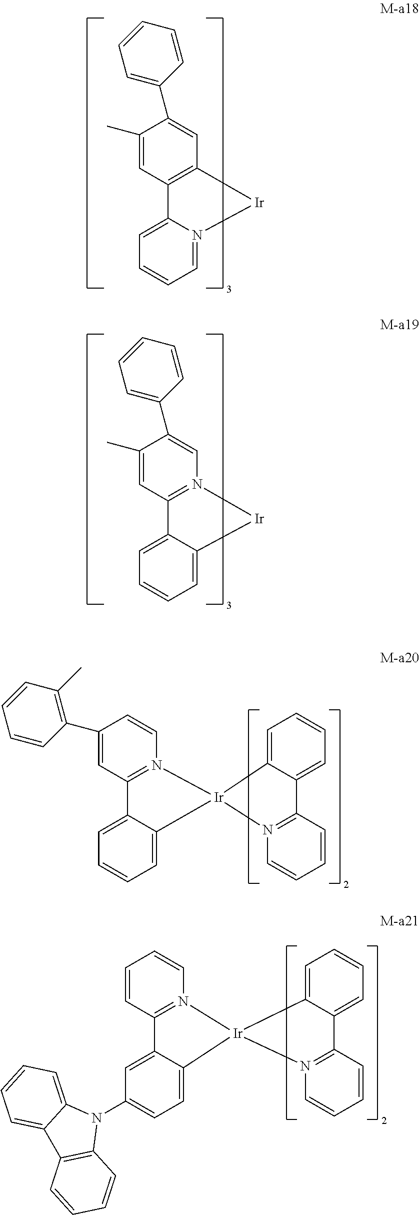

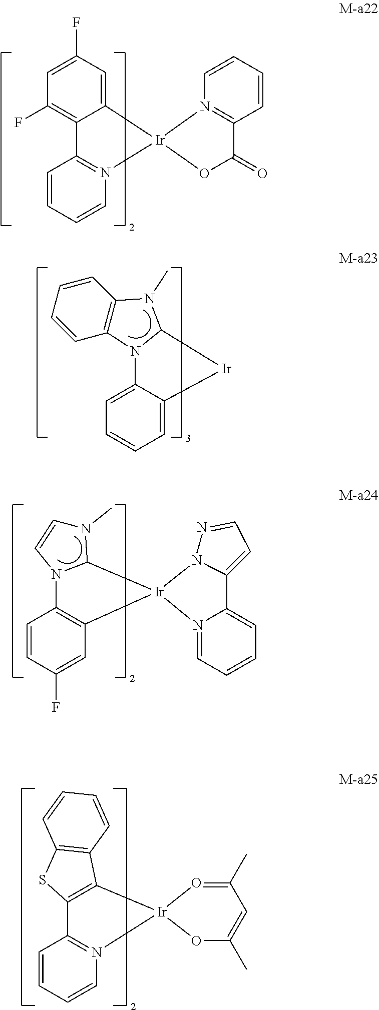

Compound M-a1 and Compound M-a2 may be used as red dopant materials, and Compound M-a3 to Compound M-a7 may be used as green dopant materials.

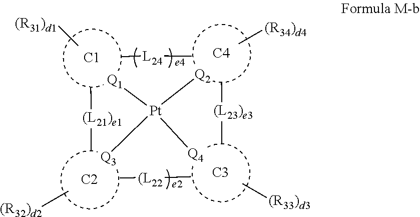

In Formula M-b, Q₁ to Q₄ may each independently be C or N, C1 to C4 may each independently be a substituted or unsubstituted hydrocarbon ring of 5 to 30 ring-forming carbon atoms, or a substituted or unsubstituted heterocycle of 2 to 30 ring-forming carbon atoms, L₂₁ to L₂₄ may each independently be a direct linkage, *—O—*, *—S—*,

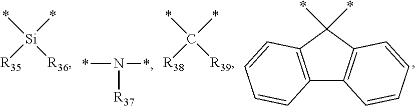

a substituted or unsubstituted divalent alkyl group of 1 to 20 carbon atoms, a substituted or unsubstituted arylene group of 6 to 30 ring-forming carbon atoms, or a substituted or unsubstituted heteroarylene group of 2 to 30 ring-forming carbon atoms, el to e4 may each independently be 0 or 1, R₃₁ to R₃₉ may each independently be a hydrogen atom, a deuterium atom, a halogen atom, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, or combined with an adjacent group to form a ring, and d1 to d4 may each independently be an integer from 0 to 4.

The compound represented by Formula M-b may be used as a blue phosphorescence dopant or a green phosphorescence dopant.

The compound represented by Formula M-b may be represented by any one selected from among the compounds below. However, the compounds are merely examples, and the compound represented by Formula M-b is not limited to the compounds represented below.

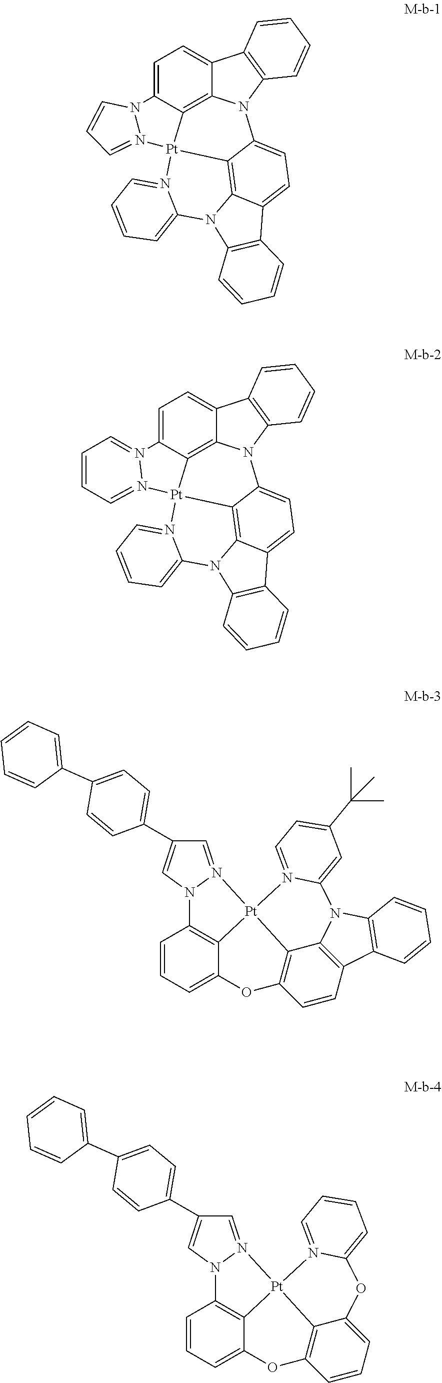

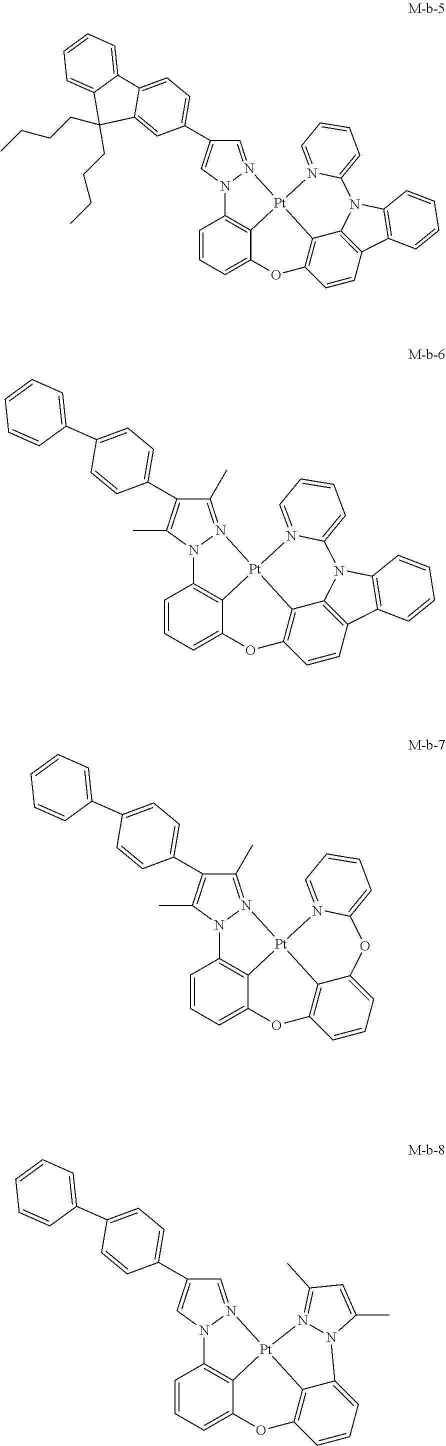

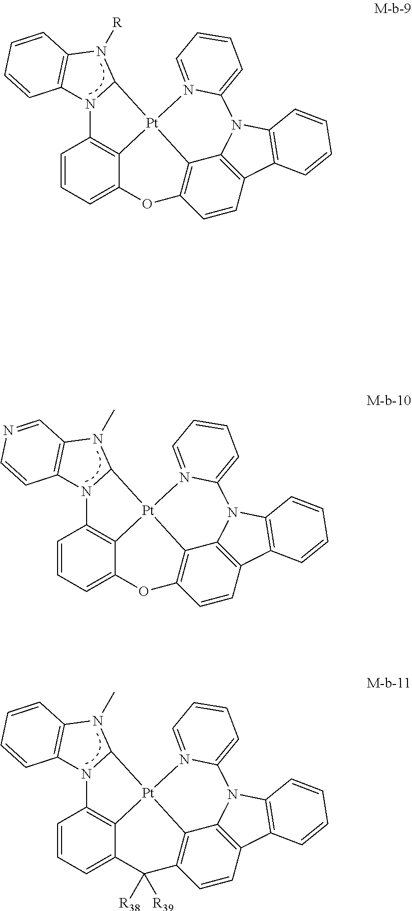

In the foregoing compounds, R, R₃₈, and R₃₉ may each independently be a hydrogen atom, a deuterium atom, a halogen atom, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms.

The emission layer EML may include any one selected from among Formula F-a to Formula F-c. The compounds represented by Formula F-a to Formula F-c may be used as fluorescence dopant materials.

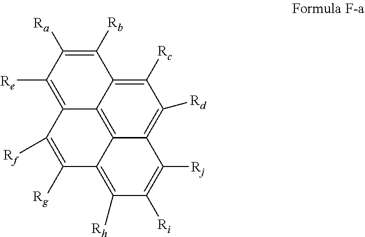

In Formula F-a, two selected from Rₐ to Rⱼ may each independently be substituted with *—NAr₁Ar₂. The remainder not substituted with *—NAr₁Ar₂ selected from among Rₐ to Rⱼ may each independently be a hydrogen atom, a deuterium atom, a halogen atom, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms.

In *—NAr₁Ar₂, Ar₁ and Ar₂ may each independently be a substituted or unsubstituted aryl group of 6 to 30 ring-forming carbon atoms, or a substituted or unsubstituted heteroaryl group of 2 to 30 ring-forming carbon atoms. For example, at least one selected from among Ar₁ and Ar₂ may be a heteroaryl group including O or S as a ring-forming atom.

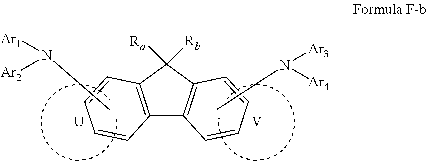

In Formula F-b, Rₐ and R_{b} may each independently be a hydrogen atom, a deuterium atom, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, or may be combined with an adjacent group to form a ring. Ar₁ to Ar₄ may each independently be a substituted or unsubstituted aryl group of 6 to 30 ring-forming carbon atoms, or a substituted or unsubstituted heteroaryl group of 2 to 30 ring-forming carbon atoms.

In Formula F-b, U and V may each independently be a substituted or unsubstituted hydrocarbon ring of 5 to 30 ring-forming carbon atoms, or a substituted or unsubstituted heterocycle of 2 to 30 ring-forming carbon atoms.

In Formula F-b, the number of rings represented by U and V may each independently be 0 or 1. For example, in Formula F-b, when the number of U or V is 1, one ring forms a fused ring at the designated part by U or V, and when the number of U or V is 0, a ring is not present at the designated part by U or V. For example, when the number of U is 0, and the number of V is 1, or when the number of U is 1, and the number of V is 0, a fused ring having the fluorene core of Formula F-b may be a ring compound with four rings. In some embodiments, when the number of both U and V is 0, the fused ring of Formula F-b may be a ring compound with three rings. In some embodiments, when the number of both U and V is 1, a fused ring having the fluorene core of Formula F-b may be a ring compound with five rings.

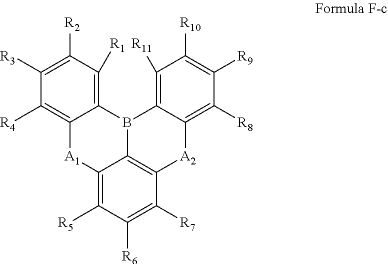

In Formula F-c, A₁ and A₂ may each independently be O, S, Se, or NRₘ, and Rₘ may be a hydrogen atom, a deuterium atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms. R₁ to R₁₁ may each independently be a hydrogen atom, a deuterium atom, a halogen atom, a cyano group, a substituted or unsubstituted amine group, a substituted or unsubstituted boryl group, a substituted or unsubstituted oxy group, a substituted or unsubstituted thio group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, or combined with an adjacent group to form a ring.

In Formula F-c, A₁ and A₂ may each independently be combined with the substituents of an adjacent ring to form a fused ring. For example, when A₁ and A₂ may each independently be NRₘ, A₁ may be combined with R₄ or R₅ to form a ring. In some embodiments, A₂ may be combined with R₇ or R₈ to form a ring.

In an embodiment, the emission layer EML may include as a generally used/generally available dopant material, styryl derivatives (for example, 1,4-bis[2-(3-N-ethylcarbazoryl)vinyl]benzene (BCzVB), 4-(di-p-tolylamino)-4′-[(di-p-tolylamino)styryl]stilbene (DPAVB), N-(4-((E)-2-(6-((E) (diphenylamino)styryl)naphthalen-2-yl)vinyl)phenyl)-N-phenylbenzenamine (N-BDAVBi), and 4,4′-bis[2-(4-(N,N-diphenylamino)phenyl)vinyl]biphenyl (DPAVBi)), perylene and the derivatives thereof (for example, 2,5,8,11-tetra-t-butylperylene (TBP)), pyrene and the derivatives thereof (for example, 1,1-dipyrene, 1,4-dipyrenylbenzene, and 1,4-bis(N,N-diphenylamino)pyrene), etc.

The EML may include a generally used/generally available phosphorescence dopant material. For example, the phosphorescence dopant may use a metal complex including iridium (Ir), platinum (Pt), osmium (Os), gold (Au), titanium (Ti), zirconium (Zr), hafnium (Hf), europium (Eu), terbium (Tb) or thulium (Tm). Particularly, iridium(III) bis(4,6-difluorophenylpyridinato-N,C2′) (Flrpic), bis(2,4-difluorophenylpyridinato)-tetrakis(1-pyrazolyl)borate iridium(III) (Fir6), or platinum octaethyl porphyrin (PtOEP) may be used as the phosphorescence dopant. However, an embodiment of the present disclosure is not limited thereto.

The EML may include a generally used/generally available phosphorescence dopant material. For example, the phosphorescence dopant may use a metal complex including iridium (Ir), platinum (Pt), osmium (Os), gold (Au), titanium (Ti), zirconium (Zr), hafnium (Hf), europium (Eu), terbium (Tb) or thulium (Tm). Particularly, iridium(III) bis(4,6-difluorophenylpyridinato-N,C2′)picolinate (Flrpic), bis(2,4-difluorophenylpyridinato)-tetrakis(1-pyrazolyl)borate iridium(III) (Fir6), or platinum octaethyl porphyrin (PtOEP) may be used as the phosphorescence dopant. However, an embodiment of the present disclosure is not limited thereto.

The emission layer EML may include a quantum dot material. The core of the quantum dot may be selected from a II-VI group compound, a III-VI group compound, a I-III-VI group compound, a III-V group compound, a III-II-V group compound, a IV-VI group compound, a IV group element, a IV group compound, and one or more combinations thereof.

The II-VI group compound may be selected from the group including (e.g., consisting of): a binary compound selected from the group including (e.g., consisting of) CdSe, CdTe, CdS, ZnS, ZnSe, ZnTe, ZnO, HgS, HgSe, HgTe, MgSe, MgS, and mixtures thereof; a ternary compound selected from the group including (e.g., consisting of) CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, MgZnS, and mixtures thereof; and a quaternary compound selected from the group including (e.g., consisting of) HgZnTeS, CdZnSeS, CdZnSeTe, CdZnSTe, CdHgSeS, CdHgSeTe, CdHgSTe, HgZnSeS, HgZnSeTe, HgZnSTe, and mixtures thereof.

The III-VI group compound may include a binary compound such as In₂S₃, and In₂Se₃, a ternary compound such as InGaS₃, and/or InGaSe₃, or one or more combinations thereof.

The I-III-VI group compound may be selected from a ternary compound selected from the group including (e.g., consisting of) AgInS, AgInS₂, CuInS, CuInS₂, AgGaS₂, CuGaS₂, CuGaO₂, AgGaO₂, AgAlO₂ and one or more compounds or mixtures thereof, and/or a quaternary compound such as AgInGaS₂, and/or CuInGaS₂.

The III-V group compound may be selected from the group including (e.g., consisting of) a binary compound selected from the group including (e.g., consisting of) GaN, GaP, GaAs, GaSb, AlN, AlP, AlAs, AlSb, InN, InP, InAs, InSb, and mixtures thereof, a ternary compound selected from the group including (e.g., consisting of) GaNP, GaNAs, GaNSb, GaPAs, GaPSb, AlNP, AlNAs, AlNSb, AlPAs, AlPSb, InGaP, InAlP, InNP, InNAs, InNSb, InPAs, InPSb, and mixtures thereof, and a quaternary compound selected from the group including (e.g., consisting of) GaAlNP, GaAlNAs, GaAlNSb, GaAlPAs, GaAlPSb, GaInNP, GaInNAs, GaInNSb, GaInPAs, GaInPSb, InAlNP, InAlNAs, InAlNSb, InAlPAs, InAlPSb, and mixtures thereof. In some embodiments, the III-V group compound may further include a II group metal. For example, InZnP, etc. may be selected as a III-II-V group compound.

The IV-VI group compound may be selected from the group including (e.g., consisting of) a binary compound selected from the group including (e.g., consisting of) SnS, SnSe, SnTe, PbS, PbSe, PbTe, and mixtures thereof, a ternary compound selected from the group including (e.g., consisting of) SnSeS, SnSeTe, SnSTe, PbSeS, PbSeTe, PbSTe, SnPbS, SnPbSe, SnPbTe, and mixtures thereof, and a quaternary compound selected from the group including (e.g., consisting of) SnPbSSe, SnPbSeTe, SnPbSTe, and mixtures thereof. The IV group element may be selected from the group including (e.g., consisting of) Si, Ge, and a mixture thereof. The IV group compound may be a binary compound selected from the group including (e.g., consisting of) SiC, SiGe, and a mixture thereof.

In this embodiment, the binary compound, the ternary compound or the quaternary compound may be present at a substantially uniform concentration in a particle (particle form) or may be present at a partially different concentration distribution state in the same particle. In some embodiments, a core/shell structure in which one quantum dot wraps (surrounds) another quantum dot may be possible. The interface of the core and the shell may have a concentration gradient in which the concentration of an element present in the shell is decreased toward the center.

In some embodiments, the quantum dot may have the above-described core-shell structure including a core including a nanocrystal and a shell wrapping (surrounding) the core. The shell of the quantum dot may play the role of a protection layer for preventing (reducing) the chemical deformation of the core to maintain semiconductor properties and/or a charging layer for imparting the quantum dot with electrophoretic properties. The shell may have a single layer or a multilayer. Examples of the shell of the quantum dot may include a metal or non-metal oxide, a semiconductor compound, or one or more combinations thereof.

For example, the metal or non-metal oxide may include a binary compound such as SiO₂, Al₂O₃, TiO₂, ZnO, MnO, Mn₂O₃, Mn₃O₄, CuO, FeO, Fe₂O₃, Fe₃O₄, CoO, Co₃O₄ and NiO, or a ternary compound such as MgAl₂O₄, CoFe₂O₄, NiFe₂O₄ and CoMn₂O₄, but an embodiment of the present disclosure is not limited thereto.

Also, the semiconductor compound may include CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, etc., but an embodiment of the present disclosure is not limited thereto.

The quantum dot may have a full width of half maximum (FWHM) of emission wavelength spectrum of about 45 nm or less, about 40 nm or less, or about 30 nm or less. Within these ranges, color purity or color reproducibility may be improved. In some embodiments, light emitted via such a quantum dot is emitted in all directions, and light view angle properties may be improved (increased).

In some embodiments, the shape of the quantum dot may be generally used shapes in the art, without limitation. More particularly, the shape of spherical, pyramidal, multi-arm, or cubic nanoparticle, nanotube, nanowire, nanofiber, nanoplate particle, etc. may be used.

The quantum dot may control the color of light emitted according to the particle size, and accordingly, the quantum dot may have one or more suitable emission colors such as blue, red and green.

In the light emitting elements ED of embodiments, as shown in FIG. 3 to FIG. 6, the electron transport region ETR is provided on the emission layer EML. The electron transport region ETR may include at least one of an electron blocking layer HBL, an electron transport layer ETL or an electron injection layer EIL. However, an embodiment of the present disclosure is not limited thereto.

The electron transport region ETR may include the above-described polycyclic compound represented by Formula 1. For example, the polycyclic compound of an embodiment may be included in a layer adjacent to the emission layer EML among the above-described multiple layers. For example, the polycyclic compound of an embodiment may be included in an electron transport layer ETL. However, an embodiment of the present disclosure is not limited thereto. In addition to the electron transport layer ETL, the polycyclic compound of an embodiment may be included in a hole blocking layer HBL or an electron injection layer EIL. For the polycyclic compound, the substantially the same explanation used for the polycyclic compound included in the emission layer EML may be applied to, and a more detailed explanation thereon will not be provided.

The polycyclic compound of an embodiment includes at least one or more bulky specific substituents represented by Formula 2 in an indolocarbazole skeleton, and electron transport properties may become excellent or suitable. Also, the indolocarbazole skeleton of 5,12-dihydroindolo[3,2-a]carbazole is included, and a high level of the lowest excitation triplet energy may be observed.

Accordingly, by including the polycyclic compound of an embodiment in an electron transport region ETR, the light emitting element ED of an embodiment may show low driving voltage properties and may achieve high efficiency. For example, by including the polycyclic compound of an embodiment in a hole transport layer HTL, the light emitting element ED of an embodiment may achieve high efficiency and low driving voltage properties. However, an embodiment of the present disclosure is not limited thereto, and multiple layers of the electron transport region ETR may include the polycyclic compound of an embodiment, without limitation.

In the present disclosure, the electron transport region ETR may have a single layer formed using a single material, a single layer formed using multiple different materials, or a multilayer structure having multiple layers formed using multiple different materials.

For example, the electron transport region ETR may have a single layer structure of an electron injection layer EIL or an electron transport layer ETL, or a single layer structure formed using an electron injection material and an electron transport material. Further, the electron transport region ETR may have a single layer structure formed using multiple different materials, or a structure stacked from the emission layer EML of electron transport layer ETL/electron injection layer EIL, or hole blocking layer HBL/electron transport layer ETL/electron injection layer EIL, without limitation. The thickness of the electron transport region ETR may be, for example, from about 1,000 Å to about 1,500 Å.

The electron transport region ETR may be formed using one or more suitable methods such as a vacuum deposition method, a spin coating method, a cast method, a Langmuir-Blodgett (LB) method, an inkjet printing method, a laser printing method, and/or a laser induced thermal imaging (LITI) method.

The electron transport region ETR may include a compound represented by Formula ET-1.

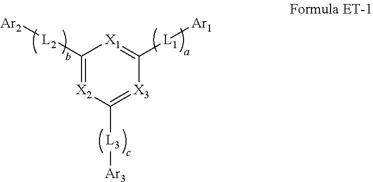

In Formula ET-1, at least one selected from among X₁ to X₃ is N, and the remainder are CRₐ. Rₐ may be a hydrogen atom, a deuterium atom, a substituted or unsubstituted alkyl of 1 to 20 carbon atoms, a substituted or unsubstituted aryl group of 6 to 30 ring-forming carbon atoms, or a substituted or unsubstituted heteroaryl group of 2 to 30 ring-forming carbon atoms. Ar₁ to Ar₃ may each independently be a hydrogen atom, a deuterium atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms.

In Formula ET-1, “a” to “c” may each independently be an integer of 0 to 10. In Formula ET-1, “L₁” to “L₃” may each independently be a direct linkage, a substituted or unsubstituted arylene group of 6 to 30 ring-forming carbon atoms, or a substituted or unsubstituted heteroarylene group of 2 to 30 ring-forming carbon atoms. In some embodiments, when “a” to “c” are integers of 2 or more, “L₁” to “L₃” may each independently be a substituted or unsubstituted arylene group of 6 to 30 ring-forming carbon atoms, or a substituted or unsubstituted heteroarylene group of 2 to 30 ring-forming carbon atoms.

The electron transport region ETR may include an anthracene-based compound. However, an embodiment of the present disclosure is not limited thereto, and the electron transport region ETR may include, for example, tris(8-hydroxyquinolinato)aluminum (Alq₃), 1,3,5-tri[(3-pyridyl)-phen-3-yl]benzene, 2,4,6-tris(3′-(pyridin-3-yl)biphenyl-3-yl)-1,3,5-triazine, 2-(4-(N-phenylbenzoimidazolyl-1-ylphenyl)-9,10-dinaphthylanthracene, 1,3,5-tri(1-phenyl-1H-benzo[d]imidazol-2-yl)benzene (TPBi), 2,9-dimethyl-4,7-diphenyl-1,10-phenanthroline (BCP), 4,7-diphenyl-1,10-phenanthroline (Bphen), 3-(4-biphenylyl)-4-phenyl-5-tert-butylphenyl-1,2,4-triazole (TAZ), 4-(naphthalen-1-yl)-3,5-diphenyl-4H-1,2,4-triazole (NTAZ), 2-(4-biphenylyl)-5-(4-tert-butylphenyl)-1,3,4-oxadiazole (tBu-PBD), bis(2-methyl-8-quinolinolato-N1,O8)-(1,1′-biphenyl-4-olato)aluminum (BAlq), berylliumbis(benzoquinolin-10-olate (Bebq₂), 9,10-di(naphthalene-2-yl)anthracene (ADN), 1,3-bis[3,5-di(pyridin-3-yl)phenyl]benzene (BmPyPhB), and/or mixtures thereof, without limitation.

The electron transport region ETR may include at least one selected from among Compounds ET1 to ET36.

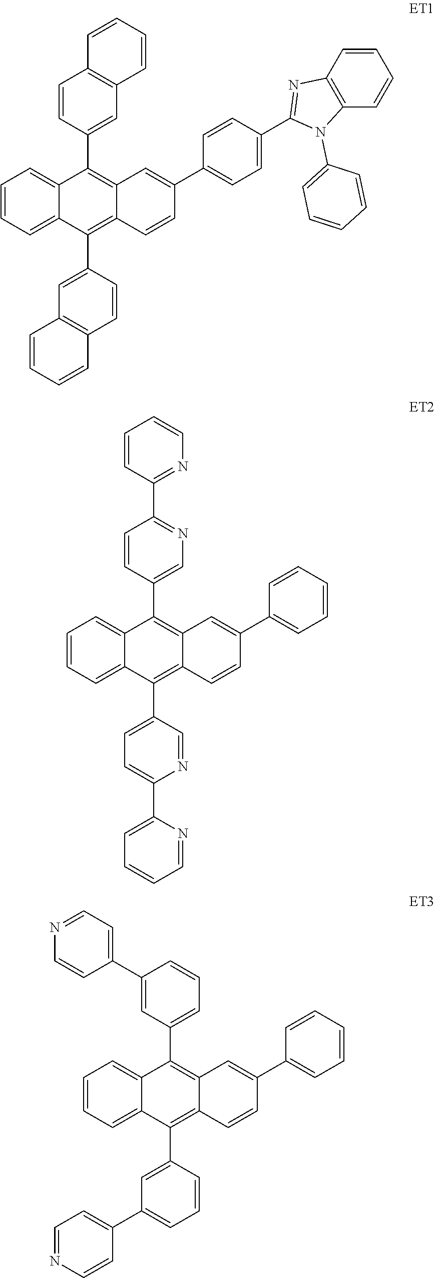 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

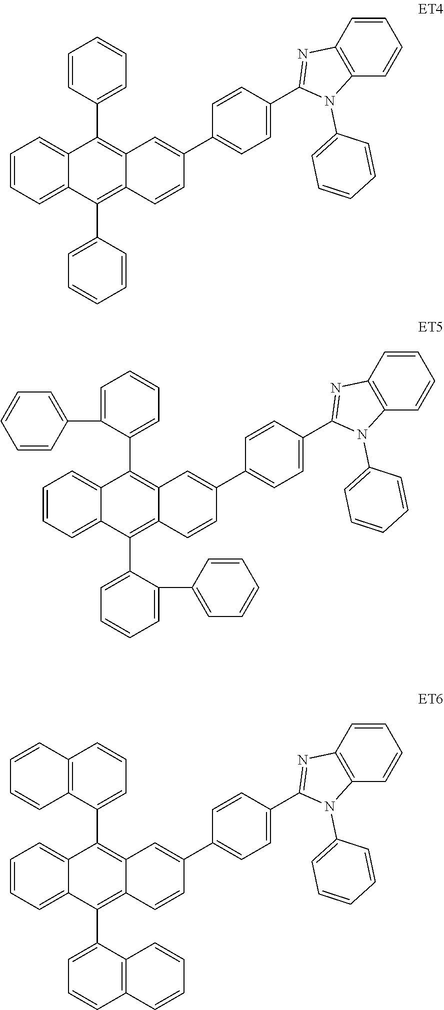 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1]

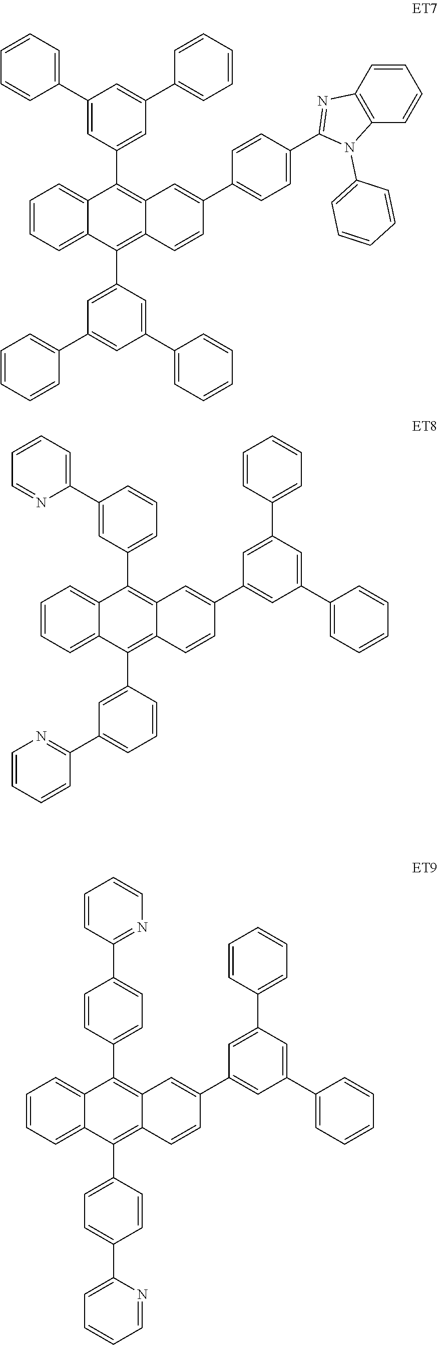

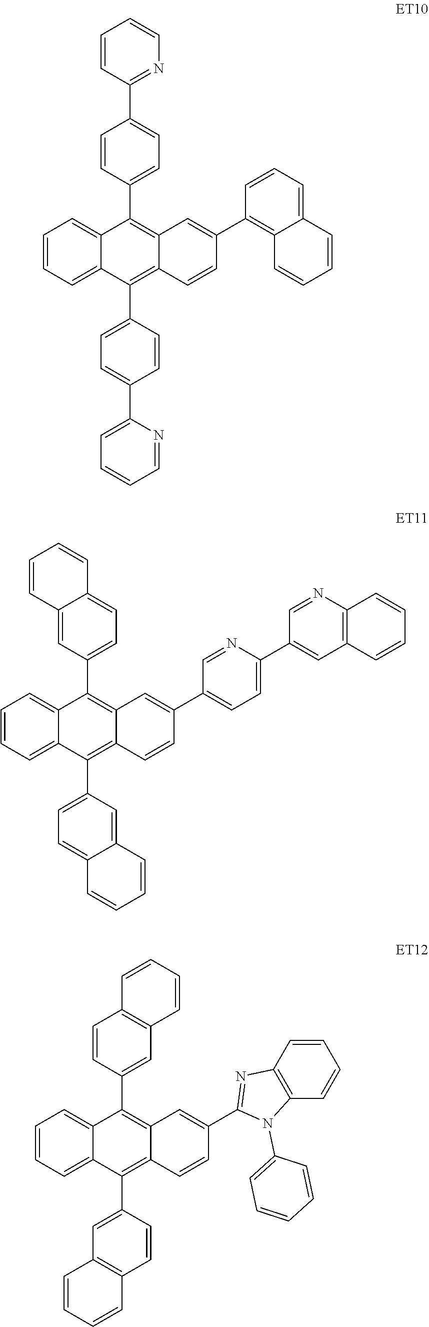 [SMILES: c1ccc(-c2ccc(-c3c4ccccc4c(-c4ccc(-c5ccccn5)cc4)c4cc(-c5cccc6ccccc56)ccc34)cc2)nc1 ; c1ccc2cc(-c3c4ccccc4c(-c4ccc5ccccc5c4)c4cc(-c5ccc(-c6cnc7ccccc7c6)nc5)ccc34)ccc2c1 ; c1ccc(-n2c(-c3ccc4c(-c5ccc6ccccc6c5)c5ccccc5c(-c5ccc6ccccc6c5)c4c3)nc3ccccc32)cc1]

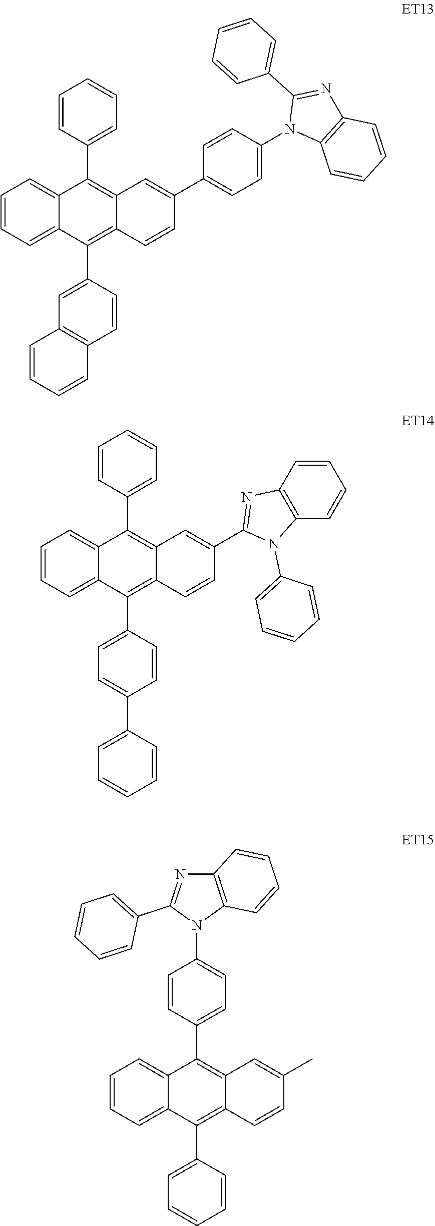 [SMILES: c1ccc(-c2c3ccccc3c(-c3ccc4ccccc4c3)c3ccc(-c4ccc(-n5c(-c6ccccc6)nc6ccccc65)cc4)cc23)cc1 ; c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1]

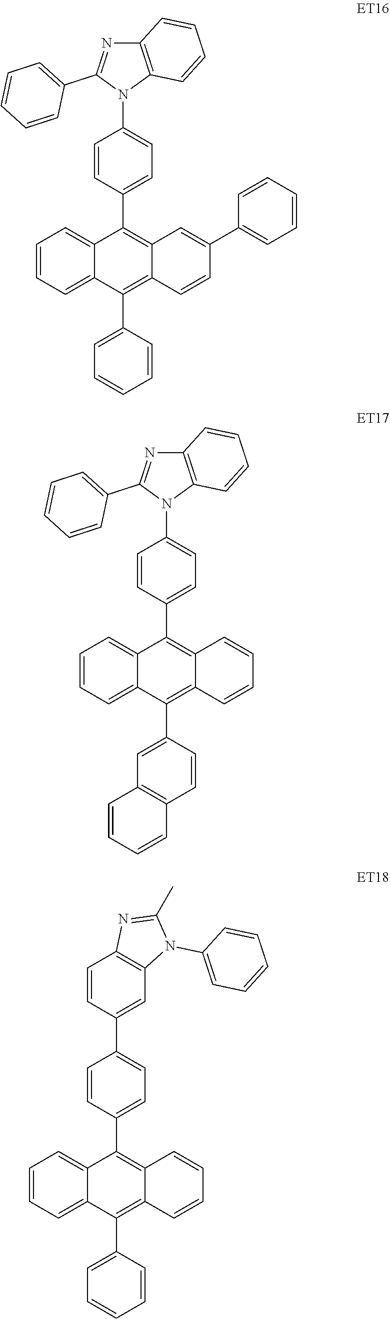 [SMILES: c1ccc(-c2ccc3c(-c4ccccc4)c4ccccc4c(-c4ccc(-n5c(-c6ccccc6)nc6ccccc65)cc4)c3c2)cc1 ; c1ccc(-c2nc3ccccc3n2-c2ccc(-c3c4ccccc4c(-c4ccc5ccccc5c4)c4ccccc34)cc2)cc1 ; Cc1nc2ccc(-c3ccc(-c4c5ccccc5c(-c5ccccc5)c5ccccc45)cc3)cc2n1-c1ccccc1]

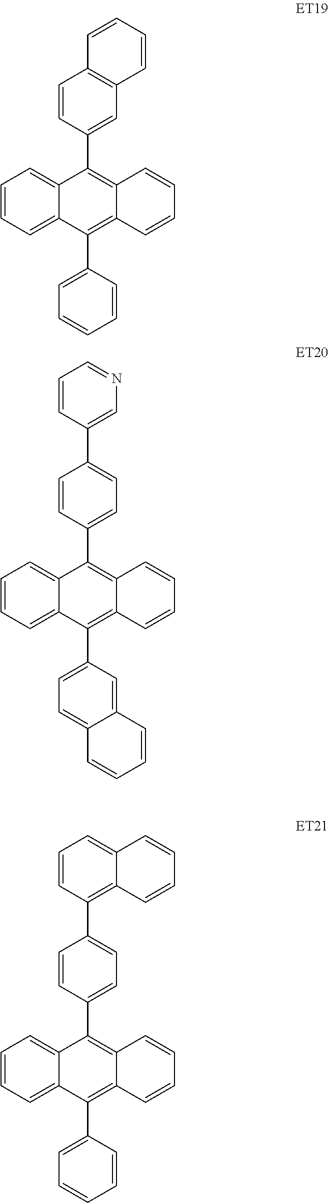

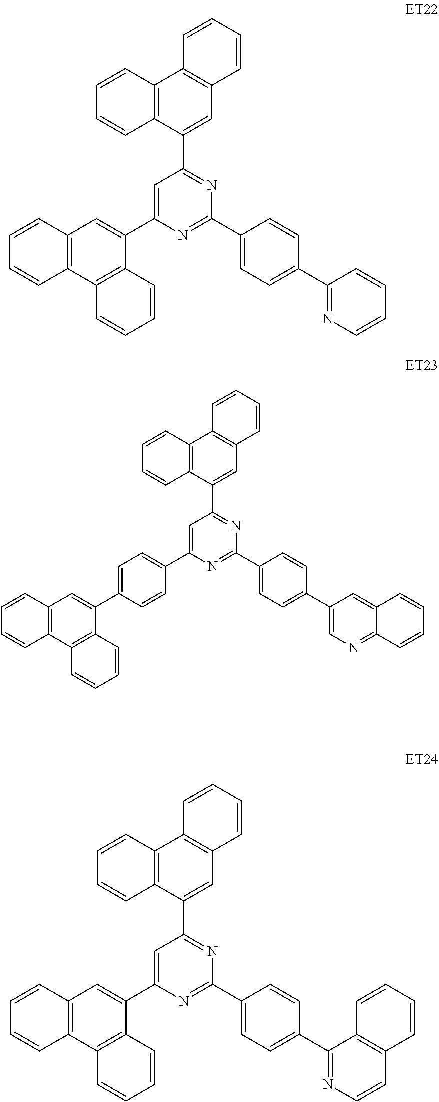 [SMILES: c1ccc(-c2ccc(-c3nc(-c4cc5ccccc5c5ccccc45)cc(-c4cc5ccccc5c5ccccc45)n3)cc2)nc1 ; c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1]

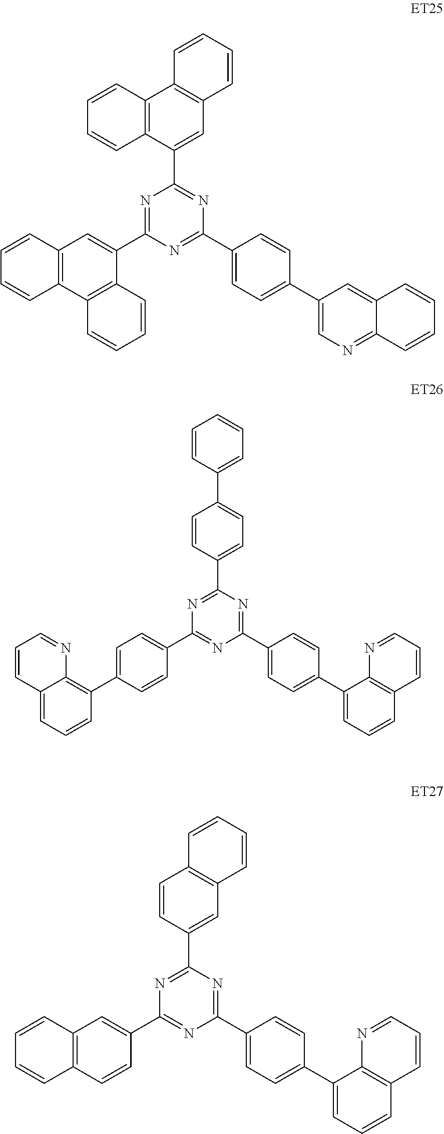 [SMILES: c1ccc2ncc(-c3ccc(-c4nc(-c5cc6ccccc6c6ccccc56)nc(-c5cc6ccccc6c6ccccc56)n4)cc3)cc2c1 ; c1ccc(-c2ccc(-c3nc(-c4ccc(-c5cccc6cccnc56)cc4)nc(-c4ccc(-c5cccc6cccnc56)cc4)n3)cc2)cc1 ; c1ccc2cc(-c3nc(-c4ccc(-c5cccc6cccnc56)cc4)nc(-c4ccc5ccccc5c4)n3)ccc2c1]

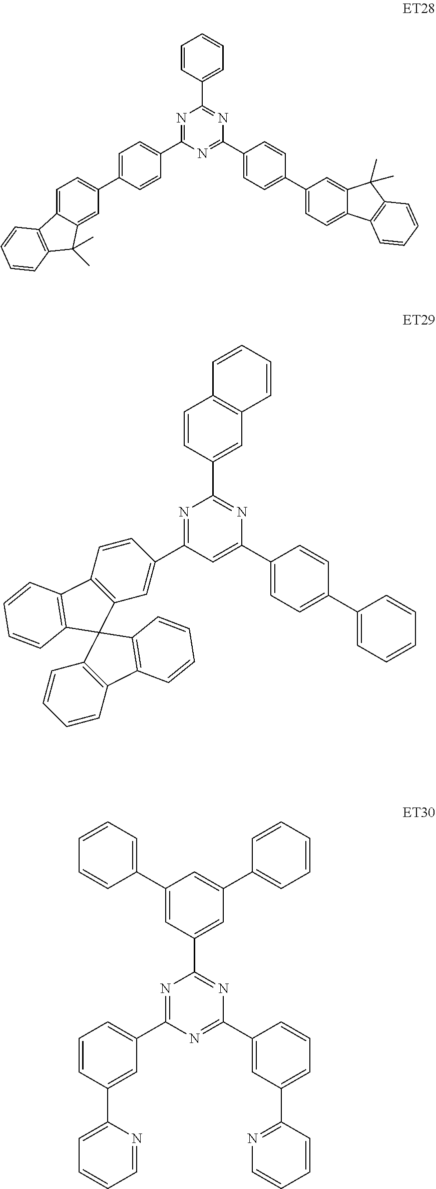 [SMILES: CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21 ; c1ccc(-c2ccc(-c3cc(-c4ccc5c(c4)C4(c6ccccc6-c6ccccc64)c4ccccc4-5)nc(-c4ccc5ccccc5c4)n3)cc2)cc1 ; c1ccc(-c2cc(-c3ccccc3)cc(-c3nc(-c4cccc(-c5ccccn5)c4)nc(-c4cccc(-c5ccccn5)c4)n3)c2)cc1]

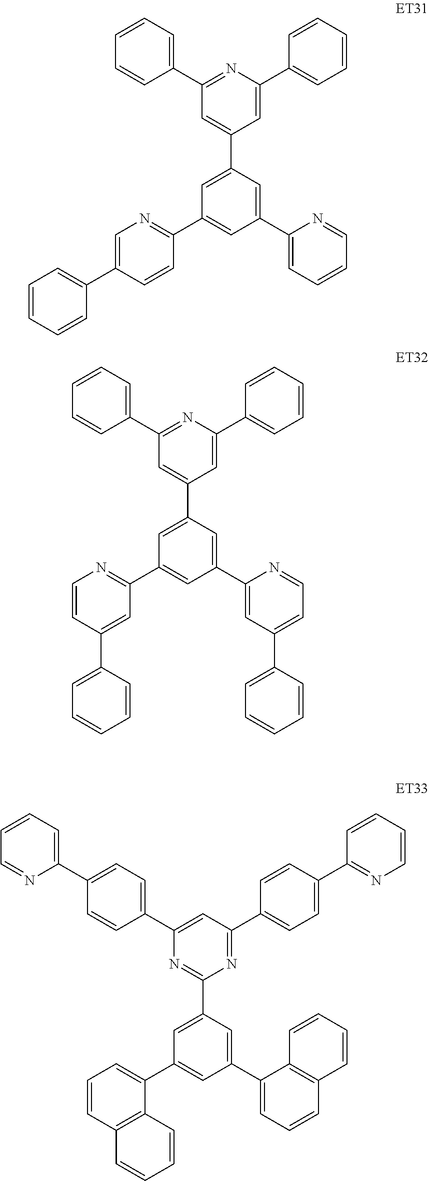 [SMILES: c1ccc(-c2ccc(-c3cc(-c4cc(-c5ccccc5)nc(-c5ccccc5)c4)cc(-c4ccccn4)c3)nc2)cc1 ; c1ccc(-c2ccnc(-c3cc(-c4cc(-c5ccccc5)nc(-c5ccccc5)c4)cc(-c4cc(-c5ccccc5)ccn4)c3)c2)cc1 ; c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1]

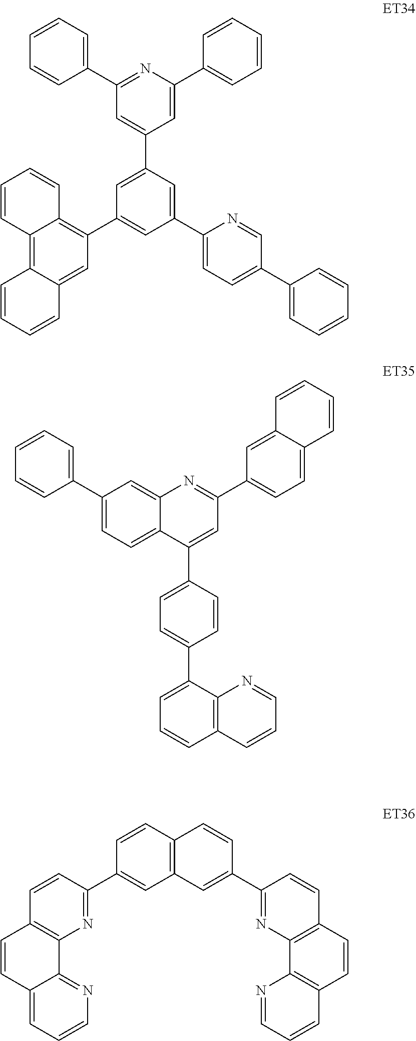 [SMILES: c1ccc(-c2ccc(-c3cc(-c4cc(-c5ccccc5)nc(-c5ccccc5)c4)cc(-c4cc5ccccc5c5ccccc45)c3)nc2)cc1 ; c1ccc(-c2ccc3c(-c4ccc(-c5cccc6cccnc56)cc4)cc(-c4ccc5ccccc5c4)nc3c2)cc1 ; c1cnc2c(c1)ccc1ccc(-c3ccc4ccc(-c5ccc6ccc7cccnc7c6n5)cc4c3)nc12]

In some embodiments, the electron transport region ETR may include a metal halide such as LiF, NaCl, CsF, RbCl, RbI, CuI and/or KI, a metal in lanthanoides such as Yb, or a co-depositing material of the metal halide and the metal in lanthanoides. For example, the electron transport region ETR may include KI:Yb, RbI:Yb, LiF:Yb, etc., as the co-depositing material. In some embodiments, the electron transport region ETR may use a metal oxide such as Li₂O and BaO, or 8-hydroxy-lithium quinolate (Liq). However, an embodiment of the present disclosure is not limited thereto. The electron transport region ETR also may be formed using a mixture material of an electron transport material and an insulating organo metal salt. The organo metal salt may be a material having an energy band gap of about 4 eV or more. For example, the organo metal salt may include, for example, metal acetates, metal benzoates, metal acetoacetates, metal acetylacetonates, or metal stearates.

The electron transport region ETR may include at least one of 2,9-dimethyl-4,7-diphenyl-1,10-phenanthroline (BCP), diphenyl(4-(triphenylsilyl)phenyl)phosphine oxide (TSPO1) or 4,7-diphenyl-1,10-phenanthroline (Bphen) in addition to the aforementioned materials. However, an embodiment of the present disclosure is not limited thereto.

The electron transport region ETR may include the compounds of the electron transport region in at least one selected from among an electron injection layer EIL, an electron transport layer ETL, and a hole blocking layer HBL.

When the electron transport region ETR includes the electron transport layer ETL, the thickness of the electron transport layer ETL may be from about 100 Å to about 1,000 Å, for example, from about 150 Å to about 500 Å. When the thickness of the electron transport layer ETL satisfies the above-described range, satisfactory (suitable) electron transport properties may be obtained without a substantial increase of a driving voltage. When the electron transport region ETR includes the electron injection layer EIL, the thickness of the electron injection layer EIL may be from about 1 Å to about 100 Å, or from about 3 Å to about 90 Å. When the thickness of the electron injection layer EIL satisfies the above described ranges, satisfactory (suitable) electron injection properties may be obtained without inducing a substantial increase of a driving voltage.

The second electrode EL2 is provided on the electron transport region ETR. The second electrode EL2 may be a common electrode. The second electrode EL2 may be a cathode or an anode, but an embodiment of the present disclosure is not limited thereto. For example, when the first electrode EL1 is an anode, the second cathode EL2 may be a cathode, and when the first electrode EL1 is a cathode, the second electrode EL2 may be an anode. The second electrode may include at least one selected from among Ag, Mg, Cu, Al, Pt, Pd, Au, Ni, Nd, Ir, Cr, Li, Ca, LiF, Mo, Ti, W, In, Sn, Zn, a compound of two or more selected therefrom, a mixture of two or more selected therefrom, and/or oxides thereof.

The second electrode EL2 may be a transmissive electrode, a transflective electrode or a reflective electrode. When the second electrode EL2 is the transmissive electrode, the second electrode EL2 may include a transparent metal oxide, for example, ITO, IZO, ZnO, ITZO, etc.

When the second electrode EL2 is the transflective electrode or the reflective electrode, the second electrode EL2 may include Ag, Mg, Cu, Al, Pt, Pd, Au, Ni, Nd, Ir, Cr, Li, Ca, LiF/Ca, LiF/Al, Mo, Ti, Yb, W, compounds including thereof, or mixtures thereof (for example, AgMg, AgYb, or MgYb). Or the second electrode EL2 may have a multilayered structure including a reflective layer or a transflective layer formed using the above-described materials and a transparent conductive layer formed using ITO, IZO, ZnO, ITZO, etc. For example, the second electrode EL2 may include the aforementioned metal materials, combinations of two or more metal materials selected from the aforementioned metal materials, or oxides of the aforementioned metal materials.

The second electrode EL2 may be connected with an auxiliary electrode. When the second electrode EL2 is connected with the auxiliary electrode, the resistance of the second electrode EL2 may decrease.

In some embodiments, on the second electrode EL2 in the light emitting element ED of an embodiment, a capping layer CPL may be further disposed. The capping layer CPL may include a multilayer or a single layer.

In an embodiment, the capping layer CPL may be an organic layer or an inorganic layer. For example, when the capping layer CPL includes an inorganic material, the inorganic material may include an alkali metal compound such as LiF, an alkaline earth metal compound such as MgF₂, SiON, SiNₓ, SiO_{y}, etc.

For example, when the capping layer CPL includes an organic material, the organic material may include α-NPD, NPB, TPD, m-MTDATA, Alq₃, Cu Pc, N4,N4,N4′,N4′-tetra(biphenyl-4-yl) biphenyl-4,4′-diamine (TPD15), 4,4′,4″-tris(carbazol sol-9-yl) triphenylamine (TCTA), etc., or includes an epoxy resin, or acrylate such as methacrylate. In some embodiments, a capping layer CPL may include at least one selected from among Compounds P1 to P5, but an embodiment of the present disclosure is not limited thereto.

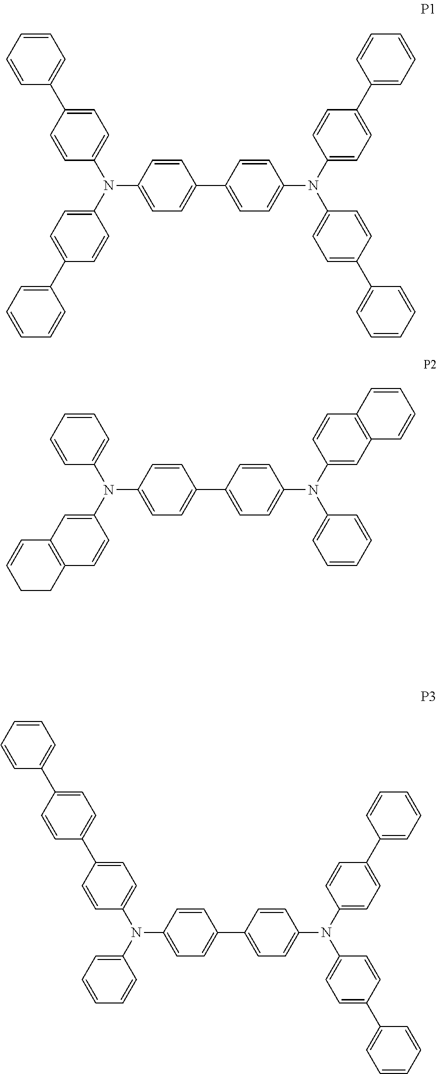 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; C1=Cc2cc(N(c3ccccc3)c3ccc(-c4ccc(N(c5ccccc5)c5ccc6ccccc6c5)cc4)cc3)ccc2CC1 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1]

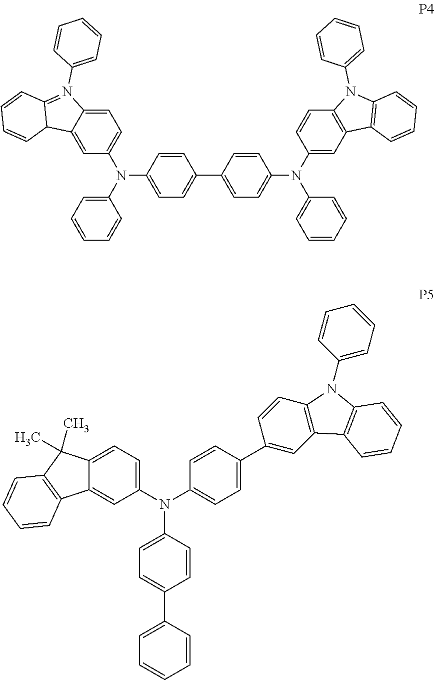

In some embodiments, the refractive index of the capping layer CPL may be about 1.6 or more. For example, the refractive index of the capping layer CPL with respect to light in a wavelength range of about 550 nm to about 660 nm may be about 1.6 or more.

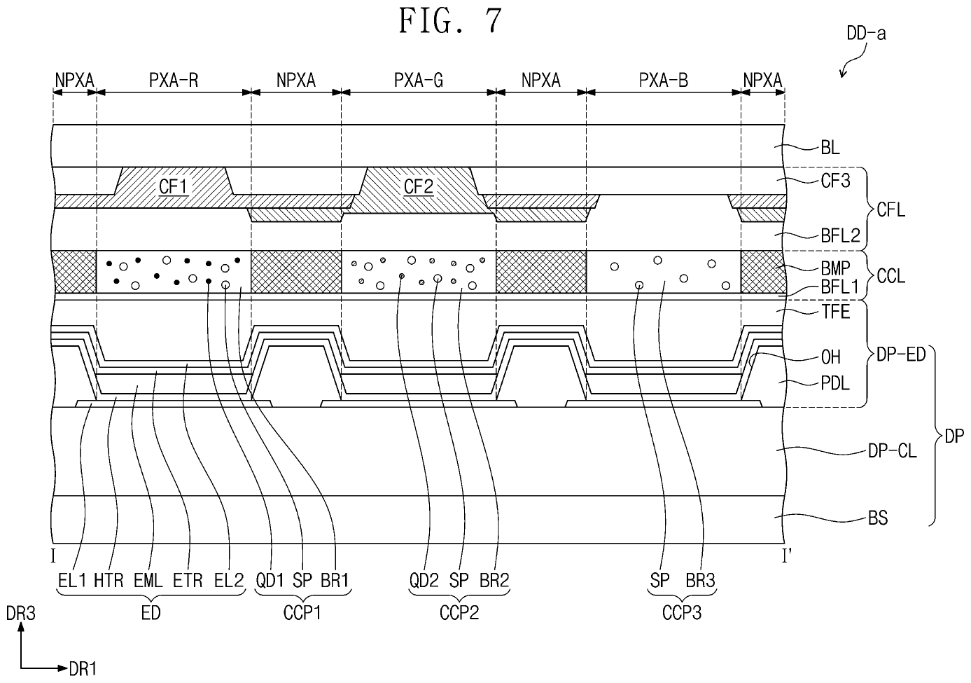

FIG. 7 to FIG. 10 are cross-sectional views on display apparatuses according to embodiments. In describing the display apparatuses of embodiments with reference to FIG. 7 to FIG. 10, the duplicated features which have been described in FIG. 1 to FIG. 6 may not be described again, but their differences will be mainly described.

Referring to FIG. 7, a display apparatus DD-a according to an embodiment may include a display panel DP including a display element layer DP-ED, a light controlling layer CCL on the display panel DP, and a color filter layer CFL.

In an embodiment shown in FIG. 7, the display panel DP includes a base layer BS, a circuit layer DP-CL provided on the base layer BS and a display element layer DP-ED, and the display element layer DP-ED may include a light emitting element ED.

The light emitting element ED may include a first electrode EL1, a hole transport region HTR on the first electrode EL1, an emission layer EML on the hole transport region HTR, an electron transport region ETR on the emission layer EML, and a second electrode EL2 on the electron transport region ETR. In some embodiments, the same structures of the light emitting elements of FIG. 3 to FIG. 6 may be applied to the structure of the light emitting element ED shown in FIG. 7.

The emission layer EML of the light emitting element ED included in the display apparatus DD-a according to an embodiment may include at least one selected from among the second compound, the third compound and the fourth compound, and the first compound of an embodiment, described above.

Referring to FIG. 7, the emission layer EML may be in an opening part OH defined in a pixel definition layer PDL. For example, the emission layer EML divided by the pixel definition layer PDL and correspondingly provided to each of luminous areas PXA-R, PXA-G and PXA-B may emit light in the same wavelength region. In the display apparatus DD-a of an embodiment, the emission layer EML may emit blue light. In some embodiments, the emission layer EML may be provided as a common layer for all luminous areas PXA-R, PXA-G and PXA-B.

The light controlling layer CCL may be on the display panel DP. The light controlling layer CCL may include a light converter. The light converter may be a quantum dot or a phosphor. The light converter may transform the wavelength of light provided and then emit. For example, the light controlling layer CCL may be a layer including a quantum dot or a layer including a phosphor.

The light controlling layer CCL may include multiple light controlling parts CCP1, CCP2 and CCP3. The light controlling parts CCP1, CCP2 and CCP3 may be separated from one another.

Referring to FIG. 7, a partition pattern BMP may be between the separated light controlling parts CCP1, CCP2 and CCP3, but an embodiment of the present disclosure is not limited thereto. In FIG. 7, the partition pattern BMP is shown not overlap with the light controlling parts CCP1, CCP2 and CCP3, but at least a portion of the edge of the light controlling parts CCP1, CCP2 and CCP3 may be overlap with the partition pattern BMP.

The light controlling layer CCL may include a first light controlling part CCP1 including a first quantum dot QD1 converting first color light provided from the light emitting element ED into second color light, a second light controlling part CCP2 including a second quantum dot QD2 converting first color light into third color light, and a third light controlling part CCP3 transmitting first color light.

In an embodiment, the first light controlling part CCP1 may provide red light which is the second color light, and the second light controlling part CCP2 may provide green light which is the third color light. The third color controlling part CCP3 may transmit and provide blue light which is the first color light provided from the light emitting element ED. For example, the first quantum dot QD1 may be a red quantum dot, and the second quantum dot QD2 may be a green quantum dot. Regarding the quantum dots QD1 and QD2, the same description used above may be applied.

In some embodiments, the light controlling layer CCL may further include a scatterer SP. The first light controlling part CCP1 may include the first quantum dot QD1 and the scatterer SP, the second light controlling part CCP2 may include the second quantum dot QD2 and the scatterer SP, and the third light controlling part CCP3 may not include (e.g., may exclude) a quantum dot but include the scatterer SP.

The scatterer SP may be an inorganic particle. For example, the scatterer SP may include at least one selected from among TiO₂, ZnO, Al₂O₃, SiO₂, and hollow silica. The scatterer SP may include at least one selected from among TiO₂, ZnO, Al₂O₃, SiO₂, and hollow silica, or may be a mixture of two or more materials selected from among TiO₂, ZnO, Al₂O₃, SiO₂, and hollow silica.

Each of the first light controlling part CCP1, the second light controlling part CCP2, and the third light controlling part CCP3 may include base resins BR1, BR2 and BR3 dispersing the quantum dots QD1 and QD2 and the scatterer SP. In an embodiment, the first light controlling part CCP1 may include the first quantum dot QD1 and the scatterer SP dispersed in the first base resin BR1, the second light controlling part CCP2 may include the second quantum dot QD2 and the scatterer SP dispersed in the second base resin BR2, and the third light controlling part CCP3 may include the scatterer particle SP dispersed in the third base resin BR3. The base resins BR1, BR2 and BR3 are mediums in which the quantum dots QD1 and QD2 and the scatterer SP are dispersed, and may be composed of one or more suitable resin compositions which may be generally referred to as a binder. For example, the base resins BR1, BR2 and BR3 may be acrylic resins, urethane-based resins, silicone-based resins, epoxy-based resins, etc. The base resins BR1, BR2 and BR3 may be transparent resins. In an embodiment, the first base resin BR1, the second base resin BR2 and the third base resin BR3 may be the same or different from each other.

The light controlling layer CCL may include a barrier layer BFL1. The barrier layer BFL1 may play the role of blocking (reducing) the penetration of moisture and/or oxygen (hereinafter, will be referred to as “humidity/oxygen”). The barrier layer BFL1 may be on the light controlling parts CCP1, CCP2 and CCP3 to block (reduce) the exposure of the light controlling parts CCP1, CCP2 and CCP3 to humidity/oxygen. In some embodiments, the barrier layer BFL1 may cover the light controlling parts CCP1, CCP2 and CCP3. In some embodiments, the barrier layer BFL2 may be provided between the light controlling parts CCP1, CCP2 and CCP3 and a color filter layer CFL.

The barrier layers BFL1 and BFL2 may include at least one inorganic layer. For example, the barrier layers BFL1 and BFL2 may be formed by including an inorganic material. For example, the barrier layers BFL1 and BFL2 may be formed by including silicon nitride, aluminum nitride, zirconium nitride, titanium nitride, hafnium nitride, tantalum nitride, silicon oxide, aluminum oxide, titanium oxide, tin oxide, cerium oxide and/or silicon oxynitride or a metal thin film securing light transmittance. In some embodiments, the barrier layers BFL1 and BFL2 may further include an organic layer. The barrier layers BFL1 and BFL2 may be composed of a single layer of multiple layers.

In the display apparatus DD-a of an embodiment, the color filter layer CFL may be on the light controlling layer CCL. For example, the color filter layer CFL may be directly on the light controlling layer CCL. In this embodiment, the barrier layer BFL2 may not be provided.

The color filter layer CFL may include filters CF1, CF2 and CF3. The color filter layer CFL may include a first filter CF1 transmitting second color light, a second filter CF2 transmitting third color light, and a third filter CF3 transmitting first color light. For example, the first filter CF1 may be a red filter, the second filter CF2 may be a green filter, and the third filter CF3 may be a blue filter. Each of the filters CF1, CF2 and CF3 may include a polymer photosensitive resin and a pigment or dye. The first filter CF1 may include a red pigment or dye, the second filter CF2 may include a green pigment or dye, and the third filter CF3 may include a blue pigment or dye. Meanwhile, an embodiment of the present disclosure is not limited thereto, and the third filter CF3 may not include the pigment or dye. The third filter CF3 may include a polymer photosensitive resin and not include (e.g., may exclude) a pigment or dye. The third filter CF3 may be transparent. The third filter CF3 may be formed using a transparent photosensitive resin.

In an embodiment, the first filter CF1 and the second filter CF2 may be yellow filters. The first filter CF1 and the second filter CF2 may be provided in one body without distinction. Each of the first to third filters CF1, CF2 and CF3 may be disposed corresponding to each of a red luminous area PXA-R, green luminous area PXA-G, and blue luminous area PXA-B.

In some embodiments, the color filter layer CFL may include a light blocking part. The color filter layer CFL may include the light blocking part disposed so as to overlap with the boundaries of the neighboring filters CF1, CF2 and CF3. The light blocking part may be a black matrix. The light blocking part may be formed by including an organic light blocking material or an inorganic light blocking material, including a black pigment or black dye. The light blocking part may divide the boundaries among adjacent filters CF1, CF2 and CF3. In an embodiment, the light blocking part may be formed as a blue filter.

On the color filter layer CFL, a base substrate BL may be disposed. The base substrate BL may be a member providing a base surface on which the color filter layer CFL, the light controlling layer CCL, etc. are disposed. The base substrate BL may be a glass substrate, a metal substrate, a plastic substrate, etc. However, an embodiment of the present disclosure is not limited thereto, and the base substrate BL may be an inorganic layer, an organic layer or a composite material layer. In some embodiments, the base substrate BL may not be provided in an embodiment.

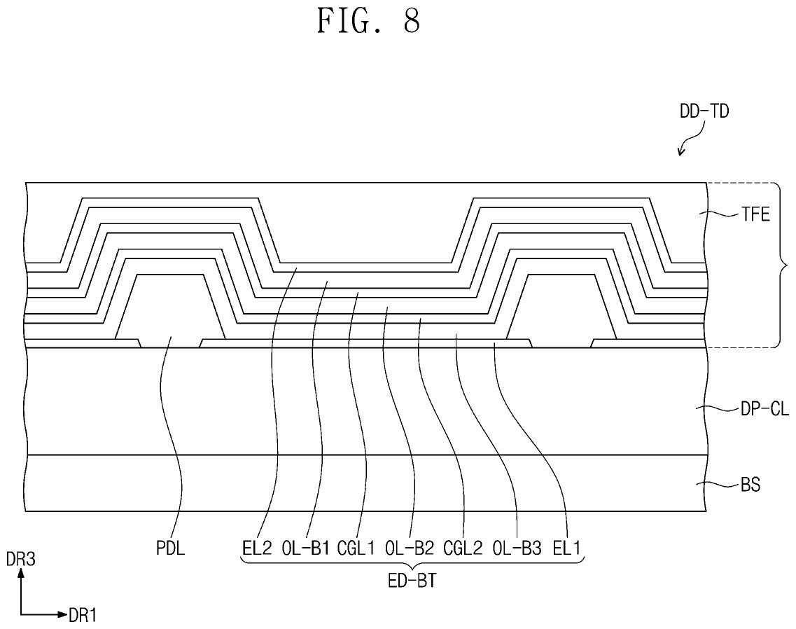

FIG. 8 is a cross-sectional view showing a portion of the display apparatus according to an embodiment. In FIG. 8, the cross-sectional view of a portion corresponding to the display panel DP in FIG. 7 is shown. In a display apparatus DD-TD of an embodiment, the light emitting element ED-BT may include multiple light emitting structures OL-B1, OL-B2 and OL-B3. The light emitting element ED-BT may include oppositely disposed first electrode EL1 and second electrode EL2, and the multiple light emitting structures OL-B1, OL-B2 and OL-B3 stacked in order in a thickness direction and provided between the first electrode EL1 and the second electrode EL2. Each of the light emitting structures OL-B1, OL-B2 and OL-B3 may include an emission layer EML (FIG. 7), a hole transport region HTR and an electron transport region ETR disposed with the emission layer EML (FIG. 7) therebetween.

For example, the light emitting element ED-BT included in the display apparatus DD-TD of an embodiment may be a light emitting element of a tandem structure including multiple emission layers.

In an embodiment shown in FIG. 8, light emitted from the light emitting structures OL-B1, OL-B2 and OL-B3 may all be blue light. However, an embodiment of the present disclosure is not limited thereto, and the wavelength regions of light emitted from the light emitting structures OL-B1, OL-B2 and OL-B3 may be different from each other. For example, the light emitting element ED-BT including the multiple light emitting structures OL-B1, OL-B2 and OL-B3 emitting light in different wavelength regions may emit white light.

Between neighboring light emitting structures OL-B1, OL-B2 and OL-B3, charge generating layers CGL1 and CGL2 may be disposed. The charge generating layers CGL1 and CGL2 may include a p-type or kind charge generating layer and/or an n-type or kind charge generating layer.

In at least one selected from among the light emitting structures OL-B1, OL-B2 and OL-B3, included in the display apparatus DD-TD of an embodiment, the first compound of an embodiment, and at least one selected from among the second compound, the third compound and the fourth compound may be included.

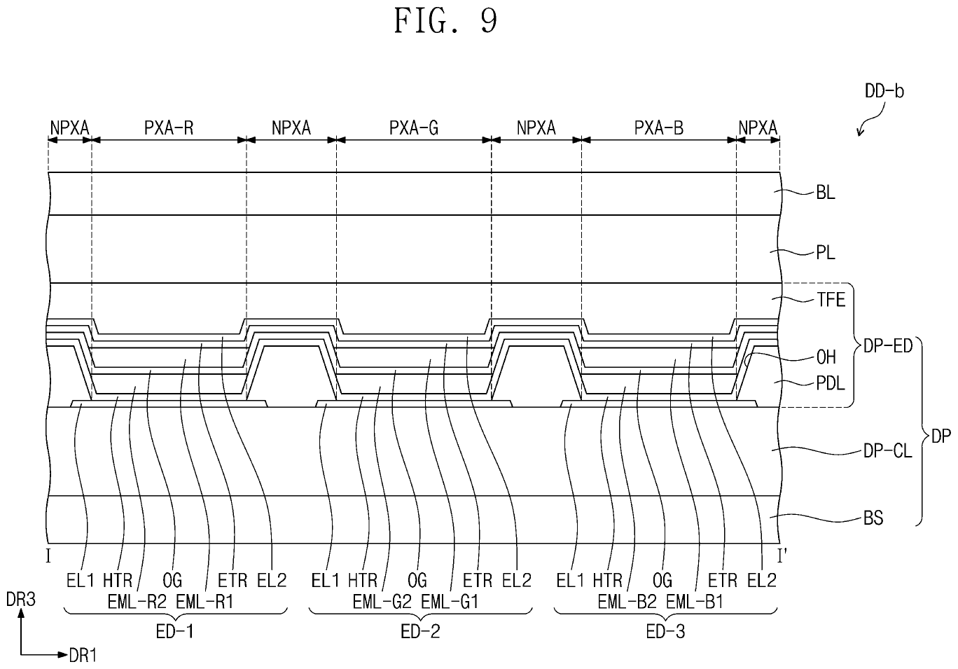

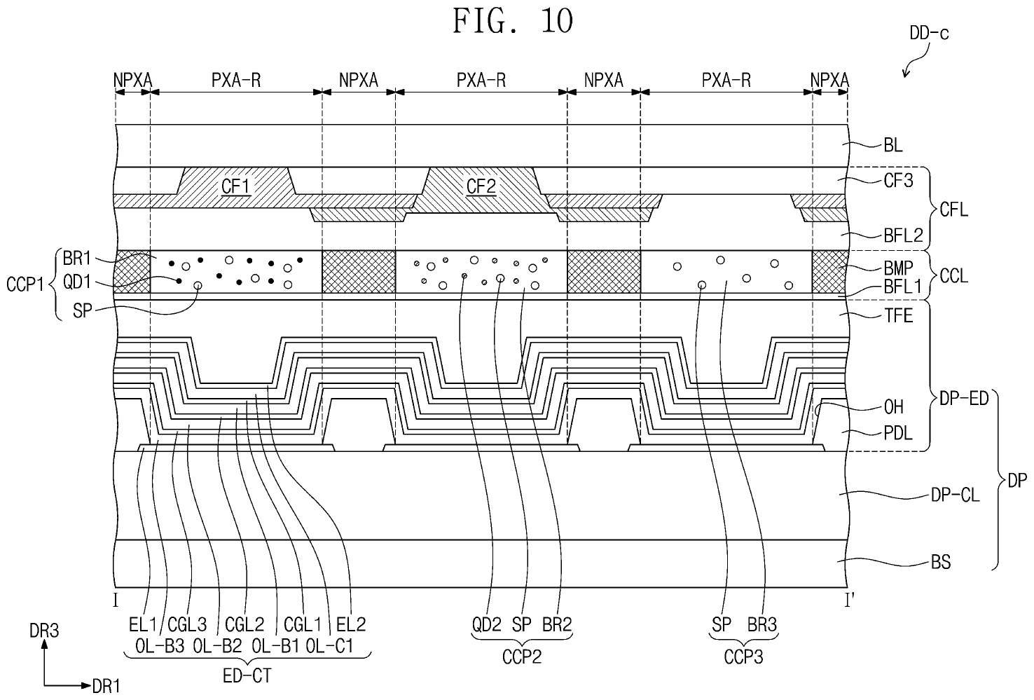

Referring to FIG. 9, a display apparatus DD-b according to an embodiment may include light emitting elements ED-1, ED-2 and ED-3, formed by stacking two emission layers. Compared to the display apparatus DD of an embodiment, shown in FIG. 2, an embodiment shown in FIG. 10 is different in that first to third light emitting elements ED-1, ED-2 and ED-3 each include two emission layers stacked in a thickness direction. In the first to third light emitting elements ED-1, ED-2 and ED-3, two emission layers may emit light in substantially the same wavelength region.

The first light emitting element ED-1 may include a first red emission layer EML-R1 and a second red emission layer EML-R2. The second light emitting element ED-2 may include a first green emission layer EML-G1 and a second green emission layer EML-G2. In some embodiments, the third light emitting element ED-3 may include a first blue emission layer EML-B1 and a second blue emission layer EML-B2. Between the first red emission layer EML-R1 and the second red emission layer EML-R2, between the first green emission layer EML-G1 and the second green emission layer EML-G2, and between the first blue emission layer EML-B1 and the second blue emission layer EML-B2, an emission auxiliary part OG may be disposed.

The emission auxiliary part OG may include a single layer or a multilayer. The emission auxiliary part OG may include a charge generating layer. For example, the emission auxiliary part OG may include an electron transport region, a charge generating layer, and a hole transport region stacked in order. The emission auxiliary part OG may be provided as a common layer in all of the first to third light emitting elements ED-1, ED-2 and ED-3. However, an embodiment of the present disclosure is not limited thereto, and the emission auxiliary part OG may be patterned and provided in an opening part OH defined in a pixel definition layer PDL.

The first red emission layer EML-R1, the first green emission layer EML-G1 and the first blue emission layer EML-B1 may be between the electron transport region ETR and the emission auxiliary part OG. The second red emission layer EML-R2, the second green emission layer EML-G2 and the second blue emission layer EML-B2 may be between the emission auxiliary part OG and the hole transport region HTR.

For example, the first light emitting element ED-1 may include a first electrode EL1, a hole transport region HTR, a second red emission layer EML-R2, an emission auxiliary part OG, a first red emission layer EML-R1, an electron transport region ETR, and a second electrode EL2, stacked in order. The second light emitting element ED-2 may include a first electrode EL1, a hole transport region HTR, a second green emission layer EML-G2, an emission auxiliary part OG, a first green emission layer EML-G1, an electron transport region ETR, and a second electrode EL2, stacked in order. The third light emitting element ED-3 may include a first electrode EL1, a hole transport region HTR, a second blue emission layer EML-B2, an emission auxiliary part OG, a first blue emission layer EML-B1, an electron transport region ETR, and a second electrode EL2, stacked in order.

In some embodiments, an optical auxiliary layer PL may be on a display element layer DP-ED. The optical auxiliary layer PL may include a polarization layer. The optical auxiliary layer PL may be on a display panel DP and may control reflected light at the display panel DP by external light. The optical auxiliary layer PL may not be provided in the display apparatus according to an embodiment.

Different from FIG. 8 and FIG. 9, a display apparatus DD-c in FIG. 10 is shown to include four light emitting structures OL-B1, OL-B2, OL-B3 and OL-C1. A light emitting element ED-CT may include oppositely disposed first electrode EL1 and second electrode EL2, and first to fourth light emitting structures OL-B1, OL-B2, OL-B3 and OL-C1 stacked in order in a thickness direction between the first electrode EL1 and the second electrode EL2. Between the first to fourth light emitting structures OL-B1, OL-B2, OL-B3 and OL-C1, charge generating layers CGL1, CGL2 and CGL3 may be disposed. Among the four light emitting structures, the first to third light emitting structures OL-B1, OL-B2 and OL-B3 may emit blue light, and the fourth light emitting structure OL-C1 may emit green light. However, an embodiment of the present disclosure is not limited thereto, and the first to fourth light emitting structures OL-B1, OL-B2, OL-B3 and OL-C1 may emit different wavelengths of light.

Charge generating layers CGL1, CGL2 and CGL3 disposed among neighboring light emitting structures OL-B1, OL-B2, OL-B3 and OL-C1 may include a p-type or kind charge generating layer and/or an n-type or kind charge generating layer.

In at least one selected from among the light emitting structures OL-B1, OL-B2, OL-B3 and OL-C1, included in the display apparatus DD-c of an embodiment, the polycyclic compound of an embodiment may be included.

The above-described polycyclic compound of an embodiment includes a structure in which a nitrogen-containing ring is bonded, and at least one or more bulky and specific substituents are bonded to an indolocarbazole skeleton. The polycyclic compound according to an embodiment has a structure represented by Formula 1, and when the polycyclic compound of an embodiment is used as the light emitting material of a light emitting element, a high efficiency of the light emitting element may be achieved, and effects of excellent or suitable electron transfer capacity and reduction of a driving voltage may be provided (achieved).

Hereinafter, referring to embodiments and comparative embodiments, the polycyclic compound according to an embodiment and the light emitting element according to an embodiment of the present disclosure will be explained in more detail. In addition, the embodiments below are merely examples to assist in the understanding of the present disclosure, but the scope of the present disclosure is not limited thereto.

### Examples

### 1. Synthesis of Polycyclic Compound of an Embodiment

First, the synthetic method of the polycyclic compound according to an embodiment will be explained more detail illustrating the synthetic methods of Compound 1, Compound 6, Compound 10, Compound 26, Compound 58, Compound 70, Compound 150, and Compound 180. In addition, the synthetic methods of the polycyclic compounds explained hereinafter are merely embodiments, and the synthetic method of the polycyclic compound according to an embodiment of the present disclosure is not limited to the embodiments below.

### (1) Synthesis of Compound 1

Polycyclic Compound 1 according to an embodiment may be synthesized, for example, by the steps of Reaction 1.

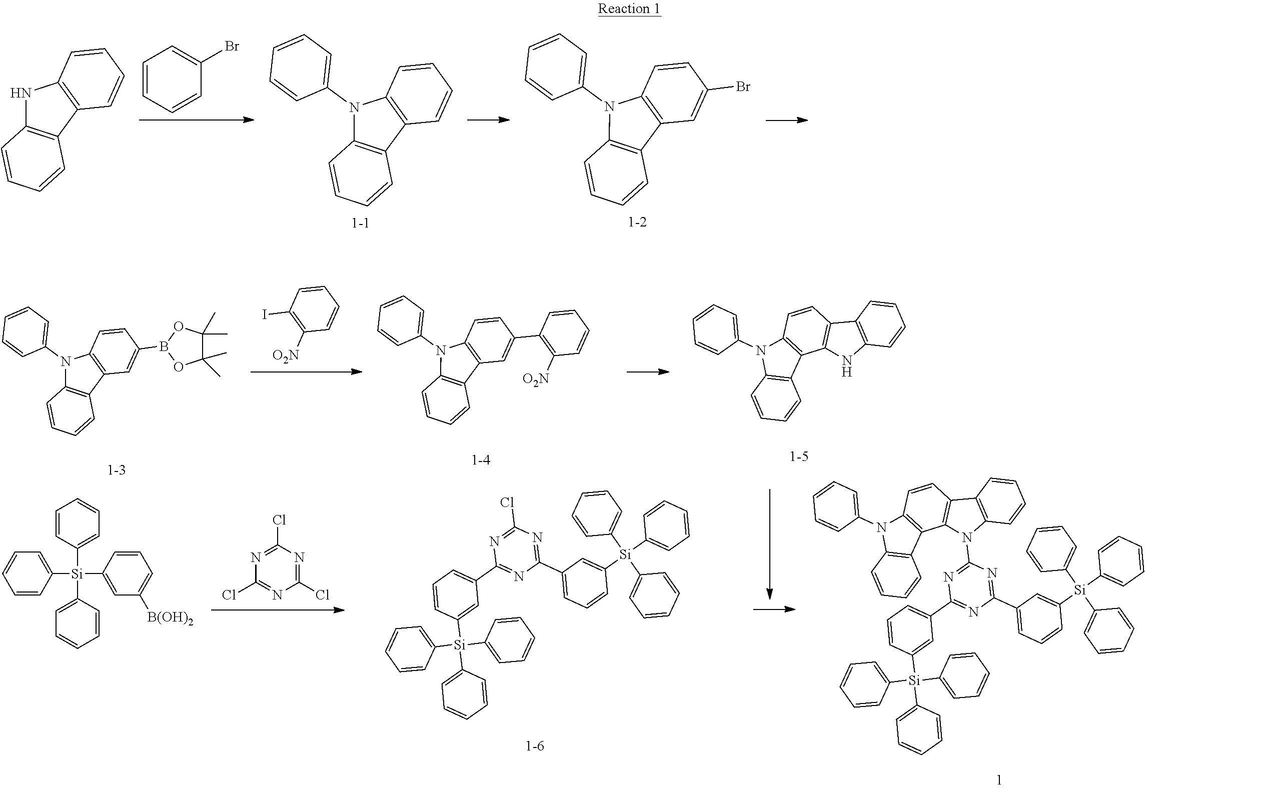

### 1) Synthesis of Intermediate 1-1

9H-carbazole (CAS #: 86-74-8) and n-BuLi were reacted, and bromobenzene (CAS #: 108-86-1) was added thereto, followed by stirring at about 70° C. overnight to obtain Intermediate 1-1. Intermediate 1-1 was identified by LC-MS.

C₁₈H₁₃N M+1: 244.11

### 2) Synthesis of Intermediate 1-2

Intermediate 1-1 and N-bromosuccinimide (CAS #: 128-08-5) were reacted to obtain Intermediate 1-2. Intermediate 1-2 was identified by LC-MS.

C₁₈H₁₂BrN M+1: 322.03

### 3) Synthesis of Intermediate 1-3

Intermediate 1-2 and bis(pinacolato)diboron (CAS #: 73183-34-3) were reacted under Pd catalyst conditions to obtain Intermediate 1-3. Intermediate 1-3 was identified by LC-MS.

C₂₄H₂₄BNO₂ M+1: 370.20

### 4) Synthesis of Intermediate 1-4

Intermediate 1-3 and 1-iodo-2-nitrobenzene (CAS #: 609-73-4) were reacted under Pd catalyst conditions to obtain Intermediate 1-4. Intermediate 1-4 was identified by LC-MS.

C₂₄H₁₆N₂O₂ M+1: 365.13

### 5) Synthesis of Intermediate 1-5

Intermediate 1-4 and triphenylphosphine (CAS #: 603-35-0) were reacted to obtain Intermediate 1-5. Intermediate 1-5 was identified by LC-MS and ¹H-NMR.

C₂₄H₁₆N2 M+1: 333.14

### 6) Synthesis of Intermediate 1-6

(3-(Triphenylsilyl)phenyl)boronic acid (CAS #: 1253912-58-1) and cyanuric chloride (CAS #: 108-77-0) were reacted under Pd catalyst conditions to obtain Intermediate 1-6. Intermediate 1-6 was identified by LC-MS.

C₅₁H₃₈ClN₃Si₂ M+1: 784.24

### 7) Synthesis of Compound 1

Intermediate 1-5 (1.36 g), Intermediate 1-6 (3.2 g), Pd₂(dba)₃ (0.15 g), P(tBu)₃ (50 wt % in xylene, 0.1 mL), NaOtBu (1.18 g), and o-xylene (50 mL) were put in RBF, followed by stirring under about 160° C. conditions overnight. After completing the reaction, the reaction solution was extracted with ethyl acetate, a collected organic layer was dried over magnesium sulfate, the solvent was evaporated, and the residue thus obtained was separated and purified by silica gel column chromatography to obtain Compound 1 (3.3 g, yield: 74%). Compound 1 was identified by LC-MS and ¹H-NMR, and the results are shown in Table 1.

### (2) Synthesis of Compound 6

Polycyclic Compound 6 according to an embodiment may be synthesized, for example, by the steps of Reaction 2.

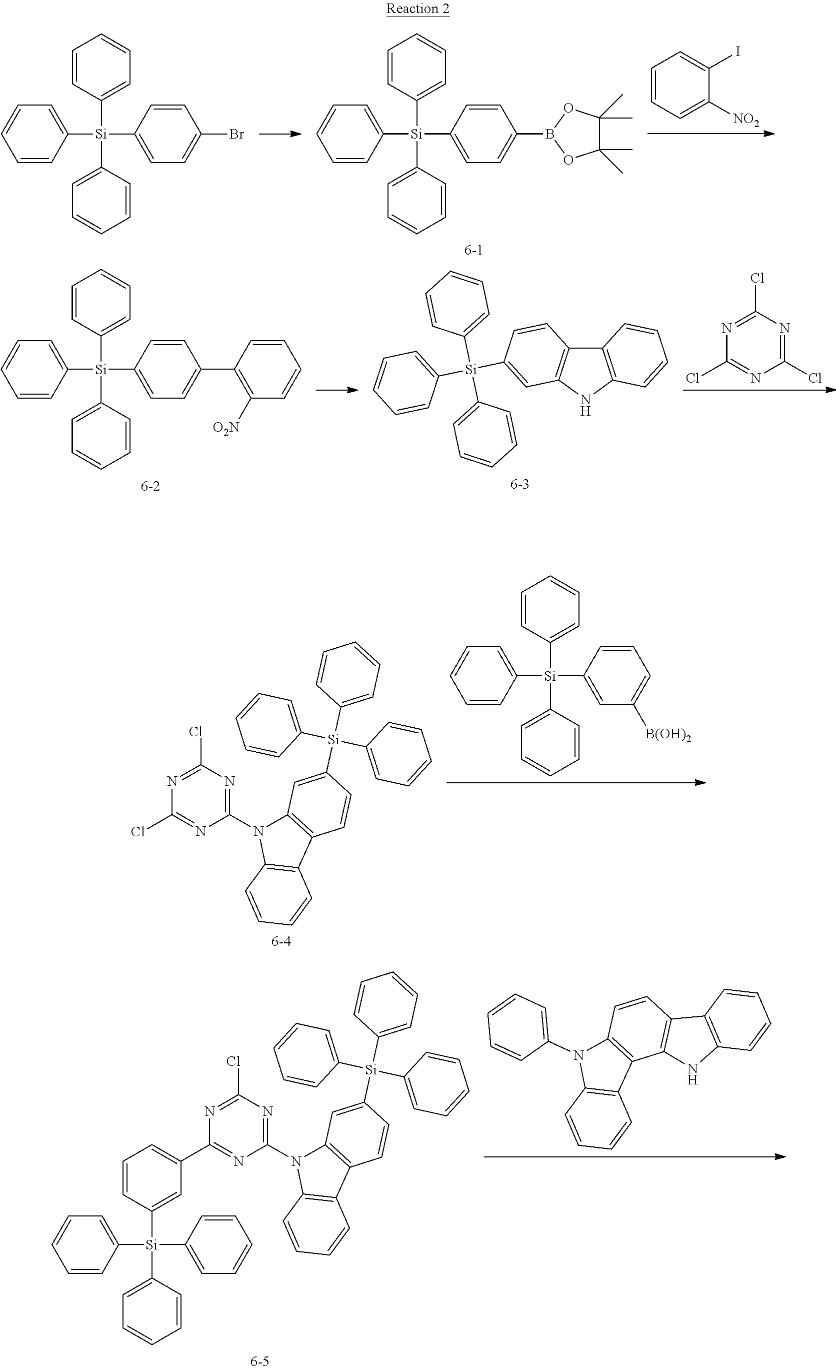

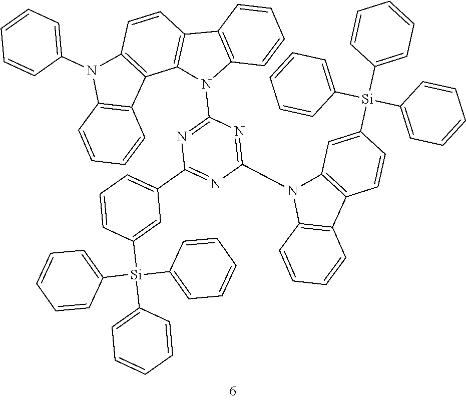 [SMILES: c1ccc(-n2c3ccccc3c3c2ccc2c4ccccc4n(-c4nc(-c5cccc([Si](c6ccccc6)(c6ccccc6)c6ccccc6)c5)nc(-n5c6ccccc6c6ccc([Si](c7ccccc7)(c7ccccc7)c7ccccc7)cc65)n4)c23)cc1]

### 1) Synthesis of Intermediate 6-1

(4-Bromophenyl)triphenylsilane (CAS #: 18737-40-1) and bis(pinacolato)diboron (CAS #: 73183-34-3) were reacted under Pd catalyst conditions to obtain Intermediate 6-1. Intermediate 6-1 was identified by LC-MS.

C₃₀H₃₁BO₂Si M+1: 463.23

### 2) Synthesis of Intermediate 6-2

Intermediate 6-1 and 1-iodo-2-nitrobenzene (CAS #: 609-73-4) were reacted under Pd catalyst conditions to obtain Intermediate 6-2. Intermediate 6-2 was identified by LC-MS.

C₃₀H₂₃NO₂Si M+1: 458.16

### 3) Synthesis of Intermediate 6-3

Intermediate 6-1 and triphenylphosphine (CAS #: 603-35-0) were reacted to obtain Intermediate 6-3. Intermediate 6-3 was identified by LC-MS.

C₃₀H₂₃NSi M+1: 426.17

### 4) Synthesis of Intermediate 6-4

Intermediate 6-3 and n-BuLi were reacted, and cyanuric chloride (CAS #: 108-77-0) was added thereto, followed by stirring under about 70° C. conditions overnight to obtain Intermediate 6-4. Intermediate 6-4 was identified by LC-MS.

C₃₃H₂₂Cl₂N₄Si M+1: 573.11

### 5) Synthesis of Intermediate 6-5

Intermediate 6-4 and (3-(triphenylsilyl)phenyl)boronic acid (CAS #: 1253912-58-1) were reacted under Pd catalyst conditions to obtain Intermediate 6-5. Intermediate 6-5 was identified by LC-MS.

C₅₇H₄₁ClN₄Si₂ M+1: 873.27

### 6) Synthesis of Compound 6

Intermediate 6-5 (3.5 g), Intermediate 1-5 (1.34 g), Pd₂(dba)₃ (0.15 g), P(tBu)₃ (50 wt % in xylene, 0.1 mL), NaOtBu (1.16 g), and o-xylene (50 mL) were put in RBF, followed by stirring under about 160° C. conditions overnight. After completing the reaction, the reaction solution was extracted with ethyl acetate, a collected organic layer was dried over magnesium sulfate, the solvent was evaporated, and the residue thus obtained was separated and purified by silica gel column chromatography to obtain Compound 6 (3.3 g, yield: 71%). Compound 6 was identified by LC-MS and ¹H-NMR, and the results are shown in Table 1.

### (3) Synthesis of Compound 10

Polycyclic Compound 10 according to an embodiment may be synthesized, for example, by the steps of Reaction 3.

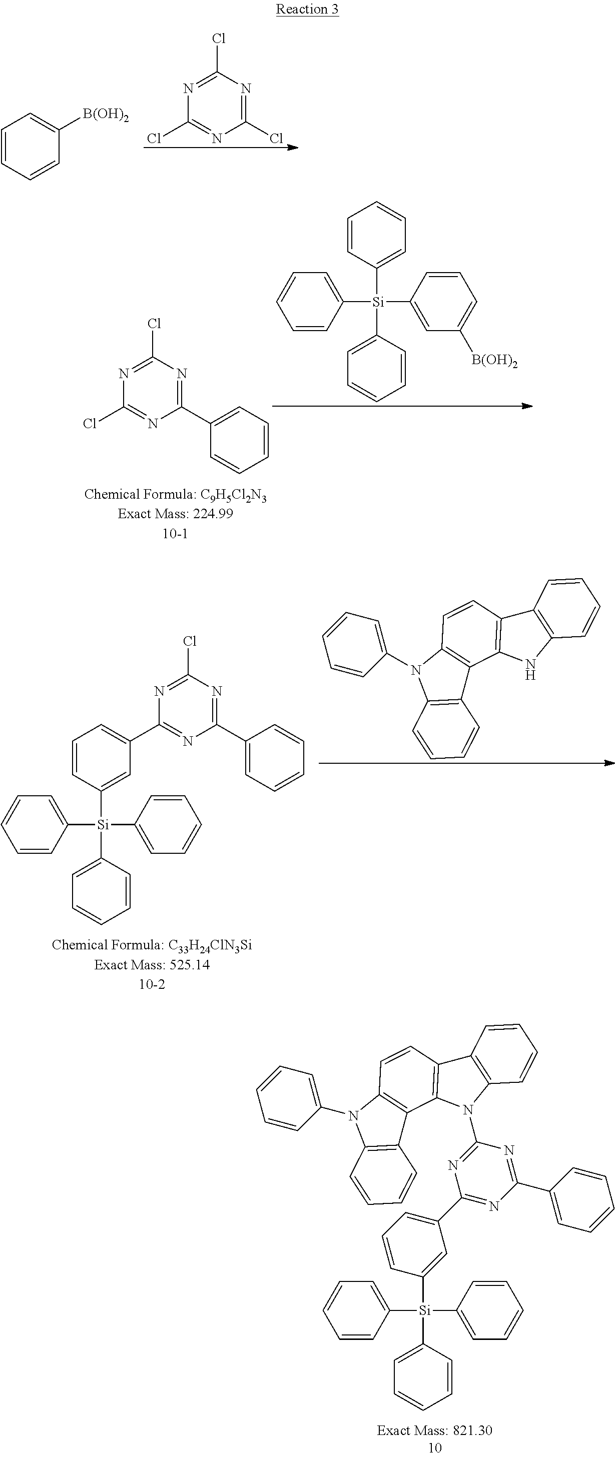

### 1) Synthesis of Intermediate 10-1

Phenylboronic acid (CAS #: 98-80-6) and cyanuric chloride (CAS #: 108-77-0) were reacted under Pd catalyst conditions to obtain Intermediate 10-1. Intermediate 10-1 was identified by LC-MS.

C₉H₅Cl₂N₃ M+1: 226.01

### 2) Synthesis of Intermediate 10-2

Intermediate 10-1 and (3-(triphenylsilyl)phenyl)boronic acid (CAS #: 1253912-58-1) were reacted under Pd catalyst conditions to obtain Intermediate 10-2. Intermediate 10-2 was identified by LC-MS.

C₃₃H₂₄ClN₃Si M+1: 526.16

### 3) Synthesis of Compound 10

Intermediate 10-2 (2.8 g), Intermediate 1-5 (1.78 g), Pd₂(dba)₃ (0.2 g), P(tBu)₃ (50 wt % in xylene, 0.1 mL), NaOtBu (1.54 g), and o-xylene (50 mL) were put in RBF (round-bottom flask), followed by stirring under about 160° C. conditions overnight. After completing the reaction, the reaction solution was extracted with ethyl acetate, a collected organic layer was dried over magnesium sulfate, the solvent was evaporated, and the residue thus obtained was separated and purified by silica gel column chromatography to obtain Compound 10 (3.3 g, yield: 75%). Compound 10 was identified by LC-MS and ¹H-NMR.

### (4) Synthesis of Compound 26

Polycyclic Compound 26 according to an embodiment may be synthesized, for example, by the steps of Reaction 4.

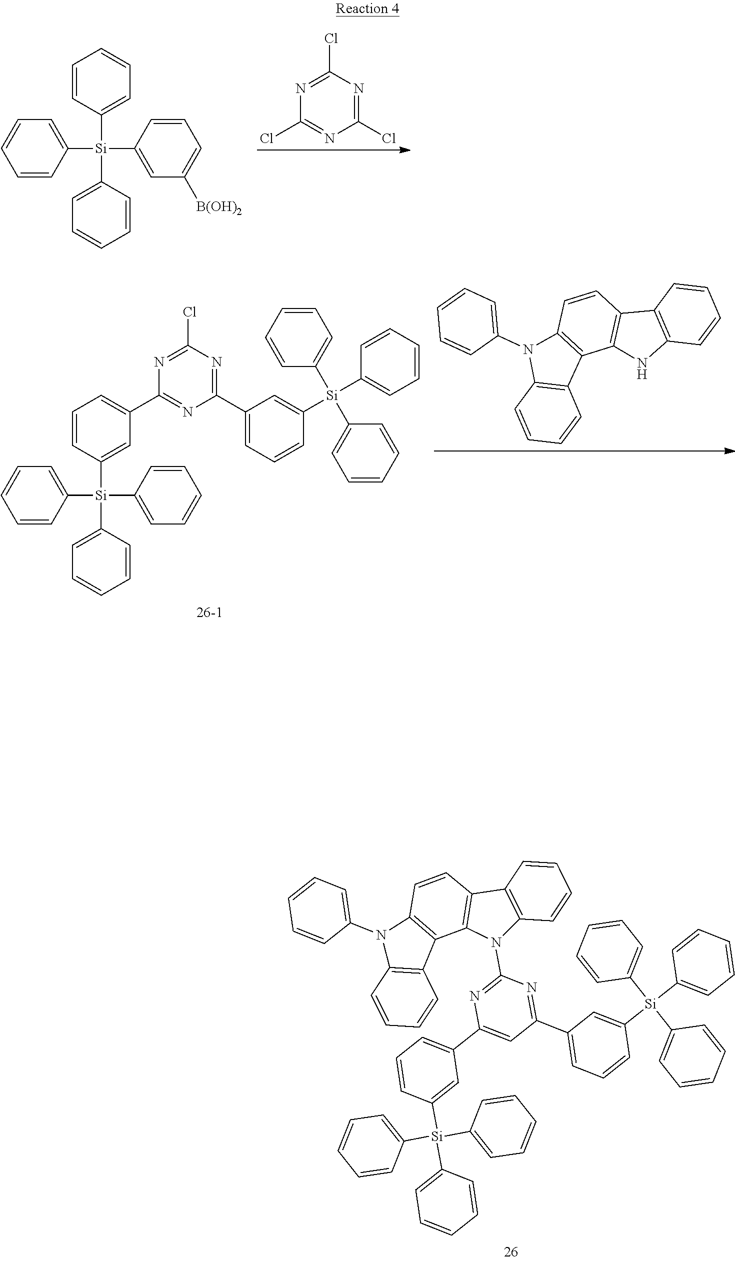

### 1) Synthesis of Intermediate 26-1

(3-(Triphenylsilyl)phenyl)boronic acid (CAS #: 1253912-58-1) and 2,4,6-trichloropyrimidine (CAS #: 3764-01-0) were reacted under Pd catalyst conditions to obtain Intermediate 26-1. Intermediate 26-1 was identified by LC-MS and ¹H-NMR.

C₅₂H₃₉ClN₂Si₂ M+1: 783.24

### 2) Synthesis of Compound 26

Intermediate 26-1 (3.3 g), Intermediate 1-5 (1.41 g), Pd₂(dba)₃ (0.15 g), P(tBu)₃ (50 wt % in xylene, 0.1 mL), NaOtBu (1.22 g), and o-xylene (50 mL) were put in RBF, followed by stirring under about 160° C. conditions overnight. After completing the reaction, the reaction solution was extracted with ethyl acetate, a collected organic layer was dried over magnesium sulfate, the solvent was evaporated, and the residue thus obtained was separated and purified by silica gel column chromatography to obtain Compound 26 (3.0 g, yield: 66%). Compound 26 was identified by LC-MS and ¹H-NMR, and the results are shown in Table 1.

### (5) Synthesis of Compound 58

Polycyclic Compound 58 according to an embodiment may be synthesized, for example, by the steps of Reaction 5.

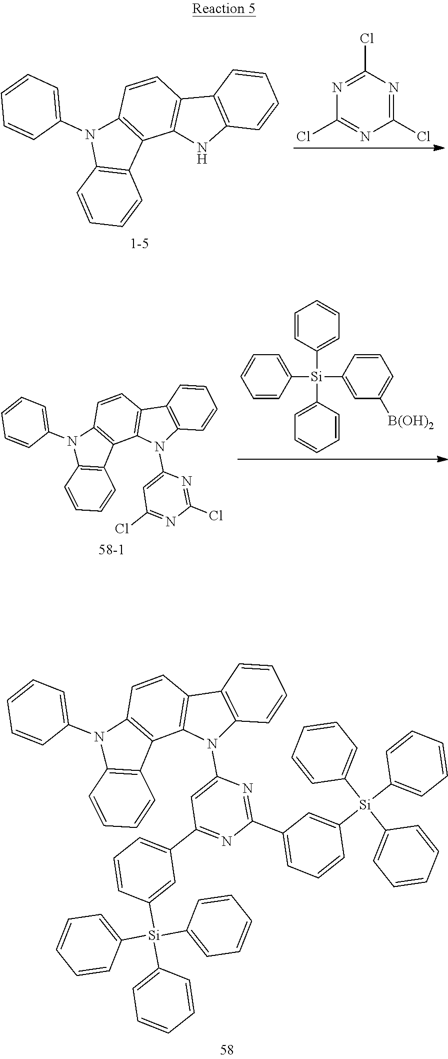

### 1) Synthesis of Intermediate 58-1

Intermediate 1-5 and n-BuLi were reacted, and 2,4,6-trichloropyrimidine (CAS #: 3764-01-0) was additionally reacted to obtain Intermediate 58-1. Intermediate 58-1 was identified by LC-MS and ¹H-NMR.

C₂₈H₁₆Cl₂N₄: 479.09

### 2) Synthesis of Compound 58

Intermediate 58-1 (2.8 g), (3-(triphenylsilyl)phenyl)boronic acid (5.34 g), Pd(PPh₃)₄ (0.54 g), K₂CO₃ (4.1 g, in 15 mL of H₂O), EtOH (15 mL), and toluene (60 mL) were put in RBF, followed by stirring under about 160° C. conditions overnight. After completing the reaction, the reaction solution was extracted with ethyl acetate, a collected organic layer was dried over magnesium sulfate, the solvent was evaporated, and the residue thus obtained was separated and purified by silica gel column chromatography to obtain Compound 58 (3.9 g, yield: 62%). Compound 58 was identified by LC-MS and ¹H-NMR, and the results are shown in Table 1.

### (6) Synthesis of Compound 70

Polycyclic Compound 70 according to an embodiment may be synthesized, for example, by the steps of Reaction 6.

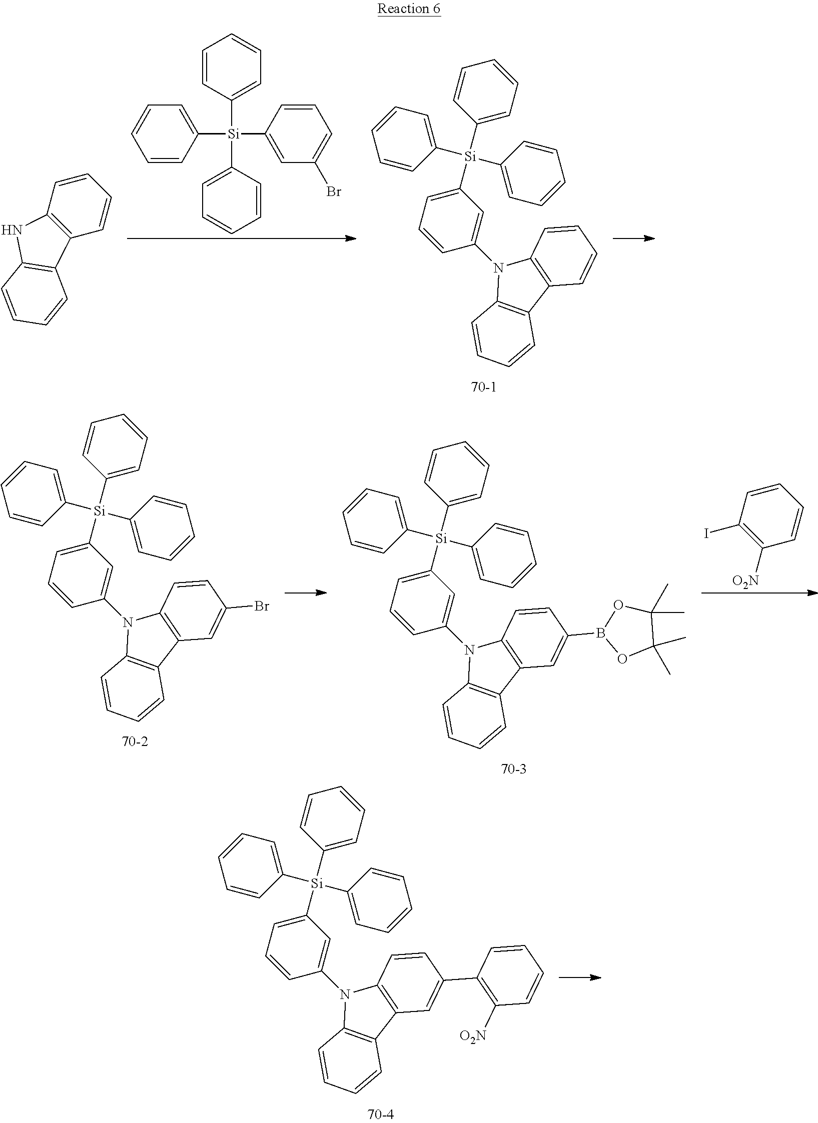

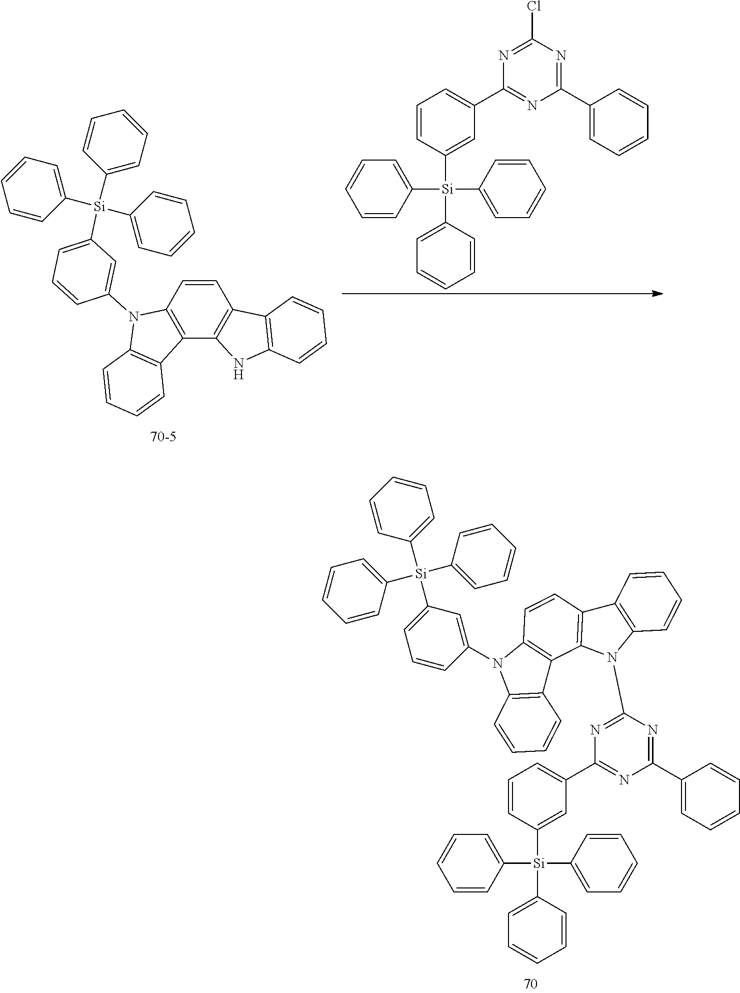 [SMILES: c1ccc([Si](c2ccccc2)(c2ccccc2)c2cccc(-n3c4ccccc4c4c5[nH]c6ccccc6c5ccc43)c2)cc1 ; Clc1nc(-c2ccccc2)nc(-c2cccc([Si](c3ccccc3)(c3ccccc3)c3ccccc3)c2)n1 ; c1ccc(-c2nc(-c3cccc([Si](c4ccccc4)(c4ccccc4)c4ccccc4)c3)nc(-n3c4ccccc4c4ccc5c(c6ccccc6n5-c5cccc([Si](c6ccccc6)(c6ccccc6)c6ccccc6)c5)c43)n2)cc1]

### 1) Synthesis of Intermediate 70-1

9H-carbazole (CAS #: 86-74-8) and n-BuLi were reacted, and (3-bromophenyl)triphenylsilane (CAS #: 185626-73-7) was added thereto, followed by stirring at about 70° C. overnight to obtain Intermediate 70-1. Intermediate 70-1 was identified by LC-MS.

C₃₆H₂₇NSi M+1: 502.20

### 2) Synthesis of Intermediate 70-2

Intermediate 70-1 and N-bromosuccinimide (CAS #: 128-08-5) were reacted to obtain Intermediate 70-2. Intermediate 70-2 was identified by LC-MS and ¹H-NMR.

C₃₆H₂₆BrNSi M+1: 580.11

### 3) Synthesis of Intermediate 70-3

Intermediate 70-2 and bis(pinacolato)diboron (CAS #: 73183-34-3) were reacted under Pd catalyst conditions to obtain Intermediate 70-3. Intermediate 70-3 was identified by LC-MS.

C₄₂H₃₈BNO₂Si M+1: 328.29

### 4) Synthesis of Intermediate 70-4

Intermediate 70-3 and 1-iodo-2-nitrobenzene (CAS #: 609-73-4) were reacted under Pd catalyst conditions to obtain Intermediate 70-4. Intermediate 70-4 was identified by LC-MS.

C₄₂H₃₀N₂O₂Si M+1: 623.22

### 5) Synthesis of Intermediate 70-5

Intermediate 70-4 and triphenylphosphine (CAS #: 603-35-0) were reacted to obtain Intermediate 70-5. Intermediate 70-5 was identified by LC-MS.

C₄₂H₃₀N₂Si M+1: 591.23

### 6) Synthesis of Compound 70

Intermediate 70-5 (3.15 g), Intermediate 10-2 (2.8 g), Pd₂(dba)₃ (0.2 g), P(tBu)₃ (50 wt % in xylene, 0.1 mL), NaOtBu (1.54 g), and o-xylene (50 mL) were put in RBF, followed by stirring under about 160° C. conditions overnight. After completing the reaction, the reaction solution was extracted with ethyl acetate, a collected organic layer was dried over magnesium sulfate, the solvent was evaporated, and the residue thus obtained was separated and purified by silica gel column chromatography to obtain Compound 70 (4.1 g, yield: 71%). Compound 70 was identified by LC-MS and ¹H-NMR, and the results are shown in Table 1.

### (7) Synthesis of Compound 150

Polycyclic Compound 150 according to an embodiment may be synthesized, for example, by the steps of Reaction 7.

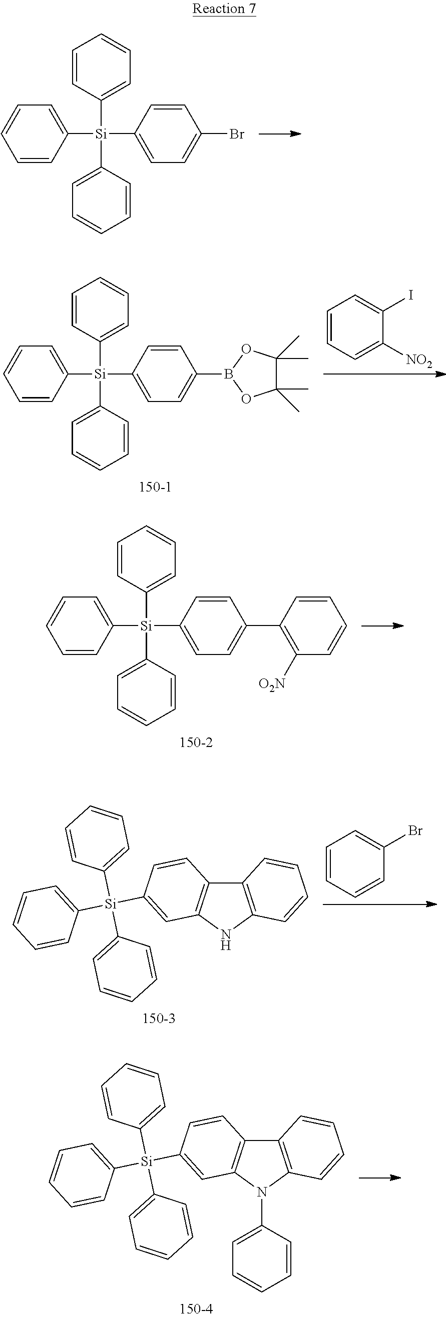

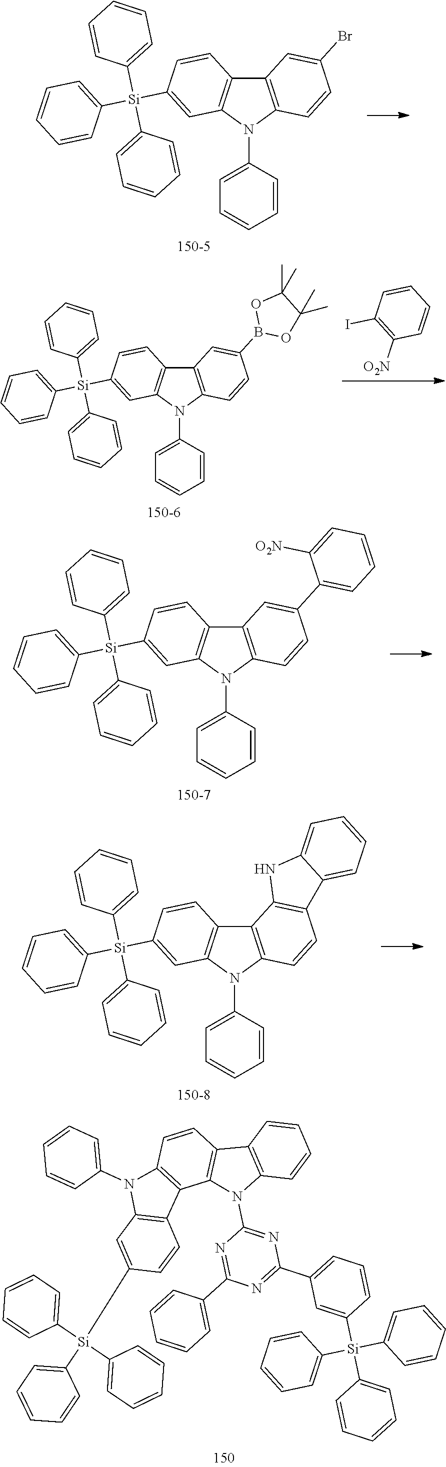

### 1) Synthesis of Intermediate 150-1

(4-Bromophenyl)triphenylsilane (CAS #: 18737-40-1) and bis(pinacolato)diboron (CAS #: 73183-34-3) were reacted under Pd catalyst conditions to obtain Intermediate 150-1. Intermediate 150-1 was identified by LC-MS.

C₃₀H₃₁BO₂Si M+1: 463.23

### 2) Synthesis of Intermediate 150-2

Intermediate 150-1 and 1-iodo-2-nitrobenzene (CAS #: 609-73-4) were reacted under Pd catalyst conditions to obtain Intermediate 150-2. Intermediate 150-2 was identified by LC-MS.

C₃₀H₂₃NO₂Si M+1: 458.16

### 3) Synthesis of Intermediate 150-3

Intermediate 150-2 and triphenylphosphine (CAS #: 603-35-0) were reacted to obtain Intermediate 150-3. Intermediate 150-3 was identified by LC-MS.

C₃₀H₂₃NSi M+1: 426.17

### 4) Synthesis of Intermediate 150-4

Intermediate 150-3 and n-BuLi were reacted, and bromobenzene (CAS #: 108-86-1) was added thereto, followed by stirring under 70° C. conditions overnight to obtain Intermediate 150-4. Intermediate 150-4 was identified by LC-MS.

C₃₆H₂₇NSi M+1: 502.20

### 5) Synthesis of Intermediate 150-5

Intermediate 150-4 and N-bromosuccinimide (CAS #: 128-08-5) were reacted to obtain Intermediate 150-5. Intermediate 150-5 was identified by LC-MS.

C₃₆H₂₆BrNSi M+1: 580.11

### 6) Synthesis of Intermediate 150-6

Intermediate 150-5 and bis(pinacolato)diboron (CAS #: 73183-34-3) were reacted under Pd catalyst conditions to obtain Intermediate 150-6. Intermediate 150-6 was identified by LC-MS.

C₄₂H₃₈BNO₂Si M+1: 628.29

### 7) Synthesis of Intermediate 150-7

Intermediate 150-6 and 1-iodo-2-nitrobenzene (CAS #: 609-73-4) were reacted under Pd catalyst conditions to obtain Intermediate 150-7. Intermediate 150-7 was identified by LC-MS.

C₄₂H₃₀N₂O₂Si M+1: 623.22

### 8) Synthesis of Intermediate 150-8

Intermediate 150-7 and triphenylphosphine (CAS #: 603-35-0) were reacted to obtain Intermediate 150-8. Intermediate 150-8 was identified by LC-MS.

C₄₂H₃₀N₂Si M+1: 591.23

### 9) Synthesis of Compound 150

Intermediate 150-8 (3.15 g), Intermediate 10-2 (2.8 g), Pd₂(dba)₃ (0.2 g), P(tBu)₃ (50 wt % in xylene, 0.1 mL), NaOtBu (1.54 g), and o-xylene (50 mL) were put in RBF, followed by stirring under about 160° C. conditions overnight. After completing the reaction, the reaction solution was extracted with ethyl acetate, a collected organic layer was dried over magnesium sulfate, the solvent was evaporated, and the residue thus obtained was separated and purified by silica gel column chromatography to obtain Compound 150 (4.0 g, yield: 70%). Compound 150 was identified by LC-MS and ¹H-NMR, and the results are shown in Table 1.

### (8) Synthesis of Compound 180

Polycyclic Compound 180 according to an embodiment may be synthesized, for example, by the steps of Reaction 8.

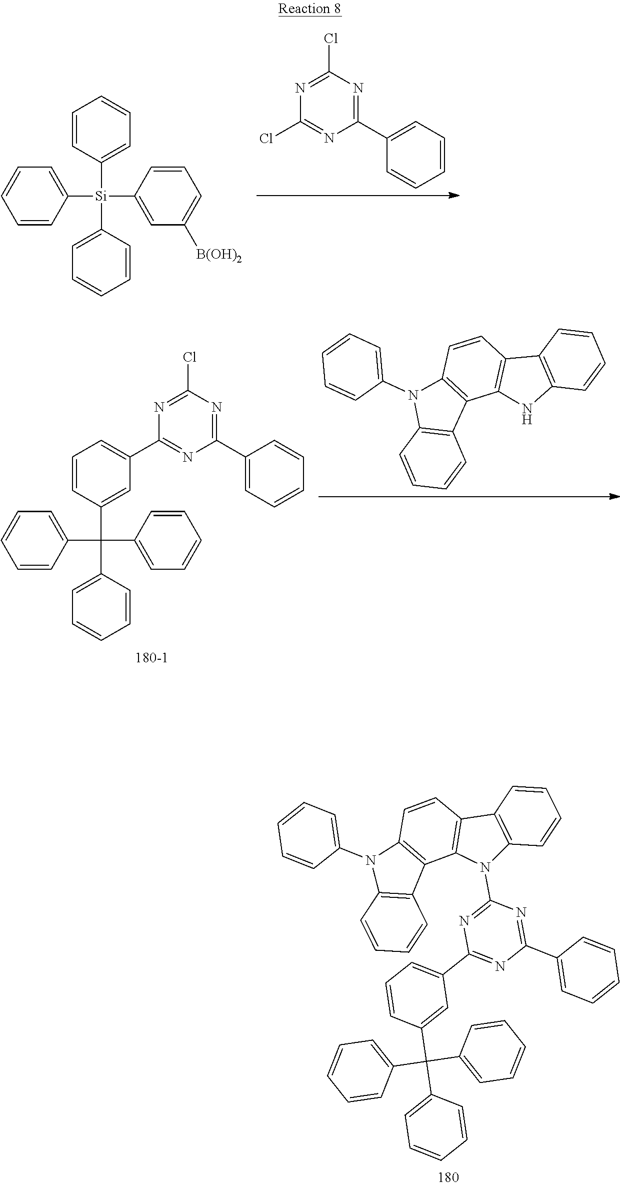 [SMILES: OB(O)c1cccc([Si](c2ccccc2)(c2ccccc2)c2ccccc2)c1 ; Clc1nc(Cl)nc(-c2ccccc2)n1 ; Clc1nc(-c2ccccc2)nc(-c2cccc(C(c3ccccc3)(c3ccccc3)c3ccccc3)c2)n1 ; c1ccc(-n2c3ccccc3c3c4[nH]c5ccccc5c4ccc32)cc1 ; c1ccc(-c2nc(-c3cccc(C(c4ccccc4)(c4ccccc4)c4ccccc4)c3)nc(-n3c4ccccc4c4ccc5c(c6ccccc6n5-c5ccccc5)c43)n2)cc1]

### 1) Synthesis of Intermediate 180-1

((3-Bromophenyl)methanetriyl)tribenzene (CAS #: 2131-59-1) and Intermediate 10-1 were reacted under Pd catalyst conditions to obtain Intermediate 180-1. Intermediate 150-1 was identified by LC-MS.

C₃₄H₂₄ClN₃ M+1: 510.18

### 2) Synthesis of Compound 180

Intermediate 180-1 (2.8 g), Intermediate 1-5 (1.83 g), Pd₂(dba)₃ (0.2 g), P(tBu)₃ (50 wt % in xylene, 0.1 mL), NaOtBu (1.59 g), and o-xylene (50 mL) were put in RBF, followed by stirring under about 160° C. conditions overnight. After completing the reaction, the reaction solution was extracted with ethyl acetate, a collected organic layer was dried over magnesium sulfate, the solvent was evaporated, and the residue thus obtained was separated and purified by silica gel column chromatography to obtain Compound 180 (3.2 g, yield: 73%). Compound 180 was identified by LC-MS and ¹H-NMR, and the results are shown in Table 1.

TABLE 1

| | | MS/FAB | |
|---|---|---|---|
| | ¹H NMR | | Found |
| Compound | (CDCl₃, 500 MHz) | Calc. | [M + 1] |

| | | | |
|---|---|---|---|
| 1 | 8.55 (d) 2H, 8.38 (m) 2, 7.88 (t) 2H, 7.63- | 1079.38 | 1080.41 |
| | 7.46 (m) 24H, 7.42-7.34 (m) 18H, 7.16- | | |
| | 7.11 (m) 4H, 7.01 (d) 1H | | |
| 6 | 8.57-8.53 (m) 3H, 8.38 (t) 1H, 7.88 (t) 1H, | 1168.41 | 1169.42 |
| | 7.73 (d) 1H, 7.68 (d) 1H, 7.63-7.46(m) | | |
| | 23H, 7.42-7.34 (m) 18H, 7.28 (d) 1H, 7.16- | | |
| | 7.11 (m) 6H, 7.00 (d) 1H | | |
| 10 | 8.55 (d) 2H, 8.40-8.34 (m) 3H, 7.88 (t) 1H, | 821.30 | 822.34 |
| | 7.63-7.46 (m) 19H, 7.42-7.36 (m) 9H, 7.16- | | |
| | 7.11 (m) 4H, 7.01 (d) 1H | | |
| 26 | 8.59 (s) 1H, 8.55 (d) 2H, 7.94 (d) 2H, 7.88 | 1078.39 | 1079.38 |
| | (t) 2H, 7.63-7.46 (m) 24H, 7.42-7.36 (m) | | |
| | 18H, 7.16-7.11 (m) 4H, 7.01 (d) 1H | | |
| 58 | 8.55 (d) 2H, 8.38 (d) 1H, 7.94 (d) 1H, 7.88 | 1078.39 | 1079.37 |
| | (t) 2H, 7.63-7.46 (m) 24H, 7.42-7.34 (m) | | |
| | 18H, 7.16-7.11 (m) 4H, 7.00 (d) 1H | | |
| 70 | 8.55 (d) 1H, 8.39-8.35(m) 3H, 7.88 (t) 1H, | 1079.38 | 1080.36 |
| | 7.66-7.42 (m) 24H, 7.41-7.36 (m) 18H, | | |
| | 7.16-7.11 (m) 4H, 7.01 (d) 1H | | |
| 150 | 8.55 (d) 1H, 8.39-8.35 (m) 3H, 8.1 (d) 1H, | 1079.38 | 1080.41 |
| | 7.88 (t) 1H, 7.68 (s) 1H, 7.42-7.36 (m) 18H, | | |
| | 7.28 (d) 1H, 7.16-7.11 (m) 2H, 7.01 (d) 1H | | |
| 180 | 8.55 (d) 2H, 8.38-8.35 (m) 2H, 8.18 (d) 1H, | 805.32 | 806.36 |
| | 7.80 (t) 1H, 7.65-7.48 (m) 12H, 7.28-7.08 | | |
| | (m) 20H, 7.00 (d) 1H | | |

### 2. Manufacture and Evaluation of Light Emitting Elements Including Polycyclic Compounds

### 1) Manufacture of Light Emitting Elements

Light emitting elements of Examples 1 to 8 were manufactured using Compounds 1, 6, 10, 26, 58, 70, 150 and 180 as the host materials of emission layers.

### Example Compounds

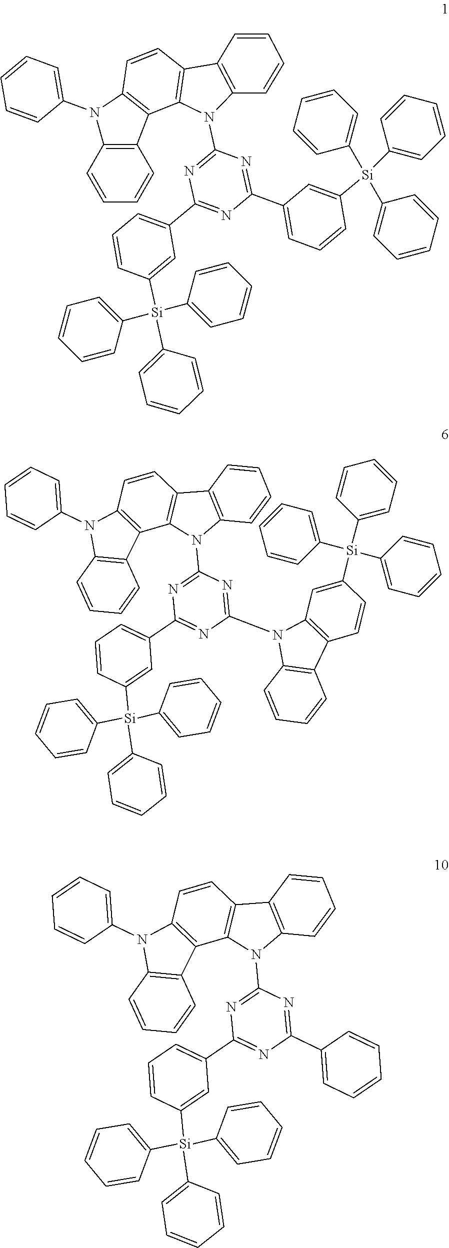 [SMILES: c1ccc(-n2c3ccccc3c3c2ccc2c4ccccc4n(-c4nc(-c5cccc([Si](c6ccccc6)(c6ccccc6)c6ccccc6)c5)nc(-c5cccc([Si](c6ccccc6)(c6ccccc6)c6ccccc6)c5)n4)c23)cc1 ; c1ccc(-n2c3ccccc3c3c2ccc2c4ccccc4n(-c4nc(-c5cccc([Si](c6ccccc6)(c6ccccc6)c6ccccc6)c5)nc(-n5c6ccccc6c6ccc([Si](c7ccccc7)(c7ccccc7)c7ccccc7)cc65)n4)c23)cc1 ; c1ccc(-c2nc(-c3cccc([Si](c4ccccc4)(c4ccccc4)c4ccccc4)c3)nc(-n3c4ccccc4c4ccc5c(c6ccccc6n5-c5ccccc5)c43)n2)cc1]

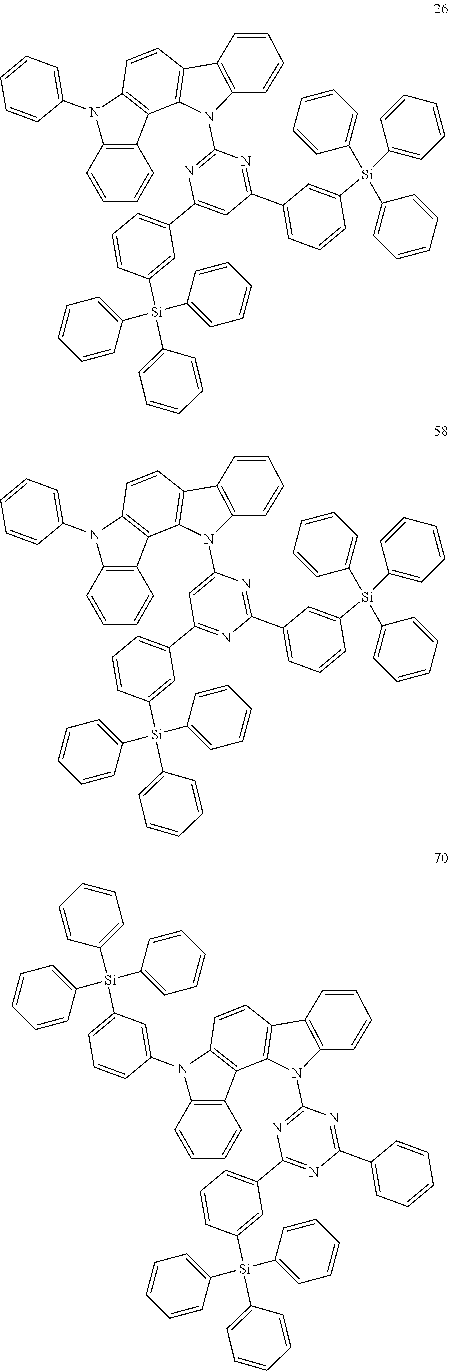 [SMILES: c1ccc(-n2c3ccccc3c3c2ccc2c4ccccc4n(-c4nc(-c5cccc([Si](c6ccccc6)(c6ccccc6)c6ccccc6)c5)cc(-c5cccc([Si](c6ccccc6)(c6ccccc6)c6ccccc6)c5)n4)c23)cc1 ; c1ccc(-n2c3ccccc3c3c2ccc2c4ccccc4n(-c4cc(-c5cccc([Si](c6ccccc6)(c6ccccc6)c6ccccc6)c5)nc(-c5cccc([Si](c6ccccc6)(c6ccccc6)c6ccccc6)c5)n4)c23)cc1 ; c1ccc(-c2nc(-c3cccc([Si](c4ccccc4)(c4ccccc4)c4ccccc4)c3)nc(-n3c4ccccc4c4ccc5c(c6ccccc6n5-c5cccc([Si](c6ccccc6)(c6ccccc6)c6ccccc6)c5)c43)n2)cc1]

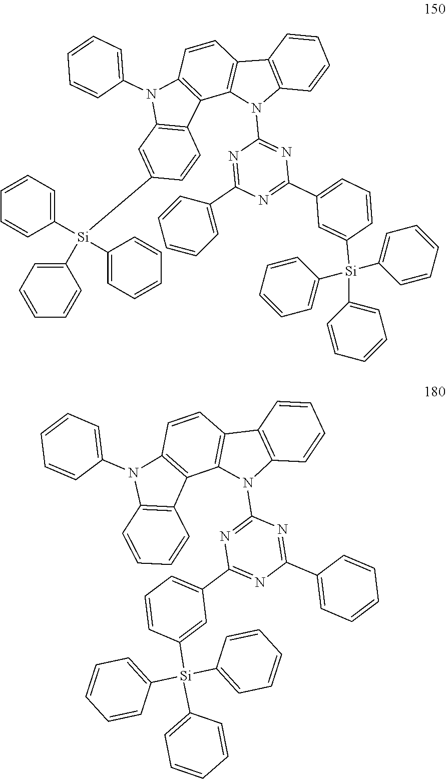 [SMILES: c1ccc(-c2nc(-c3cccc([Si](c4ccccc4)(c4ccccc4)c4ccccc4)c3)nc(-n3c4ccccc4c4ccc5c(c6ccc([Si](c7ccccc7)(c7ccccc7)c7ccccc7)cc6n5-c5ccccc5)c43)n2)cc1 ; c1ccc(-c2nc(-c3cccc([Si](c4ccccc4)(c4ccccc4)c4ccccc4)c3)nc(-n3c4ccccc4c4ccc5c(c6ccccc6n5-c5ccccc5)c43)n2)cc1]

### Comparative Compounds

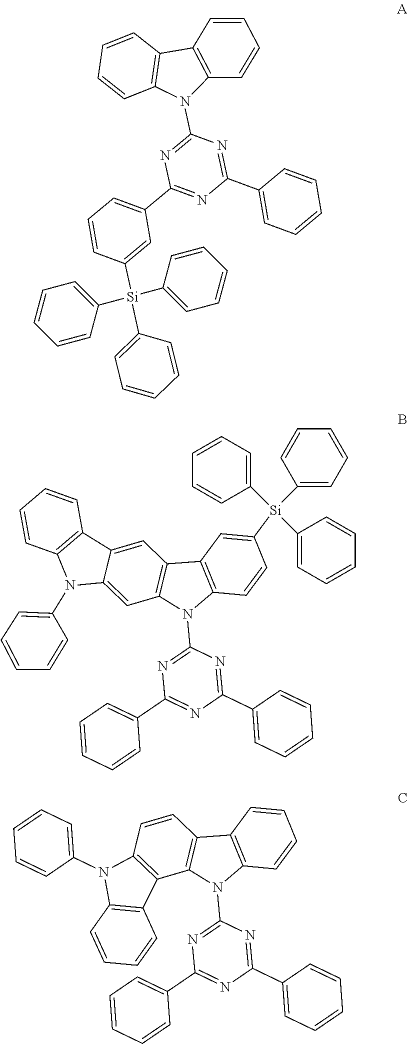 [SMILES: c1ccc(-c2nc(-c3cccc([Si](c4ccccc4)(c4ccccc4)c4ccccc4)c3)nc(-n3c4ccccc4c4ccccc43)n2)cc1 ; c1ccc(-c2nc(-c3ccccc3)nc(-n3c4ccc([Si](c5ccccc5)(c5ccccc5)c5ccccc5)cc4c4cc5c6ccccc6n(-c6ccccc6)c5cc43)n2)cc1 ; c1ccc(-c2nc(-c3ccccc3)nc(-n3c4ccccc4c4ccc5c(c6ccccc6n5-c5ccccc5)c43)n2)cc1]

The light emitting elements of the Examples (Example Compounds) and Comparative Examples (Comparative Compounds) were manufactured by a method below.

A first electrode used an ITO substrate with a thickness of about 1200 Å. The ITO substrate was prepared by washing by ultrasonic waves using isopropyl alcohol and ultrapure water for about 5 minutes each, and by cleansing by exposing to ultraviolet rays and ozone for about 30 minutes. The ITO substrate thus cleansed was installed in a vacuum deposition apparatus. Then, on the ITO substrate, N,N′-di(1-naphthyl)-N,N′-diphenylbenzidine (NPB) was vacuum deposited to form a hole injection layer. The hole injection layer was formed into a thickness of about 300 Å. Then, mCP was vacuum deposited on the hole injection layer to form a hole transport layer. The hole transport layer was formed into a thickness of about 200 Å.

Then, on the hole transport layer, the Example Compound or Comparative Compound and an Ir(pmp)₃ dopant material were co-deposited in a weight ratio of about 92:8 to form an emission layer with a thickness of about 250 Å.

Then, on the emission layer, 3-(4-biphenylyl)-4-phenyl-5-tert-butylphenyl-1,2,4-triazole (TAZ) was deposited to a thickness of about 200 Å to form an electron transport layer, and on the electron transport layer, a LiF alkaline metal halide was deposited to a thickness of about 10 Å to form an electron injection layer. Then, Al was vacuum deposited to a thickness of about 100 Å to form a second electrode. All layers were formed by a vacuum deposition method.

The compounds used for the manufacture of the light emitting elements of the Examples and Comparative Examples are shown below. The materials below were used for the manufacture of the elements after sublimation purification of commercial products.

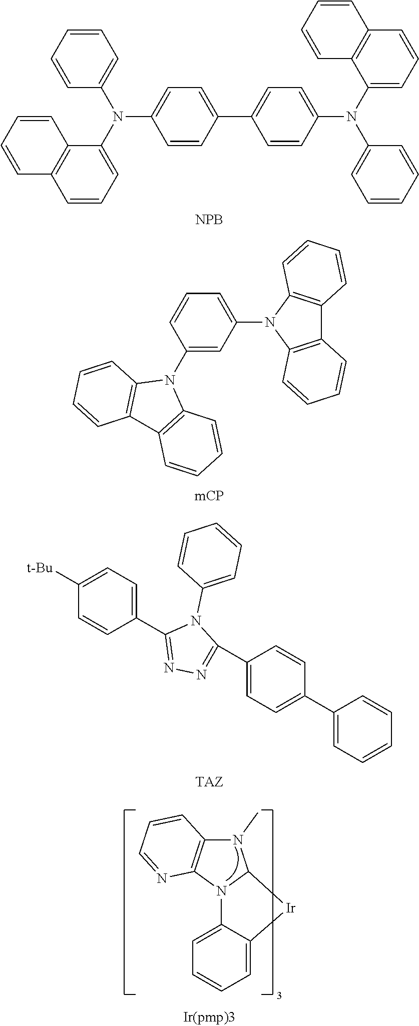

### 2) Evaluation of Properties of Light Emitting Elements

In order to evaluate the properties of the light emitting elements according to the Examples and Comparative Examples, a driving voltage at a current density of about 10 mA/cm², current density and maximum quantum efficiency were measured. The driving voltage and current density of the light emitting elements were measured using a source meter (Keithley Instrument Co., 2400 series). The maximum quantum efficiency was measured using an external quantum efficiency measurement apparatus of C9920-1 of Hamamatsu Photonics Co. For the evaluation of the maximum quantum efficiency, luminance/current density was measured using a luminance meter of which wavelength sensitivity was calibrated, and converting into maximum quantum efficiency supposing angular luminance distribution (Lambertian) assuming a perfect diffusion reflective surface. The evaluation results on the properties of the light emitting elements are shown in Table 2.

TABLE 2

| Element | | | | Maximum | |
|---|---|---|---|---|---|
| manu- | | Driving | Current | quantum | |
| facturing | Host of | voltage | density | efficiency | Emission |
| example | emission layer | (V) | (mA/cm²) | (%) | color |
| Example | Example | 4.0 | 10 | 24.5 | Blue |
| 1 | Compound 1 | | | | |
| Example | Example | 4.1 | 10 | 25.1 | Blue |
| 2 | Compound 6 | | | | |
| Example | Example | 3.9 | 10 | 26.4 | Blue |
| 3 | Compound 10 | | | | |
| Example | Example | 3.8 | 10 | 23.8 | Blue |
| 4 | Compound 26 | | | | |
| Example | Example | 3.8 | 10 | 23.6 | Blue |
| 5 | Compound 58 | | | | |
| Example | Example | 4.0 | 10 | 25.7 | Blue |
| 6 | Compound 70 | | | | |
| Example | Example | 4.1 | 10 | 24.8 | Blue |
| 7 | Compound | | | | |
| | 150 | | | | |
| Example | Example | 3.7 | 10 | 26.1 | Blue |
| 8 | Compound | | | | |
| | 180 | | | | |
| Compara- | Comparative | 4.7 | 10 | 21.8 | Blue |
| tive | Compound A | | | | |
| Example | | | | | |
| 1 | | | | | |
| Compara- | Comparative | 3.8 | 10 | 16.2 | Blue |
| tive | Compound B | | | | |
| Example | | | | | |
| 2 | | | | | |
| Compara- | Comparative | 4.0 | 10 | 19.1 | Blue |
| tive | Compound C | | | | |
| Example | | | | | |
| 3 | | | | | |

From Table 1, it could be confirmed that the light emitting elements of Example 1 to Example 8, including the polycyclic compounds of embodiments showed low driving voltages and excellent emission efficiency in a blue emission region. For example, the light emitting elements of Example 1 to Example 8 showed better quantum efficiency at lower driving voltages than the light emitting element of Comparative Example 1. In contrast, it could be confirmed that the driving voltages were substantially similar but quantum efficiency was degraded for the embodiments of Comparative Example 2 and Comparative Example 3.

The light emitting element of an embodiment includes the polycyclic compound of an embodiment, having excellent or suitable electron transfer capacity and may provide effects of a reduced driving voltage and improved emission efficiency.

The polycyclic compound of an embodiment may be used as a light emitting material for improving the driving voltage properties and emission efficiency of a light emitting element.

The use of “may” when describing embodiments of the present disclosure refers to “one or more embodiments of the present disclosure.”

As used herein, the term “substantially,” “about,” and similar terms are used as terms of approximation and not as terms of degree, and are intended to account for the inherent deviations in measured or calculated values that would be recognized by those of ordinary skill in the art. “About” or “approximately,” as used herein, is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” may mean within one or more standard deviations, or within ±30%, 20%, 10%, 5% of the stated value.

Also, any numerical range recited herein is intended to include all sub-ranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein and any minimum numerical limitation recited in this disclosure is intended to include all higher numerical limitations subsumed therein. Accordingly, Applicant reserves the right to amend this disclosure, including the claims, to expressly recite any sub-range subsumed within the ranges expressly recited herein.

The light emitting device or any other relevant devices or components according to embodiments of the present disclosure described herein may be implemented utilizing any suitable hardware, firmware (e.g., an application-specific integrated circuit), software, or a combination of software, firmware, and hardware. For example, the various components of the device may be formed on one integrated circuit (IC) chip or on separate IC chips. Further, the various components of the device may be implemented on a flexible printed circuit film, a tape carrier package (TCP), a printed circuit board (PCB), or formed on one substrate. Further, the various components of the device may be a process or thread, running on one or more processors, in one or more computing devices, executing computer program instructions and interacting with other system components for performing the various functionalities described herein. The computer program instructions are stored in a memory which may be implemented in a computing device using a standard memory device, such as, for example, a random access memory (RAM). The computer program instructions may also be stored in other non-transitory computer readable media such as, for example, a CD-ROM, flash drive, or the like. Also, a person of skill in the art should recognize that the functionality of various computing devices may be combined or integrated into a single computing device, or the functionality of a particular computing device may be distributed across one or more other computing devices without departing from the scope of the embodiments of the present disclosure.

Although the embodiments of the present disclosure have been described, it is understood that the present disclosure should not be limited to these embodiments, but one or more suitable changes and modifications can be made by one ordinary skilled in the art within the spirit and scope of the present disclosure as defined by the following claims and equivalents thereof.

What is claimed is:

1. A light emitting element, comprising:
a first electrode;
a second electrode on the first electrode; and
at least one functional layer between the first electrode and the second electrode, and comprising a polycyclic compound represented by Formula 1: 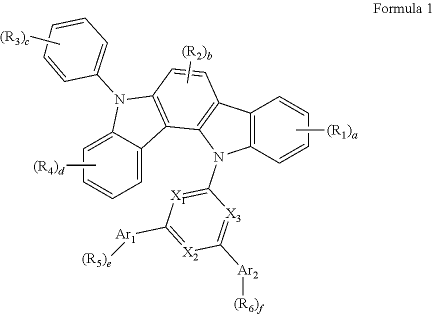
wherein in Formula 1,
X₁ to X₃ are each independently C or N, wherein at least two or more selected from among X₁ to X₃ are N,
Ar₁ and Ar₂ are each independently a substituted or unsubstituted arylene group of 6 to 60 ring-forming carbon atoms, or a substituted or unsubstituted heteroarylene group of 2 to 60 ring-forming carbon atoms,
R₁ to R₆ are each independently a hydrogen atom, a deuterium atom, or a cyano group, or represented by Formula 2, wherein at least one selected from among R₁ to R₆ is represented by Formula 2, and
a and d are each independently an integer from 0 to 4, b is an integer from 0 to 2, and c to f are integers from 0 to 5: 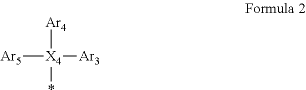
wherein in Formula 2,
X₄ is C, Si or Ge,
Ar₃ to Ar₅ are each independently a substituted or unsubstituted aryl group of 6 to 60 ring-forming carbon atoms, a substituted or unsubstituted cycloalkyl group of 3 to 60 ring-forming carbon atoms, or a substituted or unsubstituted heterocyclic group of 2 to 60 ring-forming carbon atoms, or combined with an adjacent group to form a ring, and
* represents a connection position.

2. The light emitting element of claim 1, wherein the at least one functional layer comprises an emission layer, a hole transport region between the first electrode and the emission layer, and an electron transport region between the emission layer and the second electrode, and
at least one selected from among the emission layer and the electron transport region comprises the polycyclic compound.

3. The light emitting element of claim 2, wherein the emission layer is configured to emit delayed fluorescence or phosphorescence.

4. The light emitting element of claim 2, wherein the emission layer comprises a host and a dopant, and
the host comprises the polycyclic compound.

5. The light emitting element of claim 2, wherein the electron transport region comprises an electron transport layer on the emission layer, and an electron injection layer on the electron transport layer, and
the electron transport layer comprises the polycyclic compound.

6. The light emitting element of claim 2, wherein the emission layer is configured to emit light having a central wavelength of about 430 nm to about 480 nm.

7. The light emitting element of claim 1, wherein Formula 1 is represented by any one selected from among Formula 3-1 to Formula 3-4: 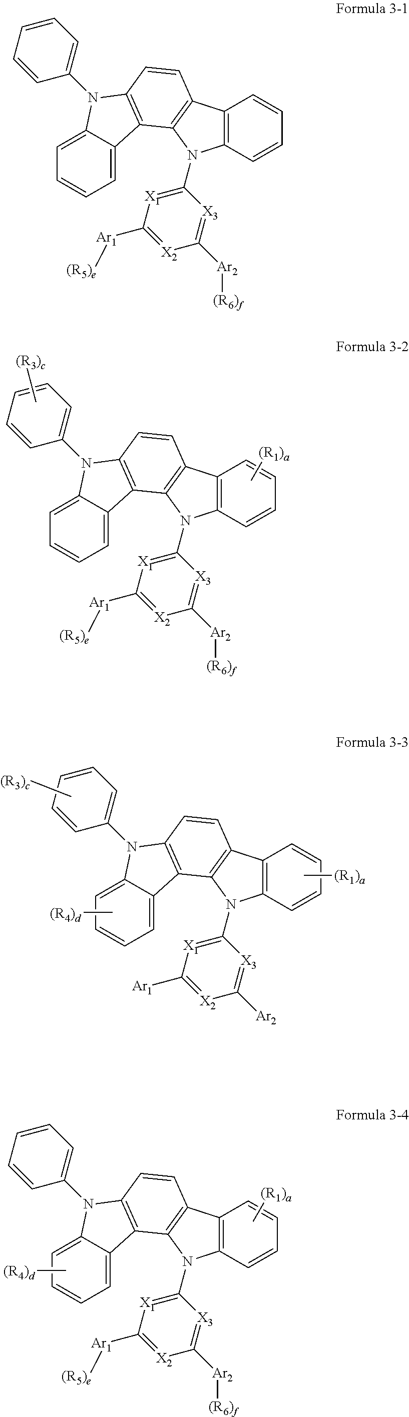
wherein in Formula 3-1 to Formula 3-4, R₁ to R₆, Ar₁, Ar₂, and X₁ to X₃ are the same as defined in Formula 1,
in Formula 3-1, at least one selected from among R₅ and R₆ is represented by Formula 2,
in Formula 3-2, at least one selected from among R₁ and R₃, and at least one selected from among R₅ and R₆ are represented by Formula 2,
in Formula 3-3, at least one selected from among R₁, R₃ and R₄ is represented by Formula 2, and
in Formula 3-4, at least one selected from among R₁, R₄, R₅ and R₆ is represented by Formula 2.

8. The light emitting element of claim 1, wherein Ar₁ and Ar₂ are each independently a substituted or unsubstituted phenylene group, a substituted or unsubstituted divalent pyridoindole group, or a substituted or unsubstituted divalent carbazole group.

9. The light emitting element of claim 1, wherein Ar₃ to Ar₅ are each independently a substituted or unsubstituted aryl group of 6 to 20 ring-forming carbon atoms, or a substituted or unsubstituted heteroaryl group of 2 to 20 ring-forming carbon atoms, or combined with an adjacent group to form a heterocycle.

10. The light emitting element of claim 9, wherein Ar₃ to Ar₅ are each independently a substituted or unsubstituted phenyl group, or a substituted or unsubstituted dibenzofuran group, or combined with an adjacent group to form a heterocycle containing O and Si as ring-forming atoms.

11. The light emitting element of claim 1, wherein Formula 2 is represented by any one selected from among 2-1 to 2-6:

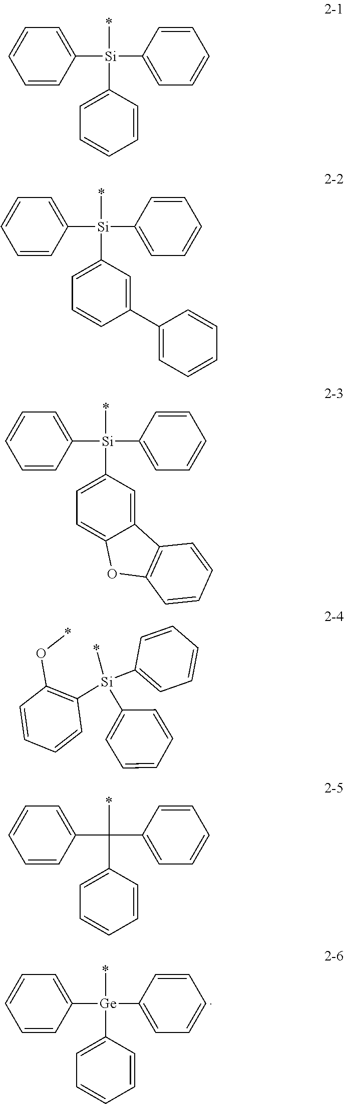

12. The light emitting element of claim 1, wherein the polycyclic compound represented by Formula 1 is represented by any one selected from among 4-1 to 4-19: 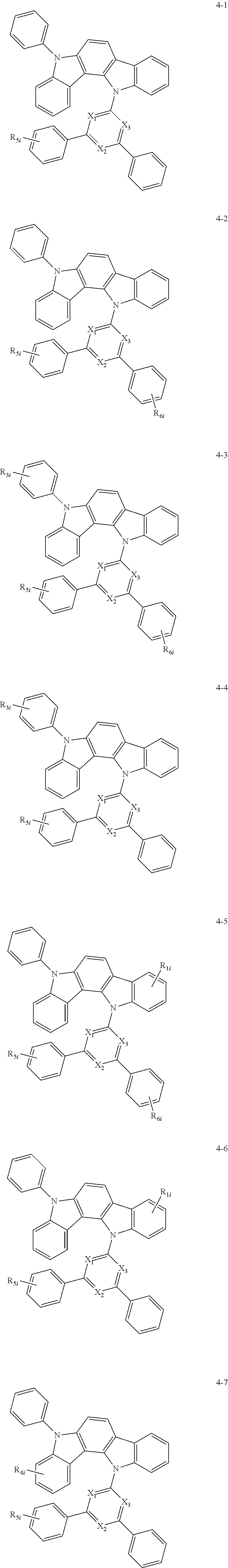 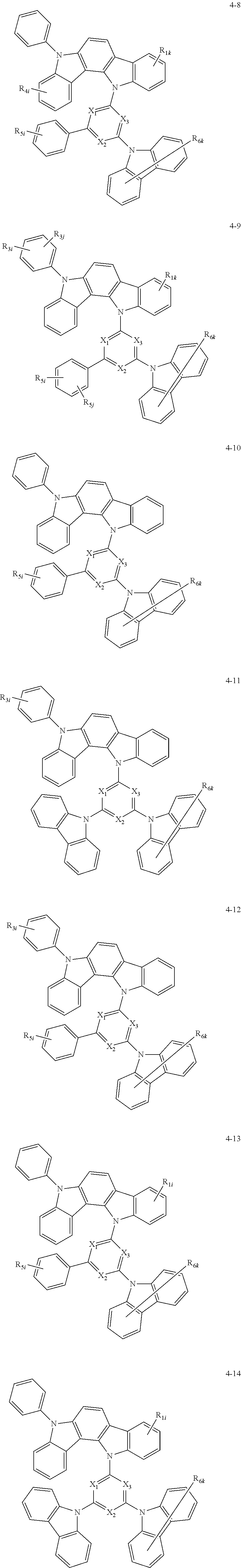 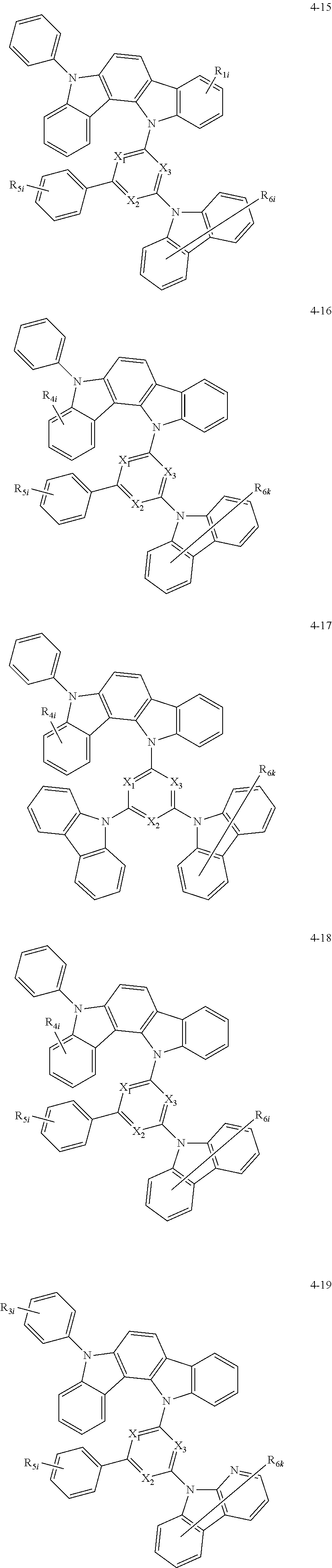
wherein in the polycyclic compounds represented by 4-1 to 4-19,
R₁ᵢ, R₃ᵢ, R₄ᵢ, R₅ᵢ and R₆ᵢ are each independently represented by Formula 2,
R₁ⱼ and R₅ⱼ are each independently a hydrogen atom or represented by Formula 2,
R₁ₖ and R₆ₖ are each independently a hydrogen atom or a cyano group, and
X₁ to X₃ are the same as defined in Formula 1.

13. The light emitting element of claim 1, wherein Formula 1 is represented by any one selected from among polycyclic compounds in Compound Group 1:

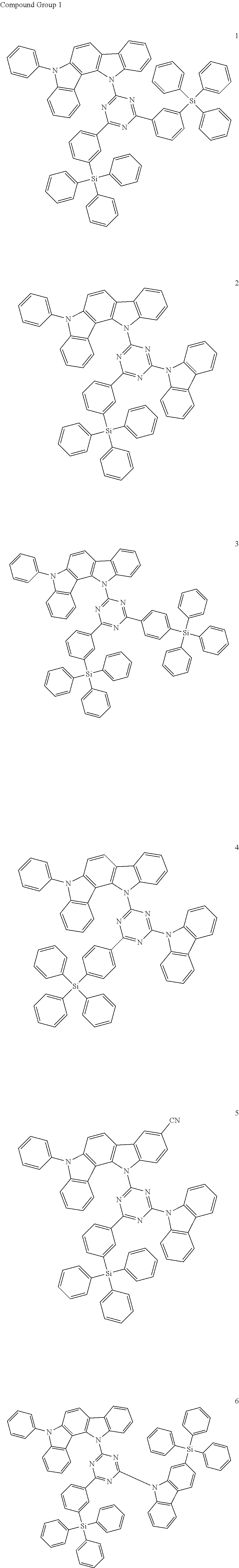

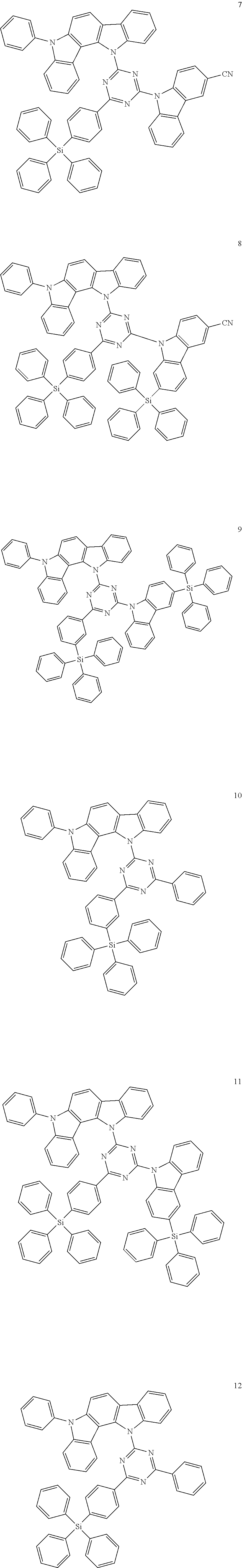

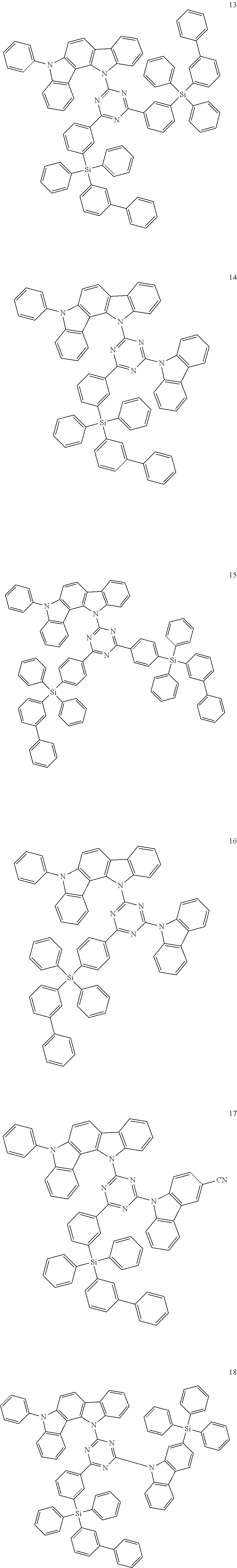

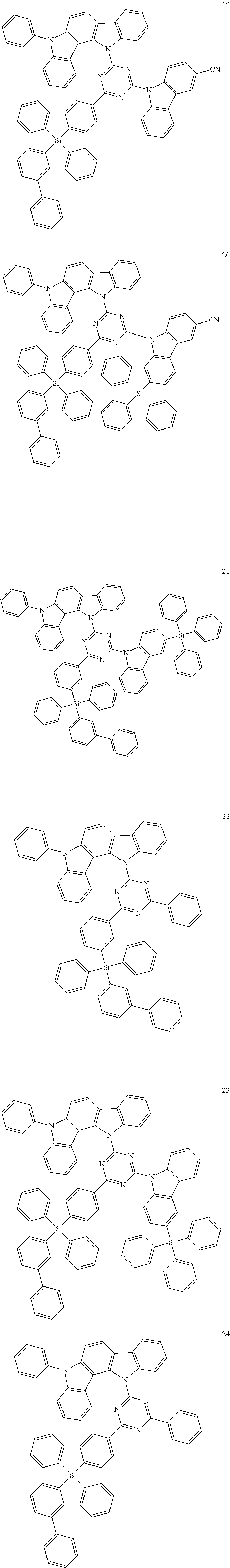

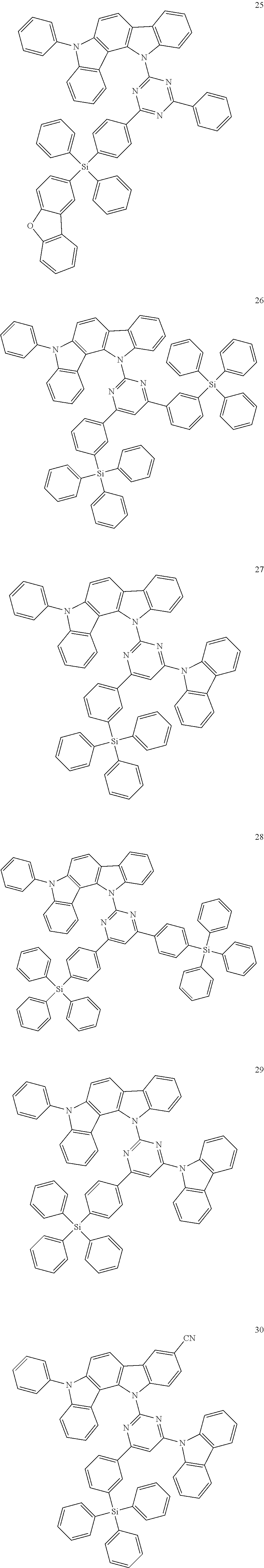

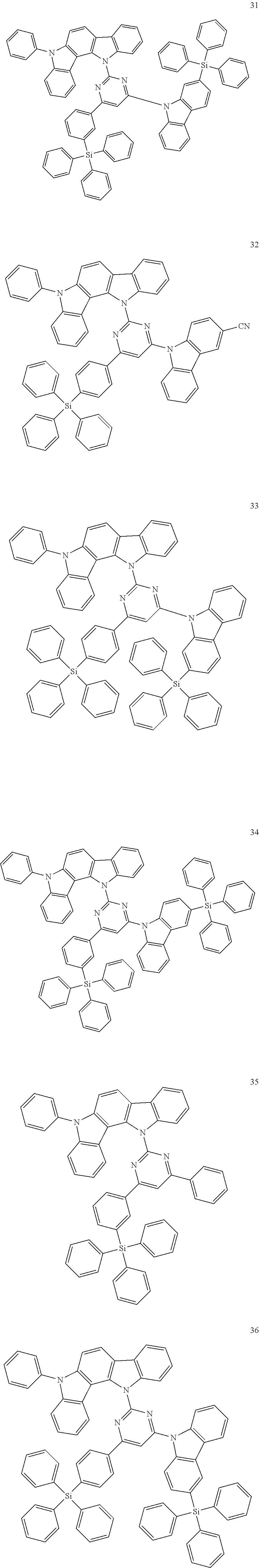

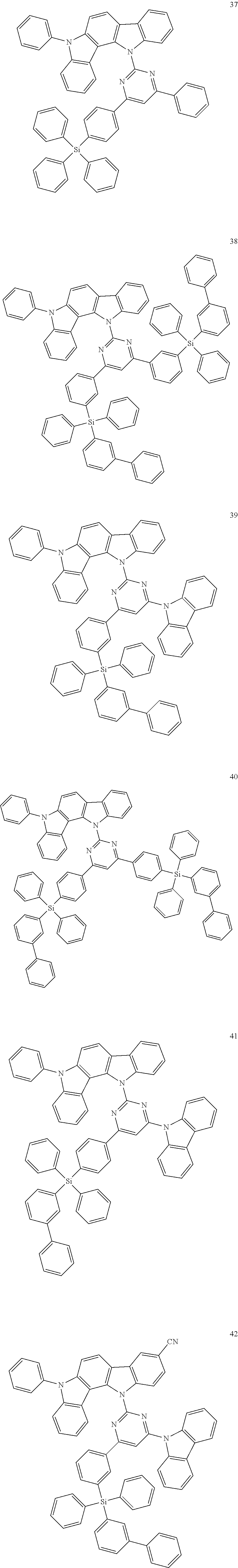

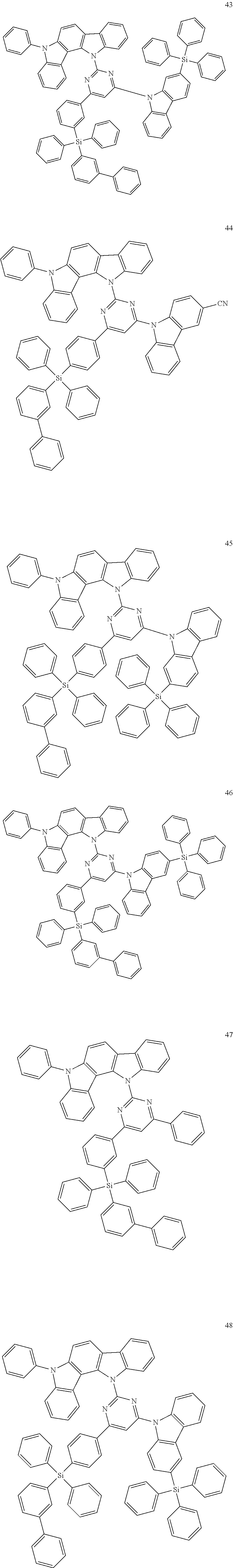

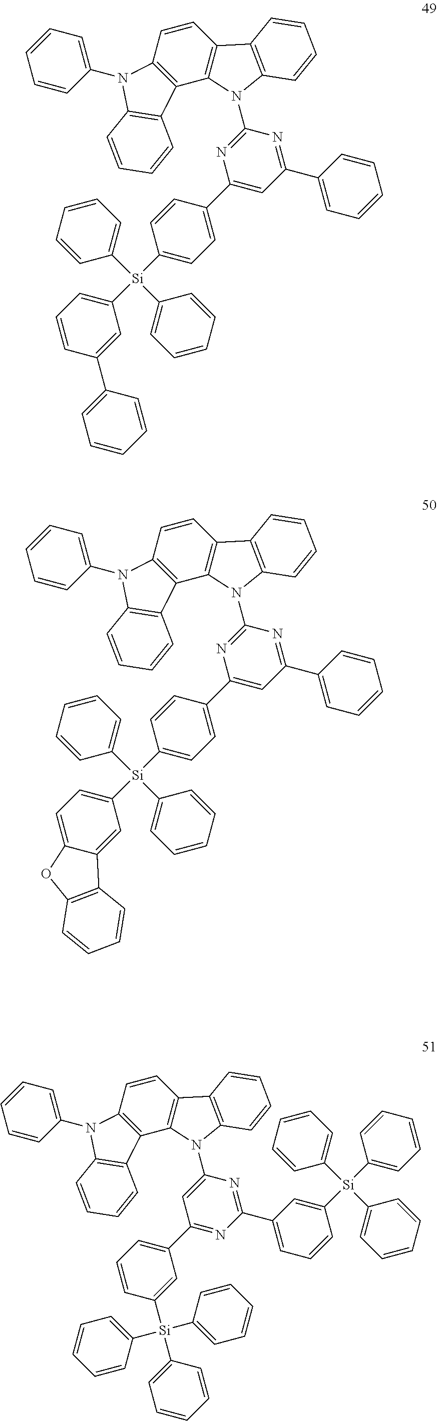 [SMILES: c1ccc(-c2cccc([Si](c3ccccc3)(c3ccccc3)c3ccc(-c4cc(-c5ccccc5)nc(-n5c6ccccc6c6ccc7c(c8ccccc8n7-c7ccccc7)c65)n4)cc3)c2)cc1 ; c1ccc(-c2cc(-c3ccc([Si](c4ccccc4)(c4ccccc4)c4ccc5oc6ccccc6c5c4)cc3)nc(-n3c4ccccc4c4ccc5c(c6ccccc6n5-c5ccccc5)c43)n2)cc1 ; c1ccc(-n2c3ccccc3c3c2ccc2c4ccccc4n(-c4cc(-c5cccc([Si](c6ccccc6)(c6ccccc6)c6ccccc6)c5)nc(-c5cccc([Si](c6ccccc6)(c6ccccc6)c6ccccc6)c5)n4)c23)cc1]

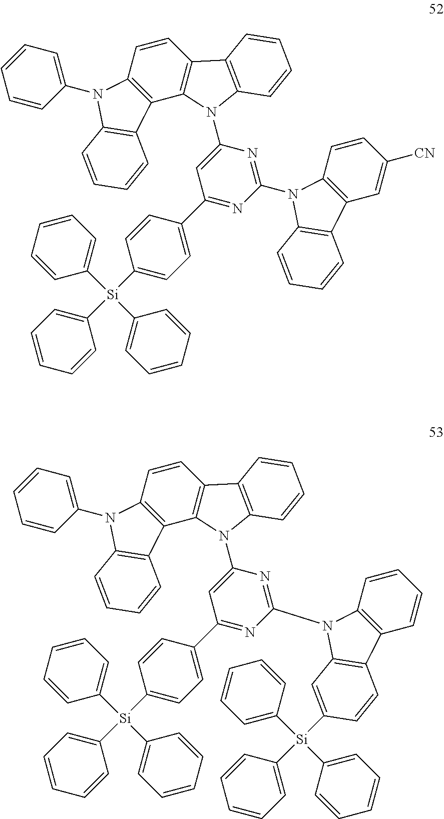 [SMILES: N#Cc1ccc2c(c1)c1ccccc1n2-c1nc(-c2ccc([Si](c3ccccc3)(c3ccccc3)c3ccccc3)cc2)cc(-n2c3ccccc3c3ccc4c(c5ccccc5n4-c4ccccc4)c32)n1 ; c1ccc(-n2c3ccccc3c3c2ccc2c4ccccc4n(-c4cc(-c5ccc([Si](c6ccccc6)(c6ccccc6)c6ccccc6)cc5)nc(-n5c6ccccc6c6ccc([Si](c7ccccc7)(c7ccccc7)c7ccccc7)cc65)n4)c23)cc1]

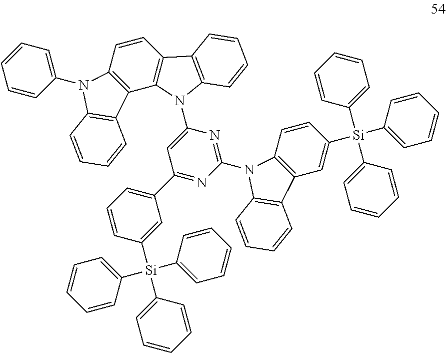 [SMILES: c1ccc(-n2c3ccccc3c3c2ccc2c4ccccc4n(-c4cc(-c5cccc([Si](c6ccccc6)(c6ccccc6)c6ccccc6)c5)nc(-n5c6ccccc6c6cc([Si](c7ccccc7)(c7ccccc7)c7ccccc7)ccc65)n4)c23)cc1]

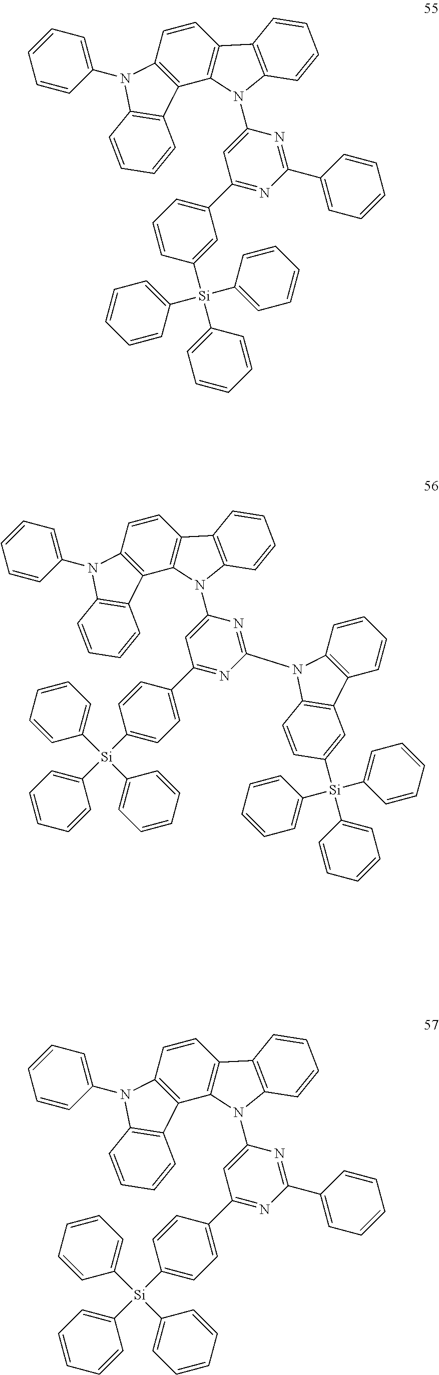 [SMILES: c1ccc(-c2nc(-c3cccc([Si](c4ccccc4)(c4ccccc4)c4ccccc4)c3)cc(-n3c4ccccc4c4ccc5c(c6ccccc6n5-c5ccccc5)c43)n2)cc1 ; c1ccc(-n2c3ccccc3c3c2ccc2c4ccccc4n(-c4cc(-c5ccc([Si](c6ccccc6)(c6ccccc6)c6ccccc6)cc5)nc(-n5c6ccccc6c6cc([Si](c7ccccc7)(c7ccccc7)c7ccccc7)ccc65)n4)c23)cc1 ; c1ccc(-c2nc(-c3ccc([Si](c4ccccc4)(c4ccccc4)c4ccccc4)cc3)cc(-n3c4ccccc4c4ccc5c(c6ccccc6n5-c5ccccc5)c43)n2)cc1]

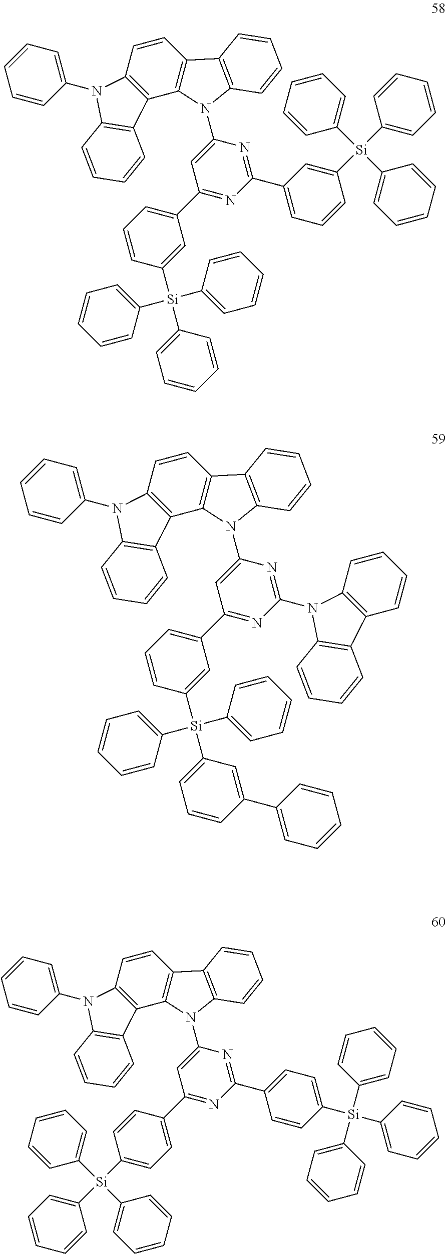 [SMILES: c1ccc(-n2c3ccccc3c3c2ccc2c4ccccc4n(-c4cc(-c5cccc([Si](c6ccccc6)(c6ccccc6)c6ccccc6)c5)nc(-c5cccc([Si](c6ccccc6)(c6ccccc6)c6ccccc6)c5)n4)c23)cc1 ; c1ccc(-c2cccc([Si](c3ccccc3)(c3ccccc3)c3cccc(-c4cc(-n5c6ccccc6c6ccc7c(c8ccccc8n7-c7ccccc7)c65)nc(-n5c6ccccc6c6ccccc65)n4)c3)c2)cc1 ; c1ccc(-n2c3ccccc3c3c2ccc2c4ccccc4n(-c4cc(-c5ccc([Si](c6ccccc6)(c6ccccc6)c6ccccc6)cc5)nc(-c5ccc([Si](c6ccccc6)(c6ccccc6)c6ccccc6)cc5)n4)c23)cc1]

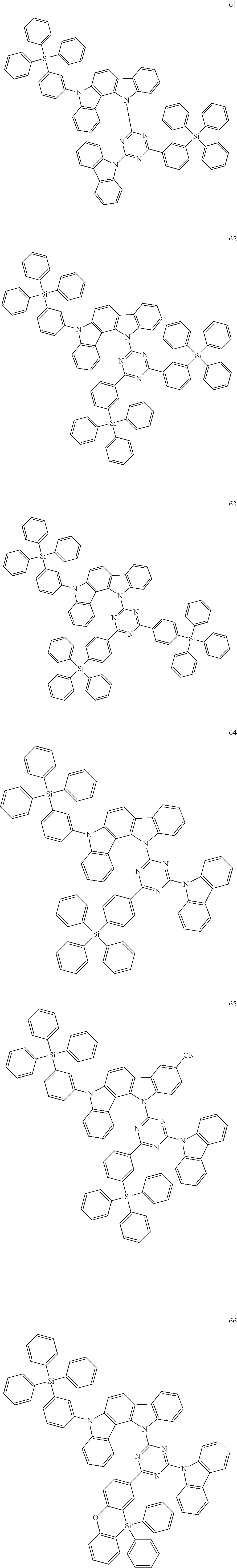

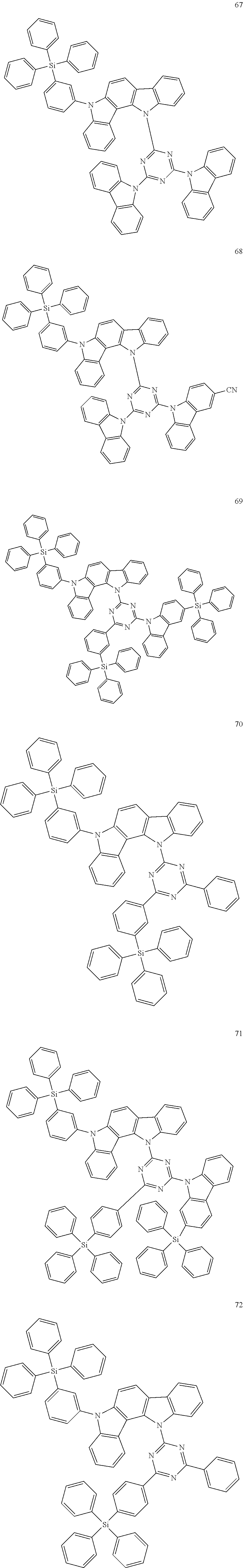

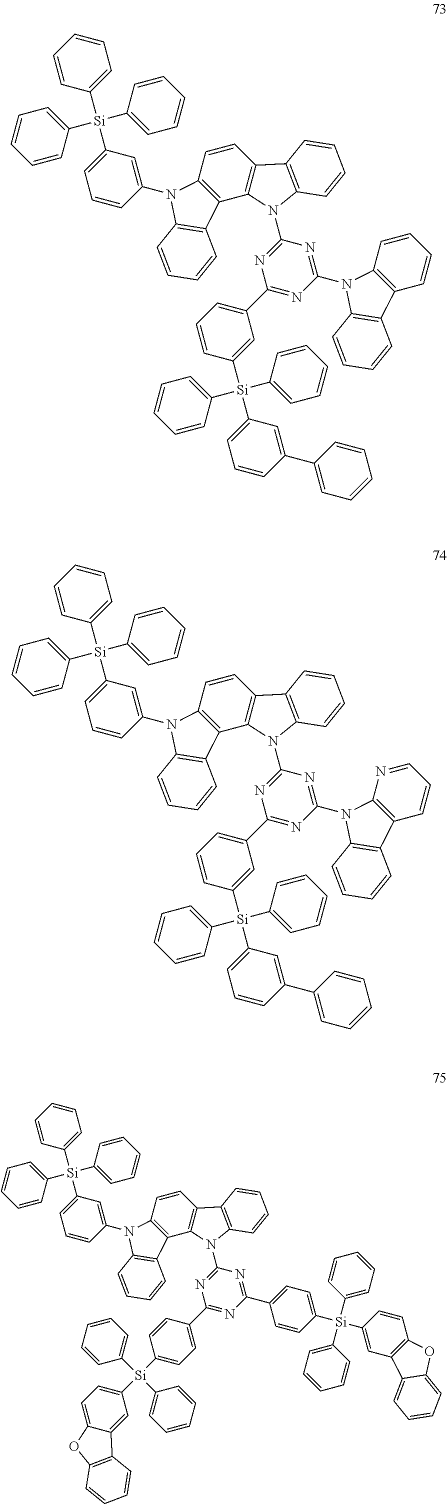

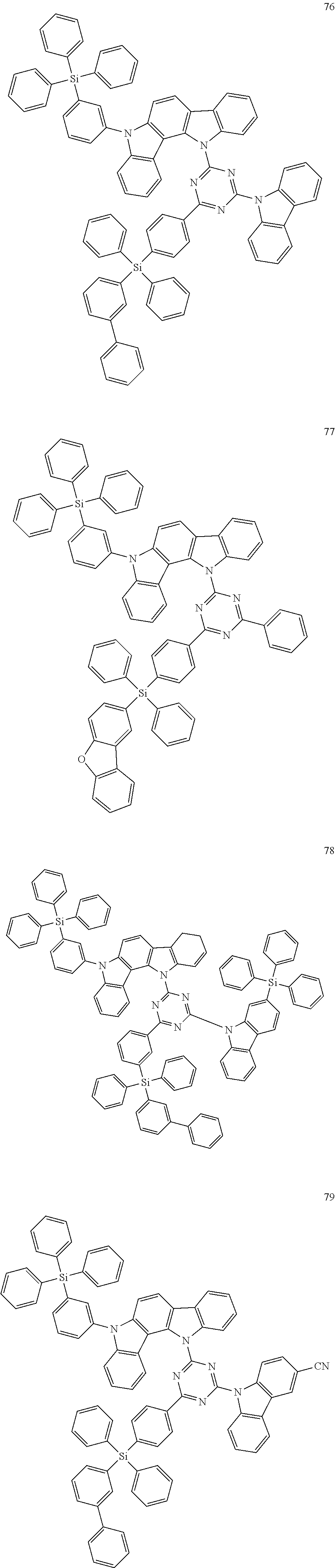

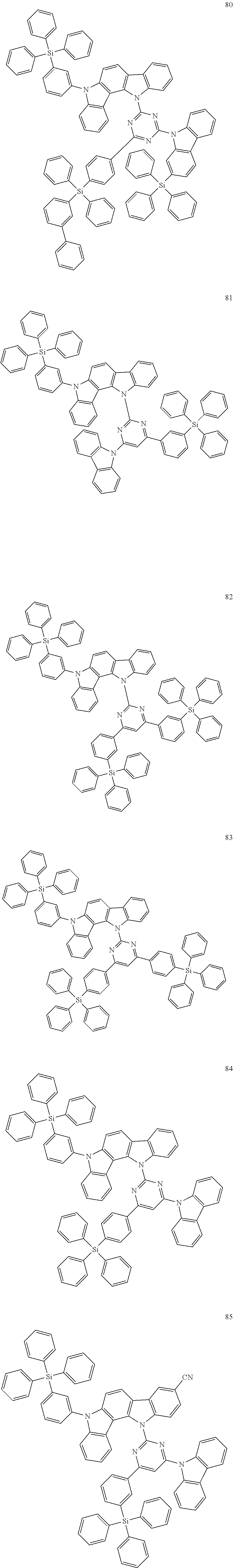

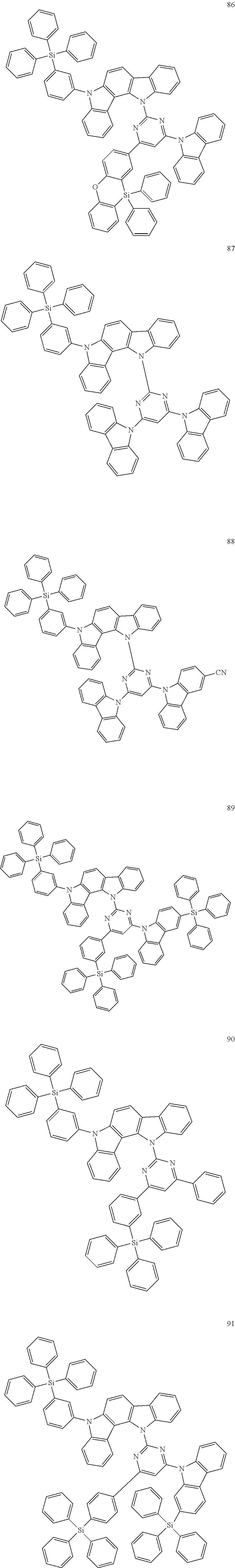

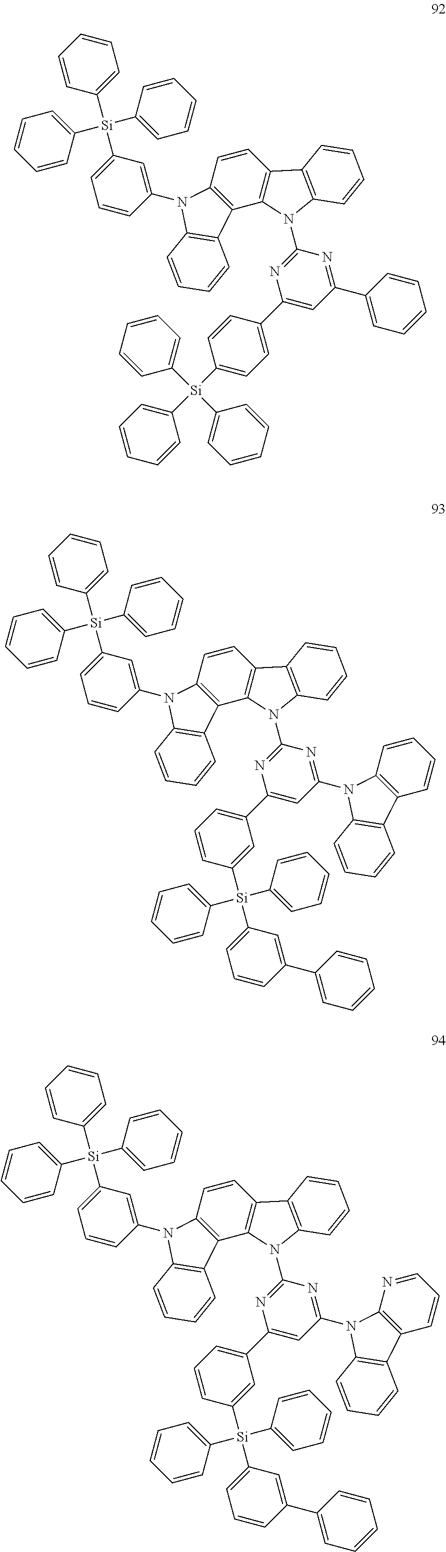 [SMILES: c1ccc(-c2cc(-c3ccc([Si](c4ccccc4)(c4ccccc4)c4ccccc4)cc3)nc(-n3c4ccccc4c4ccc5c(c6ccccc6n5-c5cccc([Si](c6ccccc6)(c6ccccc6)c6ccccc6)c5)c43)n2)cc1 ; c1ccc(-c2cccc([Si](c3ccccc3)(c3ccccc3)c3cccc(-c4cc(-n5c6ccccc6c6ccccc65)nc(-n5c6ccccc6c6ccc7c(c8ccccc8n7-c7cccc([Si](c8ccccc8)(c8ccccc8)c8ccccc8)c7)c65)n4)c3)c2)cc1 ; c1ccc(-c2cccc([Si](c3ccccc3)(c3ccccc3)c3cccc(-c4cc(-n5c6ccccc6c6cccnc65)nc(-n5c6ccccc6c6ccc7c(c8ccccc8n7-c7cccc([Si](c8ccccc8)(c8ccccc8)c8ccccc8)c7)c65)n4)c3)c2)cc1]

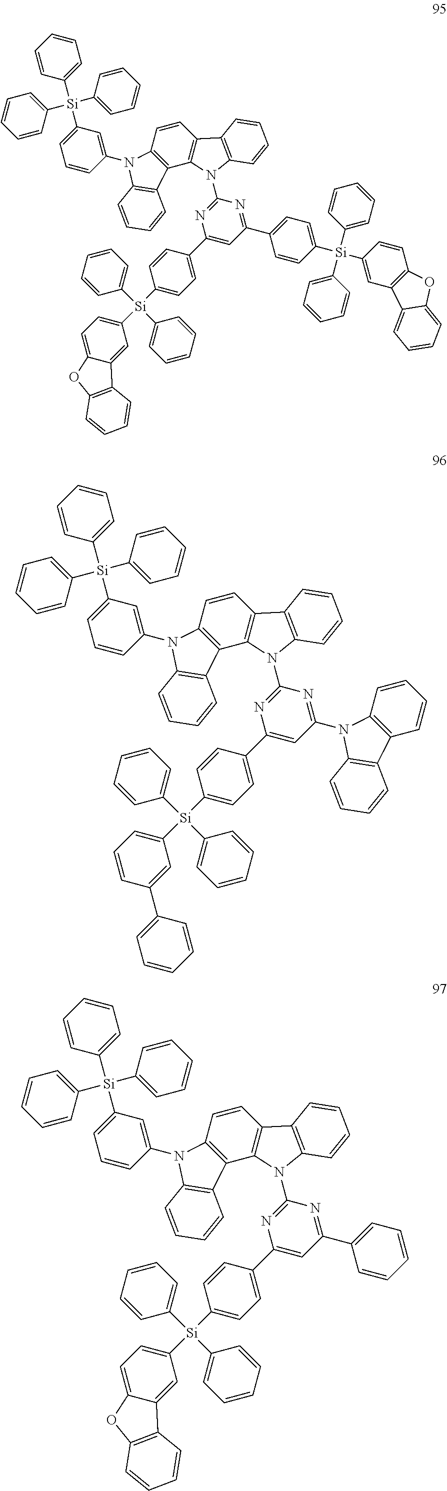

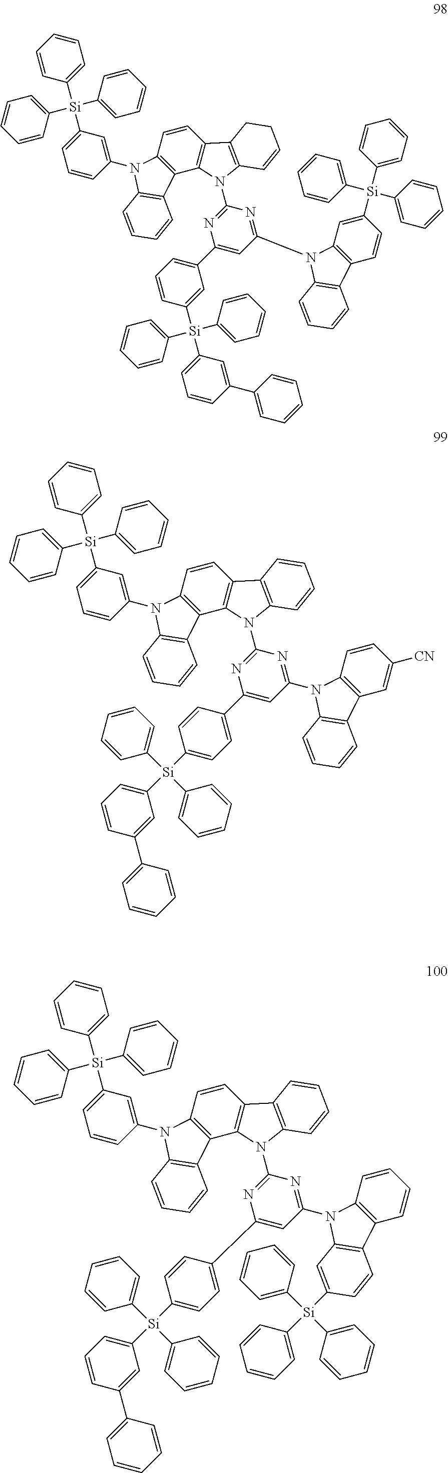

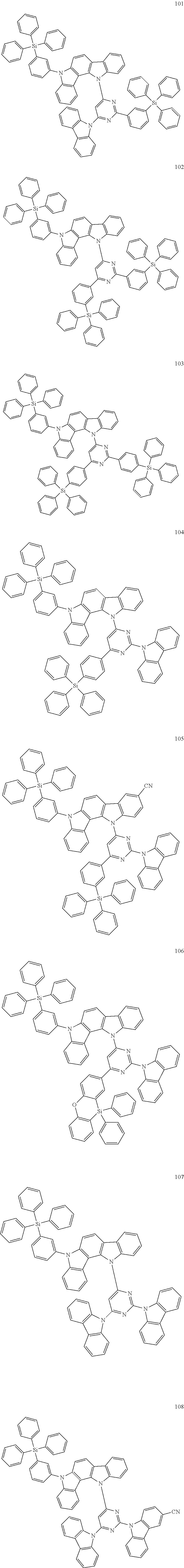

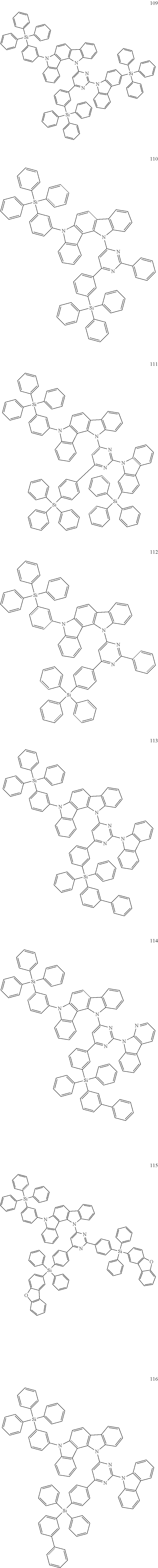

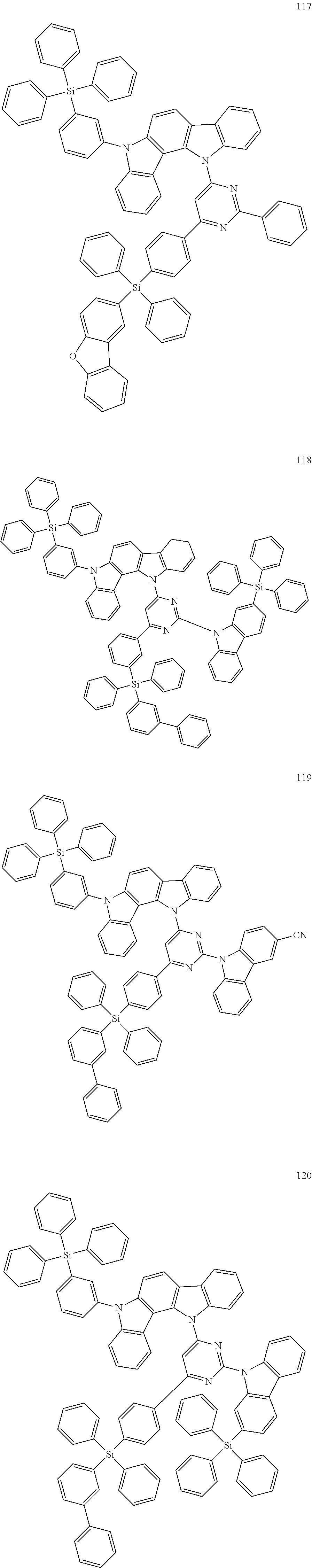

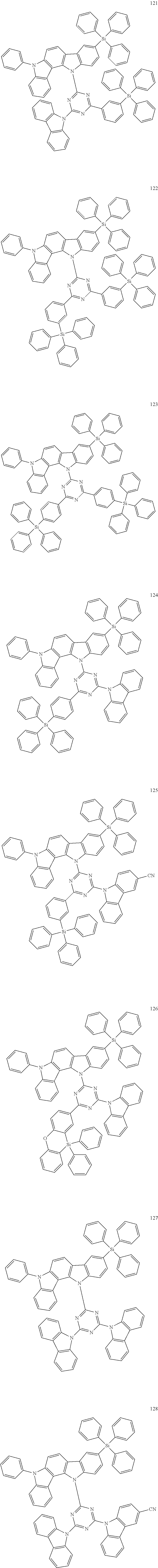

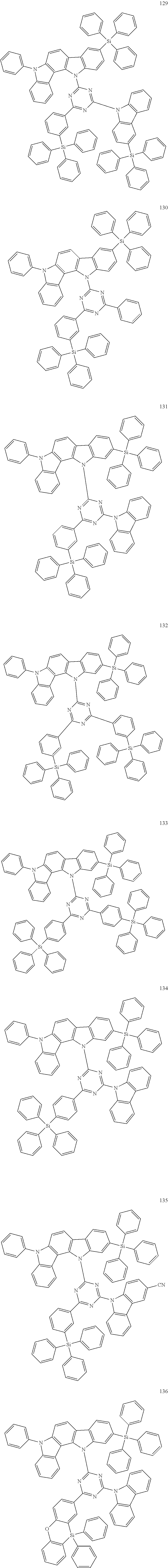

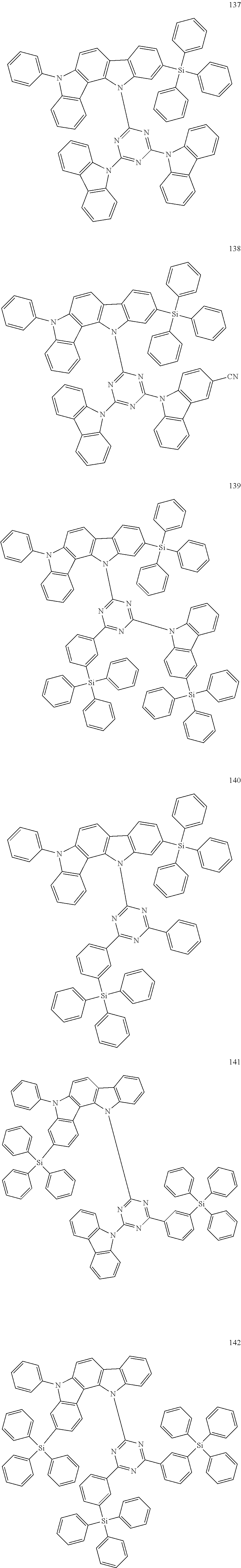

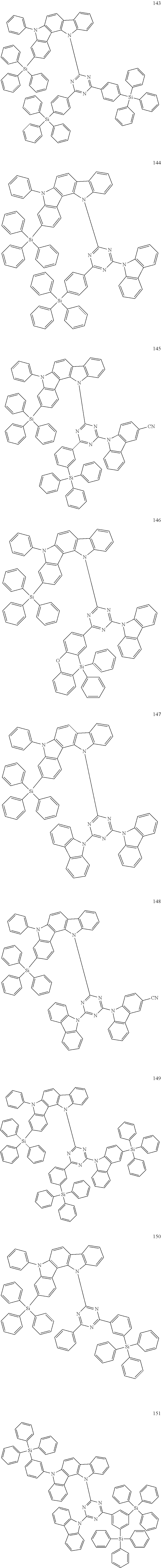

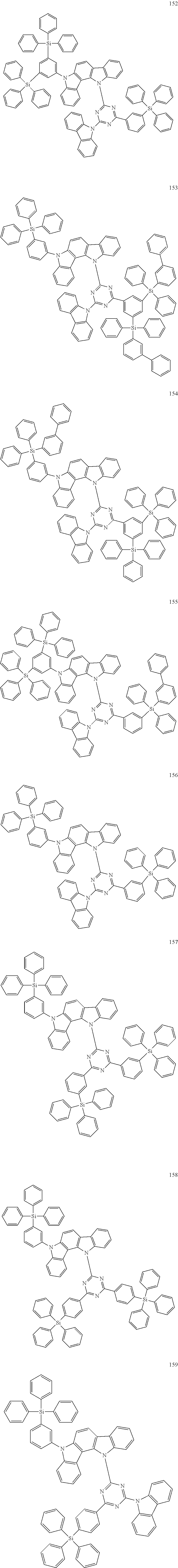

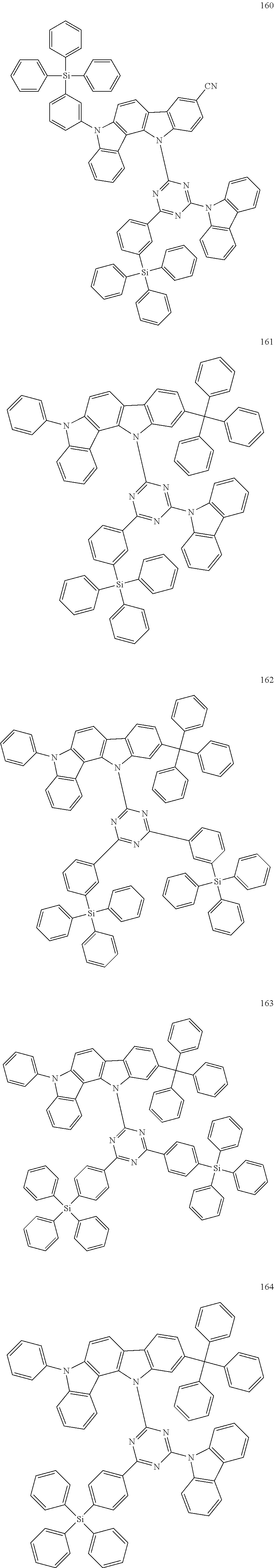

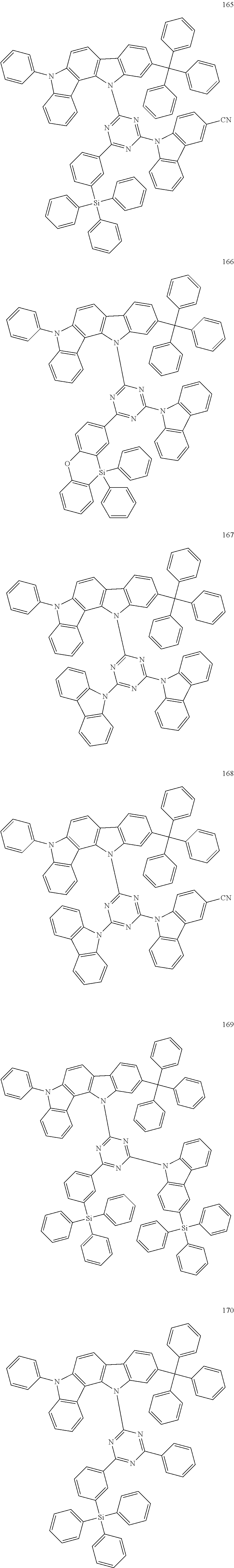

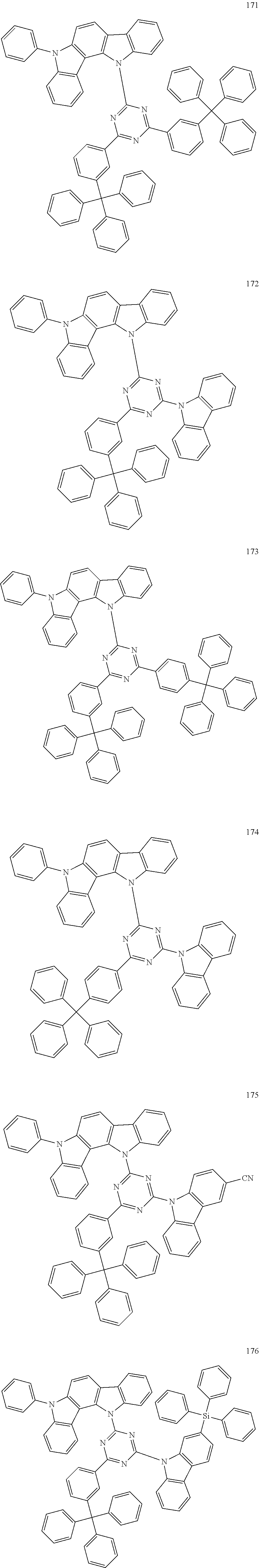

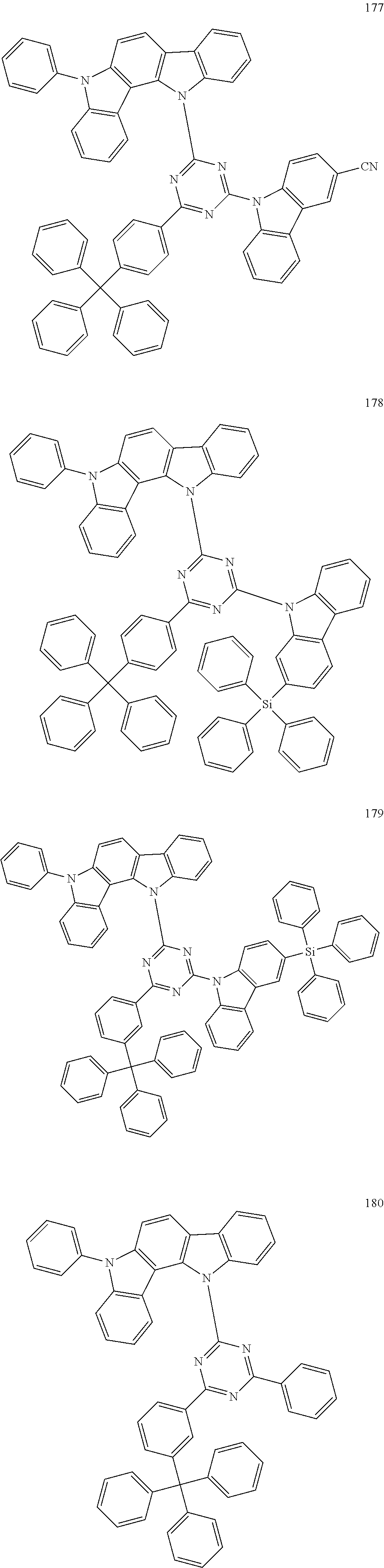

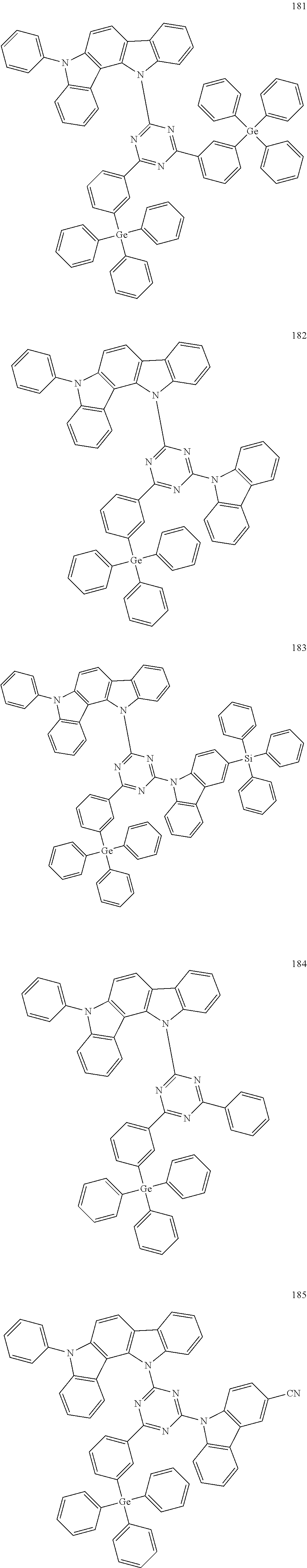

14. A polycyclic compound represented by Formula 1: 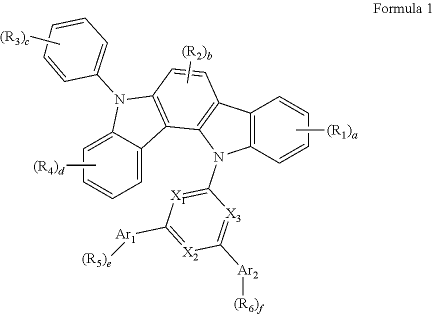
wherein in Formula 1,
X₁ to X₃ are each independently C or N, wherein at least two or more selected from among X₁ to X₃ are N,
Ar₁ and Ar₂ are each independently a substituted or unsubstituted arylene group of 6 to 60 ring-forming carbon atoms, or a substituted or unsubstituted heteroarylene group of 2 to 60 ring-forming carbon atoms,
R₁ to R₆ are each independently a hydrogen atom, a deuterium atom, or a cyano group, or represented by Formula 2, wherein at least one selected from among R₁ to R₆ is represented by Formula 2, and
a and d are each independently an integer from 0 to 4, b is an integer from 0 to 2, and c to f are integers from 0 to 5: 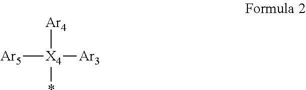
wherein in Formula 2,
X₄ is C, Si or Ge,
Ar₃ to Ar₅ are each independently a substituted or unsubstituted aryl group of 6 to 60 ring-forming carbon atoms, a substituted or unsubstituted cycloalkyl group of 3 to 60 ring-forming carbon atoms, or a substituted or unsubstituted heterocyclic group of 2 to 60 ring-forming carbon atoms, or combined with an adjacent group to form a ring, and
* represents a connection position.

15. The polycyclic compound of claim 14, wherein Formula 1 is represented by any one selected from among the following Formula 3-1 to Formula 3-4: 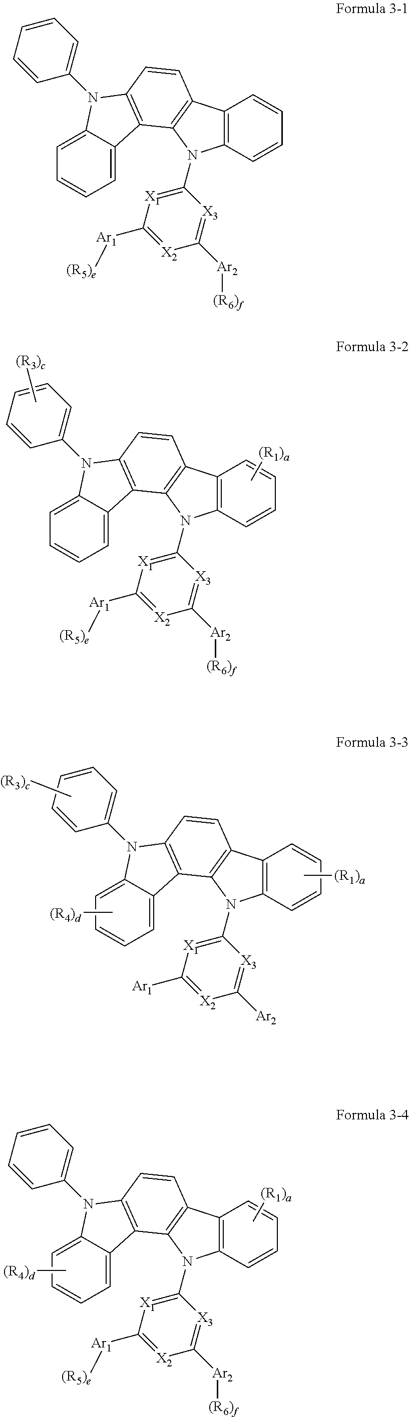
in Formula 3-1 to Formula 3-4, R₁ to R₆, Ar₁, Ar₂, and X₁ to X₃ are the same as defined in Formula 1,
in Formula 3-1, at least one selected from among R₅ and R₆ is represented by Formula 2,
in Formula 3-2, at least one selected from among R₁ and R₃, and at least one selected from among R₅ and R₆ are represented by Formula 2,
in Formula 3-3, at least one selected from among R₁, R₃ and R₄ is represented by Formula 2, and
in Formula 3-4, at least one selected from among R₁, R₄, R₅ and R₆ is represented by Formula 2.

16. The polycyclic compound of claim 14, wherein Ar₁ and Ar₂ are each independently a substituted or unsubstituted phenylene group, a substituted or unsubstituted divalent pyridoindole group, or a substituted or unsubstituted divalent carbazole group.

17. The polycyclic compound of claim 14, wherein Ar₃ to Ar₅ are each independently a substituted or unsubstituted phenyl group, or a substituted or unsubstituted dibenzofuran group, or combined with an adjacent group to form a heterocycle containing O and Si as ring-forming atoms.

18. The polycyclic compound of claim 14, wherein Formula 2 is represented by any one selected from among 2-1 to 2-6:

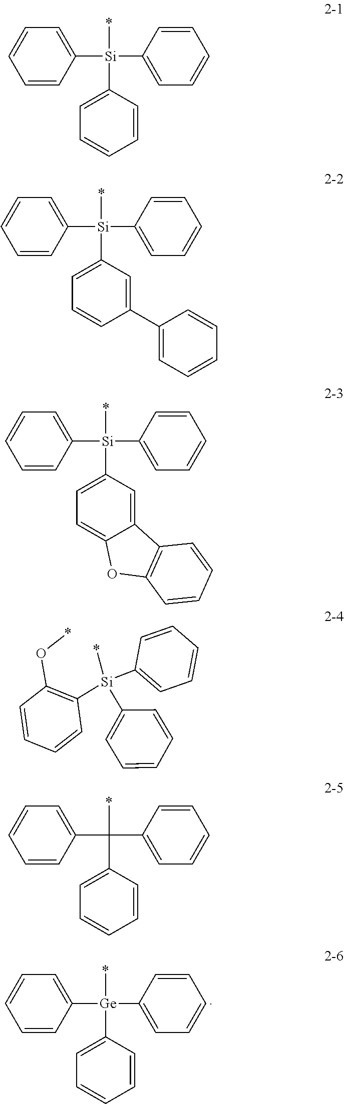

19. The polycyclic compound of claim 14, wherein the polycyclic compound represented by Formula 1 is represented by any one selected from among 4-1 to 4-19: 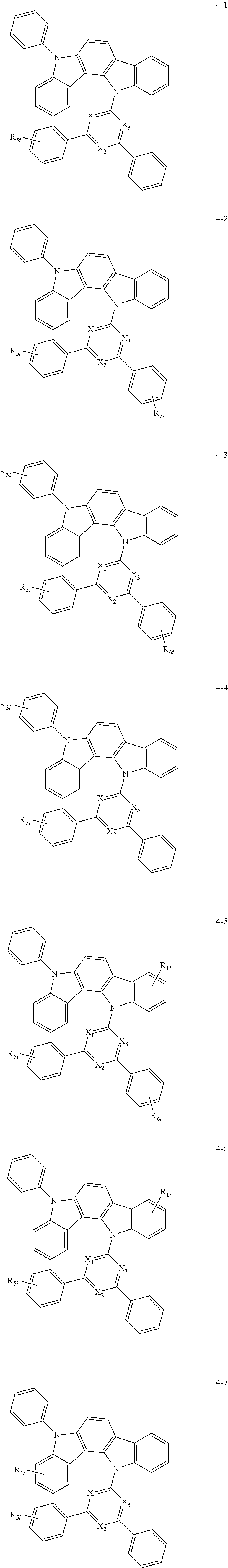 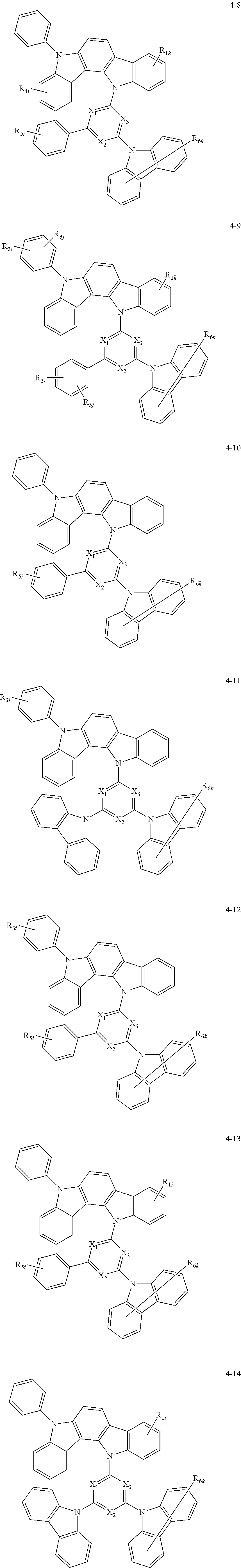 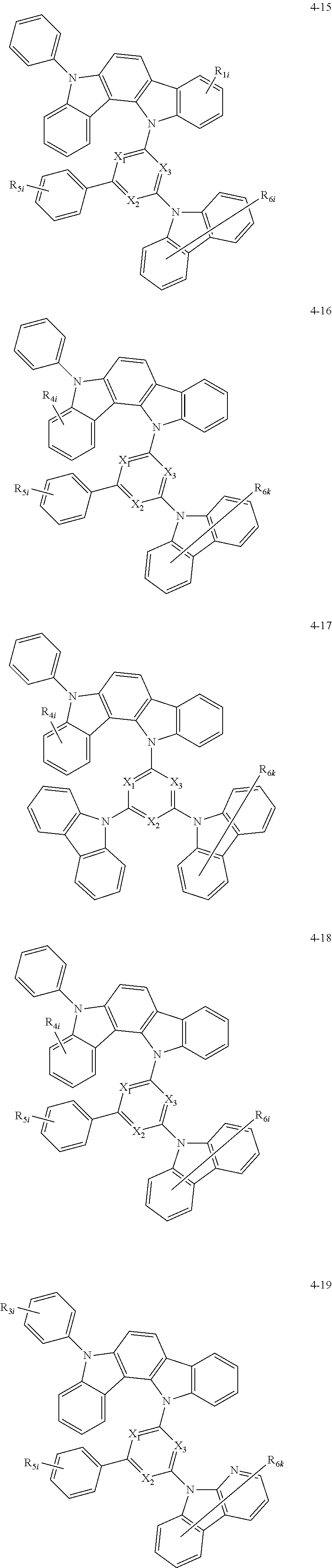
wherein in 4-1 to 4-19,
R₁ᵢ, R₃ᵢ, R₄ᵢ, R₅ᵢ and R₆ᵢ are each independently represented by Formula 2,
R₁ⱼ and R₅ⱼ are each independently a hydrogen atom or represented by Formula 2,
R₁ₖ and R₆ₖ are each independently a hydrogen atom or a cyano group, and
X₁ to X₃ are the same as defined in Formula 1.

20. The polycyclic compound of claim 14, wherein Formula 1 is represented by any one selected from among polycyclic compounds in Compound Group 1: 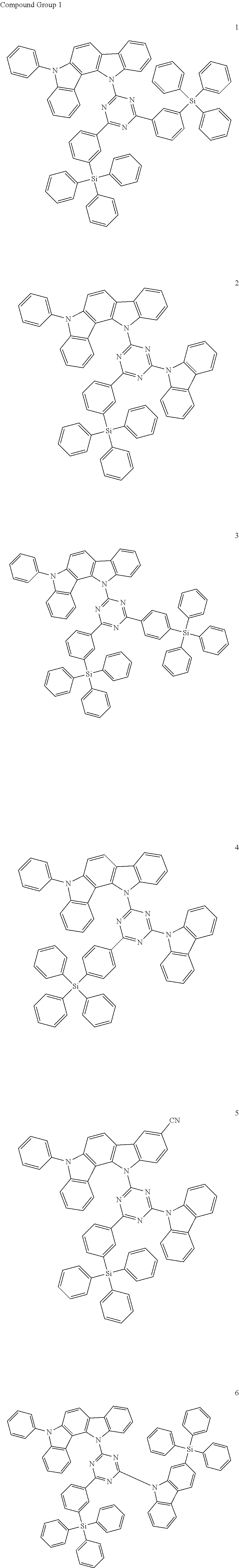 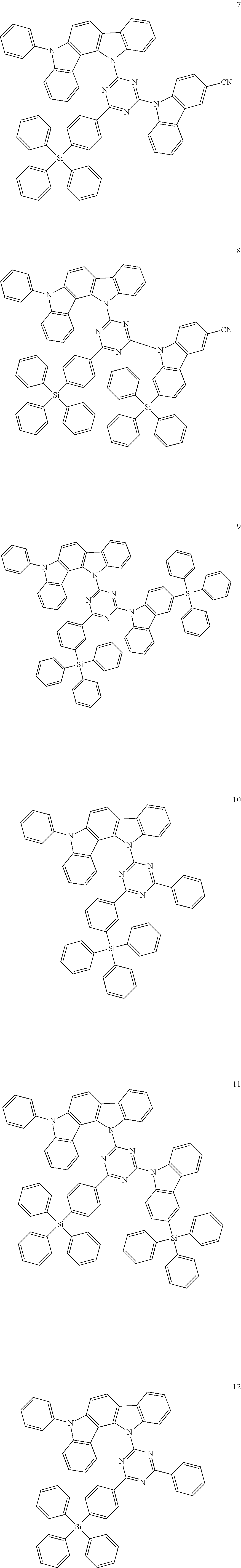 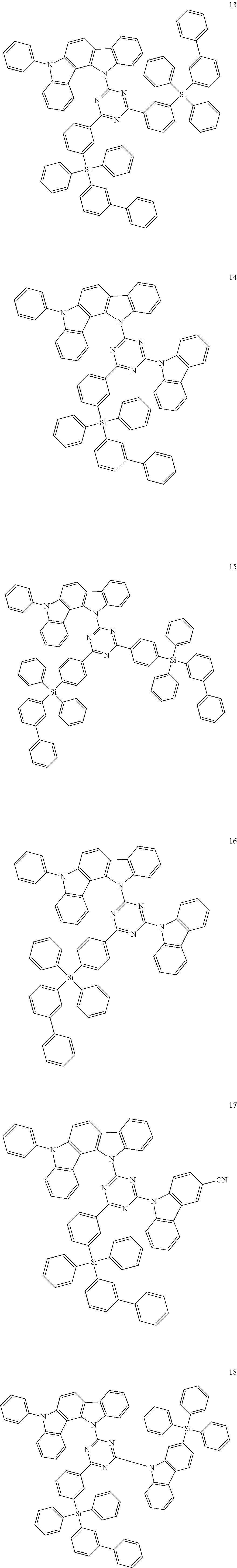 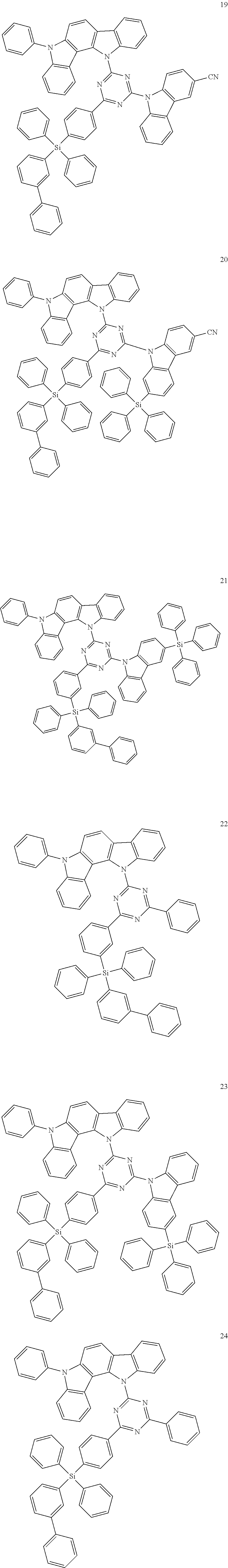 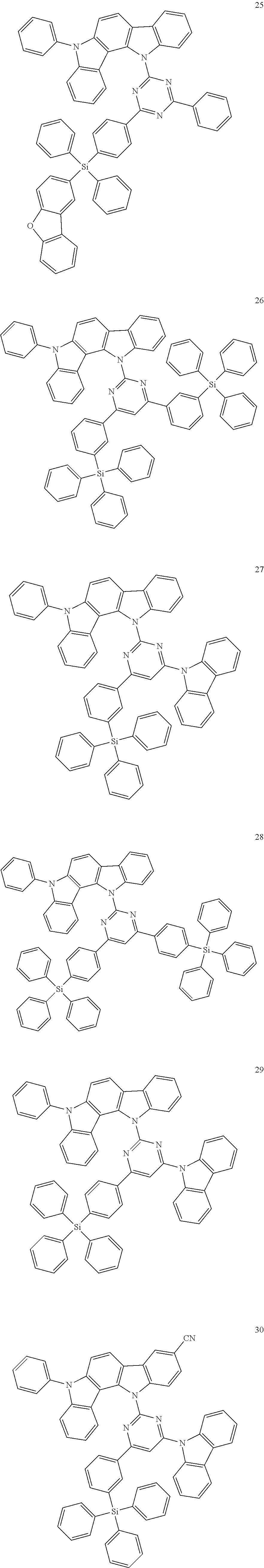 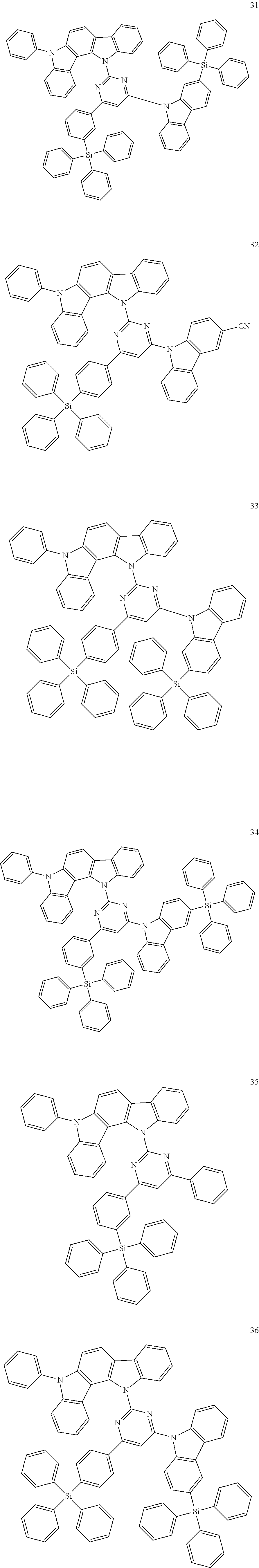 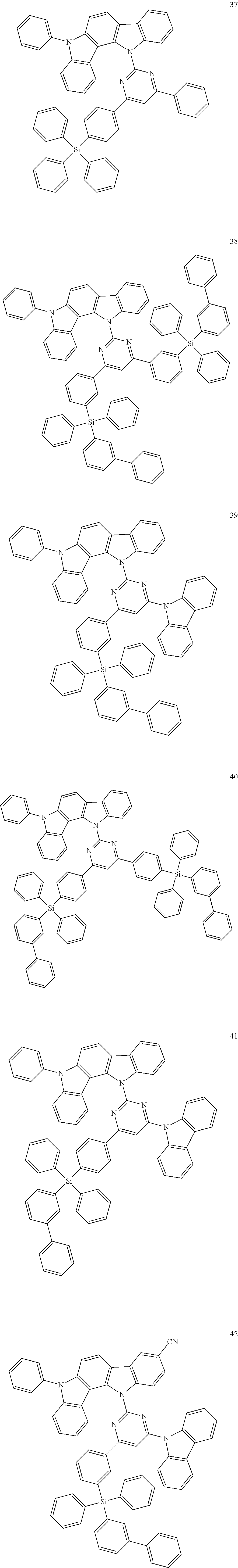 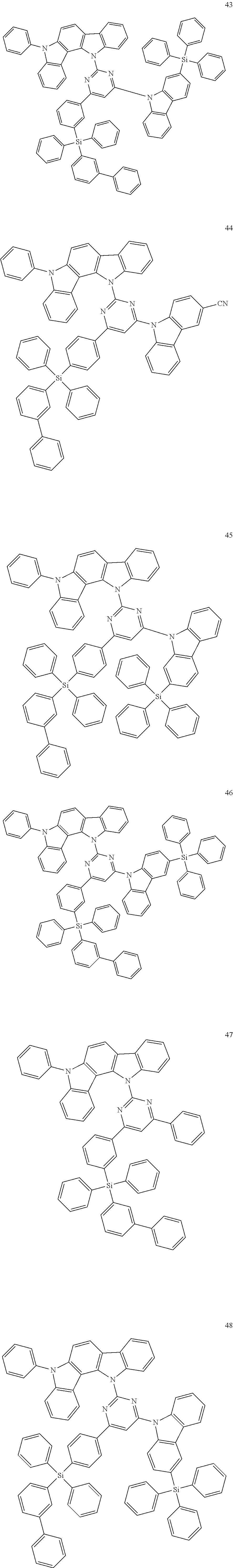 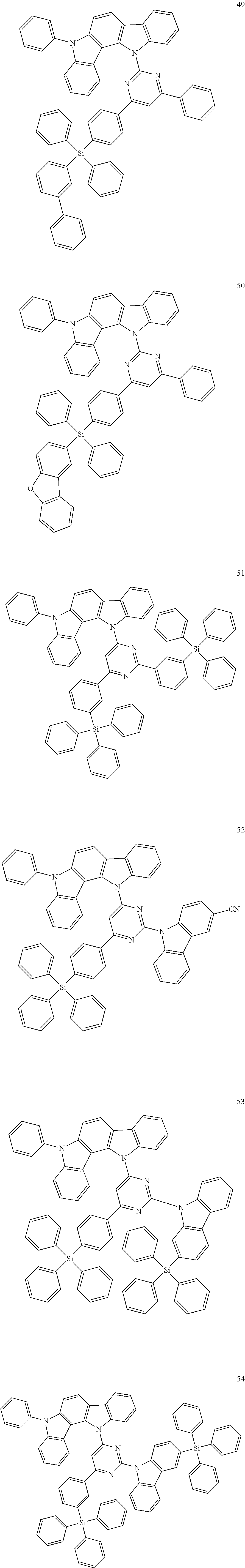 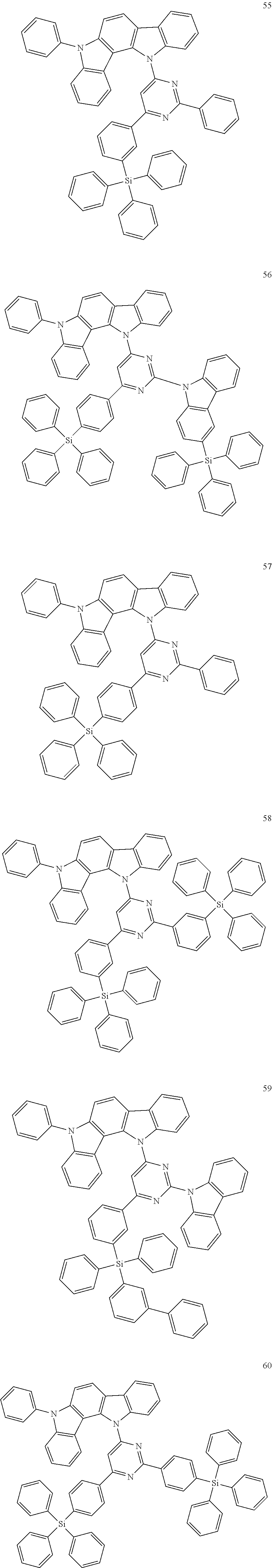 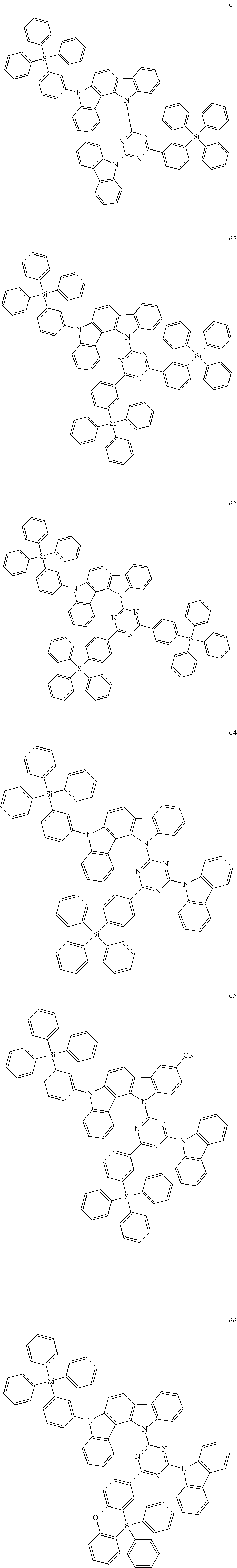 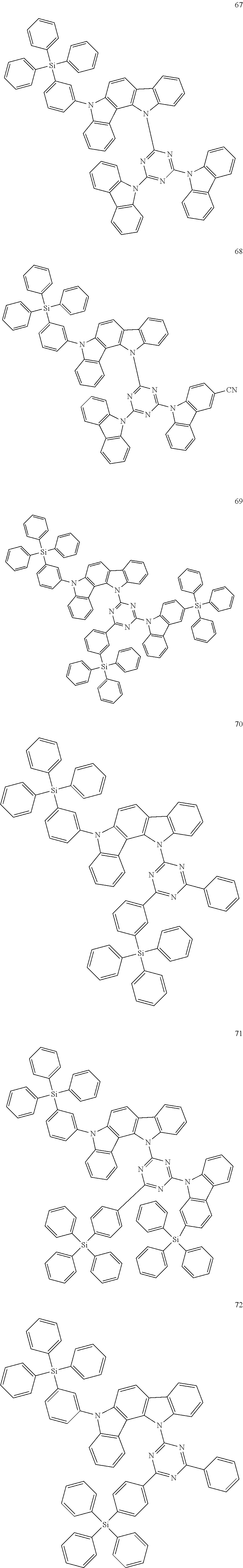 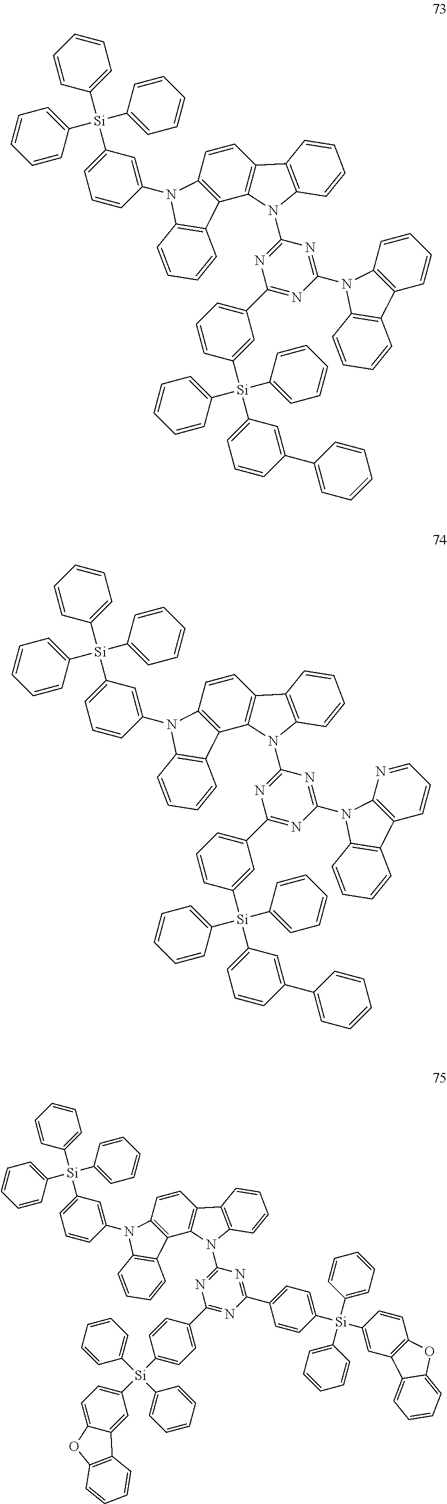 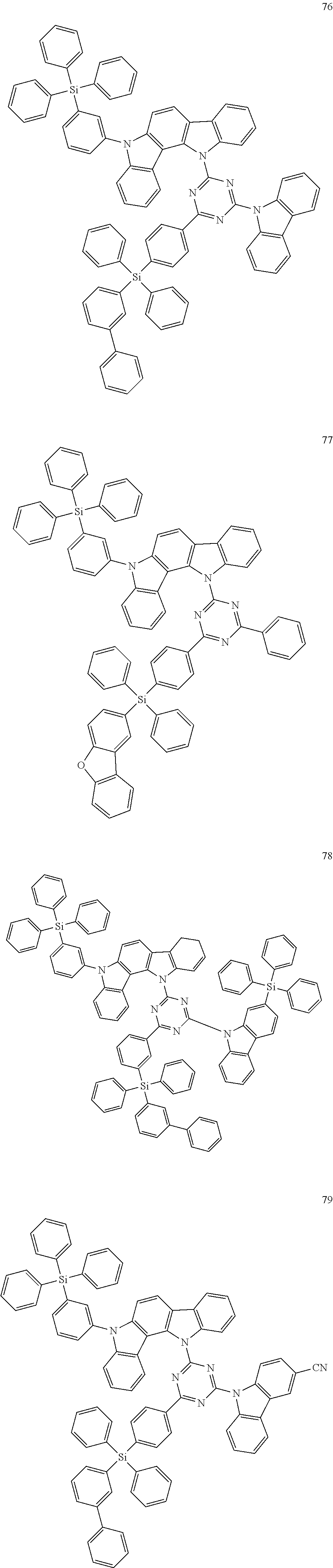 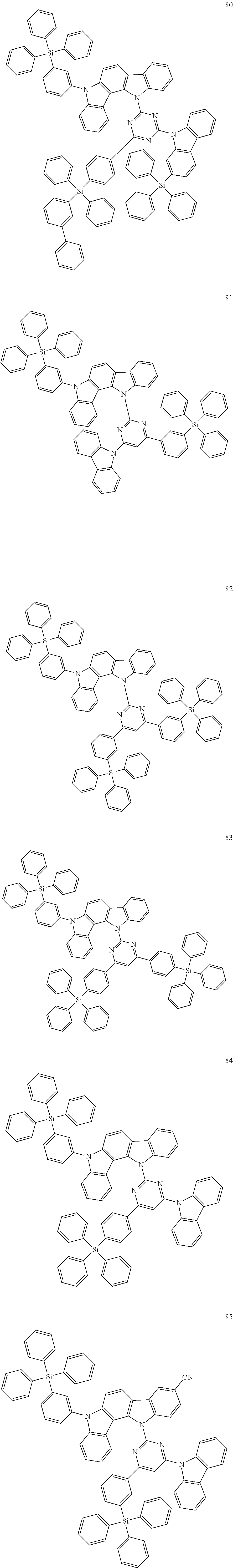 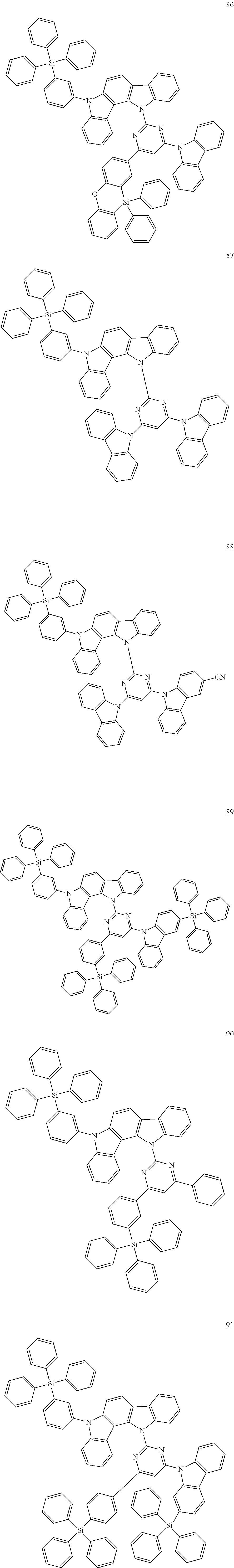 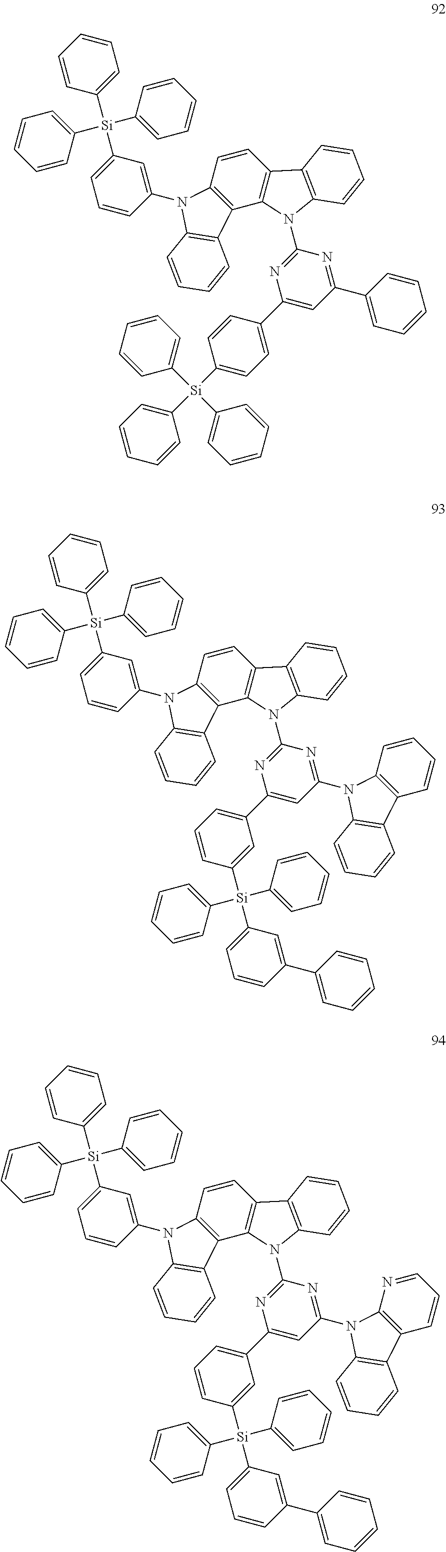 [SMILES: c1ccc(-c2cc(-c3ccc([Si](c4ccccc4)(c4ccccc4)c4ccccc4)cc3)nc(-n3c4ccccc4c4ccc5c(c6ccccc6n5-c5cccc([Si](c6ccccc6)(c6ccccc6)c6ccccc6)c5)c43)n2)cc1 ; c1ccc(-c2cccc([Si](c3ccccc3)(c3ccccc3)c3cccc(-c4cc(-n5c6ccccc6c6ccccc65)nc(-n5c6ccccc6c6ccc7c(c8ccccc8n7-c7cccc([Si](c8ccccc8)(c8ccccc8)c8ccccc8)c7)c65)n4)c3)c2)cc1 ; c1ccc(-c2cccc([Si](c3ccccc3)(c3ccccc3)c3cccc(-c4cc(-n5c6ccccc6c6cccnc65)nc(-n5c6ccccc6c6ccc7c(c8ccccc8n7-c7cccc([Si](c8ccccc8)(c8ccccc8)c8ccccc8)c7)c65)n4)c3)c2)cc1] 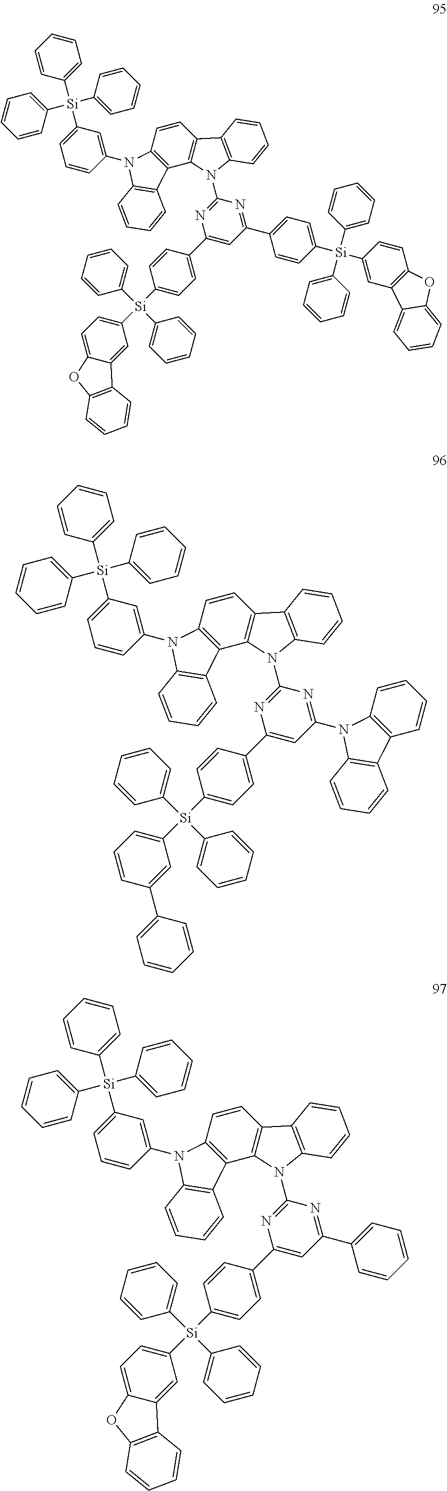 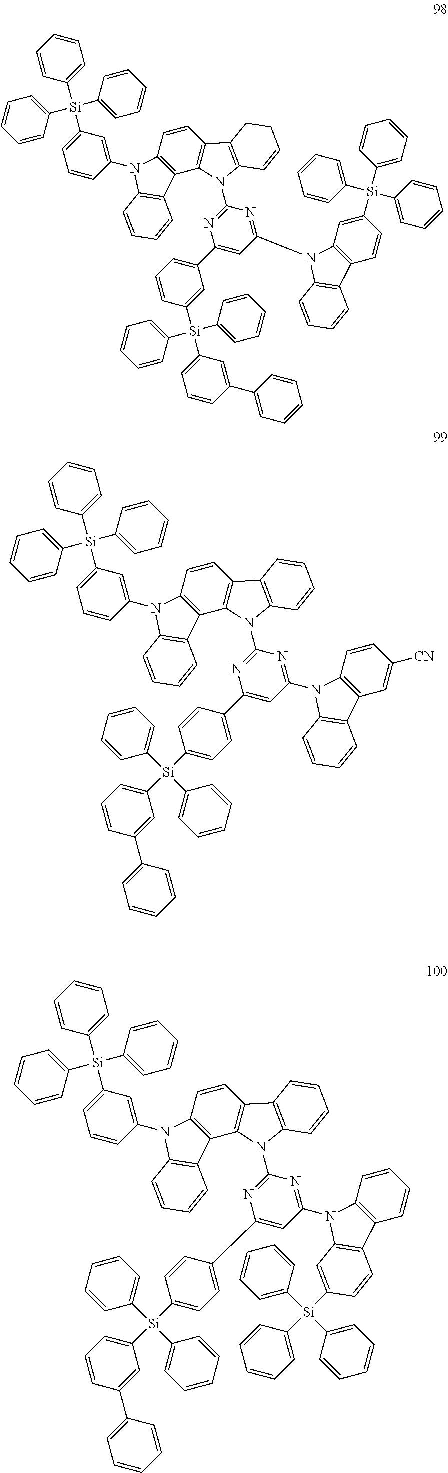 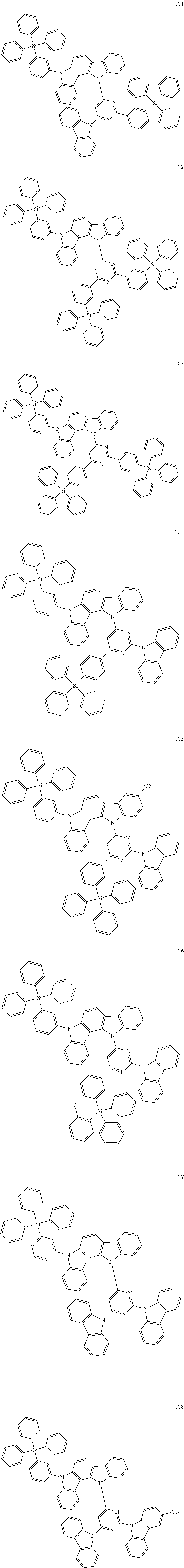 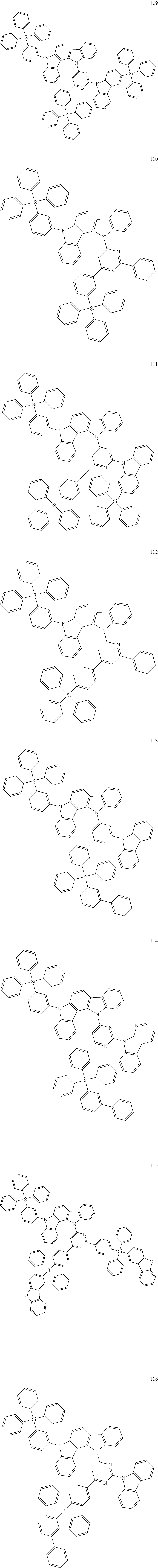 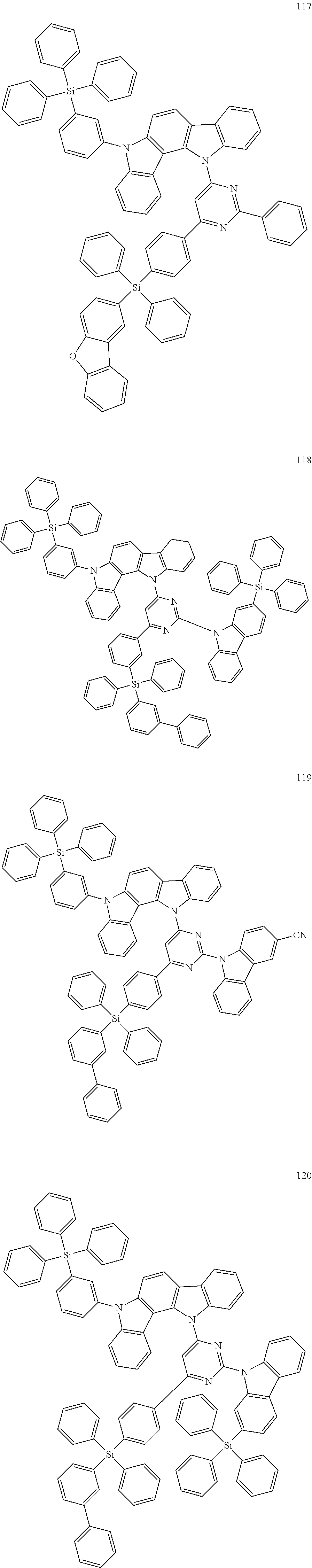 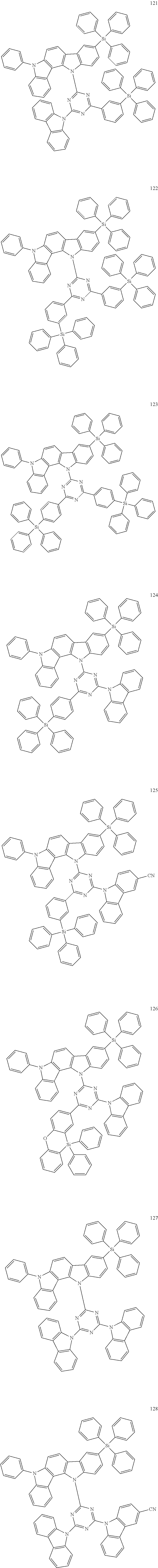 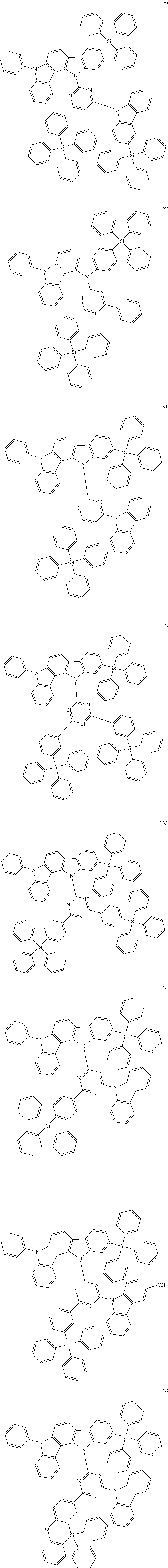 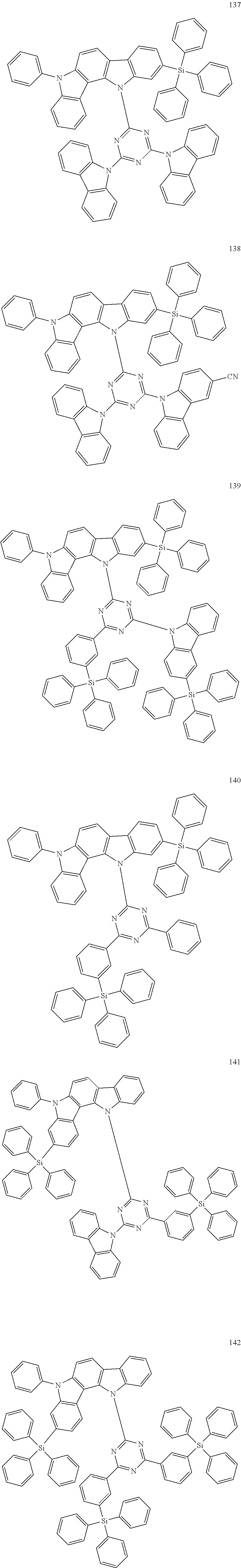 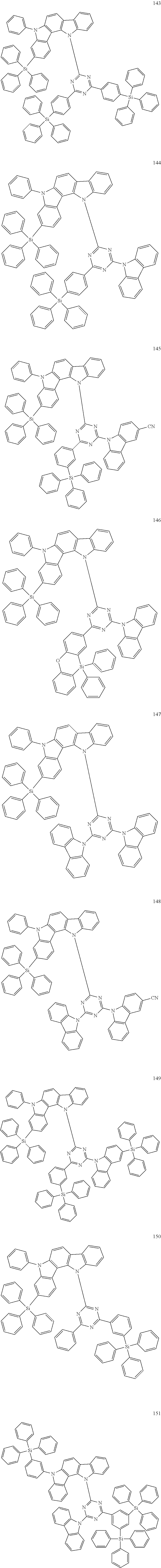 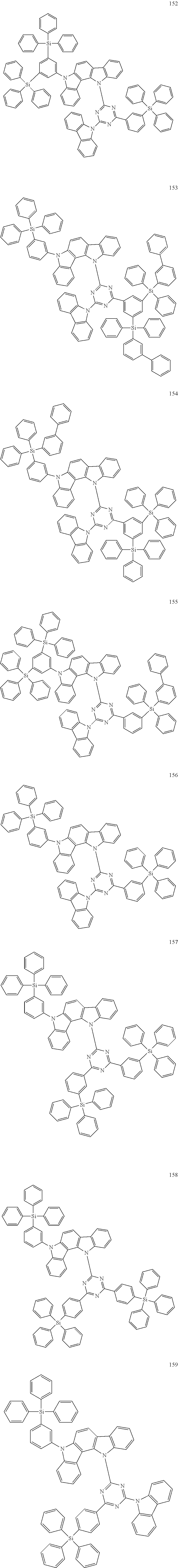 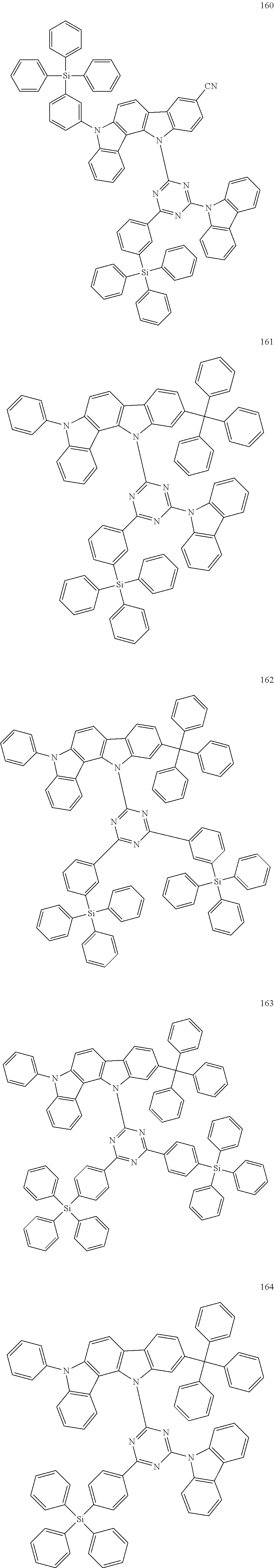 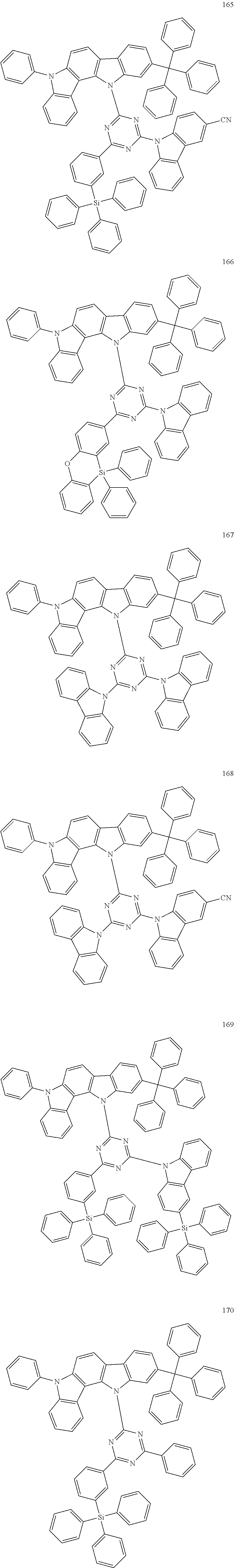 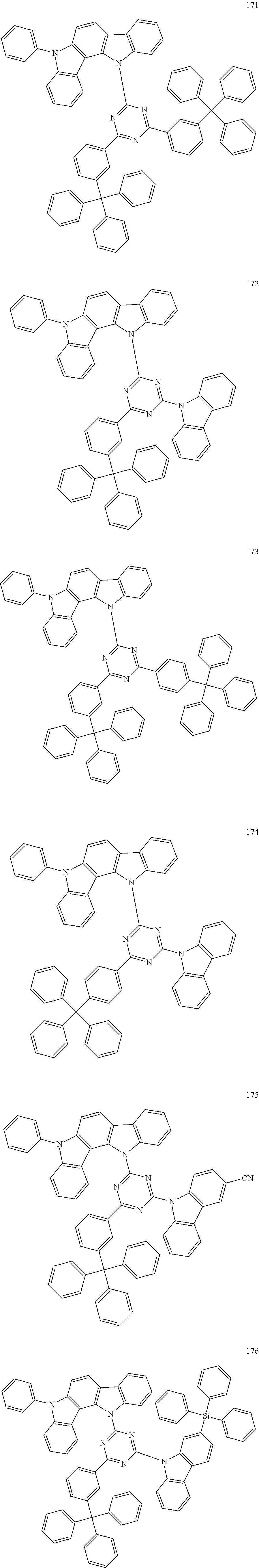 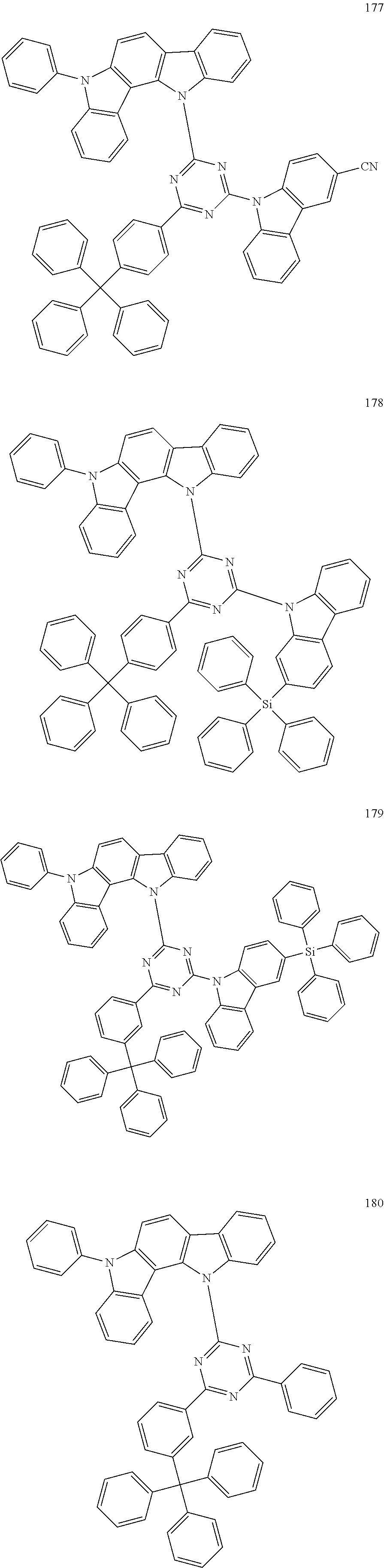 [chemical structure]
